SEQUENCE LISTING (1 sequence)

SEQ ID NO: 1 - protein, 133 aa, synthetic construct [sequence omitted]

(19) United States
(12) Patent Application Publication
(10) Pub. No.: US 2025/0049934 A1
(43) Pub. Date: Feb. 13, 2025

(54) DOSING REGIMENS FOR SELECTIVE TREG STIMULATOR RUR20kD-IL-2 AND RELATED COMPOSITIONS

(71) Applicants: Nektar Therapeutics, San Francisco, CA (US); Eli Lilly and Company, Indianapolis, IN (US)

(72) Inventors: Ali Ashrafzadeh, Indianapolis, IN (US); Steven Witt Dodd, Indianapolis, IN (US); Kimberley Anne Jackson, Indianapolis, IN (US); Stacey Masaaki Kaneshiro, Indianapolis, IN (US); Paul Alan Klekotka, Indianapolis, IN (US); Brian Leslie Kotzin, San Francisco, CA (US); Carsten Schmitz, Indianapolis, IN (US); Jonathan Zalevsky, San Francisco, CA (US)

(21) Appl. No.: 18/718,703

(22) PCT Filed: Dec. 14, 2022
(86) PCT No.: PCT/US2022/081541

§ 371 Date: Jun. 11, 2024

Related U.S. Application Data
(60) Provisional application No. 63/374,632, filed on Sep. 6, 2022.
(60) Provisional application No. 63/343,505, filed on May 18, 2022.
(60) Provisional application No. 63/325,923, filed on Mar. 31, 2022.
(60) Provisional application No. 63/323,126, filed on Mar. 24, 2022.
(60) Provisional application No. 63/289,371, filed on Dec. 14, 2021.

(51) Int. Cl. A61K 47/60; A61K 9/00; A61K 47/12; A61K 47/26; A61P 17/06; A61P 37/06; A61P 37/08

(52) U.S. Cl. CPC A61K 47/60; A61K 9/0019; A61K 47/12; A61K 47/26; A61P 17/06; A61P 37/06; A61P 37/08

(57) ABSTRACT

The instant disclosure provides selective Treg stimulator compositions, including RUR20kD-IL-2 and related compositions, and rezpegaldesleukin, and formulations and dosing regimens for methods of using these compositions, for example, for treating autoimmune diseases, including atopic dermatitis.

The instant application relates to formulations and dosing regimens for long-acting interleukin-2 receptor (IL-2 R) agonist, selective regulatory T cell (Treg) stimulator compositions, which increase the number and/or activation of regulatory T cells, and to methods of using these compositions in the treatment of autoimmune and inflammatory diseases, and/or other conditions responsive to Treg stimulatory therapy. In particular, the instant application relates to formulations and dosing regimens for selective Treg stimulator composition RUR_{20kD}-IL-2 and related compositions such as rezpegaldesleukin for the treatment of autoimmune diseases and inflammatory disorders.

### BACKGROUND

Recent efforts toward development of therapeutics for autoimmune disease have focused on inhibition of specific immune pathways potentially involved in disease pathogenesis. An alternative therapeutic concept has emerged aimed at selective upregulation of T regulatory cell mechanisms. A homeostatic imbalance of functional Tregs relative to conventional T cells (Tcons) is shared by many autoimmune diseases (Abbas A K, et. al., Revisiting IL-2: biology and therapeutic prospects. Sci Immunol. 2018; 3 (25), Sharabi A, et al., Regulatory T cells in the treatment of disease. Nat Rev Drug Discov. 2018 Oct. 12). IL-2 is a pleiotropic cytokine which impacts both Tcon and Treg development by engaging the dimeric IL-2Rβγ or trimeric IL-2Rαβγ receptor on lymphocytes and inducing downstream signaling cascades. In response to infection, or in an oncology setting, increased expression of IL-2 is essential for the generation of productive Tcon responses. At higher levels, IL-2 stimulates the proliferation, differentiation, and function of T effector cells (Teffs) and natural killer (NK) cells, which has resulted in it being used therapeutically at high doses to promote antitumor immune responses. High-dose IL-2 is administered to patients with certain cancers to produce expansion, and thereby activity, of the cytotoxic lymphocytes that can have antitumor activity. In approved cancer indications, the recommended treatment regimen for IL-2 (aldesleukin) is 600,000 International Units/kg (0.037 mg/kg), administered through intravenous (IV) infusion every 8 hours for a maximum of 14 doses (Proleukin® package insert, 2015). Recombinant IL-2 (aldesleukin) is an IL-2 analog with similar biological activity as endogenous human IL-2, and has an elimination half-life of approximately 85 minutes, necessitating dosing every 8 hours during cancer treatments (Proleukin package insert, 2015). However, high dose IL-2 therapy can cause severe adverse events (AEs) such as hypotension, vascular leak syndrome, pulmonary edema, increased risk of disseminated infections, cytokine release syndrome, inflammatory disease, heart toxicities (cardiac rhythm disturbances and angina or myocardial infarction), severe anemia/thrombocytopenia, and other toxicities (Proleukin package insert, 2015). In addition, high-dose IL-2 therapy may decrease the normal suppressive activity of Treg cells on effector T cells, potentially disrupting immune homeostasis.

However, at resting physiological levels, IL-2 is also required for the development of Tregs as well as their survival in the peripheral circulation. Due to the difference in IL-2 receptor biology, Tregs are stimulated at much lower concentrations of IL-2 than Tcons, which constitutes a therapeutic dosing window in which locally low doses of IL-2 agonists might be utilized to selectively enhance Treg responses in autoimmune patients. Considering these differential effects, low-dose recombinant human IL-2 (rhIL-2) therapy has been evaluated for its ability to selectively induce Tregs and thereby treat autoimmune diseases. Some clinical studies have demonstrated that low doses of IL-2 can mitigate the deficiency of Tregs and enhance the survival of these cells, and an open label study and a randomized, double blind, placebo-controlled study of SLE patients provided evidence that IL-2 treatment may result in improved disease activity indices (See e.g., He et al., Efficacy and safety of low dose IL 2 in the treatment of systemic lupus erythematosus: a randomized, double blind, placebo controlled trial. Ann Rheum Dis. 2019). In addition to SLE, a small study of 46 patients with various autoimmune diseases provided an indication that IL-2 may have benefit in the treatment of ulcerative colitis, psoriasis, and ankylosing spondylitis (Rosenzwajg et al., Immunological and clinical effects of low dose interleukin 2 across 11 autoimmune diseases in a single, open clinical trial. Ann Rheum Dis. 2019; 78 (2): 209-217). Despite promising early results, the usefulness of low-dose rhIL-2 treatment, such as unmodified aldesleukin, is limited by a narrow therapeutic window for Treg selectivity and a short half-life, necessitating frequent administration. There remains a high unmet need for effective and safe treatment options providing alternatives to existing Treg stimulatory approaches.

Selective Treg stimulator compositions, in particular RUR_{20kD}-IL-2 and related compositions, have been described in WO 2019/226,538, which comprise certain polyethylene glycol (PEG)ylated recombinant human interleukin 2 (rhIL-2) conjugate compositions. Compared to native IL-2, and/or other pro-effector pegylated IL-2 compositions, RUR_{20kD}-IL-2 and related compositions in contrast have the superior ability to effectively and selectively promote the expansion and activation of regulatory T cells (Tregs) over that of conventional T cells (Tcons), potentially providing therapeutic alternatives for autoimmune conditions. Improved methods of formulating, dosing, and treatment regimens are needed to enable therapeutically advantageous RUR_{20kD}-IL-2 and related composition treatments for clinical of autoimmune diseases.

### SUMMARY

In several aspects the present disclosure provides therapeutically advantageous formulations, doses, and dosing regimens, for selective Treg stimulator RUR_{20kD}-IL-2 and related compositions, in particular rezpegaldesleukin, for the treatment of autoimmune diseases including atopic dermatitis, systemic lupus erythematosus (SLE), allergy, GVHD, Crohn's disease, ulcerative colitis, rheumatoid arthritis, type-1 diabetes, multiple sclerosis, and other inflammatory and/or immune mediated diseases. The formulations, doses, and dosing regimens include a range of fixed unit doses and dosing regimens for treating autoimmune disease, and in particular, for induction and maintenance dosing to achieve effective disease treatment, maximizing patient compliance, convenience, and tolerability, while minimizing the risk of T effector stimulation, thereby enhancing autoimmune disease state management.

### DETAILED DESCRIPTION

The selective Treg stimulator compositions described herein are useful for formulations, doses, and dosing regimens of the present disclosure. Rezpegaldesleukin, an RUR_{20kD}-IL-2 Selective Treg stimulator composition described herein, has been studied in a phase 1b trial of patients with moderate to severe atopic dermatitis (NCT04081350). This Phase 1b study is a double-blind, randomized, placebo-controlled multiple-dose study of rezpegaldesleukin evaluating the safety, tolerability, and pharmacokinetics in approximately 40 adults with moderate to severe atopic dermatitis. Patients were treated with rezpegaldesleukin every two weeks subcutaneously for 12 weeks at two different dose levels. Patients had to be EASI-50 (EASI=eczema area and severity index) responders at week 19 to continue in the study.

An interim data analysis from this study indicated that rezpegaldesleukin treatment resulted in a dose dependent reduction in Eczema Area and Severity Index (EASI) scores, and at the 24 μg/kg dose (administered Q2W, according to the patient's weight) resulted in an approximately 70% maximum reduction in EASI scores at week 12. FIG. 3 illustrates interim analysis results from this phase 1b study, and represents mean percent change from baseline in EASI scores over time, and provides evidence of a change in EASI score for the 24 μg/kg dose with a more robust effect than patients in the placebo group. These observations in a subset of patients provide evidence of proof of concept for rezpegaldesleukin treatment for moderate to severe atopic dermatitis. It was surprisingly discovered that treatment in this study, over a 12-week period at the 24 μg/kg Q2W dose, provided sustained disease control for up to six months after last dose in a subset of patients (See FIG. 3). Thus, sustained disease control provided by this treatment may provide means to treat atopic dermatitis which are superior to current standards of care.

On the other hand, it has also commonly been observed that administration of rezpegaldesleukin may result in clinically undesirable injection site reactions (ISRs) in patients, characterized by localized erythema having a moderate peak size, pain, swelling, and induration, where the onset occurs between 1-5 days post-dose, have a dose related duration which may on the order of 7 to 27 days, and may resolve without treatment. Patients seeking to resolve inflammatory skin disease, and/or other autoimmune disorders, could benefit from means to effectively treat their disease, while at the same time minimizing the frequency and/or severity of the associated ISRs. Further, higher doses taken for example at a higher frequency, for a longer duration, may elevate the risk of pro-effector stimulation (e.g., excessive stimulation of NK cells, eosinophils and T effector cells), working against the desired selective Treg stimulatory effect. Thus, discovery of advantageous fixed unit doses and dosing regimens provide a critical step in the clinical management of autoimmune disease with rezpegaldesleukin. Optionally, administration of rezpegaldesleukin may be accompanied by co-administration or subsequent administration of topical corticosteroids, tacrolimus inhibitors, oral or topical antihistamines, or topical JAK inhibitors to mitigate undesirable injection site reactions (ISRs). Preferably ISRs are prevented or mitigated by administration of topical antihistamines. Topical antihistamines useful for mitigating ISRs may include but are not limited to doxepin, diphenhydramine and diphenhydramine/zinc acetate.

Rezpegaldesleukin formulations and dosing regimen embodiments of the present disclosure provide means to maximize patient therapeutic compliance, convenience, and effective disease management, by less frequent and/or adjusted dosing, comprising selected doses which provide advantageous means for treatment of autoimmune disease, including particular induction and maintenance doses and dosing regimens. Thus, the formulations and dosing regimens described herein provide improvements in clinical tolerability and are conceived to enhance compliance and promote improved clinical disease management for patients with atopic dermatitis, SLE, and/or other autoimmune diseases described herein.

### Formulations, Unit Doses, and Dosing Regimens for Treatment of Autoimmune Diseases:

### Unit Doses:

In some aspects embodiments of the present disclosure provide unit doses of selective Treg stimulator RUR_{20kD}-IL-2 and related compositions which are about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose for use in the treatment of an autoimmune disease. In some aspects embodiments of the present disclosure provide preferred unit doses of selective Treg stimulator RUR_{20kD}-IL-2 and related compositions which are about 2100 μg, about 2400 μg, about 2700 μg, about 3000 μg, about 3300 μg, or about 3600 μg per dose for use in the treatment of an autoimmune disease. Preferably, the present disclosure provides unit doses of rezpegaldesleukin, which are about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose for use in the treatment of an autoimmune disease. Preferably, the present disclosure provides unit doses of rezpegaldesleukin, which are about 300 μg, about 450 μg, about 600 μg, about 900 μg, about 1200 μg, about 1500 μg, and about 1800 μg, per dose, for use in the treatment of an autoimmune disease. Most preferably, the present disclosure provides unit doses of rezpegaldesleukin, which are about 2100 μg, about 2400 μg, about 2700 μg, about 3000 μg, about 3300 μg, or about 3600 μg per dose for use in the treatment of an autoimmune disease. Uses of these unit doses of rezpegaldesleukin and/or selective Treg stimulator RUR_{20kD}-IL-2 and related compositions described herein are further described below. See for instance below at about page 68 and/or 84, and elsewhere herein, for descriptions of rezpegaldesleukin compositions of Formulae A-F.

In an embodiment the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose of a selective Treg stimulator RUR20kD-IL-2 and related composition, wherein the composition comprises a composition of Formulae A-F. See below at about page 84 and herein for descriptions of compositions of Formulae A-F. Preferably the composition is a composition of Formula A. Preferably the composition is a composition of Formula F. Preferably the composition is a composition of Formula F.

In an embodiment the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose of a selective Treg stimulator RUR20kD-IL-2 and related composition, wherein the composition comprises a composition of Formulae A-F. Preferably the composition is a composition of Formula A. Preferably the composition is a composition of Formula B which, for instance, encompasses certain exemplified preparations of rezpegaldesleukin described herein.

In an embodiment the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose of rezpegaldesleukin.

In an embodiment the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose of rezpegaldesleukin.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin, wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, or about 1800 μg per dose, of rezpegaldesleukin, wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin, wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, or about 1800 μg per dose, of a composition of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula D, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula D, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula E, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula E, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula F, wherein the composition comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula F, wherein the composition comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said administering is carried out once every 2 weeks or once every 4 weeks. Preferably said administering is carried out once every 2 weeks. Preferably said administering is carried out once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin, wherein said administering is carried out once every 2 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin, wherein said administering is carried out once every 2 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin, wherein said administering is carried out once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin, wherein said administering is carried out once every 4 weeks.

For each of the above embodiments, the present disclosure further provides embodiments wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is selected from systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In further aspects of each the above embodiments preferably the autoimmune disease is atopic dermatitis. In each of the embodiments wherein the autoimmune disease is atopic dermatitis, a further preferred patient is one which is bio-experienced as defined herein. In further aspects of each of the above embodiments preferably the autoimmune disease is systemic lupus erythematosus (SLE). In further aspects of each of the above embodiments preferably the autoimmune disease is ulcerative colitis. In further aspects of each of the above embodiments preferably the autoimmune disease is type 1 diabetes. In further aspects of each of the above embodiments preferably the autoimmune disease is peanut allergy.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is atopic dermatitis.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is atopic dermatitis.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is atopic dermatitis.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is atopic dermatitis.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is systemic lupus erythematosus (SLE).

an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is systemic lupus erythematosus (SLE).

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is systemic lupus erythematosus (SLE).

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is systemic lupus erythematosus (SLE).

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is ulcerative colitis.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is ulcerative colitis.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is ulcerative colitis.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is ulcerative colitis.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is type 1 diabetes.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is type 1 diabetes.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is type 1 diabetes.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is type 1 diabetes.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is peanut allergy.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 2 weeks; wherein the autoimmune disease is peanut allergy.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is peanut allergy.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks; wherein the autoimmune disease is peanut allergy.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said doses further comprise a formulation of 5 mM sodium acetate, 25 mM sodium chloride, 7.5% (w/v) sucrose, pH 5.0; wherein said administering is carried out once every 2 weeks; wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said doses further comprise a formulation of 5 mM sodium acetate, 25 mM sodium chloride, 7.5% (w/v) sucrose, pH 5.0; wherein said administering is carried out once every 2 weeks; wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said doses further comprise a formulation of 5 mM sodium acetate, 25 mM sodium chloride, 7.5% (w/v) sucrose, pH 5.0; wherein said administering is carried out once every 4 weeks; wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of rezpegaldesleukin; wherein said doses further comprise a formulation of 5 mM sodium acetate, 25 mM sodium chloride, 7.5% (w/v) sucrose, pH 5.0; wherein said administering is carried out once every 4 weeks; wherein the autoimmune disease is selected from systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula D, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula D, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula E, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula E, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks.

In an embodiment, the present disclosure provides a method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of a composition of Formula F, wherein the composition comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks. Preferably administering is carried out once every 2 weeks.

In an embodiment, the present disclosure provides a method of treating a human subject having an autoimmune disease comprising administering to the human subject a dose of about 2100 μg, about 2400 μg, about 2700 μg, about 3000 μg, about 3300 μg, or about 3600 μg per dose, of a composition of Formula F, wherein the composition comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; wherein said doses further comprise a formulation of 5 mM sodium acetate, 25 mM sodium chloride, 7.5% (w/v) sucrose, pH 5.0; wherein said administering is carried out once every 2 weeks or once every 4 weeks. Preferably administering is carried out once every 2 weeks.

In further aspects of each of the above embodiments, preferably the doses of rezpegaldesleukin further comprise a formulation of 5 mM sodium acetate, 25 mM sodium chloride, 7.5% (w/v) sucrose, pH 5.0. In further aspects of each of the above embodiments preferably the rezpegaldesleukin concentration for an 1800 μg dose is selected from 0.9 or 1.8 or 3.6 mg/mL protein equivalent, depending on the autoinjector or prefilled syringe being used. In further aspects of each of the above embodiments, preferably the delivery device is an autoinjector is selected from a 0.5 mL or a 1.0 mL or a 2.0 mL auto-injector, or a pre-filled syringe corresponding to the same dose volumes. In further aspects of each of the above embodiments preferably a 3000 μg dose of rezpegaldesleukin has a concentration of 1.5 or 3.0 or 6.0 mg/mL (depending on the autoinjector or prefilled syringe). In further aspects of each of the above embodiments preferably a 3300 μg dose of rezpegaldesleukin has a concentration of 1.5 or 3.0 or 6.0 mg/mL (depending on the autoinjector or prefilled syringe). In further aspects of each of the above embodiments preferably a 3600 μg dose of rezpegaldesleukin has a concentration of 1.5 or 3.0 or 6.0 mg/mL (depending on the autoinjector or prefilled syringe).

The present disclosure provides a pharmaceutical formulation comprising a selective Treg stimulator RUR20kD-IL-2 and related composition at a concentration of about 4.0 mg/mL to about 5.0 mg/mL, sodium acetate at a concentration about 5 mM, sodium chloride at a concentration of about 25 mM, sucrose at a concentration of about 7.5% (w/v), and a pH at about 5.0. Preferably the selective Treg stimulator RUR20kD-IL-2 and related composition comprises, on a molar basis, about 5 mole percent or less mono-PEGylated IL-2 conjugates, and from about 28 mole percent to about 60 mole percent di-PEGylated IL-2 conjugates, and from about 24 mole percent to about 65 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons. Preferably the selective Treg stimulator RUR20kD-IL-2 and related composition comprises, on a molar basis, about 2 mole percent to about 4 mole percent mono-PEGylated IL-2 conjugates, and from about 35 mole percent to about 55 mole percent di-PEGylated IL-2 conjugates, and from about 35 mole percent to about 55 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons. Preferably the selective Treg stimulator RUR20kD-IL-2 and related composition comprises, on a molar basis, from about 2.5 mole percent to about 4.5 mole percent mono-PEGylated IL-2 conjugates, and from about 35 mole percent to about 50 mole percent di-PEGylated IL-2 conjugates, and from about 38 mole percent to about 46 mole percent tri-PEGylated IL-2 conjugates, and from about 3 mole percent to about 10 mole percent higher PEGylated IL-2 conjugates, and wherein the nominal average molecular weight of each branched polyethylene glycol moiety is about 20,000 daltons. Preferably the selective Treg stimulator RUR20kD-IL-2 and related composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein the nominal average molecular weight of each branched polyethylene glycol moiety is about 20,000 daltons.

In an embodiment, there is provided a method of treating an autoimmune disease comprising administering to a human in need thereof an effective dose of one of the pharmaceutical compositions described herein above. Preferably the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. In an embodiment, there is provided a method of treating SLE comprising administering to a human in need thereof an effective dose of one of the above-described compositions. In an embodiment, there is provided a method of treating atopic dermatitis comprising administering to a human in need thereof an effective dose of one of the above-described compositions. In an embodiment, there is provided a method of treating type I diabetes comprising administering to a human in need thereof an effective dose of one of the above-described compositions.

In an embodiment, there is provided one of the pharmaceutical compositions described herein above for use as a medicament.

The pharmaceutical compositions of the present invention are typically administered via injection which includes for example subcutaneous, intravenous, and intramuscular injections, as well as infusion injections. Exemplary compositions for administration to a patient include for example a subcutaneous formulation as described herein and comprise a dose described herein. The pharmaceutical compositions of the present invention are in the liquid dosage form of a solution. The pharmaceutical compositions of the present disclosure are typically administered subcutaneously. The pharmaceutical compositions are typically administered using a pre-filled, disposable pen, reusable pen, or automatic pen injector. The pharmaceutical compositions may be administered using a disposable syringe with an attached needle of appropriate gauge and length. In an embodiment, the device is an automatic injection apparatus. According to another aspect of the present invention, there is provided an article of manufacture comprising one of the above-described pharmaceutical compositions. In certain embodiments, the article of manufacture is a single-use vial. In certain embodiments, the article of manufacture is a pre-filled syringe, such as a 0.5 ml auto-injector or 1 ml auto-injector or 2 ml auto-injector as known to the skilled artisan (see for example Lange J, Richard P, Bradley N. Usability of a new disposable autoinjector platform device: results of a formative study conducted with a broad user population. Med Devices (Auckl). 2015; 8:255-264, and/or BD Physioject™ Disposable Autoinjector 1 mL, BD Medical-Pharmaceutical Systems). In certain embodiments, the article of manufacture is an automatic injection apparatus (“auto-injector”).

In further aspects of each of the above embodiments which includes 300 μg as a dose, preferably the dose of rezpegaldesleukin is about 300 μg. In further aspects of each of the above embodiments which includes 450 μg as a dose, preferably the dose of rezpegaldesleukin is about 450 μg. In further aspects of each of the above embodiments which includes 600 μg as a dose, preferably the dose of rezpegaldesleukin is about 600 μg. In further aspects of each of the above embodiments which includes 900 μg as a dose, preferably the dose of rezpegaldesleukin is about 900 μg. In further aspects of each of the above embodiments which includes 1200 μg as a dose, preferably the dose of rezpegaldesleukin is about 1200 μg. In further aspects of each of the above embodiments which includes 1500 μg as a dose, preferably the dose of rezpegaldesleukin is about 1500 μg. In further aspects of each of the above embodiments which includes 1800 μg as a dose, preferably the dose of rezpegaldesleukin is about 1800 μg. In further aspects of each of the above embodiments which includes 2100 μg as a dose, preferably the dose of rezpegaldesleukin is about 2100 μg. In further aspects of each of the above embodiments which includes 2400 μg, preferably the dose of rezpegaldesleukin is about 2400 μg. In further aspects of each of the above embodiments which includes 2700 μg as a dose, preferably the dose of rezpegaldesleukin is about 2700 μg. In further aspects of each of the above embodiments which includes 3000 μg as a dose, preferably the dose of rezpegaldesleukin is or about 3000 μg. In further aspects of each of the above embodiments which includes 3300 μg as a dose, preferably the dose of rezpegaldesleukin is or about 3300 μg. In further aspects of each of the above embodiments which includes 3600 μg as a dose, preferably the dose of rezpegaldesleukin is or about 3600 μg.

Embodiments above provide certain doses for use in autoimmune diseases and conditions and provide advantageous means to treat these diseases by maximizing the Treg stimulatory effects while at the same time minimizing the degree to which T-effector cell responses and pro-inflammatory responses are also elicited.

For embodiments described above, the patient is administered the indicated dose or doses for so long as needed to treat the autoimmune disease being treated, as determined by the skilled artisan per methods described herein of known in the art.

As used herein, “methods of treatment” are equally applicable to use of a composition for treating the diseases or disorders described herein and/or compositions for use and/or uses in the manufacture of a medicaments for treating the diseases or disorders described herein.

### Induction Phase Followed by a Maintenance Phase:

In some aspects embodiments of the present disclosure provide dosing regimens for administration of selective Treg stimulator RUR_{20kD}-IL-2 and related compositions, for example rezpegaldesleukin, wherein said compositions are administered in an induction phase followed by a maintenance phase. Unit doses of rezpegaldesleukin as described above can be used to induce a selective Treg stimulatory response for a period of weeks, for example 12-24 weeks, which may then be followed by a maintenance phase wherein the dose may be lowered and or the frequency of administration is reduced, according to embodiments described below. Such regimens provide advantageous means of treating the autoimmune conditions described herein, in that the frequency or intensity of ISRs may be reduced, and or the risk of eliciting T effector and pro-effector responses is mitigated. Certain embodiments of dosing regimens of the present disclosure are summarized in the following table:

| Induction phase | | | Maintenance phase | | |
|---|---|---|---|---|---|
| Dose | Fre- | | Dose | Fre- | |
| selected | quency | Duration | selected | quency | Duration |
| from | selected | selected | from | selected | selected |
| about: | from: | from: | about: | from: | from: |
| 300 μg, | Q1W, | 12 weeks, | 300 μg, | Q2W, | 4 weeks to 52 |
| 450 μg, | Q2W, | 16 weeks, | 450 μg, | Q4W, | weeks of |
| 600 μg, | Q3W, | 20 weeks, | 600 μg, | Q6W, | administration, |
| 900 μg, | Q4W, | 24 weeks of | 900 μg, | Q8W, | 1-5 years of |
| 1200 μg, | Q6W | administration | 1200 μg, | Q10W, | administration |
| 1500 μg, | or Q8W | | 1500 μg, | Q12, | |
| 1800 μg, | intervals | | 1800 μg, | Q16W, | |
| 2100 μg, | | | 2100 μg, | Q20W | |
| 2400 μg, | | | 2400 μg, | Q24W, | |
| 2700 μg, | | | 2700 μg, | Q36W, | |
| 3000 μg | | | or 3000 μg | Q48W, | |
| 3300 μg | | | 3300 μg | or | |
| or | | | or | Q52W | |
| 3600 μg | | | 3600 μg | intervals | |
| per dose | | | per dose | | |

In some embodiments of the induction phase rezpegaldesleukin is administered to the patient for a period of 12 to 24 weeks (e.g., about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks).

In some embodiments of the maintenance phase rezpegaldesleukin is administered to the patient for a period of 4 to 52 weeks (e.g., about 4 weeks, about 6 weeks, about 8 weeks, about 10 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks).

In an embodiment, the present disclosure provides a method of treating a human subject having an autoimmune disease comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating a human subject having an autoimmune disease comprising administering to the human subject in an induction phase a dose of about 300 μg, about 450 μg, about 600 μg, about 900 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

Preferably the dose of rezpegaldesleukin in the induction phase comprises 300 μg. Preferably the dose of rezpegaldesleukin in the induction phase comprises 450 μg. Preferably the dose of rezpegaldesleukin in the induction phase comprises 600 μg. Preferably the dose of rezpegaldesleukin in the induction phase comprises 900 μg. Preferably the dose of rezpegaldesleukin in the induction phase comprises 1200 μg. Preferably the dose of rezpegaldesleukin in the induction phase comprises 1500 μg. Preferably the dose of rezpegaldesleukin in the induction phase comprises 1800 μg. Preferably the dose of rezpegaldesleukin in the induction phase comprises 2100 μg. Preferably the dose of rezpegaldesleukin in the induction phase comprises 2400 μg. Preferably the dose of rezpegaldesleukin in the induction phase comprises 2700 μg. Preferably the dose of rezpegaldesleukin in the induction phase comprises 3000 μg. Preferably the dose of rezpegaldesleukin in the induction phase comprises 3300 μg. Preferably the dose of rezpegaldesleukin in the induction phase comprises 3600 μg. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 1200 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, or pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 1500 μg of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 g. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 1800 μg per dose of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, or pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 2100 μg per dose of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 2400 μg per dose of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 2700 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 g. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 3000 μg, about 3300 μg, or about 3600 μg, per dose of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, or pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 2100 μg, about 2400 μg, about 2700 μg, about 3000 μg, about 3300 μg, or about 3600 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks.

Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 2100 μg, about 2400 μg, about 2700 μg, about 3000 μg, about 3300 μg, or about 3600 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 2100 μg, about 2400 μg, about 2700 μg, about 3000 μg, about 3300 μg, or about 3600 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of treating a human subject having an autoimmune disease comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 2100 μg, about 2400 μg, about 2700 μg, about 3000 μg, about 3300 μg, or about 3600 μg per dose, of rezpegaldesleukin; wherein said administering is carried out once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

For each of the above embodiments, the present disclosure further provides embodiments wherein the autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

In an embodiment, the present disclosure provides a method of treating moderate to severe atopic dermatitis in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method of reducing sleep loss in a patient with moderate to severe atopic dermatitis in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration. Preferably the dose of rezpegaldesleukin in the induction phase comprises 300 μg. Preferably the dose of rezpegaldesleukin in the induction phase comprises 450 μg. Preferably the dose of rezpegaldesleukin in the induction phase comprises 600 μg. Preferably the dose of rezpegaldesleukin in the induction phase comprises 900 μg. Preferably the dose of rezpegaldesleukin in the induction phase comprises 1200 μg. Preferably the dose of rezpegaldesleukin in the induction phase comprises 1500 μg. Preferably the dose of rezpegaldesleukin in the induction phase comprises 1800 μg. Preferably the dose of rezpegaldesleukin in the induction phase comprises 2100 μg. Preferably the dose of rezpegaldesleukin in the induction phase comprises 2400 μg. Preferably the dose of rezpegaldesleukin in the induction phase comprises 2700 μg. Preferably the dose of rezpegaldesleukin in the induction phase comprises 3000 μg. Preferably the dose of rezpegaldesleukin in the induction phase comprises 3300 μg. Preferably the dose of rezpegaldesleukin in the induction phase comprises 3600 μg. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In an embodiment, the present disclosure provides a method treating moderate to severe atopic dermatitis in a patient in need thereof, wherein the patient continues to experience moderate to severe disease after treatment with topical corticosteroids, topical calcineurin inhibitors, or crisaborole, or topical Jak inhibitors, systemic steroids, cyclosporine, biologic therapies (i.e. targeting IL-4/13 pathway), oral JAK inhibitors, and/or topical PDE4 inhibitors; or topical corticosteroids, topical calcineurin inhibitors, or crisaborole, or topical JAK inhibitors, and/or topical PDE4 inhibitors, systemic steroids, cyclosporine, biologic therapies (i.e. targeting IL-4/13 pathway), oral JAK inhibitors, are medically inadvisable for the patient, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

In other embodiments the present disclosure provides a method of treating an autoimmune disease identified herein, in a patient in need thereof, wherein the patient continues to experience clinical disease symptoms after treatment with current standards of care, or wherein the patient is intolerant to current standards of care, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks.

Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks.

Preferred embodiments of the present disclosure comprise methods for treatment of moderate to severe atopic dermatitis comprising dosing regimens of rezpegaldesleukin. Determining if the moderate to severe atopic dermatitis patient is a responder to rezpegaldesleukin can be assessed by evaluating the patient's skin clearance, skin improvement, and/or improvement in itch, sleep, or quality of life. For example, skin clearance and skin improvement can be measured by Investigator Global Assessment (IGA) or Eczema Area and Severity Index (EASI) scores. Itch, sleep loss and quality of life can be measured by Pruritus Numerical Rating Scale (NRS), Sleep Loss score and DLQI (Dermatology Life Quality Index) or CDLQI (Children Dermatology Life Quality Index) scales, respectively. In some embodiments, the patient is a responder when the patient's EASI score determined after the induction period is reduced by 75% or greater compared to the patient's EASI score at the baseline. In some embodiments, the patient is a responder when the patient's IGA score is 0 or 1 after the induction period. In some embodiments, the patient is a responder when the patient's IGA score is 0 or 1 after the induction period and the patient's IGA score determined after the induction period is reduced by 2 points or greater compared to the patient's IGA score at the baseline In some embodiments, if the atopic dermatitis patient is a responder, rezpegaldesleukin is administered at about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose once every four weeks for the maintenance period. In some embodiments, the rezpegaldesleukin is administered to the patient subcutaneously.

In some embodiments, the patient has moderate to severe atopic dermatitis for at least a year at the baseline. The moderate to severe atopic dermatitis can be determined by criteria known in the art, e.g., the American Academy of Dermatology Consensus Criteria for Chronic Atopic Dermatitis. In some embodiments, the patient has an EASI score of 16 or greater, an IGA score of 3 or greater, and more than 10% of body surface area (BSA) affected by atopic dermatitis, at the baseline. In some embodiments, the patient has inadequate response to topical corticosteroids, topical calcineurin inhibitors, or crisaborole; or topical corticosteroids, topical calcineurin inhibitors, or crisaborole are medically inadvisable for the patient.

In each of the embodiments described herein for treatment of atopic dermatitis, the atopic dermatitis patient being treated with rezpegaldesleukin may be either 1) naïve to prior treatments with biological agents, including antibodies against IL-4/IL-13, and/or IL-31, including but not limited to dupilumab, lebrikizumab, or tralokinumab, or 2) atopic dermatitis patients having prior experience with these treatments. As used herein the term “bio-experienced atopic dermatitis patient” is one which has previously been treated with one or more antibody therapies targeted against IL-4/IL-13, and/or IL-31. Treatment with these agents is known to the skilled artisan (See e.g., Vikram N. Sahni, et al., (2022), The evolving atopic dermatitis management landscape, Expert Opinion on Pharmacotherapy, 23:4, 517-526). In each of the embodiments described herein for treatment of atopic dermatitis, the patient being treated with rezpegaldesleukin may be either 1) naïve to prior treatments with PDE4 inhibitors or Janus kinase (JAK) inhibitors, including but not limited to abrocitinib, baricitnib or upadacitinib, or 2) patients having prior experience with these treatments. As used herein the term “JAK inhibitor-experienced atopic dermatitis patient” is one which has previously been treated with one or more JAK inhibitor agents. In an embodiment the patient to be treated for moderate to severe atopic dermatitis is a patient naïve to prior treatments with biological agents for atopic dermatitis. In an embodiment the patient to be treated for moderate to severe atopic dermatitis is a patient that is bio-experienced, and as used herein, this means a patient who has received prior treatments with biological agents for atopic dermatitis. As used herein, bio-experienced atopic dermatitis patients are those who have received one or more course of prior treatment with an antibody including dupilumab, tralokinumab, lebrikizumab, nemolizumab, eblsakimab or cendakimab, for instance. In an embodiment the patient to be treated for moderate to severe atopic dermatitis is a patient that is jak-experienced, and as used herein, this means a patient who has received one or more prior treatments with jak-inhibitor agents for atopic dermatitis, including baricitinib, upadacinitib, or abrocitinib, for instance.

Patients having been treated with these biological or small molecule therapies may have had some significant efficacy, or not, and may find the efficacy of these agents to decrease over time to an extent where an alternative and/or more effective treatment is needed, and rezpegaldesleukin is conceived to provide efficacy for these patients due to its distinct mechanism of action providing stimulation of Treg cells. Methods of assessing the unmet needs of atopic dermatitis patients are known to the skilled artisan and described herein, and patients in need of rezpegaldesleukin treatment as described herein can be identified as those who continue to have mild, moderate, or severe atopic dermatitis after having been treated with other biological or small molecule therapies for atopic dermatitis. According to the embodiments provided herein, an atopic dermatitis patient is in need of treatment with rezpegaldesleukin, if prior atopic dermatitis therapy either did not provide adequate reduction of disease symptoms, as described herein, or provided reduced or inadequate relief. The embodiments for treatment of atopic dermatitis with the dosing regimens and formulations of rezpegaldesleukin described herein may provide a variety of advantages, such as an enhanced efficacy response, and/or an improved safety profile with respect to the risk of adverse effects. The embodiments provided herein represent means for treating bio-experienced atopic dermatitis patient with induction and maintenance dosing regimens and each embodiment for treatment of atopic dermatitis is to be understood to represent treatment of either a bio-naïve patient or a bio-experienced patient or both. Studies of atopic dermatitis patients described herein can be modified to include bio-experienced atopic dermatitis patients, for instance where the subjects in the study represent for example 75% bio-experienced atopic dermatitis patients (for example dupilumab experienced patients) and 25% bio-naïve atopic dermatitis patients. The number and ratio of the aforementioned groups can be varied based on the desired study design.

The embodiments provided herein represent means for treating JAK inhibitor-experienced atopic dermatitis patient with induction and maintenance dosing regimens and each embodiment for treatment of atopic dermatitis is to be understood to represent treatment of either a bio-naïve patient or a JAK inhibitor-experienced atopic dermatitis patient or both. Studies of atopic dermatitis patients described herein can be modified to include JAK inhibitor-experienced atopic dermatitis patients, for instance where the subjects in the study represent for example 75% JAK inhibitor-experienced atopic dermatitis patients (for example to abrocitinib or upadacitinib) and 25% JAK-naïve atopic dermatitis patients. The number and ratio of the aforementioned groups can be varied based on the desired study design.

In some embodiments, the methods described herein further comprise determining one or more of the following characteristics of the patient at baseline and during and after the induction period: the EASI score; the IGA score; the percentage of BSA affected by atopic dermatitis; pruritus NRS score; SCORAD (Scoring of Atopic Dermatitis) score; sleep loss score; POEM (Patient-Oriented Eczema Measure) total score; DLQI (Dermatology Life Quality Index) or CDLQI (Children Dermatology Life Quality Index) score; EQ-5D (European Quality of Life-5 Dimensions); ACQ-5 (Asthma Control Questionnaire-5); PROMIS (Patient-Reported Outcomes Measurement Information System) Anxiety and Depressive Symptoms.

In some embodiments, the methods described herein further comprise determining one or more of the following characteristics of the patient during and after the maintenance period: the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms.

In another aspect, provided herein are methods of reducing sleep loss in a patient with moderate to severe atopic dermatitis; such methods comprise administering to the patient rezpegaldesleukin comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration.

In some embodiments, the sleep loss is determined by the patient's sleep loss score. In some embodiments, the patient's sleep loss score after the rezpegaldesleukin treatment is reduced two point or greater compared to the patient's sleep score at baseline.

Also provided herein are methods of reducing sleep loss in a patient with moderate to severe atopic dermatitis; such methods comprise administering to the patient rezpegaldesleukin comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to rezpegaldesleukin after the induction period; and if the patient is a responder, administering a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration. In some embodiments, the sleep loss is determined by the patient's sleep loss score. In some embodiments, the patient's sleep loss score after rezpegaldesleukin treatment is reduced two points or greater compared to the patient's sleep score at baseline.

### Use of Rezpegaldesleukin, and the Formulation and Dosing Regimen Embodiments Provided Herein, to Enhance the Survival and Effectiveness of Autologous Cellular Treg Therapies:

Treg therapy has shown promise in early clinical trials for treating graft-versus-host disease, transplant rejection and autoimmune disorders, however a challenge has been to isolate sufficiently pure Tregs and expand them to a clinical dose (K. N. MacDonald, et al., Methods to manufacture regulatory T cells for cell therapy Clinical and Experimental Immunology, 197:52-63). A variety of manufacturing protocols have been tested in clinical trials and are known to the skilled artisan, as summarized by MacDonald et. al. Methods used to manufacture Tregs for administration to patients being treated for graft-versus-host disease, transplant rejection and/or autoimmune disorders are known to the skilled artisan, including the choice of cell source and protocols for cell isolation and expansion for Treg therapy (See e.g., K. N. MacDonald, et al., Methods to manufacture regulatory T cells for cell therapy Clinical and Experimental Immunology, 197:52-63). Further, methods for preparing and/or treating pro-inflammatory diseases and conditions by administration of autologous Treg's (from the affected subject), and/or Treg/Th2 hybrid cells from de-differentiated T cells, are also described in the art, for instance in WO2021231797 A1.

Despite the advances in short-term graft survival, long-term graft survival remains the main challenge considering the increased mortality and morbidity associated with chronic rejection and the toxicity of immunosuppressive drugs. Novel and safe approaches are also needed to prolong graft survival in transplant recipients (See A. H. Mansourabadi et al., Cell therapy in transplantation: A comprehensive review of the current applications of cell therapy in transplant patients with the focus on Tregs, CAR Tregs, and Mesenchymal stem cells, (2021), International Immunopharmacology 97:107669). Suppressing immune responses through the number and/or activity of polyclonal Tregs and/or antigen-specific Tregs (such as those engineered with chimeric antigen receptors (CAR Tregs)), is a further concept of the present disclosure, wherein rezpegaldesleukin is used to promote the survival, proliferation, and/or activity of these cells in the recipient for application in organ transplantation. Use of rezpegaldesleukin administered according to induction and maintenance regimens described herein are conceived to provide enhanced survival, proliferation, and/or Treg activity of cellular Treg therapies transferred to graft-versus-host disease, transplant rejection and/or autoimmune disorder patients to induce or promote tolerance and inhibit undesirable immune and/or inflammatory responses. The clinical studies and results described herein provide evidence of Treg stimulation and proliferation, as well as relief of autoimmune and or inflammatory symptoms, and support the use of rezpegaldesleukin treatment as a means to promote survival and/or extended proliferation of autologous Treg cellular therapy, and/or treat graft-versus-host disease and/or transplant rejection.

In embodiments of the present disclosure rezpegaldesleukin and formulations and compositions thereof, as described herein, are used as an adjunct or combination therapy with cellular Treg administration according to various ex-vivo Treg preparations for transfer or administration to graft-versus-host disease, transplant rejection and/or autoimmune disorder patients, as a method of cellular Treg therapy. In other embodiments, rezpegaldesleukin and formulations and compositions thereof as described herein are used to stimulate the recipients endogenous Tregs for the treatment of graft-versus-host disease and/or transplant rejection.

In an embodiment the present disclosure provides the use of rezpegaldesleukin for the treatment of patients receiving autologous Treg cellular therapy for treating graft-versus-host disease, transplant rejection and autoimmune disorders, wherein the rezpegaldesleukin treatment promotes survival and/or extended proliferation of autologous Treg cellular therapy received by the patient.

In an embodiment, the present disclosure provides a method of separate, simultaneous, or sequential combination therapy with cellular Treg administration in a patient with graft-versus-host disease, a transplant recipient, and/or an autoimmune disorder, in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration. Preferably the dose of rezpegaldesleukin in the induction phase comprises 300 μg. Preferably the dose of rezpegaldesleukin in the induction phase comprises 450 μg. Preferably the dose of rezpegaldesleukin in the induction phase comprises 600 μg. Preferably the dose of rezpegaldesleukin in the induction phase comprises 900 μg. Preferably the dose of rezpegaldesleukin in the induction phase comprises 1200 μg. Preferably the dose of rezpegaldesleukin in the induction phase comprises 1500 μg. Preferably the dose of rezpegaldesleukin in the induction phase comprises 1800 μg. Preferably the dose of rezpegaldesleukin in the induction phase comprises 2100 μg. Preferably the dose of rezpegaldesleukin in the induction phase comprises 2400 μg. Preferably the dose of rezpegaldesleukin in the induction phase comprises 2700 μg. Preferably the dose of rezpegaldesleukin in the induction phase comprises 3000 μg. Preferably the dose of rezpegaldesleukin in the induction phase comprises 3300 μg. Preferably the dose of rezpegaldesleukin in the induction phase comprises 3600 μg. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks. Preferably the patient has or is at risk of graft-versus-host disease. Preferably the patient is a transplant recipient. Preferably the patient has an autoimmune disorder.

In an embodiment, the present disclosure provides a method of treating a patient having or at risk of graft-versus-host disease, or a transplant recipient, in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration. Preferably the dose of rezpegaldesleukin in the induction phase comprises 300 μg. Preferably the dose of rezpegaldesleukin in the induction phase comprises 450 μg. Preferably the dose of rezpegaldesleukin in the induction phase comprises 600 μg. Preferably the dose of rezpegaldesleukin in the induction phase comprises 900 μg. Preferably the dose of rezpegaldesleukin in the induction phase comprises 1200 μg. Preferably the dose of rezpegaldesleukin in the induction phase comprises 1500 μg. Preferably the dose of rezpegaldesleukin in the induction phase comprises 1800 μg. Preferably the dose of rezpegaldesleukin in the induction phase comprises 2100 μg. Preferably the dose of rezpegaldesleukin in the induction phase comprises 2400 μg. Preferably the dose of rezpegaldesleukin in the induction phase comprises 2700 μg. Preferably the dose of rezpegaldesleukin in the induction phase comprises 3000 μg. Preferably the dose of rezpegaldesleukin in the induction phase comprises 3300 μg. Preferably the dose of rezpegaldesleukin in the induction phase comprises 3600 μg. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks. Preferably the patient has or is at risk of graft-versus-host disease. Preferably the patient is a transplant recipient. Preferably the patient has an autoimmune disorder.

In an embodiment the present disclosure provides the use of rezpegaldesleukin for the treatment of patients receiving transplants, such as Hematopoietic Stem Cell transplant, wherein the rezpegaldesleukin treatment stimulates proliferation and/or activity of the transplant recipients Treg's to induce tolerance (See Copsel S N, et al., (2022), Recipient Tregs: Can They Be Exploited for Successful Hematopoietic Stem Cell Transplant Outcomes?, Front. Immunol. 13:932527). Tregs comprise non-redundant regulatory compartments which maintain self-tolerance and have been found to be of potential therapeutic usefulness in autoimmune disorders and transplants including allogeneic hematopoietic stem cell transplantation (allo-HSCT). Although treatment in patients with low dose IL-2 months post-HSCT have been encouraging, manipulating Tregs in recipients early post-transplant is challenging, in part likely an indirect consequence of damage to the microenvironment required to support Treg expansion of which little is understood (See Copsel S N, et al.). Means for manipulating recipient Tregs in vivo prior to and after HSCT are needed, and an embodiment of the present invention provides the use of rezpegaldesleukin for promote survival, proliferation, and/or activity of Treg's to induce tolerance and improve HSCT outcomes. Rezpegaldesleukin administration can be used to stimulate endogenous recipient Treg's or autologous Treg's co-administered to the recipient. Methods of preparing and using Tregs capable of suppressing immune responses against a donor alloantigen or an autoantigen, optionally including natural killer (NK) cells, are described for promoting allograft acceptance in a transplant recipient and treating autoimmune disorders in WO2020123825 A1. The Tregs or mixed population of Treg and NK cells are derived from the subject's blood cells and can reduce or replace the use of broad-acting immunosuppressants.

Methods for treating or preventing GvHD in a transplant patient by administering to the patient autologous Treg cells are known to the skilled artisan and described for example in WO2020095284 A1.

Chimeric-antigen-receptor regulatory T cells (CAR-Tregs) generally exhibit limited persistence after administration to a patient. Methods of preparing and using CAR-Tregs are described for example in WO2019241549 A1. In an embodiment the present disclosure provides the use of rezpegaldesleukin for the treatment of patients receiving chimeric-antigen-receptor regulatory T cell therapy, wherein the rezpegaldesleukin treatment promotes survival and/or extended proliferation of the chimeric-antigen-receptor regulatory T cell therapy received by the patient.

In an embodiment, the present disclosure provides a method of separate, simultaneous, or sequential combination therapy with cellular Treg, NK cells, or chimeric-antigen-receptor regulatory T cells (CAR-Tregs), or a mixture thereof, administration in a patient with graft-versus-host disease, a transplant recipient, and/or an autoimmune disorder, in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration. Preferably the dose of rezpegaldesleukin in the induction phase comprises 300 μg. Preferably the dose of rezpegaldesleukin in the induction phase comprises 450 μg. Preferably the dose of rezpegaldesleukin in the induction phase comprises 600 μg. Preferably the dose of rezpegaldesleukin in the induction phase comprises 900 μg. Preferably the dose of rezpegaldesleukin in the induction phase comprises 1200 μg. Preferably the dose of rezpegaldesleukin in the induction phase comprises 1500 μg. Preferably the dose of rezpegaldesleukin in the induction phase comprises 1800 μg. Preferably the dose of rezpegaldesleukin in the induction phase comprises 2100 μg. Preferably the dose of rezpegaldesleukin in the induction phase comprises 2400 μg. Preferably the dose of rezpegaldesleukin in the induction phase comprises 2700 μg. Preferably the dose of rezpegaldesleukin in the induction phase comprises 3000 μg. Preferably the dose of rezpegaldesleukin in the induction phase comprises 3300 μg. Preferably the dose of rezpegaldesleukin in the induction phase comprises 3600 μg. Preferably the dose of rezpegaldesleukin in the maintenance phase comprises 300 μg. Preferably the dose of rezpegaldesleukin in the maintenance phase comprises 450 μg. Preferably the dose of rezpegaldesleukin in the maintenance phase comprises 600 μg. Preferably the dose of rezpegaldesleukin in the maintenance phase comprises 900 μg. Preferably the dose of rezpegaldesleukin in the maintenance phase comprises 1200 μg. Preferably the dose of rezpegaldesleukin in the maintenance phase comprises 1500 μg. Preferably the dose of rezpegaldesleukin in the maintenance phase comprises 1800 μg. Preferably the dose of rezpegaldesleukin in the maintenance phase comprises 2100 μg. Preferably the dose of rezpegaldesleukin in the maintenance phase comprises 2400 μg. Preferably the dose of rezpegaldesleukin in the maintenance phase comprises 2700 μg. Preferably the dose of rezpegaldesleukin in the maintenance phase comprises 3000 μg. Preferably the dose of rezpegaldesleukin in the maintenance phase comprises 3300 μg. Preferably the dose of rezpegaldesleukin in the maintenance phase comprises 3600 μg. Preferably the induction dose is administered once every two weeks. Preferably the induction dose is administered once every four weeks. Preferably the maintenance dose is administered once every four weeks. Preferably the maintenance dose is administered once every eight weeks. Preferably the maintenance dose is administered once every twelve weeks. Preferably the maintenance dose is administered once every twenty-four weeks. Preferably the maintenance dose is administered once every thirty-six weeks. Preferably the maintenance dose is administered once every forty-eight weeks. Preferably the maintenance dose is administered once every fifty-two weeks. Preferably the patient has or is at risk of graft-versus-host disease. Preferably the patient is a transplant recipient. Preferably the patient has an autoimmune disorder. Preferably the cellular therapy comprises Treg and NK cells. Preferably the cellular therapy comprises chimeric-antigen-receptor regulatory T cells (CAR-Tregs).

In each of the induction and maintenance embodiments above, preferably the rezpegaldesleukin composition administered is replaced by a composition defined by one of the following compositions of Formulae A-F:

A composition of Formula A, wherein the composition comprises, on a molar basis, about 5 mole percent or less mono-PEGylated IL-2 conjugates, and from about 28 mole percent to about 60 mole percent di-PEGylated IL-2 conjugates, and from about 24 mole percent to about 65 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons.

A composition of Formula A, wherein the composition comprises, on a molar basis, about 5 mole percent or less mono-PEGylated IL-2 conjugates, and from about 28 mole percent to about 60 mole percent di-PEGylated IL-2 conjugates, and from about 24 mole percent to about 65 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons; wherein the composition further comprise a formulation of 5 mM sodium acetate, 25 mM sodium chloride, 7.5% (w/v) sucrose, at a pH of about 5.0.

A composition of Formula B, wherein the composition comprises, on a molar basis, from about 2.5 mole percent to about 4.5 mole percent mono-PEGylated IL-2 conjugates, and from about 35 mole percent to about 50 mole percent di-PEGylated IL-2 conjugates, and from about 38 mole percent to about 46 mole percent tri-PEGylated IL-2 conjugates, and from about 3 mole percent to about 10 mole percent higher PEGylated IL-2 conjugates, and wherein the nominal average molecular weight of each branched polyethylene glycol moiety is about 20,000 daltons.

A composition of Formula B, wherein the composition comprises, on a molar basis, from about 2.5 mole percent to about 4.5 mole percent mono-PEGylated IL-2 conjugates, and from about 35 mole percent to about 50 mole percent di-PEGylated IL-2 conjugates, and from about 38 mole percent to about 46 mole percent tri-PEGylated IL-2 conjugates, and from about 3 mole percent to about 10 mole percent higher PEGylated IL-2 conjugates, and wherein the nominal average molecular weight of each branched polyethylene glycol moiety is about 20,000 daltons; wherein the composition further comprise a formulation of 5 mM sodium acetate, 25 mM sodium chloride, 7.5% (w/v) sucrose, at a pH of about 5.0.

A composition of Formula C, wherein the composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein the nominal average molecular weight of each branched polyethylene glycol moiety is about 20,000 daltons.

A composition of Formula C, wherein the composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein the nominal average molecular weight of each branched polyethylene glycol moiety is about 20,000 daltons; wherein the composition further comprise a formulation of 5 mM sodium acetate, 25 mM sodium chloride, 7.5% (w/v) sucrose, at a pH of about 5.0.

A composition of Formula D, wherein the composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons.

A composition of Formula D, wherein the composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons; wherein the composition further comprise a formulation of 5 mM sodium acetate, 25 mM sodium chloride, 7.5% (w/v) sucrose, at a pH of about 5.0.

A composition of Formula E, wherein the composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons.

A composition of Formula E, wherein the composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons; wherein the composition further comprise a formulation of 5 mM sodium acetate, 25 mM sodium chloride, 7.5% (w/v) sucrose, at a pH of about 5.0.

A composition of Formula F, wherein the composition comprises, on a molar basis, about 2 mole percent to about 4 mole percent mono-PEGylated IL-2 conjugates, and from about 35 mole percent to about 55 mole percent di-PEGylated IL-2 conjugates, and from about 35 mole percent to about 55 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons.

A composition of Formula F, wherein the composition comprises, on a molar basis, about 2 mole percent to about 4 mole percent mono-PEGylated IL-2 conjugates, and from about 35 mole percent to about 55 mole percent di-PEGylated IL-2 conjugates, and from about 35 mole percent to about 55 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons, wherein the composition further comprises a formulation of 5 mM sodium acetate, 25 mM sodium chloride, 7.5% (w/v) sucrose, at a pH of about 5.0.

In some embodiments, the methods described herein further comprise determining one or more of the following characteristics of the patient: the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms.

Before, during and after the treatment with rezpegaldesleukin, the patient can be assessed for one or more characteristics of the Atopic Dermatitis Disease Severity Measures (ADDSM), which determine certain signs, symptoms, features, or parameters that have been associated with atopic dermatitis and that can be quantitatively or qualitatively assessed. Exemplary ADDSM include, but are not limited to, Eczema Area and Severity Index (EASI), Investigator Global Assessment (IGA), body surface area (BSA), Scoring of Atopic Dermatitis (SCORAD), Pruritus Numerical Rating Scale (NRS), Sleep loss scale, Skin pain NRS score, Patient-Oriented Eczema Measure (POEM) total score, Dermatology Life Quality Index (DLQI) or Children Dermatology Life Quality Index (CDLQI), DLQI-Relevant (DLQI-R) score, Patient-Reported Outcomes Measurement Information System (PROMIS) Anxiety and Depressive Symptoms, EQ-5D (European Quality of Life-5 Dimensions), ACQ-5 (Asthma Control Questionnaire-5), World Health Organization-Five Well-Being Index (WHO-5) score, Recap of Atopic Eczema (RECAP) score, Treatment Satisfaction Questionnaire for Medication-9 items (TSQM-9) score. The ADDSM can be measured at baseline and at one or more time points after administration of rezpegaldesleukin. The difference between the value of the ADDSM at a particular time point following initiation of treatment and the value of the ADDSM at baseline is used to establish whether there has been an improvement (e.g., a reduction) in the ADDSM.

In some embodiments, the methods described herein further comprise determining one or more of the following characteristics of the patient at baseline and during and after the induction period: the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms.

In some embodiments, the patient's EASI score is determined after the induction period. In some embodiments, the patient's EASI score determined after the induction period is reduced by 50% or greater compared to the patient's EASI score at the baseline, which means the patient has achieved “EASI-50”. In some embodiments, the patient's EASI score determined after the induction period is reduced by 75% or greater compared to the patient's EASI score at the baseline, which means the patient has achieved “EASI-75”. In some embodiments, the patient's EASI score determined after the induction period is reduced by 90% or greater compared to the patient's EASI score at the baseline, which means the patient has achieved “EASI-90”. A patient is considered a responder to rezpegaldesleukin when the patient reached EASI-75 after the induction period.

In some embodiments, the patient's IGA score is determined after the induction period. A patient is considered a responder to rezpegaldesleukin when the patient's IGA score is 0 or 1 after the induction period. In some embodiments, a patient is considered a responder to rezpegaldesleukin when the patient's IGA score is 0 or 1 after the induction period, and the patient's IGA score after the induction period is reduced by 2 points or greater compared to the patient's IGA score determined at the baseline.

In some embodiments, as indicated, after completion of the induction period, the patient enters a maintenance period. During the maintenance period, the patient is further treated with rezpegaldesleukin. The dosing regimen for the maintenance period can be selected based on the patient's ADDSM assessment and response to rezpegaldesleukin after the induction period, e.g., the patient's IGA or EASI score after the induction period, and/or the patient's own characteristics, e.g., weight, age.

In some embodiments, the methods described herein further comprise determining one or more of the following characteristics of the patient during and after the maintenance period: the EASI score; the IGA score; the percentage of BSA affected by atopic dermatitis; pruritus NRS score; SCORAD score; sleep loss score; POEM total score; DLQI or CDLQI score; EQ-5D; ACQ-5; PROMIS Anxiety and Depressive Symptoms. Similarly, the patient's EASI score during and after the maintenance period can be evaluated to see if the patient has reached EASI-50, EASI-75, or EASI-90. The patient's IGA score during and after the maintenance period can be evaluated to see if the patient's IGA score is 0 or 1 and if the patient's IGA score is reduced by 2 points or greater.

The “Investigator Global Assessment” or “IGA” is an assessment measure used globally to rate the severity of the patient's AD (Simpson E, et al. J Am Acad Dermatol. 2020; 83 (3): 839-846). It is based on a 5-point scale ranging from 0 (clear) to 4 (severe) and a score is selected using descriptors that best describe the overall appearance of the lesions at a given time point (see Table 2). It is not necessary that all characteristics under Morphological Description be present. The IGA can be conducted prior to conducting the EASI and BSA assessments.

TABLE 2
Investigator Global Assessment (IGA)

| Score | Grade | Definition |
|---|---|---|
| 0 | Clear | Minor, residual discoloration; no erythema or |
| | | induration/papulation; no oozing/crusting; no edema. |
| 1 | Almost | Trace, faint pink erythema with barely perceptible |
| | clear | induration/papulation and no oozing/crusting; no edema. |
| 2 | Mild | Faint-pink erythema with papulation and edema |
| | | perceptible upon palpation and no oozing/crusting; |
| | | minimal induration. |
| 3 | Moderate | Pink-red erythema with definite edema of skin papules |
| | | and plaques; there may be some oozing/crusting; |
| | | palpable induration. |
| 4 | Severe | Deep/bright red erythema with significant swelling and |
| | | obvious raised borders of papules and plaques with |
| | | oozing/crusting; significant induration. |

The “Eczema Area and Severity Index” or “EASI” is a measure used in clinical settings to assess the severity and extent of AD (Hanifin et al., Exp Dermatol. 2001; 10:11-18). EASI is a composite index with scores ranging from 0 to 72, with the higher values indicating more severe and or extensive disease. The severity of erythema, induration/papulation, excoriation, and lichenification can be assessed by a clinician or other medical professional on a scale of 0 (absent) to 3 (severe) for each of the 4 body areas: head and neck, trunk, upper limbs, and lower limbs, with half points allowed. In addition, the extent of AD involvement in each of the 4 body areas can be assessed as a percentage by body surface area of head, trunk, upper limbs, and lower limbs, and converted to a score of 0 to 6. A total score (0-72) is assigned based on the sum of total scores for each of the four body region scores.

The body surface area (BSA) assessment estimates the extent of disease or skin involvement with respect to AD and is expressed as a percentage of total body surface. BSA is determined by a clinician or other medical professional using the patient palm is about 1% BSA rule.

The “Scoring of Atopic Dermatitis” or “SCORAD” is a validated clinical tool for assessing the extent and intensity of AD developed by the European Task Force on Atopic Dermatitis (Consensus report of the European Task Force on Atopic Dermatitis. Dermatology. 1993; 186 (1): 23-31). There are 3 components to the assessment: (i) the extent of AD is assessed as a percentage of each defined body area and reported as the sum of all areas, with a score ranging from 0 to 100 (assigned as “A” in the overall SCORAD calculation); (ii) the severity of 6 symptoms of AD: redness, swelling, oozing/crusting, excoriation, skin thickening/lichenification, dryness. Each item is graded as follows: none (0), mild (1), moderate (2), or severe (3) (for a maximum of 18 total points, assigned as “B” in the overall SCORAD calculation); (iii) subjective assessment of itch and of sleeplessness is recorded for each symptom using a visual analogue scale (VAS), where 0 is no itch (or sleeplessness) and 10 is the worst imaginable itch (or sleeplessness), with a maximum possible score of 20 (assigned as “C” in the overall SCORAD calculation). The SCORAD Index formula is: A/5+7B/2+C. The maximal score of the SCORAD Index is 103.

Pruritus Numerical Rating Scale (NRS) is an 11-point scale used by patients (and if applicable, with help of parents/caregiver if required) to rate their worst itch severity over the past 24 hours with 0 indicating “No itch” and 10 indicating “Worst itch imaginable” (Phan N Q, et al. Acta Derm Venereol 2012; 92:502-507). Assessments are recorded by the patient daily using an electronic diary. The baseline pruritus NRS is determined based on the average of daily Pruritus NRS during the 7 days immediately preceding baseline. A minimum of 4 daily scores out of the 7 days immediately preceding baseline is required for this calculation.

Sleep loss scale rates patient's sleep loss due to pruritus on a 5-point Likert scale (with scores ranging from 0 [not at all], 1 [a little], 2 [moderately], 3 [quite a bit], to 4 [unable to sleep at all]). Assessments will be recorded daily by the patient using an electronic diary. Sleep loss can also be monitored by actigraphy methods known to the skilled artisan.

The Skin Pain NRS is an 11-point scale completed by patients (and if applicable, with help of parents/caregiver if required) to rate their worst skin pain (for example discomfort or soreness) severity over the past 24 hours with 0 indicating “No pain” and 10 indicating “Worst pain imaginable” (Newton L, et al. J Patient Rep Outcomes. 2019 Jul. 16; 3:42). Assessments are recorded by the patient daily up until Week 16 and weekly from Week 16 onwards using an electronic diary. The baseline Skin Pain NRS is determined based on the average of daily Skin Pain NRS during the 7 days immediately preceding baseline. A minimum of 4 daily scores out of the 7 days immediately preceding baseline is required for this calculation.

The Patient-Oriented Eczema Measure (POEM) is a 7-item, validated, questionnaire completed by the patient (and if applicable, with help of parents/caregiver if required) to assess disease symptoms over the last week (Centre of Evidence Based Dermatology. POEM-Patient Oriented Eczema Measure. Available at https://www.nottingham.ac.uk/research/groups/cebd/resources/poem.aspx). Patients are asked to respond to 7 questions on skin dryness, itching, flaking, cracking, sleep loss, bleeding, and weeping. All 7 answers carry equal weight with a total possible score from 0 to 28 (answers scored as: No days=0; 1-2 days=1; 3-4 days=2; 5-6 days=3; everyday=4). A high score is indicative of a poor quality of life. POEM responses are captured weekly using an electronic diary.

The Dermatology Life Quality Index (DLQI) is a 10-item, validated questionnaire completed by the patient or caregiver, used to assess the impact of skin disease on the quality of life of the patient (Finlay, A. Y. and Khan, G. K. 1994. Clinical and Experimental Dermatology 1993 Sep. 23; 19:210-216). The 10 questions cover the following topics: symptoms, embarrassment, shopping and home care, clothes, social and leisure, sport, work or study, close relationships, sex, and treatment, over the previous week. Each question is scored from 0 to 3 (“not at all,” “a little,” “a lot,” and “very much”), giving a total score ranging from 0 to 30. A high score is indicative of a poor quality of life.

For adolescents below the age of 16, the Children DLQI (CDLQI) is employed which is based on a set of 10 questions different from those of the DLQI (Lewis-Jones M S, Finlay A Y. British Journal of Dermatology, 1995; 132:942-949).

The DLQI-Relevant (DLQI-R) is a recently developed scoring that adjusts the total score of the DLQI questionnaire for the number of not relevant responses (NRRs) indicated by a patient (Rencz F, et al. Br J Dermatol. 2020; 182 (5): 1167-1175).

Patient-Reported Outcomes Measurement Information System (PROMIS) is a set of person-centered measures that evaluates and monitors physical, mental, and social health in adults and children. The PROMIS® measures used in this study include Anxiety and Depression short forms, which assess the patients' symptoms over the previous week. Patients≤17 years will complete pediatric versions for the duration of the study.

The PROMIS Anxiety Short Form v1.0—Anxiety 8a is a participant administered questionnaire that assesses the following items in adults: self-reported fear (fearfulness, panic); anxious misery (worry, dread); hyperarousal (tension, nervousness, restlessness), and somatic symptoms related to arousal (racing heart, dizziness) (PROMIS Anxiety 2019, Published Mar. 1, 2019. Accessed Mar. 8, 2021. Available at https://www.healthmeasures.net/images/PROMIS/manuals/PROMIS_Anxiety_Scoring_Manual.pdf). Each question has 5 response options, with scores ranging from 1 to 5. The total scores range from 8 to 40, with the higher score indicating a higher level of anxiety. The adult self-report assesses anxiety “in the past 7 days.”

The PROMIS Depression Short Form v1.0—Depression 8a is a participant administered questionnaire that assesses the following items in adults: self-reported negative mood (sadness, guilt); views of self (self-criticism, worthlessness); social cognition (loneliness, interpersonal alienation), and decreased positive affect and engagement (loss of interest, meaning, and purpose) (PROMIS Depression 2019, Published Feb. 28, 2019. Accessed Mar. 8, 2021. Available at https://www.healthmeasures.net/images/PROMIS/manuals/PROMIS_Depression_Scorin g_Manual.pdf). Somatic symptoms (such as changes in appetite or sleeping patterns) are not included. This helps eliminate potential confounding effects of these items when assessing participants with co-morbid physical conditions. Each question has 5 response options, with scores ranging from 1 to 5. The total scores range from 8 to 40, with the higher score indicating a higher level of depression. The adult self-report assesses depression “in the past 7 days.”

EQ-5D (European Quality of Life-5 Dimensions) comprises five dimensions: mobility, self-care, usual activities, pain/discomfort and anxiety/depression. The EQ VAS records the patient's self-rated health on a vertical visual analogue scale. The scores on these five dimensions can be presented as a health profile or can be converted to a single summary index number (utility) reflecting preferability compared to other health profiles. EQ-5D is completed by the patient in the study clinic.

The European Quality of Life-5 Dimensions-5 Levels (EuroQol-5D-5L or EQ-5D-5L) is a participant-administered, 5 questions plus 1 visual analog scale (VAS) standardized measure of health status in adults that provides a simple, generic measure of health for clinical and economic appraisal. The EQ-5D-5L consists of 2 components: a descriptive system of the respondent's health and a rating of his or her current health state using a 0 to 100 mm VAS (20 cm). The descriptive system comprises the following 5 dimensions: mobility, self care, usual activities, pain/discomfort, and anxiety/depression. Each dimension has 5 levels: no problems, slight problems, moderate problems, severe problems, and extreme problems. The respondent is asked to indicate his or her health state by ticking (or placing a cross) in the box associated with the most appropriate statement in each of the 5 dimensions. It should be noted that the numerals 1 to 5 have no arithmetic properties and should not be used as an ordinal score. The EQ-5D-5L health states, defined by the EQ-5D-5L descriptive system, may be converted into a single summary index by applying a formula that essentially attaches values (also called weights) to each of the levels in each dimension. The VAS records the respondent's self-rated health on a vertical VAS where the endpoints are labeled “best imaginable health state” and “worst imaginable health state.” This information can be used as a quantitative measure of health outcome (Herdman et al., Qual Life Res. 2011; 20 (10): 1727-1736; EuroQol Group, EQ-5D-5L User Guide. Version 2.1. April 2015. Accessed: Jan. 14, 2021. Available at https://euroqol.org/wp-content/uploads/2016/09/EQ-5D-5L_UserGuide_2015.pdf). The self-rated health status captured by EQ-5D-5L relates to the participant's situation at the time of completion. No attempt is made to recall health status over the preceding days or weeks (EuroQol Group 2015).

ACQ-5 is Asthma Control Questionnaire. Patients who report comorbid asthma prior to enrollment will complete the Asthma Control Questionnaire (ACQ-5) in addition to other patient reported outcomes in this trial. The ACQ-5 has been shown to reliably measure asthma control and distinguish patients with well-controlled asthma (score≤0.75 points) from those with uncontrolled asthma (score ≥1.5 points). It consists of 5 questions that are scored on a 7-point Likert scale with a recall period of 1 week. The total ACQ-5 score is the mean score of all questions; a lower score represents better asthma control. ACQ-5 is completed by the patient in the study clinic.

The term “inadequate response” as used herein refers to inability to achieve good disease control of atopic dermatitis (e.g., not able to achieve IGA≤2 or EASI 75) after use of the treatment for the duration recommended by the product prescribing information, or flare of atopic dermatitis occurs while on the treatment.

The term “intolerance” or “intolerant” as used herein refers to unacceptable toxicity (e.g., elevated creatinine, elevated liver function tests, uncontrolled hypertension, paranesthesia, headache, nausea, hypertrichosis), or requirement for a drug at doses or duration beyond those specified in the prescribing information.

The term “topical corticosteroid” or “TCS”, as used herein includes Group I, Group II, Group III and Group IV topical corticosteroids. According to the Anatomical Therapeutic Chemical (ATC) Classification System of World Health Organization, the corticosteroids are classified as weak (Group I), moderately potent (Group II) and potent (Group III) and very potent (Group IV), based on their activity as compared to hydrocortisone. Group IV TCS (very potent) are up to 600 times as potent as hydrocortisone and include clobetasol and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (weak or mild) includes hydrocortisone, prednisolone, and methylprednisolone.

The term “topical calcineurin inhibitor” or “TCI”, as used herein, includes pimecrolimus, tacrolimus, and other inhibitors that suppress calcineurin activities and can be topically applied to a patient's skin.

In embodiments that refer to a methods of treatment as described herein, such embodiments are also further embodiments for use in that treatment, or alternatively for the use in the manufacture of a medicament for use in that treatment.

### Preparations and Examples of RUR_{20kD}-IL-2 Selective Treg Stimulator Composition Production

RUR_{20kD}-IL-2 selective Treg stimulator composition drug substance is a PEGylated conjugate composition consisting of recombinant human interleukin-2 (rhIL-2, aldesleukin sequence) conjugated to multiple polyethylene glycol (PEG) moieties covalently bound at lysine groups. The number of PEG moieties per rhIL-2 molecule (degree of PEGylation) is a distribution of predominantly 2 and 3 PEG moieties per molecule (di- or tri-PEGylated) with minor species containing 1 PEG (mono-PEGylated) and 4 PEG (tetra-PEGylated) and higher PEGylated molecules, resulting in an average of about 2.5 PEG moieties per rhIL-2. Each PEG moiety has a nominal molecular weight of 20 kDa, and rhIL-2 has a molecular weight of 15.3 kDa, resulting in a nominal RUR_{20kD}-IL-2 selective Treg stimulator composition molecular weight of 65 kDa. Unless specified otherwise, the concentration, quantity, and dosing levels of RUR_{20kD}-IL-2 selective Treg stimulator composition are recited on a protein basis which only counts the mass contributed by the protein component and not that contributed by the PEG chains. By using a protein basis, the effective molecular weight, when used for dosing and concentration calculations, becomes consistent at 15.3 kDa and is not affected by the distribution of PEGylated rhIL-2 molecules, since only the rhIL-2 protein is counted.

The Preparations and Examples are set forth by way of illustration and not limitation, and various modifications may be made by one of ordinary skill in the art. Methods of preparing selective Treg stimulator compositions, including RUR_{20kD}-IL-2 embodiments and related compositions, are described in WO 2019/226,538 (incorporated herein by reference), or described herein or known to the skilled artisan. The reagents and starting materials are readily available or may be readily synthesized by one of ordinary skill in the art. Suitable conditions for the steps of these methods are well known, and appropriate substitutions of buffers and reagents are within the skill of the art. Furthermore, the skilled artisan will appreciate that in some circumstances the steps and order by which compositions are produced may be modified and is well appreciated by the skilled biochemist. Likewise, it will be appreciated that preparations may be isolated and/or purified by various well-known techniques as needed or desired.

### Preparation of IL-2 Intermediate:

The IL-2 moiety can be derived from non-recombinant methods and/or from recombinant methods and the disclosure is not limited in this regard. The IL-2 moiety can be derived from human sources, animal sources, and plant sources. For example, it is possible to isolate IL-2 from biological systems and otherwise obtain IL-2 from cultured media. See, for example, the procedures described in U.S. Pat. No. 4,401,756 and in Pauly et al. (1984) J. Immunol Methods 75 (1): 73-84. Exemplary IL-2 moieties are described in the literature, and in for example, U.S. Pat. Nos. 5,116,943, 5,153,310, 5,635,597, 7,101,965 and 7,567,215 and U.S. Patent Application Publication Nos. 2010/0036097 and 2004/0175337. A preferred IL-2 moiety has the amino acid sequence provided in FIG. 2 and represents the amino acid sequence of aldesleukin as used herein.

Methods for producing and expressing recombinant polypeptides in vitro and in prokaryotic and eukaryotic host cells are well-known to those of ordinary skill in the art. See, for example, U.S. Pat. No. 5,614,185. The IL-2 moiety can be expressed in bacterial [e.g., E. coli, see, for example, Fischer et al. (1995) Biotechnol. Appl. BioIL-2m. 21 (3): 295-311], mammalian [see, for example, Kronman et al. (1992) Gene 121:295-304], yeast [e.g., Pichia pastoris, see, for example, Morel et al. (1997) Biochem. J. 328 (1): 121-129], and plant [see, for example, Mor et al. (2001) Biotechnol. Bioeng. 75 (3): 259-266] expression systems. Although recombinant based methods for preparing proteins can differ, recombinant methods typically involve constructing the nucleic acid encoding the desired polypeptide or fragment, cloning the nucleic acid into an expression vector, transforming a host cell (e.g., plant, bacteria, yeast, transgenic animal cell, or mammalian cell such as Chinese hamster ovary cell or baby hamster kidney cell), and expressing the nucleic acid to produce the desired polypeptide or fragment. Various methods of protein purification may be employed to purify a composition of the present disclosure and such methods are known in the art and described, for example, in Scopes, Protein Purification: Principles and Practice, 3rd Edition, Springer, NY (1994). To facilitate identification and purification of the recombinant polypeptide, nucleic acid sequences that encode for an epitope tag or other affinity binding sequence can be inserted or added in-frame with the coding sequence, thereby producing a fusion protein comprised of the desired polypeptide and a polypeptide suited for binding.

### Preparation of Selective Treg Stimulator Compositions, Including RUR_{20kD}-IL-2 Embodiments and Related Compositions:

An exemplary selective Treg stimulator composition of RUR_{20kD}-IL-2 is generally prepared by reacting purified IL-2 with a molar excess of PEG reagent (excess of molar equivalents with respect to IL-2), mPEG2 (20 kD)-butanoic acid, N-hydroxysuccinimide ester (or any other suitably activated ester) (1,3-bis(methoxypoly(ethylene glycol) MW 10,000 carbamoyl)-2-propanoxy)-4-succinimidyl butanoate, in a bicine solution at high pH of about 9. The reactants are mixed for about 30 minutes to about 5 hours, or from about 30 minutes to 4 hours, or from about 30 minutes to 2 hours, or from about 30 minutes to 1 hour, generally under mild conditions, e.g., from about 20° C. to about 65° C., or from about 20° C. to about 40° C., or at ambient or room temperature. The reaction is quenched by acidification to low pH by addition of a suitable acid such as acetic acid.

The PEGylated rIL-2 reaction product is then purified by a suitable method such as ion exchange chromatography. For example, when employing ion exchange chromatography, the RUR_{20kD}-IL-2 composition binds to the resin and then is eluted with a suitable gradient, such as a sodium chloride gradient. The chromatography product pool is then concentrated and diafiltered into suitable formulation buffer (for example, sodium acetate buffer with sucrose) using, for example, tangential flow filtration (TFF).

If desired, the product pool may be further separated into positional isomers by reverse phase chromatography using a reverse phase-high performance liquid chromatography (RP-HPLC) using a suitable column (e.g., a C18 column or C3 column, available commercially from companies such as Amersham Biosciences or Vydac), or by ion exchange chromatography using an ion exchange column, e.g., a Sepharose™ ion exchange column available from Amersham Biosciences. Either approach can be used to separate polymer-active agent isomers having the same molecular weight (i.e., positional isoforms).

Selective Treg stimulator compositions, including RUR_{20kD}-IL-2 embodiments and related compositions, can be characterized by various analytical and bioassay techniques described herein and/or known to the skilled artisan, including analytical HPLC, SDS-Page, LCMS, and bioassays such as CTLL-2 proliferation, and Treg induction in-vivo.

### Embodiments of RUR20kD-IL-2 Selective Treg Stimulator Compositions

RUR_{20kD}-IL-2 selective Treg stimulator compositions are mixtures of conjugates comprising recombinant human interleukin-2 (rhIL-2, and in particular the aldesleukin amino acid sequence with no additional amino acid mutations or substitutions), stably covalently conjugated to 20 kDa polyethylene glycol (PEG) moieties, wherein the mixtures have defined fractions with certain degrees of PEGylation per IL-2 moiety. Compositions of the present disclosure comprise selected mixtures of IL-2 PEG conjugates having defined fractions of predominantly di-PEGylated and tri-PEGylated IL-2, and defined lesser fractions of mono-PEGylated IL-2, and/or tetra or higher PEGylated IL-2.

Embodiments of the present disclosure provide compositions comprising conjugates of the formula:

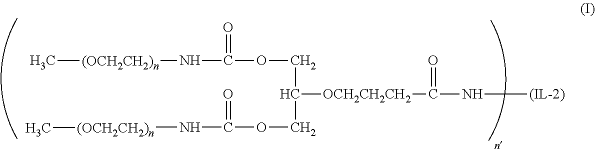

wherein IL-2 is an interleukin-2, n is an integer from about 3 to about 4000, and n′ is 1, 2 and 3.
The polymer portion of formula (I) is also referred to as 1,3-bis(methoxypoly(ethylene glycol) MW 10,000 carbamoyl)-2-propanoxy)-4-butanoyl (up to and including the carbonyl group that is covalently attached to an amino nitrogen of the IL-2 moiety).

Embodiments of the present disclosure further provide selective Treg stimulator compositions, including RUR_{20kD}-IL-2 embodiments and related compositions, and a preferred embodiment of said composition comprises aldesleukin. In a particular embodiment of said compositions, the nominal average molecular weight of each branched polyethylene glycol moiety is about 20,000 daltons. In a further particular embodiment of said compositions, PEGyalted IL-2 conjugates of the composition have a PEG moiety attached at lysine 31.

Mixture compositions in accordance with formula (I) are generally referred to herein as RUR-IL2 which encompass a range of PEG sizes.

Illustrative ranges of n include, for example, in addition to from about 3 to about 4000, from about 5-2000, or from about 10-1000, or from about 10-750, or from about 10-500, or from about 10-400, or from about 10-300, or from about 10-250, or from about 20-250. In some embodiments, n is, on average, about 226.

In some embodiments, the selective Treg stimulator composition of formula I comprises IL-2R stably covalently-linked with branched polyethylene glycol moieties, where the number of branched PEG moieties per IL-2 moiety (degree of PEGylation) is a distribution of predominantly 2 and 3-mers (di- and tri-PEGylated) in a mixture with minor fractions including 1-mers (mono-PEGylated) and 4-mers (tetra-PEGylated). Thus, in some embodiments minor fractions in the compositions according to formula I will include conjugates wherein n′ is 1, 4, 5, or higher, but not more than 11. As used herein, higher pegylated conjugates refer to 4-11, or 4 to 7.

In some preferred embodiments provided herein the compositions comprise aldesleukin conjugates with individual covalent PEG attachments having nominal molecular weights of about 20 kD total. These compositions further comprise selected mixtures of IL-2 PEG conjugates having defined fractions of predominantly di-PEGylated and tri-PEGylated IL-2, and defined lesser fractions of mono-PEGylated IL-2, and/or tetra or higher PEGylated IL-2. Particular preparations of RUR_{20kD}-IL-2 compositions are described below and throughout this application. As used herein, compositions of RUR_{20kD}-IL-2 of Formula A, compositions of RUR_{20kD}-IL-2 of Formula B, compositions of RUR_{20kD}-IL-2 of Formula C, compositions of RUR_{20kD}-IL-2 of Formula D, and/or compositions of RUR_{20kD}-IL-2 of Formula E, and/or compositions of RUR20kD-IL-2 of Formula F represent certain embodiments of selective Treg stimulator RUR_{20kD}-IL-2 and related compositions, and in these embodiments the IL-2 moiety is aldesleukin (as described herein). Optionally these compositions comprise pharmaceutically acceptable salts thereof.

In an embodiment, provided herein is a composition of RUR_{20kD}-IL-2 of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons. Preferably the composition of RUR_{20kD}-IL-2 of Formula A comprises 80 mol % or greater combined di- and tri-PEGylated IL-2 conjugates.

In an embodiment, provided herein is a composition of RUR_{20kD}-IL-2 of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons. Preferably the composition of RUR_{20kD}-IL-2 of Formula B comprises a combined total of di-PEGylated and tri-PEGylated IL-2 conjugates from about 80 to about 95 mol %.

In an embodiment, provided herein is a composition of RUR_{20kD}-IL-2 of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons. Preferably the composition of RUR_{20kD}-IL-2 of Formula C comprises a combined total of di-PEGylated and tri-PEGylated IL-2 conjugates from about 87 to about 90 mol %.

In an embodiment, provided herein is a composition of RUR_{20kD}-IL-2 of Formula D, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons. Preferably the composition of RUR_{20kD}-IL-2 of Formula D comprises a combined total of di-PEGylated and tri-PEGylated IL-2 conjugates from about 87 to about 90 mol %.

In an embodiment, provided herein is a composition of RUR_{20kD}-IL-2 of Formula E, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises mono-PEGylated IL-2 conjugates which have a PEG moiety attached at lysine K7, wherein the nominal average molecular weight of each branched polyethylene glycol moiety is about 20,000 daltons. Preferably the composition of RUR_{20kD}-IL-2 of Formula E comprises a combined total of di-PEGylated and tri-PEGylated IL-2 conjugates from about 87 to about 90 mol %.

In an embodiment, provided herein is a composition of RUR20kD-IL-2 of Formula F, wherein the composition comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons. Preferably the composition of RUR20kD-IL-2 of Formula F comprises 80 mol % or greater combined di- and tri-PEGylated IL-2 conjugates.

As used herein, “RUR_{20kD}-IL-2 and related compositions” may refer to one or more compositions according to any one of an RUR_{20kD}-IL-2 of Formula A, and/or an RUR_{20kD}-IL-2 of Formula B, and/or an RUR_{20kD}-IL-2 of Formula C, and/or an RUR_{20kD}-IL-2 of Formula D, and/or an RUR_{20kD}-IL-2 of Formula E, and/or an RUR20kD-IL-2 of Formula F and/or pharmaceutically acceptable salts of these compositions. Preparations of Example 1 and/or Example 1A are non-limiting examples of an “RUR_{20kD}-IL-2 and related composition” of the present disclosure.

### Further Embodiments of the Selective Treg Stimulator Compositions Provided Herein:

The compositions provided herein may comprise conjugates where n equals 2, e.g., a di-PEGylated conjugates wherein two branched polyethylene glycol polymers, each having the 1,3-bis(methoxypoly(ethylene glycol)_{10kD}carbamoyl)-2-propanoxy)-4-butanoyl structure shown above, are attached at the same relative locations for substantially all di-PEGylated IL-2 conjugates in the composition. Alternatively, a di-PEGylated conjugate may comprise a mixture of di-PEGylated conjugates, e.g., a mixture of di-PEGylated conjugates where attachment of the branched polyethylene glycol moiety occurs at two sites on IL-2, where the particular attachment sites are not the same for all of the di-PEGylated IL-2 conjugates comprised in the composition. Thus, such di-PEGylated compositions are homogeneous in terms of the degree of PEGylation, in particular the number of branched PEG moieties attached (e.g., 2-mers), but are heterogeneous in terms of the locations of PEG attachment on the IL-2 molecule and in this case represent positional isomers of PEG attachment.

The compositions may also comprise single conjugates where n equals 3, e.g., a tri-PEGylated conjugate wherein three branched polyethylene glycol moieties are attached at the same relative locations for substantially all IL-2 conjugates in the composition. Alternatively, a tri-PEGylated conjugate may comprise a mixture of tri-PEGylated conjugates, e.g., a mixture of tri-PEGylated conjugates where the site of attachment of the branched polyethylene glycol moiety occurs at different sites on IL-2 for the conjugates comprised in the composition. Thus, such tri-PEGylated compositions are homogeneous in terms of the degree of PEGylation, in particular the number of branched PEG moieties attached, but are heterogeneous in terms of the locations of PEG attachment on the IL-2 molecule and in this case represent positional isomers of PEG attachment. As used herein, “higher pegylated IL-2 conjugates” refers to more than three branched polyethylene glycol moieties being attached to IL-2, for instance ranging from 4-11 attached moieties.

The compositions may also comprise single conjugates where n equals 1, e.g., a mono-PEGylated conjugate wherein one branched polyethylene glycol moieties is attached at the same relative location for substantially all IL-2 conjugates in the composition. Alternatively, a mono-PEGylated conjugate may comprise a mixture of mono-PEGylated conjugates, e.g., a mixture of mono-PEGylated conjugates where the site of attachment of the branched polyethylene glycol moiety occurs at different sites on IL-2 for the conjugates comprised in the composition. Thus, such mono-PEGylated compositions are homogeneous in terms of the degree of PEGylation, in particular the number of branched PEG moieties attached, but are heterogeneous in terms of the location of PEG attachment on the IL-2 molecule and in this case represent positional isomers of PEG attachment.

Certain locations of PEG attachment on the IL-2 molecule are more prevalent in the compositions described herein. For instance, lysines K7 or K8 or K31 or K75, are commonly PEGylated sites. Compositions of RUR_{20kD}-IL-2 and related compositions may comprise conjugates wherein lysines K7 or K8 or K31 or K75 are PEGylated sites. Compositions of RUR_{20kD}-IL-2 and related compositions may comprise mono-PEGylated conjugates wherein lysines K7 or K8 or K31 or K75 are PEGylated sites. Compositions of RUR_{20kD}-IL-2 and related compositions may comprise mono-PEGylated conjugates wherein lysine K7 is a PEGylated site. Compositions of RUR_{20kD}-IL-2 and related compositions may comprise mono-PEGylated conjugates wherein lysine K31 is a PEGylated site.

In some embodiments, the composition contains no more than about 20 mol %, and preferably no more than about 15 mol % of conjugates, when considered collectively, encompassed by formula (I), where n′ is an integer selected from 1, 4, 5, or an integer greater than 5, where the mole percentage is based upon total PEG-IL-2 conjugates. In some embodiments, the composition contains no more than about 10 mol % of conjugates, when considered collectively, encompassed by formula (I), where n′ is an integer selected from 1, 4, 5, or an integer greater than 5, where the mole percentage is based upon total PEG-IL-2 conjugates. In some additional embodiments, the composition contains no more than about 10 mol % of monomers, and preferably no more than about 7 mol % monomers, or no more than about 5 mol percent monomers (i.e., in accordance with structure (I) where n equals 1). In some further embodiments, the composition contains no more than about 10 mol % of tetramers, and preferably no more than about 7 mol % tetramers, or no more than about 5 mol percent tetramers (i.e., in accordance with structure (I) where n equals 4). In certain additional embodiments, the composition comprises no more than about 10 mol % of monomers and no more than about 10 mol % of tetramers. Alternatively, the composition comprises no more than about 7 mol % of monomers and no more than about 7 mol % of tetramers, or may comprise no more than about 5 mol % of monomers and no more than about 5 mol % of tetramers.

In some embodiments, with respect to the PEGylated IL-2 in the composition, the composition will generally satisfy one or more of the following characteristics: at least about 80% of the conjugates in the composition will comprise a mixture of di-PEGylated and tri-PEGylated conjugates, some having 2 and some having 3 branched polymers having the structure shown in formula (I) above attached to the IL-2 moiety; at least about 85% of the conjugates in the composition will comprise a mixture of di-PEGylated and tri-PEGylated conjugates, some having 2 and some having 3 branched polymers having the structure shown in formula (I) above attached to the IL-2 moiety; at least about 90% of the conjugates in the composition will comprise a mixture of di-PEGylated and tri-PEGylated conjugates, some having 2 and some having 3 branched polymers having the structure shown in formula (I) above attached to the IL-2 moiety; and at least about 95% of the conjugates in the composition will comprise a mixture of di-PEGylated and tri-PEGylated conjugates, some having 2 and some having 3 branched polymers having the structure shown in formula (I) above attached to the IL-2 moiety; no more than about 20% of the conjugates in the composition will have from 1, or 4 or more branched polymers having the structure shown in formula (I) above attached to the IL-2 moiety; no more than about 15% of the conjugates in the composition will have from 1, or 4 or more branched polymers having the structure shown in formula (I) above attached to the IL-2 moiety; no more than about 10% of the conjugates in the composition will have from 1, 4 or more branched polymers having the structure shown in formula (I) above attached to the IL-2 moiety; no more than about 7% of the conjugates in the composition will have from 1, or 4 or more branched polymers having the structure shown in formula (I) above attached to the IL-2 moiety.

In some embodiments, the composition contains no more than about 20 mol %, and preferably no more than about 15 mol % of compounds, when considered collectively, encompassed by formula (I), where n′ is an integer selected from 1, 4, 5, or an integer greater than 5, where the mole percentage is based upon total PEG-IL-2 conjugates. In some embodiments, the composition contains no more than about 10 mol % of conjugates, when considered collectively, encompassed by formula (I), where n′ is an integer selected from 1, 4, 5, or an integer greater than 5, where the mole percentage is based upon total PEG-IL-2 conjugates. In some additional embodiments, the composition contains no more than about 10 mol % of monomers, and preferably no more than about 7 mol % monomers, or no more than about 5 mol percent monomers (i.e., in accordance with structure (I) where n equals 1). In some further embodiments, the composition contains no more than about 10 mol % of tetramers, and preferably no more than about 7 mol % tetramers, or no more than about 5 mol percent tetramers (i.e., in accordance with structure (I) where n equals 4). In certain additional embodiments, the composition comprises no more than about 10 mol % of monomers and no more than about 10 mol % of tetramers. Alternatively, the composition comprises no more than about 7 mol % of monomers and no more than about 7 mol % of tetramers, or may comprise no more than about 5 mol % of monomers and no more than about 5 mol % of tetramers.

In some further embodiments, the composition comprises approximately equimolar amounts of

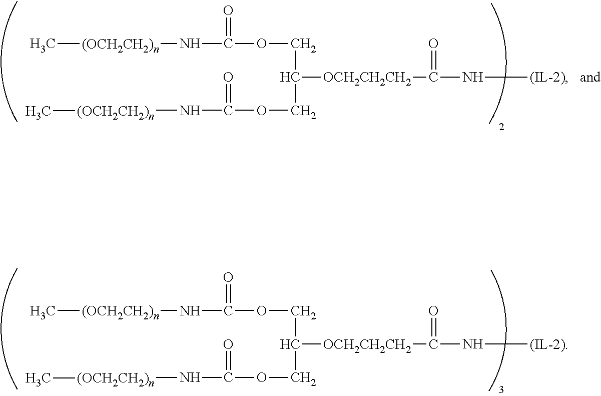

For example, illustrative compositions may comprise any one or more of the following approximate ratios of di-PEGylated species to tri-PEGylated species: 1.4:1; 1.3:1; 1.2:1; 1.1:1; 1:1; 1:1.1; 1:1.2; 1:1.3; or 1:1.4. The average number of PEG moieties per IL-2 for such compositions is selected from, for example, 2; 2.1; 2.2; 2.3; 2.4; 2.5; 2.6; 2.7; 2.8; 2.9; and 3. In certain embodiments, the average number of PEG moieties per IL-2 is about 2.5.

For example, in some embodiments, the compositions comprise no more than about 20 mole percent (mol %) of IL-2 conjugates, when considered collectively, encompassed by the formula

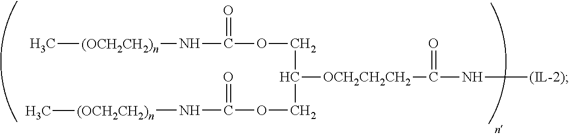

wherein n′ is selected from 1, 4, 5, or an integer greater than 5.

Yet in some additional embodiments, the compositions comprise no more than about 15 mole percent (mol %) of IL-2 conjugates, that when considered collectively, are encompassed by the formula

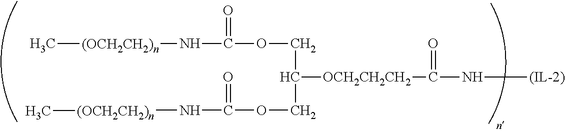

and have n′ selected from 1, 4, 5, or an integer greater than 5.

Yet in some further embodiments, the compositions comprise no more than about 10 mole percent (mol %) of IL-2 conjugates, that when considered collectively, are encompassed by the formula

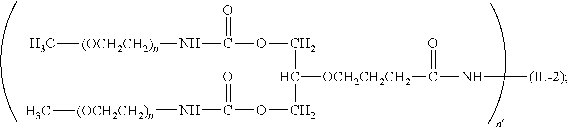

and have n′ selected from 1, 4, 5, or an integer greater than 5.

In some additional embodiments of the foregoing, the composition comprises no more than about 10 mol % of IL-2 conjugates and having n′ equal to 1. In yet some other embodiments, the composition comprises no more than about 7 mol % of IL-2 conjugates having n′ equal to 1.

In yet some further embodiments, the compositions comprise no more than about 5 mol % of IL-2 conjugates n′ equal to 1. In yet some alternative embodiments, the composition comprises less than about 5 mol % of IL-2 conjugates having n′ equal to 1.

In some further embodiments, related to any one or more of the foregoing, the composition comprises no more than about 10 mol % of IL-2 conjugates having n′ equal to 4. Or, in some other embodiments, the composition comprises no more than about 7 mol % of IL-2 conjugates having n′ equal to 4. In yet some further embodiments, the composition comprises no more than about 5 mol % of IL-2 conjugates having n′ equal to 4.

Also provided herein is a composition comprising approximately equimolar amounts of

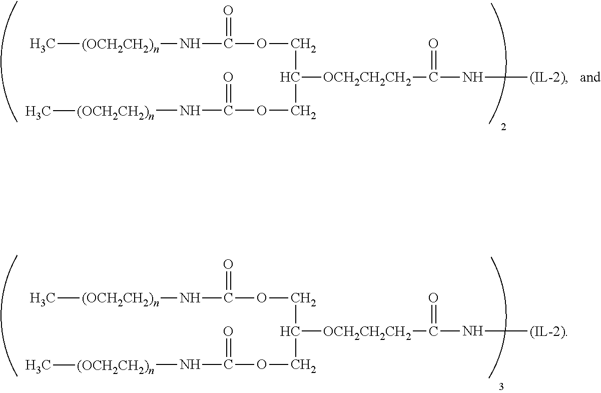

In yet additional embodiments, provided herein is a composition comprising IL-2 conjugates of formula

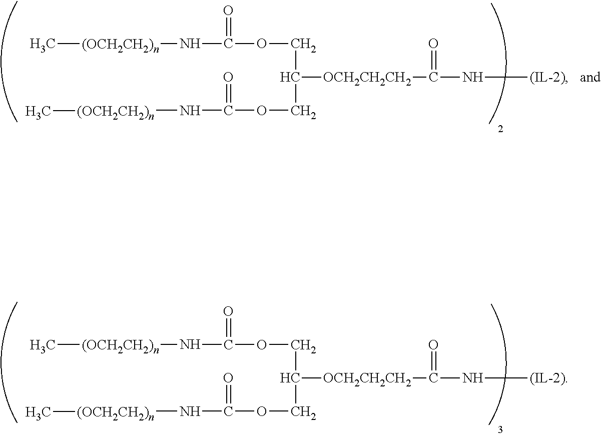

wherein the molar ratio of diPEG/triPEG conjugates is selected from the group consisting of 1.4:1; 1.3:1; 1.2:1; 1.1:1; 1:1; 1:1.1; 1:1.2; 1:1.3; and 1:1.4.

In yet some further embodiments, the composition has an average number of branched polyethylene glycol moieties (having a structure as shown above) per IL-2 residue selected from the group consisting of 2; 2.1; 2.2; 2.3; 2.4; 2.5; 2.6; 2.7; 2.8; 2.9; and 3. In a particular embodiment, the average number of branched polyethylene glycol moieties (having a structure as shown above) per IL-2 moiety is about 2.5. In some embodiments related to one or more of the foregoing, the value of n ranges from 5-2000. In some other embodiments, the value of n ranges from 10-1000. In yet some additional embodiments, the value of n ranges from 10-750. In some embodiments the value of n ranges from 10-500, or from 20-250.

The value of n in the embodiments provided herein can vary independently at each occurrence. In one or more embodiments described herein, the value of n in each of the polyethylene glycol arms of the branched polymer is substantially the same. In some further embodiments, the value of n in each of the polymer arms comprising the branched polymer ranges from about 170 to 285. In yet some further embodiments, the value of n in each of the polymer arms comprising the branched polymer ranges from about 204 to about 250. In one or more particular embodiments, the value of n in each of the polymer arms comprising the branched polymer is about 226.

In one or more embodiments related to any one or more of the aspects or embodiments provided herein, the nominal average molecular weight of each branched polyethylene glycol moiety is in a range of from about 250 daltons to about 90,000 daltons. In some other embodiments, the nominal average molecular weight of each branched polyethylene glycol moiety is in a range of from about 1000 daltons to about 60,000 daltons. In yet further embodiments, the nominal average molecular weight of each branched polyethylene glycol moiety is in a range of from about 5,000 daltons to about 60,000 daltons. In some other embodiments, the nominal average molecular weight of each branched polyethylene glycol moiety is in a range of from about 10,000 daltons to about 55,000 daltons.

In yet some additional embodiments, the nominal average molecular weight of each branched polyethylene glycol moiety is in a range of from about 15,000 daltons to about 25,000 daltons. In yet one or more further embodiments, the nominal average molecular weight of each branched polyethylene glycol moiety is in a range of from about 18,000 daltons to about 22,000 daltons. In yet some further embodiments, the nominal average molecular weight of each branched polyethylene glycol moiety is about 20,000 daltons.

Additional exemplary compositions comprise compositions in accordance with the above formulae wherein the overall polymer portion of the molecule has a nominal average molecular weight in a range of from about 250 daltons to about 90,000 daltons. Additional suitable ranges for the polymer portion of the molecule include nominal average molecular weights in a range selected from about 1,000 daltons to about 60,000 daltons, in a range of from about 5,000 daltons to about 60,000 daltons, in a range of about 10,000 daltons to about 55,000 daltons, in a range of from about 15,000 daltons to about 50,000 daltons, and in a range of from about 20,000 daltons to about 50,000 daltons.

Additional illustrative weight-average molecular weights for the polyethylene glycol polymer portion include about 200 daltons, about 300 daltons, about 400 daltons, about 500 daltons, about 600 daltons, about 700 daltons, about 750 daltons, about 800 daltons, about 900 daltons, about 1,000 daltons, about 1,500 daltons, about 2,000 daltons, about 2,200 daltons, about 2,500 daltons, about 3,000 daltons, about 4,000 daltons, about 4,400 daltons, about 4,500 daltons, about 5,000 daltons, about 5,500 daltons, about 6,000 daltons, about 7,000 daltons, about 7,500 daltons, about 8,000 daltons, about 9,000 daltons, about 10,000 daltons, about 11,000 daltons, about 12,000 daltons, about 13,000 daltons, about 14,000 daltons, about 15,000 daltons, about 20,000 daltons, about 22,500 daltons, about 25,000 daltons, about 30,000 daltons, about 35,000 daltons, about 40,000 daltons, about 45,000 daltons, about 50,000 daltons, about 55,000 daltons, about 60,000 daltons, about 65,000 daltons, about 70,000 daltons, and about 75,000 daltons. In some preferred embodiments, the weight-average molecular weight of the branched polyethylene glycol polymer is about 20,000 daltons. In some particular embodiments in which each branched PEG moiety has a nominal molecular weight of about 20,000 daltons, the resulting molecular weight range of the composition is from about 55 to 75 kDa, when characterized for the overall composition.

Further embodiments of the selective Treg stimulator compositions provided herein comprise pharmaceutically acceptable salts thereof. As described above, the IL-2 conjugate compositions may be in the form of a pharmaceutically acceptable salt. Typically, such salts are formed by reaction with a pharmaceutically acceptable acid or an acid equivalent. The term “pharmaceutically acceptable salt” in this respect, will generally refer to the relatively non-toxic, inorganic and organic acid addition salts. These salts can be prepared in situ in the administration vehicle or the dosage form manufacturing process, or by separately reacting a long-acting interleukin-2 composition as described herein with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, oxylate, mesylate, glucoheptonate, lactobionate, and laurylsulphonate salts and the like. (See, for example, Berge et al. (1977) “Pharmaceutical Salts”, J. Pharm. Sci. 66:1-19). Thus, salts as described may be derived from inorganic acids such as hydrochloride, hydrobromic, sulfuric, sulfamic, phosphoric, nitric, and the like; or prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. As used herein, the term “composition” or “compositions”, including the RUR_{20kD}-IL-2 embodiments and related compositions described herein, comprise any and/or all pharmaceutically acceptable salts of the PEGylated IL-2 conjugates. This description applies whether the term “or pharmaceutically acceptable salt thereof” is added to the description of the composition or not.

### BRIEF DESCRIPTION OF THE DRAWINGS

FIGS. 1A and 1B are representative reverse phase HPLC plots illustrating the general composition of an RUR_{20kD}-IL-2 composition, the preparation of which is described in Examples 1 and 1A. Moving from left to right along the x-axis (elution times, minutes), the purified conjugate composition comprises primarily di-PEGylated and tri-PEGylated rIL-2.

FIG. 2 is the amino acid sequence of aldesleukin (125-L-serine-2-133 interleukin-2, a recombinant non-glycosylated interleukin-2 expressed in E. coli).

FIG. 3 illustrates results from an interim analysis of a phase 1b study of patients with moderate-to-severe atopic dermatitis (NCT04081350) treated with rezpegaldesleukin, an RUR20kD-IL-2 Selective Treg stimulator composition, and represents mean percent change from baseline in EASI scores over time in a subset of patients. Moderate to severe atopic dermatitis patients being treated with the 24 μg/kg dose of rezpegaldesleukin every two weeks subcutaneously for 12 weeks, demonstrate a change in EASI score with a more robust effect than patients in the placebo group. Patients had to be EASI-50 responders at week 19 to continue in the study. (EASI=eczema area and severity index) See also Example 2.

FIG. 4 and FIG. 5 provide Study Schedule Protocol JIP-MC-KFAD: Treatment and Initial Follow-up.

FIG. 6 Study Schedule Protocol JIP-MC-KFAD: Clinical Response Follow-up for EASI 50 Responders at Week 19 Safety Follow-up.

FIG. 7 Phase 1b double-blind, placebo-controlled study KFAD (NCT04081350): change from baseline in EASI Score.

FIG. 8 Change from baseline in (A) total Treg cells and (B) CD25bright Treg cells in the PBO, 12 μg/kg and 24 μg/kg Cohorts.

FIG. 9 (A) Change from Baseline in PASI Score from W12 to W19, in the PBO and 24 μg/kg Cohorts. The proportion of patients who achieved (B) PASI-50 and (C) PASI-75 response up to W19 in the PBO and 24 μg/kg Cohorts.

FIG. 10 Change from baseline in (A) total Treg cells and (B) CD25bright Treg cells, and (C) Tcon cells in the PBO 24 μg/kg Cohorts.

FIG. 11 Schema of Study JIP-MC-KFAJ, a Phase 2 study to evaluate the efficacy and safety of rezpegaldesleukin in adults with systemic lupus erythematosus.

FIGS. 12A and 12B Study schema for EXAMPLE 5 A Phase 2, Randomized, Double-Blind, Parallel-Group, Placebo-Controlled, 52 Week Study to Evaluate the Efficacy of Rezpegaldesleukin (LY3471851, NKTR-358) in the Treatment of Adult Patients with Moderate-to-Severe Atopic Dermatitis.

FIG. 13 Study design overview of KFAD. (a) Full study design is not shown; the LY3471851 10 μg/kg cohort is not included in this analysis. (b) Total of 7 doses/patient EASI50=50% improvement from baseline in Eczema Area and Severity Index; PBO-placebo; Q2W-once every 2 weeks; SC=subcutaneous; W=Week.

FIG. 14 Atopic Dermatitis Study KFAD results-A dose-dependent trend in EASI percent change from baseline was observed with rezpegaldesleukin (LY3471851) vs. PBO up to Week 48. n=number of patients with assessments at the visit, EASI=Eczema Area and Severity Index; PBO=placebo; SD=standard deviation.

FIG. 15 Atopic Dermatitis Study KFAD results-A dose-dependent trend in EASI75 responders was seen with rezpegaldesleukin (LY3471851) vs. PBO up to Week 48. n=number of responders, EASI75-75% improvement from baseline in Eczema Area and Severity Index score; NRI=non-responder imputation; PBO-placebo.

FIG. 16 Atopic Dermatitis Study KFAD results-A dose-dependent trend in vIGA-AD (0,1) responders was seen with rezpegaldesleukin (LY3471851) vs. PBO up to Week 48.

FIG. 17 Atopic Dermatitis Study KFAD results-vIGA-AD (0,1) Durability in Week 16 Responders. A high percentage of week 16 vIGA-AD (0,1) responders maintained response to week 48 with LY3471851 24 μg/kg. Analysis includes patients who achieved vIGA-AD (0,1) at Week 16, n=number of responders, PBO=placebo.

FIG. 18 Atopic Dermatitis Study KFAD results—Itch NRS ≥4-Point Improvement. A dose-dependent trend in Itch NRS responders was seen with LY3471851 vs. PBO up to Week 48. N=number of responders, NRI=non-responder imputation; NRS=Numeric Rating Scale; PBO=placebo.

FIG. 19 Study design overview of KFAC. Total of 7 doses/patient, PBO=placebo; Q2W=once every 2 weeks; SC=subcutaneous; W=Week.

FIG. 20 Psoriasis Study KFAC results-PASI percent change from baseline. PASI improved with LY3471851 vs. PBO and was maintained up to Week 19.

FIG. 21 Study schema for EXAMPLE 5 A Phase 2, Randomized, Double-Blind, Parallel-Group, Placebo-Controlled, 52 Week Study to Evaluate the Efficacy of Rezpegaldesleukin (LY3471851, NKTR-358) in the Treatment of Adult Patients with Moderate-to-Severe Atopic Dermatitis including Bio-naïve and Bio-experienced patients.

### Preparations and Examples of RUR_{20kD}-IL-2 Selective Treg Stimulator Compositions and Formulations for Use in Dosing Regimens of the Present Disclosure EXAMPLES

It is to be understood that the foregoing description as well as the examples that follow are intended to illustrate and not limit the scope of the disclosure(s) provided herein. Other aspects, advantages and modifications within the scope of the disclosure will be apparent to those skilled in the art to which the disclosure pertains. The embodiments described in the following Preparations and Examples of RUR20kD-IL-2 selective Treg Stimulator Compositions, the formulations thereof, are useful as compositions for use in the treatment and or prevention dosing regimens described herein.

### Materials and Methods

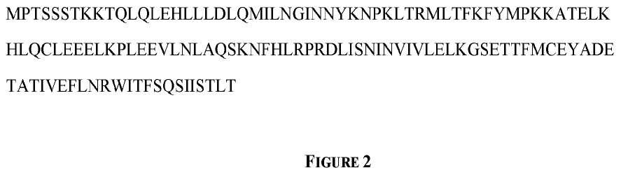

Recombinant human IL-2 having an amino acid sequence identical to that of aldesleukin (des-alanyl-1, serine-125 human interleukin-2, See FIG. 2) is cloned and expressed and used to prepare the exemplary selective Treg stimulator compositions referred to herein as RUR_{20kD}-IL-2. The sequence excludes amino acid #1 (alanine) from the native mature human IL-2 sequence, and there is a cysteine to serine amino acid mutation at amino acid #125. The first amino acid in the sequence is a methionine for direct bacterial expression (no signal peptide encoded). Upon expression, the N-terminal methionine is removed by the host methionine amino peptidase. A single disulfide linkage is formed between the cysteines at amino acid positions #58 and #105. The protein is not glycosylated as it is derived from E. coli. In some descriptions the conjugated IL-2 compositions can be described in some respects as (1,3-bis(methoxypoly(ethylene glycol) carbamoyl)-2-propanoxy)-4-butanamide) interleukin-2), noting this nomenclature does not fully describe the PEGylation pattern or mixture.

Polyethylene glycol reagent, mPEG2 (20 kD)-butanoic acid, N-hydroxysuccinimide ester (1,3-bis(methoxypoly(ethylene glycol)_{10kD}carbamoyl)-2-propanoxy)-4-succinimidyl butanoate (also referred to herein as mPEG2-ru-20K NHS), is prepared as described in Example 2 of U.S. Pat. No. 7,887,789. Appearance: white to off-white granular powder; molecular weight (Mn) 18-22 kDa (due to polymer polydispersity). The structure of 1,3-bis(methoxypoly(ethylene glycol)_{10kD}carbamoyl)-2-propanoxy)-4-succinimidyl butanoate is shown below.

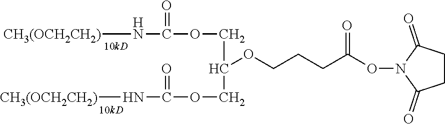

Unless specified otherwise, the concentration, quantity, and dosing levels of the selective Treg stimulator compositions, including RUR_{20kD}-IL-2 embodiments and related compositions provided herein, are reported on a protein basis which only counts the mass contributed by the protein component and not that contributed by the PEG moieties. By using a protein basis, the effective RUR_{20kD}-IL-2 composition molecular weight used for calculations is 15.3 kDa, even for a mixture of conjugated rIL-2 molecules having various degrees of PEGylation, since only the rIL-2 protein is counted.

An RUR_{20kD}-IL-2 related composition is a PEGylated conjugate mixture composition consisting of rhIL-2 (preferably aldesleukin sequence), conjugated to multiple polyethylene glycol (PEG) moieties covalently bound at the lysine groups. The number of PEG moieties per rhIL-2 molecule (degree of PEGylation) is a distribution of predominantly 2 and 3 PEG moieties per molecule (di- or tri-PEGylated) with minor species containing 1 PEG (mono-PEGylated) and 4 PEG (tetra-PEGylated) and/or higher PEGylated molecules, resulting in an average of about 2.5 PEG moieties per rhIL-2. Preferably each PEG moiety has a nominal molecular weight of 20 kDa, and rhIL-2 has a molecular weight of 15.3 kDa, resulting in a nominal RUR_{20kD}-IL-2 molecular weight of about 65 kDa.

### Example 1

### Preparation of RUR_{20kD}-IL-2 and Related Compositions

A stock solution (100 mg/mL) of mPEG2-ru-20K NHS is prepared in 2 mM HCl. A typical PEGylation reaction of IL-2 is carried out as follows: 115 mL of IL-2 (aldesleukin) stock solution (1.3 mg/mL) is transferred to a 250 mL plastic bottle and 15 mL of 0.5 M Bicine (N,N-bis(2-hydroxyethyl)glycine), pH 9.2 and 0.5 mL water are added to the solution of IL-2. PEGylation is initiated by drop-wise addition of 19.5 mL of mPEG2-ru-20K NHS stock solution to the IL-2-containing solution. The resultant reaction mixture contains 1 mg/mL IL-2, 50 mM Bicine and 10 molar equivalents of mPEG2-ru-20K NHS (with respect to protein) and has a pH of 8.7. The reaction is allowed to proceed at ambient temperature for 40 min under gentle stirring. The reaction is terminated by adding 2.2 mL acetic acid to reduce the reaction pH to 4.1.

The resulting IL-2 conjugate product is purified by cation exchange chromatography using SP FF Sepharose. Upon completion of the conjugation reaction, the reaction mixture is dialyzed against 20 volumes of 10 mM sodium acetate buffer (pH 4.0). The dialyzed sample is diluted 1:4 with water and loaded onto a column packed with SP FF Sepharose resin. Buffers used for the cation exchange chromatography are as follows: Buffer A: 10 mM sodium acetate (pH 4.0), and Buffer B: 10 mM sodium acetate, 1.0 M sodium chloride (pH 4.0). The resin is washed with Buffer B and equilibrated with Buffer A prior to sample loading. After loading, the resin is washed with 3 column volumes of Buffer A. Conjugated and non-conjugated IL-2 are eluted using a four-step gradient consisting of 0 to 50% Buffer B over 5 column volumes, 25% to 50% Buffer B over 1 column volume, 50% Buffer B over 1 column volume, 50% to 100% Buffer B over 1 column volume and 100% Buffer B over 1 column volume at a flow rate of 28 cm/h. Fractions containing IL-2 conjugates having a degree of PEGylation (dP) of 2 and 3 (i.e., di-mers and tri-mers) are identified by SDS-PAGE and pooled.

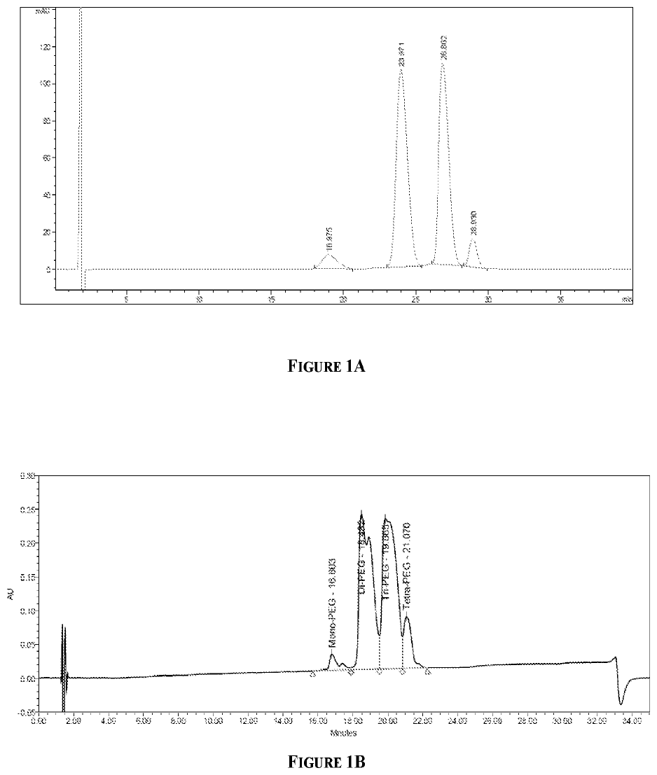

The pooled fractions containing di-mers and tri-mers are concentrated using a stirred ultrafiltration cell (Amicon) and nitrogen gas. The composition of the final product is determined by RP-HPLC using mobile phases: A, 0.09% TFA in water and B, 0.04% TFA in acetonitrile. An Intrada WP-RP C18 column (3×150 mm) is used with a flow rate of 0.5 ml/min and a column temperature of 50° C. The purified conjugate mixture is determined to comprise about 4.6% (mol) of mono-PEGylated rIL-2, about 47.7% (mol) of di-PEGylated rIL-2, about 42.9% (mol) of tri-PEGylated rIL-2 and about 4.8% (mol) of tetra-PEGylated IL-2. See FIG. 1, where elution times are provided on the x-axis. The average degree of PEGylation of the final product mixture is determined to be 2.48 (i.e., about 2.5). No free IL-2 is detected in the final product mixture. This preparation is an example of a composition of RUR_{20kD}-IL-2 of Formula A.

### Example 1-A

### Alternative Preparations of RUR_{20kD}-IL-2 and Related Compositions

Preparation of a desired RUR_{20kD}-IL-2 and related composition consists of fermentation and purification of the rhIL-2 protein process intermediate, conjugation of rhIL-2 with the PEG reagent starting material mPEG2-ru-20K NHS, purification of IL-2 conjugate fractions having specified degrees of PEGylation, and final formulation of the PEGylated rhIL-2 conjugates to generate the RUR_{20kD}-IL-2 composition of the desired distribution according to the embodiments described herein.

The desired RUR_{20kD}-IL-2 composition is prepared by reacting 1,3-bis(methoxypoly(ethylene glycol)_{10kD}carbamoyl)-2-propanoxy)-4-succinimidyl butanoate (also referred to herein as mPEG2-ru-20K NHS) with lysine residues on the interleukin-2 (IL-2) protein (aldesleukin sequence), resulting in a distribution of PEGylated IL-2 conjugates. The product contains predominately di-PEGylated and tri-PEGylated species, with lower amounts of mono- and/or tetra-PEGylated species.

Frozen IL-2 starting material (purified recombinant IL-2 (aldesleukin sequence) in 10 mM acetate, 5% trehalose, pH 4.5 buffer that had been stored at −70° C.) is thawed to room temperature. The PEG reactant, mPEG2-ru-20K NHS (powder), is solubilized by addition to a 2 mM HCl solution at ˜90 g/L at room temperature and agitated for a minimum of 15 minutes. The solution is then charged to the reaction vessel. The thawed IL-2 is added to the reaction vessel, diluted appropriately with water, followed by addition of 0.75 M bicine pH 9.7 buffer. The final IL-2 concentration in the reaction mixture is approximately 1.0 g/L, and the bicine concentration is approximately 50 mM to reach a target pH of 8.7. Generally, the PEG:rhIL-2 mass ratio is about 10:1 to 13:1 in a bicine buffered solution at pH 8.5 to 9.5 to PEGylate the protein. The reaction is incubated with continued agitation for 40 minutes at 22° C. as measured from the completion of the mPEG2-ru-20K NHS solution addition. At the end of the incubation period, the reaction is quenched with addition of 1 N acetic acid to rapidly lower the pH, and immediately followed by further stepwise titration to pH 4.0 using additional 1 N acetic acid. The quenched reaction is diluted 10× by addition of water. The diluted quenched reaction is filtered through a 0.22 μm filter to provide crude product.

SP SEPHAROSE® Fast Flow cation exchange chromatography is then conducted on the crude product to partially separate PEGylated reaction fractions. The SP SEPHAROSE® Fast Flow cation exchange chromatography column is equilibrated and the feed loaded at room temperature at a residence time of ˜5 minutes, followed by 5 CV (column volumes) of wash with loading buffer. The PEGylated rhIL-2 binds to the resin while free PEG is washed out. The product is then eluted using a linear gradient elution with 0-500 mM sodium chloride in 10 mM sodium acetate pH 4.0 buffer background. Fractions are collected of 0.15 CV each, starting ˜1 CV into the elution. Fraction collection is ended when absorbance at 280 nm was <5% of peak max. PEGylated fraction concentrations (i.e., mono-PEGylated IL-2 (monomer), di-PEGylated IL-2 (dimer), tri-PEGylated IL-2 (trimer), tetra-PEGylated IL-2 (tetramer), etc., in each of the fractions is measured by absorbance at a wavelength of 280 nm. The distribution of PEGylated fractions is measured by RP-HPLC as described herein or otherwise known to skilled artisan, and the fractions containing mono-PEG, di-PEG, tri-PEG, and higher components, are identified, and used to determine the re-pooling of the necessary fractions to generate compositions that will have the target PEGylated fraction distribution profile, as described in an RUR_{20kD}-IL-2 composition as provided herein, and particular in Formulae A-F. Aliquots of selected fractions of identified composition, e.g. di-PEG-IL-2 and tri-PEG-IL-2, and/or mono-PEG or higher PEG, are calculated so as to achieve the target profile as provided herein, and are then re-pooled as needed to obtain an RUR_{20kD}-IL-2 composition having a product with the desired distribution of PEGylated fractions. Alternatively, purification schemes can be devised whereby the elution and collection may provide the desired profile according to the embodiments descried herein without the need for re-pooling. The desired (and/or re-pooled) chromatography purified preparation is then concentrated and diafiltered into 10 mM sodium acetate, 150 mM sodium chloride, 2% w/v sucrose, pH 5.0 using tangential flow filtration (TFF), to achieve a final target concentration of 1 mg/mL (protein basis) of an RUR_{20kD}-IL-2 composition drug substance.

Re-pooled and/or target products are analyzed and the composition distribution is verified by methods described herein, including RP-HPLC, to assess the profile of PEG fractions. Preparations of compositions according to the specifications herein for an RUR_{20kD}-IL-2 composition of Formulae A-F are illustrated by the example product batches numbered 1-4 listed in Table 1 below. Assays for attributes are known to the skilled artisan, and/or described in assays methods described herein, or for instance in WO 2019/226,538. Appropriate historical reference sample compositions are established and are used for comparison in subsequent preparations.

TABLE 1
Summary of Illustrative Analyses of Samples from Different Batches
of an RUR_{20kD}-IL-2 composition by RP-HPLC and SEC-HPLC

| | Fraction | | | | |
|---|---|---|---|---|---|
| | (where | | | | |
| Attribute | applicable) | Batch 1 | Batch 2 | Batch 3 | Batch 4 |
| Appearance | NA | Clear, | Slightly | Clear, | Clear, |
| | | colorless | opalescent, | colorless | colorless |
| of sample | | liquid | colorless | liquid | liquid |
| | | | liquid | | |
| pH | NA | 5.1 | 5.0 | 5.1 | 5.1 |
| Identity by | NA | Conforms | Conforms | Conforms | Conforms |
| SDS-PAGE | | | to | to | to |
| | | | reference | reference | reference |
| Identity by | NA | Conforms | Conforms | Conforms | Conforms |
| RP-HPLC | | | to | to | to |
| | | | reference | reference | reference |
| Protein | NA | 1.58 | 0.96 mg/ | 1.00 mg/ | 1.12 |
| Content by | | mg/mL | mL | mL | mg/mL |
| BCA | | | | | |
| (vs. rhIL-2) | | | | | |
| Purity | Free IL-2 | <0.1% | ND (not | ND (not | ND (not |
| RP-HPLC | | | more | more than | more than |
| | | | than 0.3%) | 0.3%) | 0.3%) |
| | Mono- | 3.0% | 3.5% | 3.1% | 3.1% |
| | PEG | | | | |
| | Di-PEG | 42.4% | 45.8% | 46.8% | 44.4% |
| | Tri-PEG | 46.6% | 42.6% | 42.4% | 44.8% |
| | Higher | 8.0% | 8.1% | 7.7% | 7.7% |
| | PEGylated | | | | |
| | Others | ND | ND (not | ND (not | ND (not |
| | | | more | more than | more than |
| | | | than 0.5%) | 0.5%) | 0.5%) |
| | Di- | 89.0% | 88.4% | 89.2% | 89.2% |
| | PEG/Tri- | | | | |
| | PEG Total | | | | |
| Purity | Low | 3.0% | 3.0% | 2.4% | 1.4% |
| SEC-HPLC | Molecular | | | | |
| | Weight | | | | |
| | (Mono) | | | | |
| | Di-PEG | 47.0% | 48.6% | 50.7% | 47.2% |
| | Tri-PEG | 45.0% | 42.5% | 41.2% | 45.6% |
| | High | 4.9% | 5.8% | 5.8% | 5.8% |
| | Molecular | | | | |
| | Weight | | | | |
| | Di- | 92.0% | 91.1% | 91.9% | 92.8% |
| | PEG/Tri- | | | | |
| | PEG Total | | | | |
| Free PEG | | <0.1% | ND (NMT | ND (NMT | ND (NMT |
| (HPLC/ELS | | | 0.1%) | 0.1%) | 0.1%) |
| D) | | | | | |

ND is not detectable, NMT is not more than.

In some embodiments the RUR_{20kD}-IL-2 composition product will contain, on a molar basis, less than 1% free, unconjugated IL-2 (more preferably no detectable free IL-2), 5% or less mono-PEGylated IL-2, from 28% to about 60% di-PEGylated IL-2, from about 24% to about 65% tri-PEGylated IL-2, 12% or less of higher PEGylated IL-2 species, and 80% or greater combined di- and tri-PEGylated IL-2 species.

In some embodiments, the RUR_{20kD}-IL-2 composition product will contain, for example, less than 0.5 mol % free IL-2, from about 2.5 to about 4.5 mol % mono-PEGylated IL-2, from about 35 to about 50 mol % di-PEGylated IL-2, from about 38 to about 46 mol % tri-PEGylated IL-2, from about 3 to about 10 mol % higher PEGylated IL-2 species, and a combined total of di-PEGylated and tri-PEGylated IL-2 from about 80 to about 95 mol %.

In some embodiments, the RUR_{20kD}-IL-2 composition product will contain, for example, on a molar basis, 5% or less mono-PEGylated IL-2, and from 28% to about 60% di-PEGylated IL-2, and from about 24% to about 65% tri-PEGylated IL-2, and 12% or less of higher PEGylated IL-2 species. Preferably the composition comprises 80% or greater combined di- and tri-PEGylated IL-2 species.

In some embodiments, the RUR_{20kD}-IL-2 composition product will contain, for example, about 2.5 to about 4.5 mol % comprises mono-PEGylated IL-2, and from about 35 to about 50 mol % comprises di-PEGylated IL-2, and from about 38 to about 46 mol % comprises tri-PEGylated IL-2, and from about 3 to about 10 mol % comprises higher PEGylated IL-2 species. Preferably the composition comprises a combined total of di-PEGylated and tri-PEGylated IL-2 from about 80 to about 95 mol %.

In some embodiments, the RUR_{20kD}-IL-2 composition product will contain, for example, from about 2.8 to about 3.8 mol % comprises mono-PEGylated IL-2, and from about 44 to about 48 mol % comprises di-PEGylated IL-2, and from about 41 to about 44 mol % comprises tri-PEGylated IL-2, and from about 7 to about 9 mol % comprises higher PEGylated IL-2 species. Preferably the composition comprises a combined total of di-PEGylated and tri-PEGylated IL-2 from about 87 to about 90 mol %.

In some embodiments, the RUR_{20kD}-IL-2 composition product will contain, for example, about 2.8 to about 3.8 mol % comprises mono-PEGylated IL-2, and from about 44 to about 48 mol % comprises di-PEGylated IL-2, and from about 41 to about 44 mol % comprises tri-PEGylated IL-2, and from about 7 to about 9 mol % comprises higher PEGylated IL-2 species, and wherein said composition comprises a mixture of mono-PEGylated IL-2 conjugates which have a PEG moiety attached at one of lysine K7 or K8 or K31 or K75. Preferably the composition comprises a combined total of di-PEGylated and tri-PEGylated IL-2 from about 87 to about 90 mol %.

In some embodiments, the RUR_{20kD}-IL-2 composition product will contain, for example, about 2.8 to about 3.8 mol % comprises mono-PEGylated IL-2, and from about 44 to about 48 mol % comprises di-PEGylated IL-2, and from about 41 to about 44 mol % comprises tri-PEGylated IL-2, and from about 7 to about 9 mol % comprises higher PEGylated IL-2 species, and wherein said composition comprises mono-PEGylated IL-2 conjugates which have a PEG moiety attached at lysine K7. Preferably the composition comprises a combined total of di-PEGylated and tri-PEGylated IL-2 from about 87 to about 90 mol %.

In some embodiments, the RUR_{20kD}-IL-2 composition product will contain, for example, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons.

In some embodiments, an RUR_{20kD}-IL-2 and related composition embodiment is referred to herein as “rezpegaldesleukin”, which is prepared according to methods described herein, and/or described in WO 2019/226,538 (incorporated herein by reference), and “rezpegaldesleukin” compositions fall within, for example, a composition of Formula A, and comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; and/or “rezpegaldesleukin” compositions fall within, for example, a composition of Formula F, and comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons; and/or “rezpegaldesleukin” compositions are illustrated, for instance, by Batches 1-4 described in Table 1 above. “Rezpegaldesleukin” conjugate compositions have been provided CAS registration number 2641686-76-0.

### Example 1-B

### Purity and Characterization of an RUR_{20kD}-IL-2 Composition Via Reverse Phase High Performance Liquid Chromatography

Reverse phase high performance liquid chromatography (RP-HPLC) is used to assess the chromatographic purity and identity of samples of an RUR_{20kD}-IL-2 composition using an Agilent 1200 series instrument equipped with a diode array detector (UV at 215 nm). The column used can be an ACE 3 Phenyl-300 column (Mac-Mod Analytical Inc.) (or other suitable column) with an eluent flow rate of 0.6 mL/min. RP-HPLC is carried out using a gradient containing mixtures of two mobile phases: (1) Mobile Phase A, a solution of 0.1% formic acid in water, and (2) Mobile Phase B, a solution of 0.1% formic acid in acetonitrile. The linear gradient ranged from 60% Mobile Phase A/40% Mobile Phase B to 40% Mobile Phase A/60% Mobile Phase B, to 20% Mobile Phase A/80% Mobile Phase B, to 60% Mobile Phase A/40% Mobile Phase B. The components of the diluent/formulation buffer are 10 mM sodium acetate, 200 mM sodium chloride, 2% sucrose, at a pH of 5.0.

Frozen RUR_{20kD}-IL-2 composition reference material and samples are thawed and diluted to 1.0 mg/mL with formulation buffer. At least one blank control of formulation buffer is first subjected to RP-HPLC via injection to ensure no interference with analysis of RUR_{20kD}-IL-2-composition related peaks. Next, RUR_{20kD}-IL-2 composition reference material or control was injected five times. RUR_{20kD}-IL-2 composition samples are next injected. RUR_{20kD}-IL-2 composition reference material/control is injected after every six sample injections and at the end of the injection sequence.

The % relative standard deviation (RSD) of retention time for the first five reference material injections comprising di-PEGylated (di-PEG) and tri-PEGylated (tri-PEG) RUR_{20kD}-IL-2 compositions are not more than 2.0%. The % RSD area percent for all reference material RUR_{20kD}-IL-2 composition injections of the di-PEG and tri-PEG components are not more than 5.0%. All RUR_{20kD}-IL-2 composition peaks from reference and sample injections are integrated. Specifically, for a 1.0 mg/mL concentration of an RUR_{20kD}-IL-2 composition, the di-PEG and tri-PEG RUR_{20kD}-IL-2 composition species above a 0.5% limit of detection (LOD) and the rhIL-2 peak above a 0.3% LOD are respectively integrated. For a 1.0 mg/mL concentration of RUR_{20kD}-IL-2, the limit of quantitation (LOQ) is 1.0% for di-PEG and tri-PEG RUR_{20kD}-IL-2 species and 0.5% for rhIL-2. Results from analyses are shown in Table 2 (6 samples) and Table 3 (12 samples) below.

TABLE 2
Area percent for Mono-PEG, Di-PEG, Tri-PEG, Tetra-PEG,
and Penta-PEG fractions from six RUR_{20 kD}-IL-2
composition replicate samples analyzed by RP-HPLC

| | | Mono- | Di- | Tri- | Tetra- | Penta- |
|---|---|---|---|---|---|---|
| | | PEG | PEG | PEG | PEG | PEG |
| Sample | Result | (% | (% | (% | (% | (% |
| Name | Id | Area) | Area) | Area) | Area) | Area) |
| Sample 1 | 1084 | 2.93 | 42.54 | 46.69 | 7.10 | 0.75 |
| Sample 2 | 1085 | 2.98 | 42.48 | 46.67 | 7.17 | 0.70 |
| Sample 3 | 1086 | 2.98 | 42.60 | 46.65 | 7.21 | 0.56 |
| Sample 4 | 1087 | 3.04 | 42.58 | 46.50 | 7.18 | 0.71 |
| Sample 5 | 1088 | 2.97 | 42.52 | 46.62 | 7.33 | 0.56 |
| Sample 6 | 1089 | 2.88 | 42.51 | 46.69 | 7.34 | 0.58 |

| | | | | | |
|---|---|---|---|---|---|
| Mean | 2.96 | 42.54 | 46.64 | 7.22 | 0.64 |
| % RSD (not more than | 1.8 | 0.1 | 0.2 | 1.3 | 13.3 |
| 2.0% for Di-PEG | | | | | |
| and Tri-PEG) | | | | | |

TABLE 3
Area percent for Mono-PEG, Di-PEG, Tri-PEG, Tetra-PEG,
and Penta-PEG fractions from twelve RUR_{20 kD}-IL-2
composition replicate samples analyzed by RP-HPLC

| | | Mono- | Di- | Tri- | Tetra- | Penta- |
|---|---|---|---|---|---|---|
| | | PEG | PEG | PEG | PEG | PEG |
| Sample | Result | (% | (% | (% | (% | (% |
| Name | Id | Area) | Area) | Area) | Area) | Area) |
| Sample 7 | 2323 | 3.30 | 42.37 | 46.20 | 7.84 | 0.29 |
| Sample 8 | 2324 | 3.07 | 42.36 | 46.55 | 7.76 | 0.26 |
| Sample 9 | 2325 | 3.10 | 42.30 | 46.59 | 7.69 | 0.30 |
| Sample 10 | 2326 | 3.02 | 42.37 | 46.59 | 7.75 | 0.26 |
| Sample 11 | 2327 | 3.10 | 42.29 | 46.59 | 7.75 | 0.26 |
| Sample 12 | 2328 | 3.05 | 42.33 | 46.58 | 7.78 | 0.26 |
| Sample 13 | 1084 | 2.93 | 42.54 | 46.69 | 7.10 | 0.75 |
| Sample 14 | 1085 | 2.98 | 42.48 | 46.67 | 7.17 | 0.70 |
| Sample 15 | 1086 | 2.98 | 42.60 | 46.65 | 7.21 | 0.56 |
| Sample 16 | 1087 | 3.04 | 42.58 | 46.50 | 7.18 | 0.71 |
| Sample 17 | 1088 | 2.97 | 42.52 | 46.62 | 7.33 | 0.56 |
| Sample 18 | 1089 | 2.88 | 42.51 | 46.69 | 7.34 | 0.58 |

| | | | | | |
|---|---|---|---|---|---|
| Mean | 3.04 | 42.44 | 46.58 | 7.49 | 0.46 |
| % RSD (not more than | 3.5 | 0.3 | 0.3 | 3.9 | 44.4 |
| 2.0% for Di-PEG | | | | | |
| and Tri-PEG) | | | | | |

### Example 1-C

### Purity and Characterization of an RUR_{20kD}-IL-2 Composition Via Size Exclusion High Performance Liquid Chromatography

Size exclusion high performance liquid chromatography (SEC-HPLC) can also be used to determine the purity and characterize an RUR_{20kD}-IL-2 composition using an Agilent 1200 series instrument fitted with a diode array detector (UV at 280 nm) and a Yarra SEC-2000 column (Phenomenex), and an eluent flow rate of 0.225 mL/minute. The mobile phase is 0.2M ammonium acetate (pH 5.5) at a volume ratio of 80:20 with acetonitrile. The diluent/formulation buffer contained 10 mM sodium acetate, 200 mM sodium chloride, 2% sucrose, at a pH of 5.0.

Frozen RUR_{20kD}-IL-2 composition reference material and analytical samples are thawed and diluted to 1.0 mg/mL with formulation buffer. Samples are stable up to 5 days at 5° C. in solution.

Procedurally, at least one blank control of formulation buffer is first subjected to RP-HPLC via injection to ensure no interference with analysis of RUR_{20kD}-IL-2-related peaks. Next, the RUR_{20kD}-IL-2 composition, system suitability solution, is injected to ensure that aggregates or higher molecular weight species are resolved from tetra-PEG RUR_{20kD}-IL-2 fractions. RUR_{20kD}-IL-2 composition reference material or control is subsequently injected five times. RUR_{20kD}-IL-2 composition samples are next injected. RUR_{20kD}-IL-2 composition reference material/control is injected after every six sample injections and at the end of the injection sequence.

The % RSD of retention time of di-PEG and tri-PEG RUR_{20kD}-IL-2 fractions, for the first five reference material injections, is not more than 2.0%. The % RSD area percent of di-PEG and tri-PEG RUR_{20kD}-IL-2 for all reference material injections is not more than 5.0%. All RUR_{20kD}-IL-2 fraction peaks from reference and sample injections are integrated. Specifically, for a 1.0 mg/mL concentration of RUR_{20kD}-IL-2 composition, the di-PEG and tri-PEG RUR_{20kD}-IL-2 fractions above a 1.0% limit of detection (LOD) are integrated. For a 1.0 mg/mL concentration of RUR_{20kD}-IL-2, only di-PEG and tri-PEG RUR_{20kD}-IL-2 above a 3.0% LOQ were reported.

Analyses of replicate samples of RUR_{20kD}-IL-2 compositions are shown below in Tables 4 and 5, where peak areas are provided for mono-PEG, di-PEG, tri-PEG, tetra-PEG, and penta-PEG fractions of the RUR_{20kD}-IL-2 composition.

TABLE 4
% Peak areas for Mono-PEG, Di-PEG, Tri-PEG, Tetra-PEG,
and Penta-PEG components of RUR_{20 kD}-IL-2 by SEC-HPLC

| | | RT | | | | | | RT | |
|---|---|---|---|---|---|---|---|---|---|
| | | (min) | Area % | RT | | RT | | (min) | Area % |
| Sample | Result | Tetra- | Tetra- | (min) | Area % | (min) | Area % | Mono- | Mono- |
| Name | Id | PEG | PEG | Tri-PEG | Tri-PEG | Di-PEG | Di-PEG | PEG | PEG |
| Sample 1 | 1119 | 21.62 | 4.59 | 22.40 | 46.73 | 23.91 | 46.99 | 28.65 | 1.70 |
| Sample 2 | 1120 | 21.62 | 4.53 | 22.41 | 46.67 | 23.91 | 47.11 | 28.66 | 1.69 |
| Sample 3 | 1121 | 21.62 | 4.45 | 22.41 | 46.75 | 23.92 | 47.04 | 28.75 | 1.76 |
| Sample 4 | 1122 | 21.63 | 4.66 | 22.42 | 46.68 | 23.92 | 46.91 | 28.76 | 1.75 |
| Sample 5 | 1123 | 21.64 | 4.63 | 22.44 | 46.81 | 23.94 | 46.80 | 28.76 | 1.77 |
| Sample 6 | 1124 | 21.64 | 4.54 | 22.43 | 46.79 | 23.94 | 46.87 | 28.86 | 1.80 |
| Mean | NA | 21.63 | 4.57 | 22.42 | 46.74 | 23.92 | 46.95 | 28.74 | 1.74 |
| % RSD | NA | 0.0 | 1.6 | 0.0 | 0.1 (not | 0.1 | 0.2 (not | 0.3 | 2.4 |
| (Acceptance | | (NA) | (NA) | (NA) | more | (NA) | more | (NA) | (NA) |
| Criteria) | | | | | than | | than | | |
| | | | | | 10.0%) | | 10.0%) | | |

TABLE 5
% Peak areas for Mono-PEG, Di-PEG, Tri-PEG, Tetra-PEG, and Penta-PEG
fractions of RUR_{20 kD}-IL-2 composition samples by SEC-HPLC

| | | RT | | | | | | RT | |
|---|---|---|---|---|---|---|---|---|---|
| | | (min) | Area % | RT | | RT | | (min) | Area % |
| Sample | Result | Tetra- | Tetra- | (min) | Area % | (min) | Area % | Mono- | Mono- |
| Name | Id | PEG | PEG | Tri-PEG | Tri-PEG | Di-PEG | Di-PEG | PEG | PEG |
| Sample 7 | 2021 | 23.32 | 4.93 | 24.12 | 45.21 | 25.58 | 46.82 | 29.39 | 3.04 |
| Sample 8 | 2022 | 23.32 | 4.83 | 24.10 | 45.67 | 25.58 | 46.97 | 29.42 | 2.52 |
| Sample 9 | 2023 | 23.32 | 4.74 | 24.11 | 45.08 | 25.59 | 46.66 | 29.44 | 3.52 |
| Sample 10 | 2024 | 23.32 | 4.90 | 24.11 | 45.05 | 25.59 | 46.74 | 29.41 | 3.31 |
| Sample 11 | 2025 | 23.31 | 4.89 | 24.11 | 45.00 | 25.58 | 46.72 | 29.41 | 3.39 |
| Sample 12 | 2026 | 23.32 | 4.86 | 24.11 | 45.12 | 25.59 | 46.90 | 29.48 | 3.12 |
| Sample 13 | 1119 | 21.62 | 4.59 | 22.40 | 46.73 | 23.91 | 46.99 | 28.65 | 1.70 |
| Sample 14 | 1120 | 21.62 | 4.53 | 22.41 | 46.67 | 23.91 | 47.11 | 28.66 | 1.69 |
| Sample 15 | 1121 | 21.62 | 4.45 | 22.41 | 46.75 | 23.92 | 47.04 | 28.75 | 1.76 |
| Sample 16 | 1122 | 21.63 | 4.66 | 22.42 | 46.68 | 23.92 | 46.91 | 28.76 | 1.75 |
| Sample 17 | 1123 | 21.64 | 4.63 | 22.44 | 46.81 | 23.94 | 46.80 | 28.76 | 1.77 |
| Sample 18 | 1124 | 21.64 | 4.54 | 22.43 | 46.79 | 23.94 | 46.87 | 28.86 | 1.80 |
| Mean | NA | 22.47 | 4.71 | 23.26 | 45.96 | 24.75 | 46.88 | 29.08 | 2.45 |
| % RSD | NA | 3.9 | 3.5 | 3.8 | 1.8 (not | 3.5 | 0.3 (not | 1.2 | 31.6 |
| (Acceptance | | (NA) | (NA) | (NA) | more | (NA) | more | (NA) | (NA) |
| Criteria) | | | | | than | | than | | |
| | | | | | 10.0%) | | 10.0%) | | |

A summary of representative analyses of different samples of an RUR_{20kD}-IL-2 composition by both RP-HPLC and SEC-HPLC is shown in Table 1. As can be seen, RUR_{20kD}-IL-2 composition preparations are demonstrate good batch-to-batch consistency with respect to the mixtures of PEGylated fractions (i.e., mono-PEGylated, di-PEGylated, tri-PEGylated, tetra-PEGylated, penta-PEGylated, etc.).

### Example 1-D

### SDS-Page

SDS-PAGE is utilized for the confirmation of an RUR_{20kD}-IL-2 composition identity. Samples of an RUR_{20kD}-IL-2 composition, a molecular weight marker, and an appropriate RUR_{20kD}-IL-2 composition reference material are loaded onto a NuPAGE Bis-Tris gel and migrated through the gel. Following electrophoresis, the gels are stained using GelCode™ Blue Safe Protein Stain. Comparison of the gel migration banding pattern to the reference material and confirmation of no new bands in the sample confirms the identity of the samples. The two most intense bands will correspond to the tri-PEGylated & the di-PEGylated fractions. The upper most band in the lanes corresponds to higher PEGylated variants and the lowest band corresponds to the mono-PEGylated variants.

### Example 1-E

### CTLL-2 Cell Proliferation Assay

In the cell proliferation assay, cell growth is measured in vitro using CTLL-2 cells following incubation of sample and reference for ˜26 hours where cell proliferation is measured via luminescence adenosine triphosphate-based assay (CellTiter-Glo®, Promega, WI). For example, this cell-based proliferation assay uses the CTLL-2 cell line, which exhibits a dose-dependent proliferation response to rhIL-2 protein. rhIL-2 is used as the assay control and is prepared at a different concentration range from an RUR_{20kD}-IL-2 composition in this method. This assay is performed in a 96-well plate format. CTLL-2 cells are starved of rhIL-2 in starvation media and incubated overnight for 20±3 hours in a 37° C. and 5% CO₂ incubator. Starved cells are plated in 96-well plates and a dilution series of RUR_{20kD}-IL-2 composition is fed to the cells and incubated for another 25±3 hours in a 37° C. and 5% CO₂ incubator. RUR_{20kD}-IL-2 composition induced cell growth in each well is measured using a CellTiter Glo® detection kit by Promega. CellTiter Glo® generates a luminescent signal proportional to the amount of ATP present in each well, which is directly proportional to the viable cells present. The luminescence signal is read on a SpectraMax M5 plate reader. A dose response curve of RUR_{20kD}-IL-2 composition reference material and each sample is generated by plotting luminescent signal (y-axis) to concentrations (x-axis). The plot is fitted to a 4-parameter logistic nonlinear regression model. Parallel Line Analysis (PLA) software is used to assess the Equivalence Test for Difference of Slopes (parallelism), Significance of Regression, and to calculate the potency ratio of the sample in relation to the reference material in the same plate.

### Example 1-F

### Phospho-STAT5 Activation

In the phospho-STAT5 assay following receptor binding, downstream cell signaling can then activate Signal Transducer and Activator of Transcription 5 (STAT5) through phosphorylation to promote gene expression to induce cell proliferation. The activation of phosho-STAT5 is measured in CTLL-2 cells, an IL-2-dependent murine T lymphocyte cell line, using the phospho-STAT5/total STAT5 multiplexed assay (Meso Scale Discovery, MD) in response to sample and reference treatment for ˜10 minutes.

### RUR_{20kD}-IL-2 Selective Treg Stimulator Composition Formulations and Delivery Devices

In yet one or more embodiments provided herein is a selective Treg stimulator composition, including RUR_{20kD}-IL-2 embodiments and related compositions, comprising an IL-2 conjugate composition as described herein, and a pharmaceutically acceptable excipient.

“Pharmaceutically acceptable excipient” or “pharmaceutically acceptable carrier” refers to a component that may be included in the compositions described herein and causes no significant adverse toxicological effects to a subject. Additional suitable pharmaceutically acceptable excipients include those described, for example, in the Handbook of Pharmaceutical Excipients, 7^{th} ed., Rowe, R. C., Ed., Pharmaceutical Press, 2012. The compositions of the present disclosure are preferably formulated as pharmaceutical compositions administered by any route that makes the composition bioavailable, such as parenteral administration, including intravenous, intramuscular or subcutaneous. Subcutaneous administration is preferred for the embodiments of the present disclosure. Such pharmaceutical compositions and processes for preparing same are well known in the art (See, e.g., Remington: The Science and Practice of Pharmacy (D. B. Troy, Editor, 21 st Edition, Lippincott, Williams & Wilkins, 2006)). Useful formulations are described in WO 2019/226,538. Additional and preferred formulations are described below.

In some embodiments, rezpegaldesleukin or a pharmaceutical formulation comprising the rezpegaldesleukin, such as 5 mM sodium acetate, 25 mM sodium chloride, 7.5% (w/v) sucrose, pH 5.0, is administered to the patient using a subcutaneous administration device. The subcutaneous administration device can be selected from a prefilled syringe, disposable pen injection device, microneedle device, microinfuser device, needle-free injection device, or autoinjector device. Various subcutaneous administration devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic).

A preferred formulation of the selective Treg stimulator compositions, including RUR_{20kD}-IL-2 embodiments and related compositions provided herein, including rezpegaldesleukin, is 5 mM sodium acetate, 25 mM sodium chloride, 7.5% (w/v) sucrose, pH 5.0. An RUR_{20kD}-IL-2 composition can be stored in sterile single-use polycarbonate bottles of appropriate volume with a polypropylene cap with a silicone liner, supplied sterile and ready-to-use. The drug product API (e.g. rezpegaldesleukin) concentration for a 1800 μg dose is selected from 0.9 or 1.8 or 3.6 mg/mL protein equivalent, depending on the autoinjector or prefilled syringe being used. A preferred delivery device is an autoinjector is selected from a 0.5 ml auto-injector or a 1 ml auto-injector or a 2 ml auto-injector as suitable for the selected dose, or a pre-filled syringe corresponding to the same dose volumes. For a 3000 μg dose the drug product API (e.g. rezpegaldesleukin) concentration would be 1.5 or 3.0 or 6.0 mg/mL (depending on the autoinjector or prefilled syringe). Other concentrations needed to achieve the dose and volumes needed can be determined by methods described herein and or known to the skilled artisan.

Rezpegaldesleukin is being studied in several ongoing or completed trials, including a Phase 2 trial of rezpegaldesleukin in patients with SLE (NCT04433585), a Phase 2 trial of Rezpegaldesleukin in patients with moderately-to-severely active ulcerative colitis (NCT 04677179) and a Phase 1b study in patients with atopic dermatitis (NCT04081350) and psoriasis (NCT04119557). Embodiments of the present disclosure described herein can be examined clinically by methods described herein or know to the skilled artisan, for instance the trial methods described in WO 2019/226,538 for patients with SLE. The fixed unit doses and or induction and maintenance dosing regimens described herein can be evaluate in trials with modifications being made to adapt to the limitations of the disclosed embodiments for dose, formulation, means of administration, patient selection, dosing frequency, dosing duration, and changes embodied by the induction and maintenance regimens of the present disclosure.

### Example 2

A Phase 1, Double-Blind, Randomized, Placebo-Controlled, Multiple-Dose Study to Evaluate the Safety, Tolerability, and Pharmacokinetics of Subcutaneous Rezpegaldesleukin in Patients with Atopic Dermatitis

Rationale: Study JIP-MC-KFAD is a Phase 1 study designed to evaluate the safety, tolerability, and pharmacokinetics (PK) of multiple subcutaneous (SC) doses of rezpegaldesleukin in patients with atopic dermatitis (AD). Rezpegaldesleukin is recombinant human interleukin 2 (rhIL-2) with stable covalently attached polyethylene glycol (PEG) moieties. IL-2 has pleiotropic immunoregulatory functions and has a role in the control of the proliferation and survival of regulatory T (Treg) cells, which are impaired in various autoimmune diseases and inflammatory skin diseases, including AD. Analysis of skin lesions from patients with AD has shown either a decrease in Treg cell numbers or a reduction in immunosuppressive functions of Treg cells (Nedoszytko et al. 2017). Moreover, filaggrin null mutations correlate with altered circulating Tregs in patients with AD (Moosbrugger-Martinz et al. 2018). The proliferative and functional deficit of Treg cells is hypothesized to contribute to the pathogenesis of AD and other skin-related inflammatory diseases, and treatment with a low-dose IL-2 conjugate therapy (rezpegaldesleukin) offers a potential path to overcome the imbalance between conventional T (Tcon) cells and Treg cells. The goal of the rezpegaldesleukin therapy is to increase Treg cell number and function with minimal effect on Tcon cells in patients with inflammatory diseases such as AD, which could translate to a beneficial clinical outcome.

### Objective(s)/Endpoints:

| Objectives | Endpoints |
|---|---|
| Primary | Incidence of adverse |
| To evaluate the safety and tolerability of | events, treatment- |
| multiple SC doses of rezpegaldesleukin | emergent adverse |
| administered to patients with AD | events, and serious |
| | adverse events |
| Secondary | Cmax, Tmax, and AUC |
| To quantify rezpegaldesleukin plasma | after the first dose |
| concentrations following multiple SC | and troμgh concentrations |
| doses in patients with AD | after repeated |
| | dosing. |

Abbreviations: AD = atopic dermatitis; AUC = area under the plasma concentration versus time curve; Cmax = maximum observed plasma concentration; SC = subcutaneous; Tmax = time to maximum observed plasma concentration.

This is a double-blind, randomized, placebo-controlled study to evaluate the safety, tolerability, PK, and PD effects of multiple doses of rezpegaldesleukin in up to 2 cohorts of patients with AD.
Treatment Period (through Week 12): rezpegaldesleukin or placebo will be administered SC every 2 weeks for a total of 7 doses per patient. The treatment period will be 12 weeks, during which blood and skin samples will be collected for PK and PD measurements, as well as physician- and patient-assessed outcome and safety measures. The key exploratory disease activity assessment will be done at Day 85 (Week 12), prior to the final dose of study drug.
Follow-up Period (from Week 12 through Week 19): All patients will be monitored through Day 134 (Week 19), which is 50 days after the last dose of study drug, for safety, PK, PD, and disease activity assessment. Patients who do not meet an Eczema Area and Severity Index (EASI) 50 response on Day 134 will be considered to have completed the study.
Follow-up Period for Sustained EASI 50 Responders (from Week 19 through Week 48): To evaluate durability of response, patients who are EASI 50 (patient's EASI score reduced by at least 50% relative to their baseline score) responders on Day 134 and agree to continue, will continue through Week 48 or until they fail to meet an EASI 25 response. Patients who continue will visit every 4 weeks starting at Week 24 to evaluate disease activity and PK through Day 337 (Week 48).
Approximately 50 patients with active AD are planned to be randomly assigned to either rezpegaldesleukin or placebo. Each cohort is planned to have up to 20 patients randomly assigned to receive rezpegaldesleukin and up to 5 patients randomly assigned to receive placebo (matching saline solution). This will allow for approximately 40 patients completing the study with both cohorts. End of the study is the date of the last patient visit or last scheduled procedure shown in the Schedule of Activities for the last patient.
Statistical Analysis: The safety population will consist of all patients who receive at least 1 dose of study drug according to the randomization. The PK population will consist of all randomized patients who receive rezpegaldesleukin and have adequate PK data to permit a meaningful analysis. Adverse events will be classified according to the Medical Dictionary for Regulatory Activities system organ class and preferred term and will be summarized by treatment. Other safety data including vital signs, and clinical laboratory tests will be summarized by treatment. Pharmacokinetic parameters will be calculated from plasma concentration-time data after the first dose using standard noncompartmental methods of analysis and summarized using descriptive statistics. Data listings and summary statistics will be provided for safety by treatment group over time. For continuous variables, summary statistics will include the mean, standard deviation, minimum, maximum, median, and number of observations. For categorical variables, frequency counts and percentages will be provided.

### Schedule of Activities

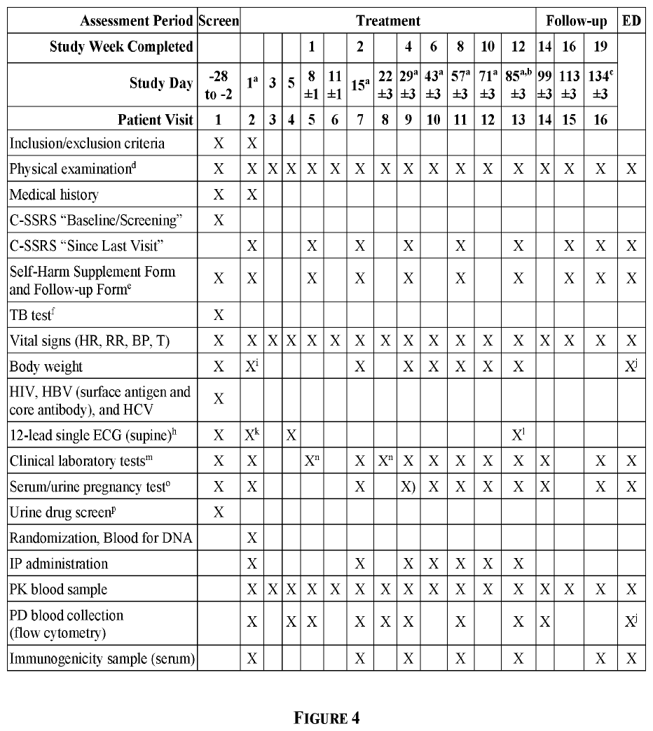

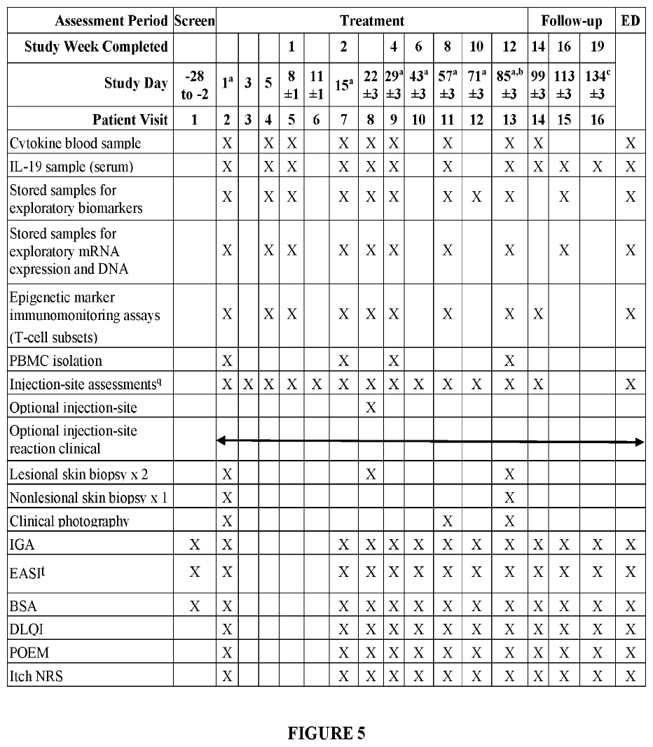

### Study Schedule Protocol J1P-MC-KFAD: Treatment and Initial Follow-Up (See FIGS. 4 and 5)

Concomitant medications recording and Adverse events recording occur throughout the study.

### Abbreviations for FIGS. 4 and 5:

BP=blood pressure; BSA=body surface area; C—SSRS=Columbia Suicide Severity Rating Scale; DLQI=Dermatology Life Quality Index; DNA=deoxyribonucleic acid; EASI=Eczema Area and Severity Index; EASI 50=patient's EASI score reduced by at least 50% relative to their baseline score; ECG=electrocardiogram; ED=early discontinuation; HBV=hepatitis B virus; HCV=hepatitis C virus; HIV=human immunodeficiency virus; HR=heart rate; IGA=Investigator's Global Assessment; IL=interleukin; IP=investigational product; mRNA=messenger ribonucleic acid; NRS=Numeric Rating Scale; PBMC=peripheral blood mononuclear cell;
PD=pharmacodynamics; POEM=Patient-Oriented Eczema Measure; PK=pharmacokinetics; RR=respiratory rate; T=temperature; TB=tuberculosis; VAS=visual analog scale.
^{a} On days when study drug is administered, all procedures should be completed before the dose is given.
^{b} Day 85 (Week 12): Key exploratory disease activity assessment and administration of last dose.
^{c} Day 134 (Week 19): Last Study Visit for Week 12 EASI 50 non-responders and Week 12 responders who are no longer responding at the time of this visit.
^{d} Full physical examination, excluding breast and genital examinations, at screening and the visit following the last dose of study drug (or ED), which means a full physical examination is required at Visit 14 as well. Symptom-directed physical examinations will be conducted at all other scheduled times, or at times deemed necessary by the investigator.
^{e} The Self-Harm Follow-up Form is required only if triggered by the Self-Harm Supplement Form.
^{f} Perform chest radiograph and either a tuberculin skin test or a QuantiFERON®-TB Gold test.
^{g} On days when study drug is administered, measure vital signs predose and at 2 hours±15 minutes postdose.
^{h} On days having concurrent ECG or vital signs with blood sampling, these should occur at approximately the same time. ECG recording and vital sign measurements (in either order) should occur prior to any blood draw. Patients must be supine for approximately 5 to 10 minutes before ECG or vital sign collection and remain supine but awake during ECG collection.
ⁱ The patient's body weight on Day 1 (baseline) will be used throughout the study to calculate their investigational product dose.
^{j} Collect only if ED occurs prior to Week 12 (Day 85).
^{k} Collect ECG predose and 2 hours (+15 minutes) postdose to look for acute cardiac effects.
^{l} Collect ECG predose to look for cardiac effects at steady state.
^{m} Additional clinical laboratory tests, including local tests, may be performed at any time as determined by the investigator for immediate patient management/safety or as required by local regulations.
ⁿ Only 3 mL blood sample for hematology on Days 8 and 22.
^{o} Serum pregnancy test will be employed for screening visit; urine pregnancy test thereafter.
^{p} A drug screen can be repeated at the discretion of the investigator.
^{q} At the end of each designated visit, the investigator will ask the patient if they have any injection-site concern, and record the patient response per the Injection Site Assessment and Pain VAS Tools, including any new or ongoing injection-site reactions or concerns. The injection-site assessment and pain VAS tools are only completed if there is a new injection-site concern and/or reaction, or an ongoing injection-site reaction or concern from the previous visit.
^{r} If an injection-site reaction occurs, an optional biopsy may be taken, and may be taken at any visit as required, but Visit 8 (Day 22) is preferred. Prior to the collection of the ISR biopsy, consent must be obtained using the ISR biopsy consent form.
^{s} If an injection-site reaction occurs, clinical photography may be used to image the injection site and may be taken at any visit as required.
^{t} EASI 50 responders will be followed-up on to Week 48.

### Study Schedule Protocol J1P-MC-KFAD: Clinical Response Follow-Up for EASI 50 Responders at Week 19 Safety Follow-Up

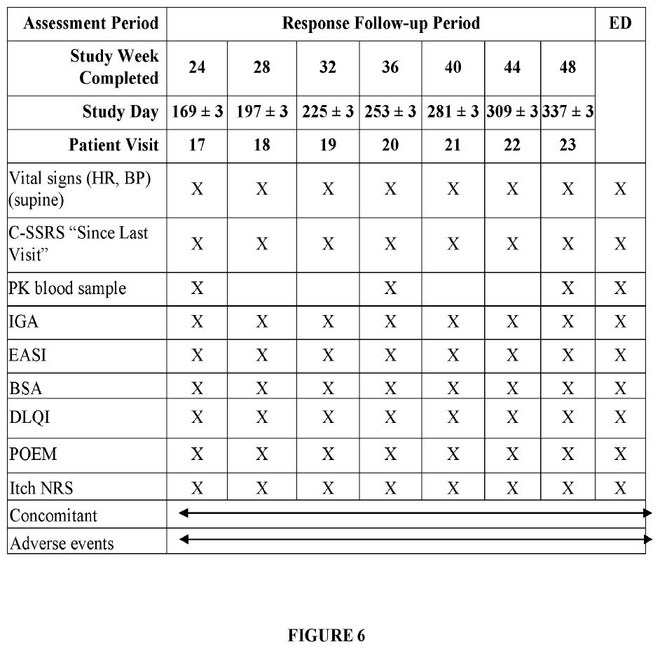

### (See FIG. 6)

Abbreviations: BP=blood pressure; BSA=body surface area; C—SSRS=Columbia Suicide Severity Rating Scale; DLQI=Dermatology Life Quality Index; EASI=Eczema Area and Severity Index; ED=early discontinuation; HR=heart rate; IGA=Investigator's Global Assessment; NRS=Numeric Rating Scale; POEM=Patient-Oriented Eczema Measure; PK=pharmacokinetics.

### Study Population:

Eligibility of patients for the study will be based on the results of screening medical history, physical examination, vital signs, clinical laboratory tests, and ECG. Vital sign measurements may be repeated once if any measurement is out of range. The nature of any conditions present at the time of the physical examination and any preexisting conditions will be documented. Screening may occur up to 28 days prior to enrollment. Patients who are not enrolled within 28 days of screening may be subjected to an additional medical assessment and/or clinical measurements to confirm their eligibility. Prospective approval of protocol deviations to recruitment and enrollment criteria, also known as protocol waivers or exemptions, is not permitted.

### Inclusion Criteria:

Patients are eligible for inclusion in the study only if they meet all of the following criteria at screening and/or enrollment:
1. present with a diagnosis of AD at least 12 months prior to screening, as defined by the American Academy of Dermatology: Guidelines of care for the management of atopic dermatitis; Section 1. Diagnosis and assessment of moderate to severe atopic dermatitis (Eichenfield et al. 2014), including all of the following:
2. EASI score ≥16 at baseline (Day 1)
3. Investigator Global Assessment (IGA) score of ≥3 at baseline (Day 1)
4. ≥10% of body surface area (BSA) involvement at baseline (Day 1)
5. history, documented by a physician and/or investigator, of inadequate response to existing topical medications within 6 months preceding screening, or history of intolerance to topical therapy as defined by at least 1 of the following:
6. inability to achieve good disease control defined as mild disease or better (e.g., IGA≤2) after use of at least a medium potency topical corticosteroid (TCS) for at least 4 weeks, or for the maximum duration recommended by the product prescribing information (e.g., 14 days for super potent TCS), whichever is shorter,
   (Note: a TCS may be used with or without topical calcineurin inhibitors [TCNIs]).
   or
7. documented history of clinically significant adverse reactions with the use of TCS, such as skin atrophy, allergic reactions, or systemic effects that, in the opinion of the investigator, outweigh the benefits of retreatment, and/or
8. failed systemic therapies intended to treat AD, such as cyclosporine, methotrexate, azathioprine, and mycophenolate mofetil, within 6 months preceding screening (will be considered as having inadequate response to topical therapy)
9. agree to discontinue use of the following excluded medications for at least 2 weeks prior to baseline (Day 1) and throughout the study:
10. topical corticosteroids or topical immune modulators (e.g., tacrolimus or pimecrolimus), and
11. topical phosphodiesterase type 4 (PDE 4) inhibitor (crisaborole)
12. have applied emollients daily for at least 14 days prior to baseline (Day 1) and agree to use emollient daily throughout the treatment period
13. males and females, aged 18 through 70 years at the time of screening, who agree to adhere to the contraception requirements
14. body mass index (BMI) of 18.0 to 45.0 kg/m2, inclusive
15. clinical laboratory test results within normal reference range for the population or study site, or results with acceptable deviations that are judged to be not clinically significant by the investigator
16. venous access sufficient to allow for blood sampling
17. reliable and willing to make themselves available for the duration of the study and are willing to follow study procedures
18. willing and able to undergo skin lesion biopsies on study Days 1, 22, and 85
19. willing and able to give signed informed consent, and
20. willing and able to participate in the study for a duration of up to 6 months.

### Exclusion Criteria:

Patients will be excluded from study enrollment if they meet any of the following criteria at screening and/or enrollment:
21. study site personnel directly affiliated with this study and their immediate families (a spouse, biological or legal guardian, child, or sibling)
22. Lilly employees or are employees of a third-party organization involved with the study
23. currently enrolled in a clinical study involving an investigational product (IP) or any other type of medical research judged not to be scientifically or medically compatible with this study
24. previously completed or withdrawn from this study or any other study investigating rezpegaldesleukin, and have previously received the IP
25. known allergies to rezpegaldesleukin, related compounds, or any components of the formulation
26. clinically significant ECG abnormalities including QT interval corrected using Fridericia's formula >450 msec for males and >470 msec for females
27. history of additional risk factors for Torsades de Pointes (e.g., heart failure, hypokalemia, family history of long QT syndrome)
28. use of concomitant medications (excluding medications which could be used to treat depression) that prolong the QT/QTc interval
29. clinically relevant abnormal blood pressure (BP) and/or heart rate (HR) as determined by the investigator
30. evidence of significant liver or kidney dysfunction, including any of the following at screening:
   1. estimated glomerular filtration rate of ≤60 mL/min, using the Modification of Diet in Renal Disease equation:
      1. glomerular filtration rate (mL/min/1.73 m2)=175×(Scr)-1.154×(Age)-0.203×(0.742 if female)× (1.212 if African American)
   2. aspartate aminotransferase (AST), alanine aminotransferase (ALT), or alkaline phosphatase (ALP)>1.5× upper limit of normal (ULN); or
   3. total bilirubin level (TBL)>1.5×ULN
31. history of any significant cardiovascular disease (e.g., myocardial infarction, congestive heart failure, uncontrolled hypertension, cerebrovascular accident), thrombotic episode, or any severe medical illness, in the opinion of the investigator, within the previous 1 year.
32. evidence of significant hematologic dysfunction, including any of the following at screening:
   1. hemoglobin <10.0 g/dL
   2. absolute neutrophil count <1.8×109/L
   3. total white blood cell (WBC)<3.6×109/L
   4. absolute lymphocyte count≤0.8×109/L
   5. platelet count <100×109/L, or
   6. eosinophil count >3000 μL
33. currently experiencing or have a history of other concomitant skin conditions (e.g., PsO or cutaneous lupus) that would interfere with evaluations of the effect of study drug on AD
34. patients who, in the opinion of the investigator, are currently experiencing or have a history of erythrodermic, refractory, or unstable skin disease that requires frequent hospitalizations and/or IV treatment for skin infections that may interfere with participation in the study
35. history of eczema herpeticum within 12 months prior to screening
36. history of 2 or more episodes of eczema herpeticum
37. patients who are currently experiencing a skin infection that requires treatment, or is currently being treated, with topical or systemic antibiotics
   Note:
   Patients may not be rescreened until at least 4 weeks after the date of their previous screen failure and at least 2 weeks after resolution of the infection.
38. any serious concomitant illness that is anticipated to require the use of systemic corticosteroids or otherwise interfere with study participation or require active frequent monitoring (e.g., unstable chronic asthma)
39. history of any disease apart from AD that has required treatment with oral or parenteral corticosteroids for more than 2 weeks within the past 24 weeks prior to signing the informed consent form (ICF)
40. history of a primary immunodeficiency, splenectomy, or any underlying condition that predisposes the subject to infection
41. exclusion criterion has been deleted
42. history of major surgery within 12 weeks of screening or will require major surgery during the study
43. are considered by the investigator to be at significant risk for suicide based on the following criteria:
   the ideation or behavior occurred within the past 6 months, and
   have answered “yes” to either Question 4 or Question 5 on the “Suicidal Ideation” portion of the Columbia Suicide Severity Rating Scale (C—SSRS), or
   have answered “yes” to any of the suicide-related behaviors on the “suicidal behavior” portion of the C—SSRS
44. regularly use known drugs of abuse and/or show positive findings from drug screening
45. history of alcohol or other drug abuse within the last year
46. evidence of HIV infection and/or positive human HIV antibodies
47. evidence of hepatitis B and/or positive hepatitis B surface antigen and/or are positive for hepatitis B core antibody (even if negative for hepatitis B surface antigen) at screening
48. evidence of hepatitis C and/or positive hepatitis C antibody at screening
Note
Patients with a previous diagnosis of hepatitis C who have been treated with antiviral therapy and achieved a sustained virological response may be eligible for inclusion in the study, provided they have no detectable hepatitis C virus (HCV) RNA on the screening test for this protocol. A sustained virological response is defined as an undetectable HCV RNA 24 weeks after completion of a full, documented course of an approved antiviral therapy for HCV.
Patients who have spontaneously cleared HCV infection, defined as:
   a. a positive HCV antibody test and
   b. a negative HCV RNA test, with no history of anti-HCV treatment,
may be eligible for inclusion in the study, provided they have no detectable HCV RNA on screening for this study. Based on the judgment of the investigator, any patient exhibiting behaviors that would put them at risk for re-infection with HCV may be discontinued from the study.
Any patient with a history of HCV infection who develops elevated ALT level ≥3×ULN during the study will be tested for HCV RNA in addition to a full liver evaluation. Anyone diagnosed with hepatitis C during the study will be discontinued from the study and should receive appropriate follow-up medical care.
49. women who are breastfeeding
50. donated blood of more than 500 mL within the previous 4 weeks of study screening
51. received blood products within 6 months prior to screening
52. symptomatic herpes zoster within 3 months of screening
53. show evidence of active or latent TB, as documented through medical history and examination chest x-ray (posterior-anterior; read by a radiologist, pulmonologist, or designee; a lateral chest x-ray may be performed if clinically or radiologically indicated) and/or positive TB testing, defined as: either a positive tuberculin skin test (TST), defined as a skin induration >5 mm at 48 to 72 hours, regardless of prior bacille Calmette-Guerin (BCG) TB vaccination, or
a positive or 2 consecutive indeterminate QuantiFERON®-TB Gold test results
   Note
   The choice to perform a TST or a QuantiFERON-TB Gold test will be made by the investigator according to local licensing and standard of care. The QuantiFERON-TB Gold test can only be used in countries where it is licensed.
54. received live vaccine(s) (including live attenuated vaccines) within 4 weeks of screening or intend to receive during the study and for 5 drug half-lives after the last dose of the study drug (through week 19)
55. received BCG vaccine within 12 months of screening or intend to during the study and for 5 drug half-lives after the last dose of study drug
56. received treatment with biologic agents (such as monoclonal antibodies, including marketed drugs) within 3 months or 5 half-lives (whichever is longer) prior to dosing
   Note
Patients who have been treated with dupilumab must have not received it within 6 months of baseline (Day 1).
57. received any small molecule IP within 4 weeks or 5 half-lives before screening, whichever is greater
58. history of clinically significant drug hypersensitivity reactions that in the opinion of the investigator may predispose the patient to a clinically significant hypersensitivity reaction to rezpegaldesleukin or its formula excipients, or
59. intolerance to TCS
60. received any parenteral corticosteroid administered through intra-articular injection or intramuscular or IV injection within 2 weeks prior to study entry or within 6 weeks prior to planned baseline (Day 1) or are anticipated to require parenteral injection of corticosteroids during the study
Note
   Intranasal or inhaled steroid use is allowed during the trial.
61. received aldesleukin or any investigational IL-2 analog at any time
62. received the following medications/treatments within 4 weeks prior to baseline (Day 1) or plan to continue use throughout the study:
63. oral systemic corticosteroids and leukotriene inhibitors
64. systemic immunomodulators, including, but not limited to, cyclosporine, methotrexate, mycophenolate mofetil, azathioprine, and Janus kinase (JAK) inhibitors (tofacitinib, ruxolitinib)
65. any other systemic therapy used to treat AD or symptoms of AD (approved or off-label use), or
66. phototherapy, includes therapeutic phototherapy (psoralen plus ultraviolet A, ultraviolet B), excimer laser, or tanning beds
67. history of organ or bone marrow transplant
68. lymphoma, leukemia, or any malignancy within the past 5 years, with the following exceptions:
basal cell or squamous epithelial carcinomas of the skin that have been resected with no evidence of metastatic disease for 3 years, or
cervical carcinoma in situ, with no evidence of recurrence within the 5 years prior to baseline
69. breast cancer within the past 10 years, or
70. in the opinion of the investigator or sponsor, are unsuitable for inclusion in the study.
71. have a current or recent acute active infection. For at least 30 days prior to screening, patients must have no significant symptoms including fever of 100.5° F. (38° C.) or above, at screening or baseline, and/or signs of confirmed or suspected infection, and must have completed any appropriate anti-infective treatment.
72. have had, within 6 months of screening, any of the following types of infection:
   1) Serious infections (requiring hospitalization, and/or IV antibiotic treatment)
   2) Opportunistic infections (as defined in Winthrop et al. 2015)
   3) Herpes zoster that is considered active and ongoing until all vesicles are dry and crusted over.
   4) Chronic infections (duration of symptoms, signs, and/or treatment of 6 weeks or longer)
   5) Recurring infection (including, but not limited to, herpes simplex, herpes zoster, recurring cellulitis, chronic osteomyelitis)
      Patients with only recurrent, mild, and uncomplicated orolabial and/or genital herpes may be discussed with Lilly or Lilly-designated medical monitor for possible exemption from this exclusion criterion.

### Treatment:

Rezpegaldesleukin drug product is a sterile solution for SC injection. The placebo dosing solution is 0.9% sodium chloride for injection (US Pharmacopeia). The rezpegaldesleukin drug product and placebo dose preparation for injection will be conducted by the unblinded pharmacist.
Study drug will be administered as an SC injection.
The drug product will be administered using no more than 4 injections, with a volume of no more than 2.0 mL per injection (maximum total volume 8.0 mL).
c. Injection sites selected for SC administration should be in the abdominal region approximately 5 cm from the umbilicus, and the injections should be administered with the needle applied at approximately 45° with pinching of the skin.
d. When more than 1 injection will be administered on a given day, each additional injection should be given to the next abdominal quadrant following a clockwise sequence (only 1 injection per quadrant), and all planned injections to deliver the full intended dose should be administered within a maximum of 5 minutes.
e. If there is a lingering ISR in one abdominal area, that site is not used until the ISR has resolved (so as not to confuse the AE assessment).
f. Subcutaneous administration of study drug should be performed by a limited number of individuals for consistency.

All IPs will be stored, inventoried, reconciled, and destroyed according to applicable regulations. Clinical trial materials are manufactured in accordance with current good manufacturing practices. Rezpegaldesleukin is supplied for clinical trial use as a solution in vial. Each 0.5-mL vial is manufactured to deliver 0.5 mg of rezpegaldesleukin. Vials will be supplied in cartons, with the appropriate quantity specific to the planned dispensing schedule of the IP. Placebo for all cohorts is 0.9% sodium chloride. When prepared for dosing according to instructions, it will not be possible to distinguish between rezpegaldesleukin and placebo. Investigational products will be prepared by an unblinded pharmacy staff or pharmacist who is not involved in any other study-related procedures.

On Day 1 of each cohort period, the patients who meet all screening and eligibility criteria will be randomized in a 4:1 ratio to either rezpegaldesleukin or placebo within the dose group. Assignment to treatment groups will be determined by a computer-generated random sequence using an interactive web-response system (IWRS). The block sizes will not be known to the investigator.

The investigator, patients, and all study site and sponsor personnel involved in study activities will be blinded, with the following exceptions:
those involved in drug preparation and accountability
the Statistical Analysis Center (individuals from the statistical and PK group [which may include both sponsor and CRO personnel]) who will produce tables, figures, and listings for the interim analysis, and
the assessment committee [AC]

The individuals or functional groups who are planned to be unblinded will be identified in the study unblinding plan. Unblinding will follow sponsors procedures for unblinding and associated documentation. In case of an emergency, the investigator has the sole responsibility for determining if unblinding of a patient's treatment assignment is warranted for medical management of the event. The patient's safety must always be the first consideration in making such a determination. If the investigator decides that unblinding is warranted, it is the responsibility of the investigator to promptly document the decision and rationale and notify sponsor as soon as possible.

All doses of rezpegaldesleukin or placebo will be administered at the site by appropriately qualified and trained clinical staff. Rezpegaldesleukin will be prepared for injection by an unblinded pharmacist, or other qualified unblinded personnel, according to the instructions in the Pharmacy Manual. Individual patients may receive up to 3 injections to achieve the necessary dose. The preparation of the exact dose and volume injected will be recorded for each individual patient.

Medications taken in the year prior to screening must be documented in the source documents and on the concomitant medications log eCRF with the following: the name of the medication (generic name), indication, dose, frequency of administration, route of administration, and start and stop dates of administration.
Any medication taken by a patient during the course of the study and the reason for its use must be documented in the source documents and the concomitant medications log eCRF with the following: the name of the medication (generic name), indication, dose, frequency of administration, route of administration, and date of administration.
Treatment with concomitant therapies for AD during the study is permitted only as described below.
Daily use of emollients is required as background treatment. Moisturizers with additives such as antipruritics or antiseptics are not permitted. If daily applications are missed, it will not be considered a protocol violation.
Patients should not apply emollients on the day of their study visit prior to the procedures to allow adequate assessment of skin dryness.
Oral and topical antihistamines are allowed.
   All medications and treatments (e.g., IV solution, blood transfusion) for AEs and SAEs must be documented in the source documents and on the concomitant medications log through the Last Study Visit (Day 134 for EASI 50 non-responders and Day 169 for EASI 50 responders).
   The following listed treatments are prohibited. If a prohibited treatment listed here is required, the study drug should be discontinued.
Topical treatments: TCS, topical immune modulators (e.g., tacrolimus or pimecrolimus) or PDE-4 inhibitor (e.g., crisaborole) except when given as rescue therapy
Systemic corticosteroids: oral or parenteral corticosteroids (intramuscular, intra-articular or IV). Note: Intranasal or inhaled steroid use is allowed during the trial.
Synthetic (oral) immunomodulators, including, but not limited to
JAK inhibitors (e.g., tofacitinib, ruxolitinib)
cyclosporine
methotrexate
mycophenolate mofetil, or
azathioprine
Biologics: Immunomodulating monoclonal antibodies (including but not limited to dupilumab, ustekinumab, omalizumab). Inactivated influenza and pneumococcal vaccinations are allowed. SARS-COV-2 vaccinations are allowed, as well as biologic treatments for COVID-19, such as bamlanivimab, remdesivir, baricitinib, casirivimab, and imdevimab, where authorized by local regulatory bodies.
Leukotriene inhibitors
Phototherapy, including
therapeutic phototherapy (psoralen ultraviolet-A, ultraviolet-B)
excimer laser, or
self-treatment with tanning beds
Any investigational therapy that is not rezpegaldesleukin
Bleach baths
Allergen immunotherapy
Receipt of any investigational study drug within 4 weeks or 5 half-lives (whichever is greater) before screening, and any other investigational study drug other than rezpegaldesleukin through the Last Study Visit (Day 134 for EASI 50 non-responders and Day 169 for EASI 50 responders).
Received aldesleukin or any investigational IL-2 analog at any time.
   Rescue therapy is permitted for patients at any time after Day 21 according to investigator's judgment.
   Should a treatment-emergent SAE, or certain AEs occur, dosing and enrollment for the individual patient will be suspended and the study team will consult the AC.
   Adverse events requiring stopping dosing in an individual include:
The patient experiences any of the following after receiving the first dose:
a treatment-emergent SAE that in the opinion of the investigator is related to the study drug or
a severe or life-threatening treatment-emergent AE that in the opinion of the investigator is related to the study drug.
The patient experiences clinical manifestations of moderate-to-severe cytokine release syndrome, defined by a constellation of symptoms including fever, nausea, chills, hypotension, tachycardia, rash, headache, chest discomfort, fatigue/generalized weakness, and dyspnea/shortness of breath, which typically occur in close temporal relationship with study drug administration in the absence of another obvious cause (e.g. infection)
The patient has 1 of the following laboratory abnormalities detected and confirmed, with no other obvious cause in the opinion of the investigator:
hemoglobin <8.0 g/dL
neutrophils <0.75×10⁹/L
WBCs <2.0×10⁹/L
total lymphocytes <0.4× 10⁹/L
platelets <50×10⁹/L
The patient has eosinophil counts as follows:
Asymptomatic patients with an eosinophil count >3000 cells/uL should have a repeat measurement to confirm the count, and if confirmed, should be discontinued from study treatment, or
Patients with eosinophil counts >1500 cells/uL and signs and symptoms of target organ involvement (e.g., involving the skin, airway, gastrointestinal tract, liver, cardiac or nervous system) should be discontinued from study medication and undergo appropriate medical evaluation.
Any other AE/clinical findings that in the view of the sponsor warrants convening the AC.
   Discontinuation of the investigational product for abnormal liver tests should be considered by the investigator when a patient meets 1 of the following conditions after consultation with the sponsor designated medical monitor:
ALT (alanine aminotransferase) AST (aspartate aminotransferase)>[5× for healthy subjects, 8× for patients] ULN (upper limit of normal)
ALT or AST >[3×ULN for healthy subjects, 5×ULN for patients] sustained for more than 2 weeks or
ALT or AST >3×ULN and total bilirubin level (TBL)>2×ULN or INR >1.5 or
ALT or AST >3×ULN the appearance of fatigue, nausea, vomiting, right upper-quadrant pain or tenderness, fever, rash, and/or eosinophilia (>5%)
ALP (alkaline phosphatase)>3×ULN ALP>2.5×ULN and TBL >2×ULN
ALP>2.5 ULN with the appearance of fatigue, nausea, vomiting, right quadrant pain or tenderness, fever, rash, and/or eosinophilia (>5%).

### Study Assessments and Procedures

The Schedule of Activities details the study procedures and their timing. Tests that will be performed for this study are described below.

Disease Activity Measures for Patients with Atopic Dermatitis
The following disease activity measures described will be collected at the times shown in the Schedule of Activities. Only the EASI score, BSA, and the IGA score will be collected at screening.
Eczema Area and Severity Index: The EASI assesses the extent of disease at 4 body regions and measures 4 clinical signs: (1) erythema, (2) induration/papulation, (3) excoriation, and (4) lichenification each on a scale of 0 to 3. The EASI confers a maximum score of 72. The EASI evaluates 2 dimensions of AD: extent of disease and clinical signs (Hanifin et al. 2001).
Percentage BSA affected by AD will be derived as part of the EASI assessment.
Body surface area is the percentage involvement of AD on a scale from 0% (no involvement) to 100% (full involvement), where 1% corresponds to the size of the patient's hand (including the palm and fingers) (Scarisbrick and Morris 2013).
Validated Investigator Global Assessment for Atopic Dermatitis (vIGA-AD): The IGA used in this study, the vIGA-AD (referred to as the IGA throughout the protocol) measures the IGA of the patient's overall severity of their AD, based on a static, numeric 5-point scale from 0 (clear skin) to 4 (severe disease). The score is based on an overall assessment of the degree of erythema, papulation/induration, oozing/crusting, and lichenification.
   Patient-Reported Outcome/Quality-of-Life Measures for Patients with Atopic Dermatitis
   The following patient-reported outcomes/quality-of-life (QoL) measures described will be collected at the times shown in the Schedule of Activities.
Dermatology Life Quality Index (DLQI): The DLQI is a simple, patient-administered, 10-item, validated, QoL questionnaire that covers 6 domains including symptoms and feelings, daily activities, leisure, work and school, personal relationships, and treatment. The recall period of this scale is over the “last week.” Response categories include “not at all,” “a lot,” and “very much,” with corresponding scores of 1, 2, and 3, respectively, and unanswered (“not relevant”) responses scored as 0. Scores range from 0 to 30 with higher scores indicating greater impairment of QoL. A DLQI total score of 0 to 1 is considered as having no effect on a patient's health-related QoL (Hongbo et al. 2005), and a 4-point change from baseline is considered as the minimal clinically important difference threshold (Khilji et al. 2002; Basra et al. 2015).
Patient-Oriented Eczema Measure (POEM): The POEM is a simple, 7-item, patient-administered scale that assesses disease severity in children and adults. Patients respond to questions about the frequency of 7 symptoms (itching, sleep disturbance, bleeding, weeping/oozing, cracking, flaking, and dryness/roughness) over the past week. Response categories include “No days,” “1-2 days,” “3-4 days,” “5-6 days,” and “Every day” with corresponding scores of 0, 1, 2, 3, and 4, respectively. Scores range from 0 to 28 with higher total scores indicating greater disease severity (Charman et al. 2004).
Itch Numerical Rating Scale (Itch NRS): The Itch NRS is a patient-administered, 11-point horizontal scale anchored at 0 and 10, with 0 representing “no itch” and 10 representing “worst itch imaginable.” Overall severity of a patient's itching is indicated by selecting the number that best describes the worst level of itching in the past 24 hours (Naegeli et al. 2015; Kimball et al. 2016).
   Skin Biopsies for Patients with Atopic Dermatitis
   Skin biopsies will be required for all patients and will be performed at baseline (Day 1), Day 22, and Day 85. On Days 1 and 85, lesional and nonlesional biopsies will be collected. On Day 22, only lesional biopsies will be collected.
   Skin biopsies will be collected at the times shown in the Schedule of Activities. A local anesthetic will be applied, and two 4-mm skin-punch biopsies will be obtained from the same target lesion on Days 1, 22, and 85. If the lesion has resolved, biopsy should be taken from the cleared skin in that area. The biopsies will be used for histological, immunohistochemical, mRNA, and epigenetic marker immunomonitoring analyses.
   Detailed instructions for handling the biopsy tissue at the study site will be provided in a laboratory manual by the sponsor. These biopsies will be analyzed in relation to the changes in disease activity measures following treatment with rezpegaldesleukin.
   Biopsies will be retained for a maximum of 15 years after the last patient visit, or for a shorter period, if local regulations and ethical review boards (ERBs) allow, at a facility selected by the sponsor. This retention period enables use of new technologies, response to regulatory questions, and investigation of variable response that may not be observed until later in the development of the compound. Samples may be used for research on rezpegaldesleukin, disease process, pathways associated with disease, mechanism of action, response to treatment with rezpegaldesleukin, and/or research method, or in validating diagnostic tools or assay(s).
   Technologies are expected to improve during the 15-year storage period and therefore cannot be specifically named. Existing approaches, including mutation profiling, copy number variability analysis, gene expression assays, multiplex assays, and/or immunohistochemistry may be performed on these tissue samples to assess potential associations between these biomarkers and clinical outcomes.

### Clinical Photography

Documentation of the clinical response will be permitted through sequential photography of a selected lesion before, during, and after treatment with rezpegaldesleukin for all patients. Clinical photographs will be collected at the times shown in the Schedule of Activities. Detailed instructions for obtaining clinical photographs will be provided in a study manual.

### Adverse Events

The investigator will record all relevant AE/SAE information in the eCRF, whether reported by the patient or observed by study staff. Investigators are responsible for monitoring the safety of patients who have entered this study and for alerting sponsor or its designee to any event that seems unusual, even if this event may be considered an unanticipated benefit to the patient.

The investigator is responsible for the appropriate medical care of patients during the study. Investigators must document their review of each laboratory safety report. The investigator remains responsible for following, through an appropriate health care option, AEs that are serious or otherwise medically important, considered related to the IP or the study, or that caused the patient to discontinue the IP before completing the study. The patient should be followed up until the event resolves, stabilizes with appropriate diagnostic evaluation, or is reasonably explained. The frequency of follow-up evaluations of the AE is left to the discretion of the investigator.
Severity and seriousness of an AE are not synonymous. Severity is grading the intensity of an event. Seriousness of an event is based on the subject/event outcome.
In this study, AEs will be graded as mild, moderate, or severe using the following definitions.
1. Mild: Condition does not interfere with activities of daily living. Use of a concomitant therapy can still be consistent with a mild severity as long as the patient is able to carry out activities of daily living.
2. Moderate: Condition interferes with activities of daily living, but patient is able to compensate and do the daily activities that must be done (e.g., go to work, school, shop for groceries, etc.)
3. Severe: Condition prevents patients from completing activities of daily living, confined to bed or must miss work or school.
   Adverse events will be reported with an individual start and stop date for each AE severity grade. Please refer to the eCRF Completion Guidelines for detailed reporting instructions.
   Clinical laboratory tests consist of hematology, chemistry, and urinalysis (clean catch). Hepatic function (including AST, ALT, gamma-glutamyl transferase, ALP, lactate dehydrogenase, and TBL) will be tested as part of the chemistry panel. Blood and urine samples will be collected at the times specified in the Schedule of Activities. Instructions for the collection and processing of samples will be provided in a Laboratory Manual.

### Injection-Site Assessments

According to the Schedule of Activities, injection-site assessments will be performed at the end of each visit on Day 1 through Day 99 and at the ED visit, if occurs. The investigator will ask the patient if she/he had any injection-site concern since the preceding visit or, when assessed on Day 1, since the first injection. Patient's responses will be recorded per the Injection Site Assessment and Pain Visual Analog Scale tools to capture specific information relating to an injection site if there is injection-site concern and/or a reaction. Any event relating to an injection site will be captured as a study exploratory endpoint and not be recorded as an AE. Any new and/or ongoing ISR symptoms from previous visits require injection-site assessment and visual analog scale to be completed.
If an injection site reaction occurs, 1 optional biopsy per patient may be taken from up to 8 patients. Biopsies may be taken at any time, but Visit 8 (Day 22) is preferred. Detailed instructions for handling the biopsy tissue at the study site will be provided in a laboratory manual by the sponsor. Biopsies will be collected, analyzed, and retained in the same manner as the skin biopsies.
Optional clinical photographs of ISRs may be collected at any time. Two photographs per patient may be taken from up to 8 patients. Detailed instructions for obtaining clinical photographs will be provided in a study manual.
At the visits and times specified in the Schedule of Activities, venous blood samples will be collected to determine the plasma concentrations of rezpegaldesleukin. A maximum of 3 samples may be collected at additional time points during the study if warranted and agreed upon between both the investigator and sponsor. Instructions for the collection and handling of blood samples will be provided by the sponsor. The actual date and time (24-hour clock time) of each sampling will be recorded.
At the visits and times specified in the Schedule of Activities, venous blood samples will be collected to determine antibody production against rezpegaldesleukin. To interpret the results of immunogenicity, a venous blood PK sample will be collected at the same time points to determine the plasma concentrations of rezpegaldesleukin. All samples for immunogenicity during the treatment period will be taken predose. Instructions for the collection and handling of blood samples will be provided by the sponsor. The actual date and time (24-hour clock time) of each sampling will be recorded.
Treatment-emergent ADAs (ADAs): Patients who are TE-ADA positive at the last scheduled assessment or discontinuation visit may have additional samples taken at 3, 6, 9 (optional), and 12 months post-last assessment until the titer returns to within 2 fold of baseline titer or for up to 1 year, whichever is less. Immunogenicity will be assessed by a validated assay designed to detect ADAs in the presence of rezpegaldesleukin at a laboratory approved by the sponsor. Antibodies may be further characterized and/or evaluated for their ability to neutralize the activity of rezpegaldesleukin.

### Cohorts:

Approximately 25 patients with AD will be enrolled in each cohort for a maximum of 2 cohorts (50 patients). This will allow approximately 20 patients completing the study for a maximum of 2 cohorts (40 patients). The sample size is customary for Phase 1 studies evaluating safety and PK. Patients who discontinue the study before completing the Day 85 assessment may be replaced at the discretion of the sponsor. The replacement patient should be assigned to the same treatment allocation as the discontinued patient. A key clinical assessment is the percentage change from baseline in EASI at Week 12. With a sample size of approximately 20 patients completing the study per cohort, randomized in a 4:1 ratio to rezpegaldesleukin or placebo, the half-width of the 95% confidence interval of the percentage change from baseline in EASI between rezpegaldesleukin and placebo will be within 22% with a standard deviation assumption of 20%.
All randomized patients who receive at least 1 dose of study drug will be included according to the randomized treatment, regardless of whether they have completed all protocol requirements. The PK analysis population will consist of all randomized patients who receive rezpegaldesleukin and have adequate PK data to permit a meaningful analysis. Pharmacodynamic analyses will be conducted on the full analysis set, which includes all evaluable data from all patients receiving at least 1 dose of study drug according to the randomized treatment. Pharmacodynamics, immunogenicity, cytokine, and disease activity measures will be analyzed on this population. Demographics and baseline characteristics including age, race, ethnicity, weight, height, BMI, and sex at birth will be summarized descriptively.

Data listings and summary statistics will be provided for safety, PD, PK, and clinical data by treatment group over time. For continuous variables, summary statistics include the mean, standard deviation, minimum, maximum, median, and number of observations. For categorical variables, frequency counts and percentages will be provided. The data from placebo groups will be pooled across cohorts as 1 placebo group. Additional exploratory analyses of the data will be conducted as deemed appropriate. The safety analysis will be based on the safety population. For patients who receive placebo in either cohort, the safety data will be pooled. Adverse events will be classified according to the Medical Dictionary for Regulatory Activities (MedDRA). Treatment-emergent adverse events will be defined as AEs that occur on or after receiving the first dose of study drug. The frequency of TEAEs will be tabulated using MedDRA by system organ class and preferred terms and treatment. In addition, by -patient listings will be provided for TEAEs and SAEs. Clinical laboratory results, vital signs, and ISRs will also be summarized. The following PK parameters will be calculated from plasma concentration-time data after the first dose using standard noncompartmental methods of analysis, as data permit: AUC, Cₘₐₓ, and time to maximum observed plasma concentration (Tₘₐₓ).

Clinical endpoints over time will be summarized by treatment. Treatment comparisons of continuous clinical endpoints will be explored using mixed effects for repeated measures. For categorical clinical data, endpoints will be explored using Fisher exact test. The disease activity data are exploratory measures. A first interim review is planned when approximately 8 patients from Cohort 2 have had the opportunity to complete, at a minimum, the Week 6 visit. The second interim review is planned when approximately 16 patients from Cohort 2 have had the opportunity to complete, at a minimum, the Week 12 visit. Safety/tolerability, PD, PK, and disease activity measure data will be included in these interim reviews as described in the KFAD Assessment Committee Charter. The purpose of the interim reviews is to support the doses being evaluated for each cohort by reviewing available safety/tolerability, PD, PK, and disease activity measures. The AC is authorized to evaluate unblinded interim analysis. The AC, which includes sponsors clinical development representative(s) and statistical, PK/PD, and biomarker functions will review the PK, clinical, and peripheral blood cell type and histology data.

| Term | Definition |
|---|---|
| AC | assessment committee |
| AD | atopic dermatitis |
| ADA | antidrμg antibody |
| AE | adverse event: Any untoward medical |
| | occurrence in a patient or clinical |
| | investigation patient administered a |
| | pharmaceutical product that does not |
| | necessarily have a causal relationship |
| | with this treatment. An AE can |
| | therefore be any unfavorable and unintended |
| | sign (including an abnormal |
| | laboratory finding), symptom, or disease |
| | temporally associated with the use |
| | of a medicinal (investigational) product, |
| | whether or not related to the |
| | medicinal (investigational) product. |
| ALP | alkaline phosphatase |
| ALT | alanine aminotransferase |
| AST | aspartate aminotransferase |
| AUC | area under the plasma concentration |
| | versus time curve |
| BCG | bacille Calmette-Guerin |
| BMI | body mass index |
| BP | blood pressure |
| BSA | body surface area |
| Cₘₐₓ | maximum observed plasma concentration |
| CP | clinical pharmacologist |
| CRP | clinical research physician |
| CRS | cytokine release syndrome |
| C-SSRS | Columbia Suicide Severity Rating Scale |
| DLQI | Dermatology Life Quality Index |
| DLT | dose-limiting toxicity |
| EASI | Eczema Area and Severity Index |
| EASI 50 | patient EASI score reduced by at least |
| | 50% relative to their baseline score |
| ECG | electrocardiogram |
| eCRF | electronic case report form |
| ERB | ethical review board |
| FDA | Food and Drμg Administration |
| FoxP3 | forkhead box P3 |
| GCP | good clinical practice |
| HCV | hepatitis C virus |
| HIV | human immunodeficiency virus |
| HR | heart rate |
| IB | Investigator's Brochure |
| ICF | informed consent form |
| ICH | International Council for Harmonisation |
| IGA | Investigator Global Assessment |
| IL-2 | interleukin 2 |
| IP | investigational product |
| IRB | Institutional Review Board |
| ISR | injection-site reaction |
| IV | intravenous |
| IWRS | interactive web-response system |
| MAD | multiple-ascending dose |
| MedDRA | Medical Dictionary for Regulatory Activities |
| NK | natural killer |
| NOAEL | no-observed-adverse-effect level |
| NRS | Numeric Rating Scale |
| PD | pharmacodynamic(s) |
| PDE 4 | phosphodiesterase type 4 |
| PEG | polyethylene glycol |
| PK | pharmacokinetic(s) |
| POEM | Patient-Oriented Eczema Measure |
| PsO | psoriasis |
| QoL | quality of life |
| RR | respiratory rate |
| SAD | single-ascending dose |
| SAE | serious adverse event |
| SAP | statistical analysis plan |
| SC | subcutaneous |
| SLE | systemic lupus erythematosus |
| TB | tuberculosis |
| TBL | total bilirubin level |
| TCNI | topical calcineurin inhibitor |
| Tcon | conventional T |
| TCS | topical corticosteroid |
| TEAE | treatment emergent adverse event |
| TRAE | treatment-related AE |
| Tₘₐₓ | time to maximum observed plasma concentration |
| Treg | regulatory T |
| TST | tuberculin skin test |
| ULN | upper limit of normal |
| USP | United States Pharmacopeia |
| VAS | visual analog scale |
| VIGA-AD | Validated Investigator Global |
| | Assessment for Atopic Dermatitis |
| WBC | white blood cell |

### Clinical Laboratory Tests:

| | | | Drug and | Pregnancy (for |
|---|---|---|---|---|
| | | | Alcohol | Women of Child- |
| Hematology | Chemistry | Serology | Screening | bearing Potential) |
| 1. Hemoglobina | 73. AST (SGOT)^{b} | 2. Hepatitis B | 4. Opioids (urine) | Serum human |
| 2. Hematocrit^{a} | 74. ALT (SGPT)^{b} | surface antigen | 5. Cocaine (urine) | chorionic |
| 3. RBC | 75. GGT^{b} | (HBsAg) | 6. Amphetamines | gonadotropin |
| 4. WBCª | 76. TBL^{b} | 3. Hepatitis B core | (urine) | (hCG; mandatory |
| 5. Platelet countª | 77. Alkaline phosphatase^{b} | antibody | 7. Cannabinoids | for screening) |
| 6. Neutrophils | 78. LDH^{b} | (HBcAb+) | (urine) | Urine |
| (absolute)^{a} | 79. Albumin | 4. Hepatitis C virus | 8. Alcohol (urine) | |
| 7. Lymphocytes | 80. Creatinine | antibody | | |
| (absolute) | 81. Glucose | (anti-HCV) | | |
| 8. Monocytes | 82. Total protein | 5. Human | | |
| (absolute) | 83. Sodium | immunodeficiency | | |
| 9. Eosinophils | 84. Potassium | virus (HIV) | | |
| (absolute) | 85. Chloride | antibody | | |
| 10. Basophils | 86. CO₂ content or bicarbonate | | | |
| (absolute) | 87. Urea nitrogen | | | |
| 11. Mean corpuscular | | | | |
| volume (MCV) | | | | |
| 12. Mean corpuscular | | | | |
| hemoglobin | | | | |
| (MCH) | | | | |
| 13. Mean corpuscular | | | | |
| hemoglobin | | | | |
| concentration | | | | |
| (MCHC) | | | | |
| 14. Peripheral blood | | | | |
| mononuclear cell | | | | |
| (PBMC) isolation | | | | |

Urinalysis (by dipstick)

| | |
|---|---|
| | For positive protein, WBC or blood, a microscopic examination including: |
| 9. Specific gravity | 17. RBC |
| 10. pH | 18. WBC |
| 11. Glucose | 19. Epithelial cells |
| 12. Protein | 20. Bacteria |
| 13. Bilirubin | 21. Crystals |
| 14. Ketones | 22. Casts |
| 15. Leukocytes | |
| 16. Blood | |

Abbreviations: ALT = alanine aminotransferase; AST = aspartate aminotransferase; GGT = gamma-glutamyl transferase, LDH = lactate dehydrogenase; RBC = red blood cell; SGOT = serum glutamic oxaloacetic transaminase; SGPT = serum glutamic pyruvic transaminase; TBL = total bilirubin level; WBC = white blood cell.

### Validated Investigator Global Assessment Scale for Atopic Dermatitis-vIGA-AD™ Instructions:

The IGA score is selected using the descriptors below that best describe the overall appearance of the lesions at a given time point. It is not necessary that all characteristics under Morphological Description be present.

| Score | Morphological Description |
|---|---|
| 0-Clear | No inflammatory signs of atopic |
| | dermatitis (no erythema, no induration/ |
| | papulation, no lichenification, no oozing/crusting). |
| | Post-inflammatory hyperpigmentation |
| | and/or hypopigmentation may be present. |
| 1-Almost | Barely perceptible erythema, barely perceptible |
| clear | induration/papulation, and/or minimal |
| | lichenification. No oozing or crusting. |
| 2-Mild | Slight but definite erythema (pink), slight but definite |
| | induration/papulation, and/or slight but definite |
| | lichenification. No oozing or crusting. |
| 3-Moderate | Clearly perceptible erythema |
| | (dull red), clearly perceptible |
| | induration/papulation, and/or clearly perceptible |
| | lichenification. Oozing and crusting may be present. |
| 4-Severe | Marked erythema (deep or bright red), marked |
| | induration/papulation, and/or marked lichenification. |
| | Disease is widespread in extent. Oozing |
| | or crusting may be present. |

Notes:
In indeterminate cases, please use extent to differentiate between scores.
For example: Patient with marked erythema (deep or bright red), marked papulation and/or marked lichenification that is limited in extent, will be considered “3-Moderate”.
Excoriations should not be considered when assessing disease severity.
Source: http://www.eczemacouncil.org/wp-content/uploads/2018/02/Validated-Investigator-Global-Assessment-Scale_vIGA-AD_2017.pdf

### REFERENCES

Abbas A K, Trotta E, R Simeonov D, Marson A, Bluestone J A. Revisiting IL-2: biology and therapeutic prospects. Sci Immunol. 2018; 3 (25). pii: eaat1482. doi: 10.1126/sciimmunol.aat1482.
Basra M K, Salek M S, Camilleri L, Sturkey R, Finlay A Y. Determining the minimal clinically important difference and responsiveness of the Dermatology Life Quality Index (DLQI): further data. Dermatology. 2015; 230 (1): 27-33.
Boyman O, Krieg C, Homann D, Sprent J. Homeostatic maintenance of T cells and natural killer cells. Cell Mol Life Sci. 2012; 69 (10): 1597-1608.
Charman C R, Venn A J, Williams, HC. The Patient-Oriented Eczema Measure. Arch Dermatol. 2004; 140:1513-1519.
Eichenfield L F, Tom W L, Chamlin S L, Feldman S R, Hanifin J M, Simpson E L, Berger T G, Bergman J N, Cohen D E, Cooper K D, Cordoro K M, Davis D M, Krol A, Margolis D J, Paller A S, Schwarzenberger K, Silverman R A, Williams H C, Elmets C A, Block J, Harrod C G, Smith Begolka W, Sidbury R. Guidelines of care for the management of atopic dermatitis: section 1. Diagnosis and assessment of atopic dermatitis. J Am Acad Dermatol. 2014; 70 (2): 338-351.
Hanifin J M, Thurston M, Omoto M, Cherill R, Tofte S J, Graeber M. The eczema area and severity index (EASI): assessment of reliability in atopic dermatitis. EASI Evaluator Group. Exp Dermatol. 2001; 10 (1): 11-18.
Hongbo Y, Thomas C L, Harrison M A, Salek M S, Finlay A Y. Translating the science of quality of life into practice: what do dermatology life quality index scores mean? J Invest Dermatol. 2005; 125 (4): 659-664.
ICH S6 (R1) Guideline: Preclinical safety evaluation of biotechnology-derived pharmaceuticals. June 2011. Available at: https://www.ich.org/products/guidelines/safety/article/safety-guidelines.html. Accessed Jun. 12, 2019.
Khilji F A, Gonzalez M, Finlay A Y. Clinical meaning of change in Dermatology Life Quality Index scores. Br J Dermatol. 2002; 147 (suppl 62): 25-54.
Kimball A B, Naegeli A N, Edson-Heredia E, Lin C Y, Gaich C, Nikai E, Wyrwich K, Yosipovitch G. Psychometric properties of the Itch Numeric Rating Scale in patients with moderate-to-severe plaque psoriasis. Br J Dermatol. 2016; 175 (1): 157-162.
Klatzmann D, Abbas A K. The promise of low-dose interleukin-2 therapy for autoimmune and inflammatory diseases. Nat Rev Immunol. 2015; 15 (5): 283-294.
Malek T R, Castro I. Interleukin-2 receptor signaling: at the interface between tolerance and immunity. Immunity. 2010; 33 (2): 153-165.
Mizui M. Natural and modified IL-2 for the treatment of cancer and autoimmune diseases. Clin Immunol. 2019. Available at: https://doi.org/10.1016/j.clim.2018.11.002. Accessed Apr. 13, 2020.
Moon B—I, Kim T H, Seoh J-Y. Functional modulation of regulatory T cells by IL-2. PLoS ONE. 2015; 10 (11): e0141864. doi: 10.1371/journal.pone.0141864.
Moosbrugger-Martinz V, Gruber R, Ladstätter K, Belluti M, Blunder S, Schmuth M, Dubrac S. Filaggrin null mutations are associated with altered circulating Tregs in atopic dermatitis. J Cell Mol Med. 2018 (in press). Available at: https://doi.org/10.1111/jcmm.14031. Accessed Jun. 12, 2019.
Naegeli A N, Flood E, Tucker J, Devlen J, Edson-Heredia E. The Worst Itch Numeric Rating Scale for patients with moderate to severe plaque psoriasis or psoriatic arthritis. Int J Dermatol. 2015; 54 (6): 715-722.
Nedoszytko B, Lange M, Sokołowska-Wojdyło M, Renke J, Trzonkowski P, Sobjanek M, Szczerkowska-Dobosz A, Niedoszytko M, Górska A, Romantowski J, Czarny J, Skokowski J, Kalinowski L, Nowicki R. The role of regulatory T cells and genes involved in their differentiation in pathogenesis of selected inflammatory and neoplastic skin diseases. Part II: The Treg role in skin diseases pathogenesis. Postepy Dermatol Alergol. 2017; 34 (5): 405-417.
Proleukin [package insert]. San Diego, CA: Prometheus Laboratories Inc; 2015. Available at: https://www.proleukin.com/downloads/proleukin-pi.pdf. Accessed Jun. 12, 2019.
Scarisbrick J J, Morris S. How big is your hand and should you use it to score skin in cutaneous T-cell lymphoma? Br J Dermatol. 2013; 169 (2): 260-265.
Sharabi A, Tsokos M G, Ding Y, Malek T R, Klatzmann D, Tsokos G C. Regulatory T cells in the treatment of disease. Nat Rev Drug Discov. 2018 Oct. 12. doi: 10.1038/nrd.2018.148. [Epub ahead of print]
Sim G C, Radvanyi L. The IL-2 cytokine family in cancer immunotherapy. Cytokine Growth Factor Rev. 2014; 25:377-390.
Winthrop K L, Novosad S A, Baddley J W, Calabrese L, Chiller T, Polgreen P, Bartalesi F, Lipman M, Mariette X, Lortholary O, Weinblatt M E, Saag M, Smolen J. Opportunistic infections and biologic therapies in immune-mediated inflammatory diseases: consensus recommendations for infection reporting during clinical trials and postmarketing surveillance. Ann Rheum Dis. 2015; 74 (12): 2107-2116.

### Cell-Subset Abbreviations and Definitions:

Several different cell subsets as defined by markers are referenced in this document, and for simplicity, are abbreviated with naming conventions. These naming conventions and their associated marker definitions are shown in Table KFAD 1.

TABLE KFAD.1
Cell-Subset Abbreviations Defined by Associated Markers

| Abbreviation | Definition by Cell Markers |
|---|---|
| Tcon cellsª | Tcon-Cell Subsets |
| | 1. CD3+ CD4+ CD8− CD25+ FoxP3− |
| | 2. CD3+ CD4+ CD8− CD25− FoxP3+ |
| | 3. CD3+ CD4+ CD8− CD25− FoxP3− |
| | 4. CD3+ CD4− CD8+ |
| Total Treg cells | CD3+ CD4+ CD8− CD25+ FoxP3+ |
| CD25bright Treg cells^{b} | CD3+ CD4+ CD8− CD25bright FoxP3+ |
| NK cells^{c} | CD3-CD16+ CD56+ |
| Proliferating cells | Ki67+ |

Abbreviations: CD = cluster of differentiation; FoxP3 = forkhead box P3; NK = natural killer; Tcon = conventional T; Treg = regulatory T.
^{a}Tcon cells refers to all 4 subsets.
^{b}Cd25^{bright} is defined by an established gate in the flow cytometry analysis, selecting the 0.5% (at baseline) highest expressing total Treg cells. The gate established at baseline as 0.5% is carried over the time course.
^{c}Only the CD56+ NK cells were measured in some prior studies.

### Results-Atopic Dermatitis Study KFAD-Phase 1b Double-Blind, Placebo-Controlled (NCT04081350):

A decrease in Treg cells numbers or a reduction in immunosuppressive functions of Tregs have been identified as key immunological defects contributing to the pathogenesis of multiple autoimmune and inflammatory diseases including atopic dermatitis (AD). The purpose of rezpegaldesleukin (abbreviated as “rezpeg.”) therapy is to increase Treg cell expression and activation in patients with inflammatory diseases such as AD, which could result in a beneficial clinical outcome. In this Phase 1b double-blind, placebo-controlled study (NCT04081350), patients were randomized to either 12 μg/kg rezpegaldesleukin (N=16), 24 (N=17) μg/kg rezpegaldesleukin, or placebo (PBO) (N=11) dose bi-weekly, via subcutaneous injections, up to week (W) 12 with a follow-up period of W12-48. Inclusion criteria consisted of patients with moderate-to-severe AD involving ≥10% body surface area in the affected skin, a Validated Investigator's Global Assessment (vIGA)>=3, and an Eczema Area and Severity Index (EASI) score of ≥16. Efficacy was assessed, up to W12, through EASI. Safety assessments here include Treatment-Emergent Adverse Effects (TEAE), Death, Discontinuation, Hematology, and Injection Site Reactions (ISR). Pharmacodynamics (PD) were evaluated using flow cytometry.

Demographic and disease baseline characteristics are presented in Table KFAD 2.

### KFAD Interim Results:

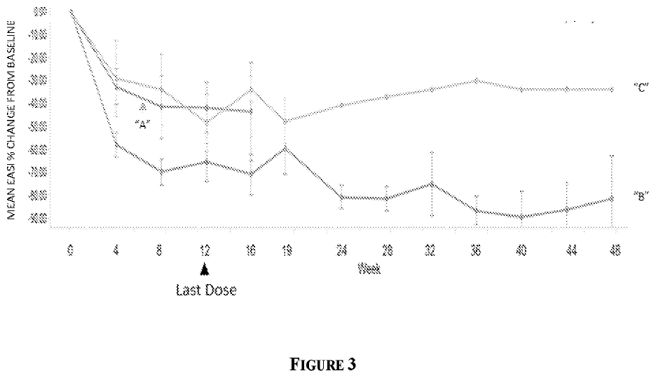

FIG. 3 illustrates results from an interim analysis of the phase 1b study of patients with moderate-to-severe atopic dermatitis (NCT04081350) treated with rezpegaldesleukin, an RUR20kD-IL-2 Selective Treg stimulator composition and represents mean percent change from baseline in EASI scores over time in a subset of patients. Moderate to severe atopic dermatitis patients being treated with rezpegaldesleukin every two weeks subcutaneously for 12 weeks at two different dose levels, in which the higher dose demonstrated a change in EASI score with a more robust effect than patients in the placebo group. Patients had to be EASI-50 (EASI=eczema area and severity index) responders at week 19 to continue in the study.

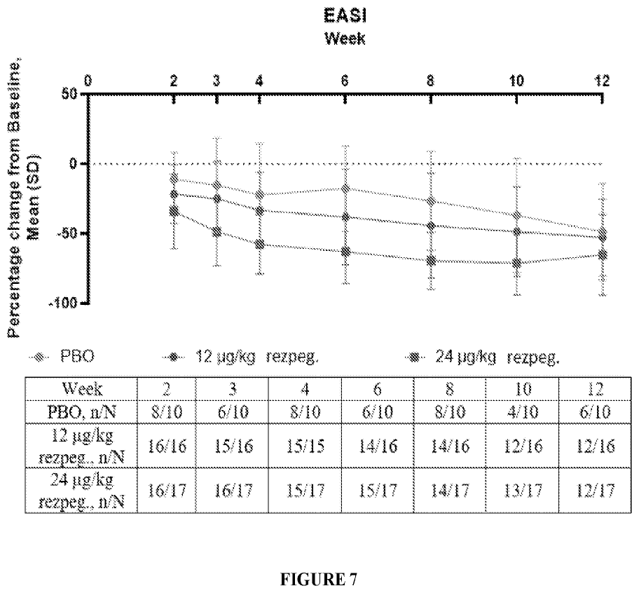

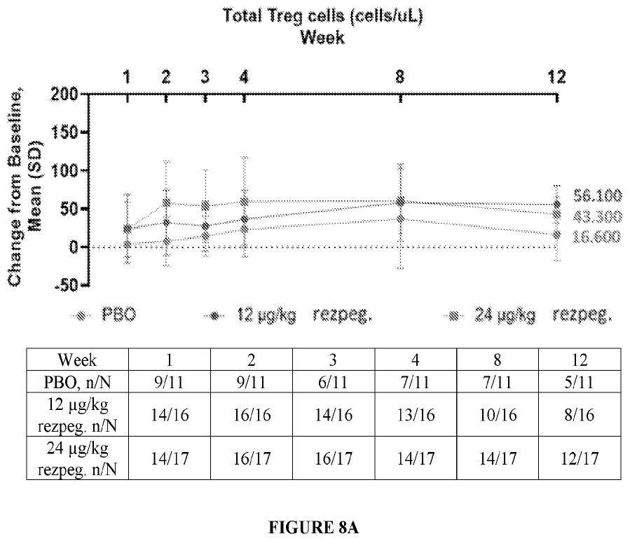

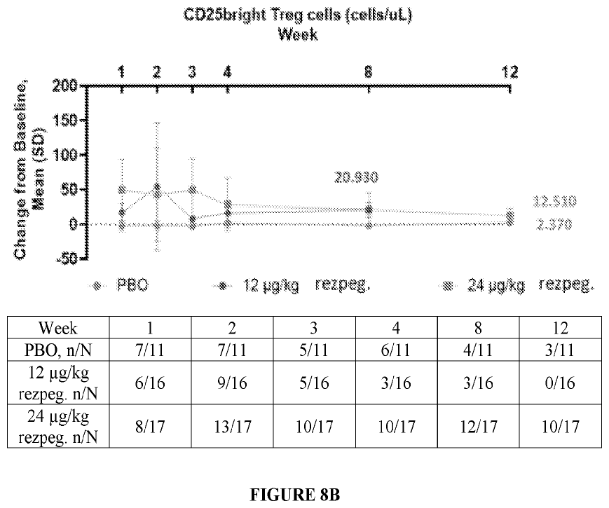

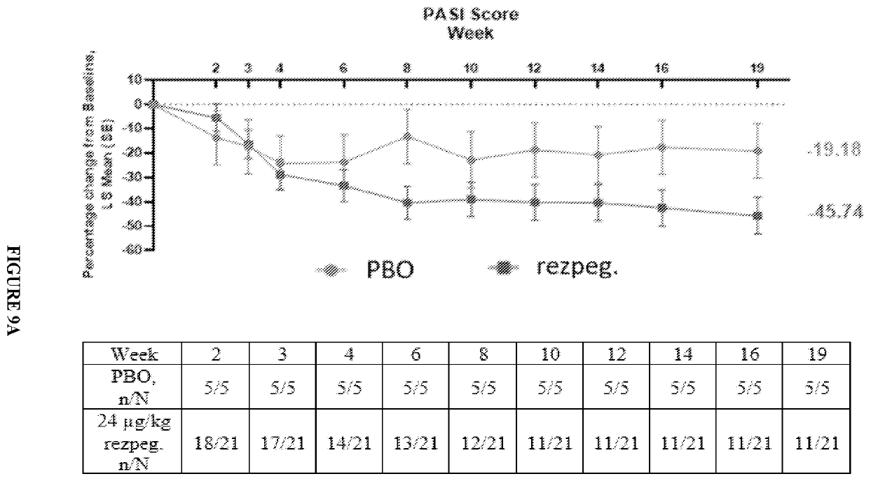

FIG. 7 illustrates results showing the 24 μg/kg cohort separates from placebo on change from baseline in EASI score from weeks 4-10 (FIG. 7). The 12 μg/kg dose does not separate from placebo (FIG. 7). A summary of safety variables and ISR events for PBO, 12 and 24 μg/kg rezpegaldesleukin are presented in Table KFAD 3. In patients treated with rezpegaldesleukin, 16 TEAEs occurred in the 12 μg/kg dose and 28 in the 24 μg/kg dose. No severe or serious TEAEs were reported in rezpegaldesleukin treated patients. In patients treated with rezpegaldesleukin, 1 TEAE lead to discontinuation in the 12 μg/kg cohort (Headache) and 3 TEAEs lead to discontinuation in the 24 μg/kg cohort (Abscess limb, Eosinophil count increase, and Urticaria). No deaths were reported in patients treated in any treatment group. The most commonly occurring ISR reported PBO was bruising (10%), for rezpegaldesleukin treated patients ISR included Erythema (75.0%) in the 12 μg/kg cohort and (52.9%) in the 24 μg/kg cohort. Eosinophil count, at W12, increased, versus PBO, from baseline in 12 and 24 μg/kg rezpegaldesleukin treated patients (0.070 vs. 0.152 and 0.418 GI/L). In rezpegaldesleukin treated patients, versus PBO, Treg cells increased from baseline up to W12, and CD25bright Treg cells up to W8 (FIG. 8). NK cells were increase at W12, versus PBO, from baseline in 12 and 24 μg/kg rezpegaldesleukin treated patients (32.0 vs. 400.25 and 1019.25 cells/uL). At W12, Tcon cells increased, versus PBO, from baseline in 24 μg/kg rezpegaldesleukin treated patients (309.3 vs. 474.34 cells/μL). In patients with AD, rezpegaldesleukin in the dose range tested was well-tolerated and safe, with does dependent improvements to EASI and Itch NRS outcomes by W12, relative to PBO.

TABLE KFAD.2
Demographic and Disease Baseline Characteristics

| | | 12 μg/kg | 24 μg/kg |
|---|---|---|---|
| | PBO | rezpeg. | rezpeg. |
| Characteristic | (N = 10) | (N = 16) | (N = 17) |
| Sex, n (%) | | | |
| Female | 6 (60.0) | 11 (68.8) | 7 (41.2) |
| Male | 4 (40.0) | 5 (31.3) | 10 (58.8) |
| Race | | | |
| White | 6 (60.0) | 11 (68.8) | 14 (82.4) |
| Black or African | 3 (30.0) | 3 (18.8) | 3 (17.6) |
| American | | | |
| Asian | 1 (10.0) | 2 (12.5) | 0 |
| American Indian or | 0 | 0 | 0 |
| Alaska Native | | | |
| Native Hawaiian or | 0 | 0 | 0 |
| Other Pacific | | | |
| Islander | | | |
| Other | 0 | | 0 |
| Ethnicity, n (%) | | | |
| Hispanic or Latino | 0 | 3 (18.8) | 7 (41.2) |
| Not Hispanic or | 10 (100) | 13 (81.3) | 10 (58.8) |
| Latino | | | |
| Age (years), Mean (SD) | 42.5 (19.76) | 47.9 | 37.5 (16.36) |
| | | (17.49) | |
| EASI (score), Mean (SD) | 24.8 (7.13) | 23.4 | 22.1 (6.31) |
| | | (11.32) | |
| vIGA-AD | | | |
| 0-Clear | 0 | 0 | 0 |
| 1-Almost clear | 0 | 0 | 0 |
| 2-Mild | 0 | 0 | 0 |
| 3-Moderate | 5 (50.0) | 9 (56.3) | 11 (64.7) |
| 4-Severe | 5 (50.0) | 7 (43.8) | 6 (35.3) |
| Itch NRS (score), | 8.5 (1.27) | 7.8 (2.10) | 7.4 (2.50) |
| Mean (SD) | | | |
| Any Prior | 10 (100) | 15 (93.8) | 13 (76.5) |
| Medications, n (%) | | | |

AD: Atopic Dermatitis; EASI: Eczema Area and Severity Index; IGA: Investigator's Global Assessment; NRS: Numeric Rating Scale

TABLE KFAD.3
Summary of safety variables and ISR events for PBO, 12 and 24 μg/kg
rezpegaldesleukin (abbreviated as “rezpeg.”)

| | | 12 μg/kg | 24 μg/kg |
|---|---|---|---|
| | PBO | rezpeg. | rezpeg. |
| Category | (N = 10) | (N = 16) | (N = 17) |
| Adverse Events [n (%) m] | 8 (80.0) 13 | 9 (56.3) 16 | 13 (76.5) 28 |
| Infections and infestations | 2 (20.0) 2 | 4 (25.0) 5 | 7 (41.2) 11 |
| Postoperative wound infection | 0 | 0 | 1 (5.9) 1 |
| Gastrointestinal disorders | 3 (30.0) 4 | 1 (6.3) 1 | 3 (17.6) 3 |
| Gastrooesophageal reflux disease | 1 (10.0) 1 | 0 | 0 |
| Investigationsª | 0 | 0 | 4 (23.5) 4 |
| Nervous system disorders | 0 | 2 (12.5) 2 | 2 (11.8) 2 |
| General disorders and | 1 (10.0) 1 | 1 (6.3) 1 | 2 (11.8) 2 |
| administration site conditions | | | |
| Injection site discolouration | 0 | 1 (6.3) 1 | 0 |
| Blood and lymphatic system | 0 | 0 | 0 |
| disorders | | | |
| Eye disorders | 0 | 2 (12.5) 2 | 1 (5.9) 1 |
| Skin and subcutaneous tissue | 0 | 1 (6.3) 1 | 1 (5.9) 1 |
| disorders | | | |
| Immune system disorders | 0 | 1 (6.3) 1 | 0 |
| Injury, poisoning and procedural | 2 (20.0) 2 | 1 (6.3) 1 | 0 |
| complications | | | |
| Metabolism and nutrition disorders | 1 (10.0) 1 | 1 (6.3) 1 | 0 |
| Musculoskeletal and connective | 1 (10.0) 2 | 0 | 1 (5.9) 1 |
| tissue disorders | | | |
| Renal and urinary disorders | 0 | 0 | 1 (5.9) 1 |
| Respiratory, thoracic and | 0 | 0 | 1 (5.9) 1 |
| mediastinal disorders | | | |
| Severe Adverse Events [n (%) m] | 3 (30.0) 4 | 0 | 0 |
| Adverse Events with Outcome | 0 | 0 | 0 |
| of Death [n (%) m] | | | |
| Serious Adverse Events [n (%) m] | 2 (20.0) 3 | 0 | 0 |
| Adverse Events Leading to | 0 | 1 (6.3) 2 | 3 (17.6) 3 |
| Discontinuation of Study [n (%) m] | | | |
| Injection Site Reactions [m] | 1 | 43 | 34 |

n = Number of subjects;
m = Number of events
For each category, subjects are included only once, even if they experienced multiple eve nts in that category.
ªRefers to: Increase in Eosinophil count, Hepatic enzyme, Lymphocyte count, and Weight.

Results from KFAD-A Phase 1 Randomized Atopic Dermatitis Study:

### Efficacy and Safety of Rezpegaldesleukin, a Selective Regulatory T-Cell Inducing IL-2 Conjugate, in the Treatment of Atopic Dermatitis:

Rezpegaldesleukin (LY3471851, NKTR-358) is a polyethylene glycol conjugate of recombinant human interleukin (IL)-2, described herein, which in human studies has been shown to selectively stimulate Treg expansion and suppressive function. This activity is conceived to result in beneficial clinical outcomes in patients with inflammatory diseases such as AD. The following is a report of the efficacy, safety, and biologic effects of rezpegaldesleukin in a Phase 1b, double-blind, placebo-controlled study (NCT04081350) of patients with AD.

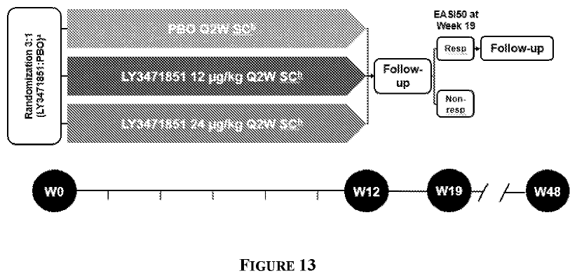

The general study design is shown in FIG. 13. (a) Full study design is not shown; the LY3471851 10 μg/kg cohort is not included in this analysis. (b) Total of 7 doses/patient EASI50=50% improvement from baseline in Eczema Area and Severity Index; PBO=placebo; Q2W-once every 2 weeks; SC=subcutaneous; W=Week. “Resp” represents responder. Key Eligibility Criteria were; Aged 18-70 years, Moderate-to-severe AD involving ≥10% body surface area in the affected skin, History of inadequate response or intolerance to topical medications, vIGA-AD™ ≥3, and Eczema Area and Severity Index (EASI) ≥16. Assessments and Statistical Analyses were:

### Assessments:

Efficacy:
   Change from baseline in EASI
   Proportion of patients who achieved 75% improvement from baseline in EASI score (EASI75)
   Proportion of patients who achieved vIGA-AD of 0 (clear) or 1 (almost clear)
   Durability of vIGA-AD (0,1) response in Week 16 vIGA-AD (0,1) responders
   Proportion of patients who achieved ≥4-point improvement in Itch Numeric Rating Scale
Safety: Treatment-emergent adverse effects and injection site reactions
Pharmacodynamics: Flow cytometry and epigenetic markers

### Statistical Analyses

Analyses were from interim data cutoff of May 10, 2022 for efficacy and Sep. 17, 2021 for pharmacodynamics and safety using descriptive statistics
Response rates used non-responder imputation for missing data

### Demographics and Baseline Characteristics:

| | | LY3471851 | LY3471851 |
|---|---|---|---|
| | PBO | 12 μg/kg | 24 μg/kg |
| | (N = 10)^{a} | (N = 16) | (N = 17) |
| Age, years | 42.5 (19.8) | 47.9 (17.5) | 37.5 (16.4) |
| Sex, n (%) | | | |
| Female | 6 (60.0) | 11 (68.8) | 7 (41.2) |
| Male | 4 (40.0) | 5 (31.3) | 10 (58.8) |
| Race, n (%) | | | |
| White | 6 (60.0) | 11 (68.8) | 14 (82.4) |
| Black | 3 (30.0) | 3 (18.8) | 3 (17.6) |
| Asian | 1 (10.0) | 2 (12.5) | 0 |
| American Indian or | 0 | 0 | 0 |
| Alaska Native | | | |
| Native Hawaiian or Other | 0 | 0 | 0 |
| Pacific Islander | | | |
| Other | 0 | 0 | 0 |
| Ethnicity, n (%) | | | |
| Hispanic or Latino | 0 | 3 (18.8) | 7 (41.2) |
| Not Hispanic or Latino | 10 (100) | 13 (81.3) | 10 (58.8) |
| EASI | 24.8 (7.1) | 23.4 (11.3) | 22.1 (6.3) |
| vIGA-AD, n (%) | | | |
| 0 (Clear) | 0 | 0 | 0 |
| 1 (Almost clear) | 0 | 0 | 0 |
| 2 (Mild) | 0 | 0 | 0 |
| 3 (Moderate) | 5 (50.0) | 9 (56.3) | 11 (64.7) |
| 4 (Severe) | 5 (50.0) | 7 (43.8) | 6 (35.3) |
| Itch NRS | 8.5 (1.3) | 7.8(2.1) | 7.4 (2.5) |
| Prior medications, n (%) | 10 (100) | 15 (93.8) | 13 (76.5) |

Data are mean (standard deviation) unless stated otherwise
^{a}1 site was terminated due, to quality issues and subjects were excluded from the analyses (1 subject in the PBO group)
EASI = Eczema Area and Severity Index; NRS = Numeric Rating Scale; PBO = placebo

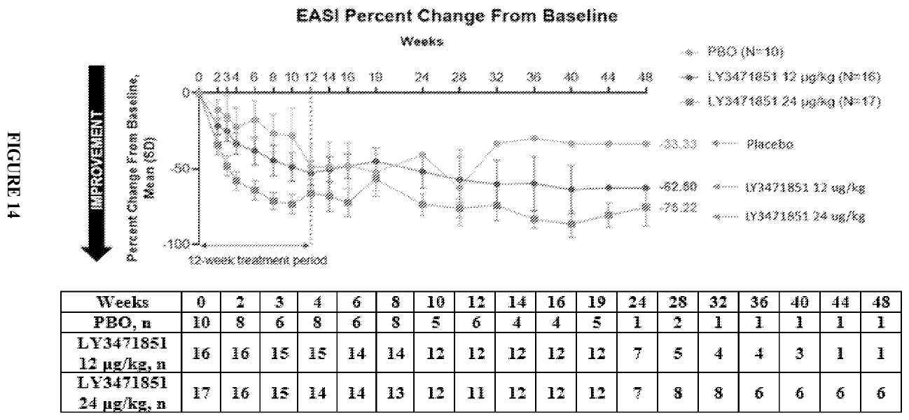

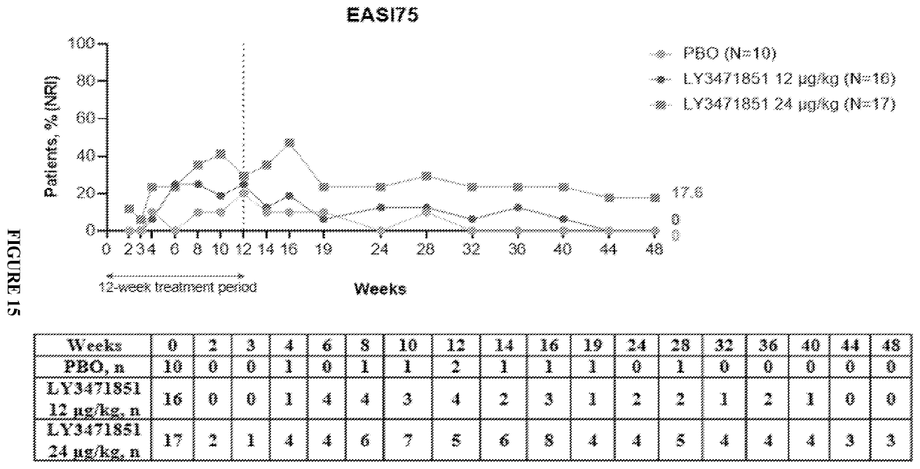

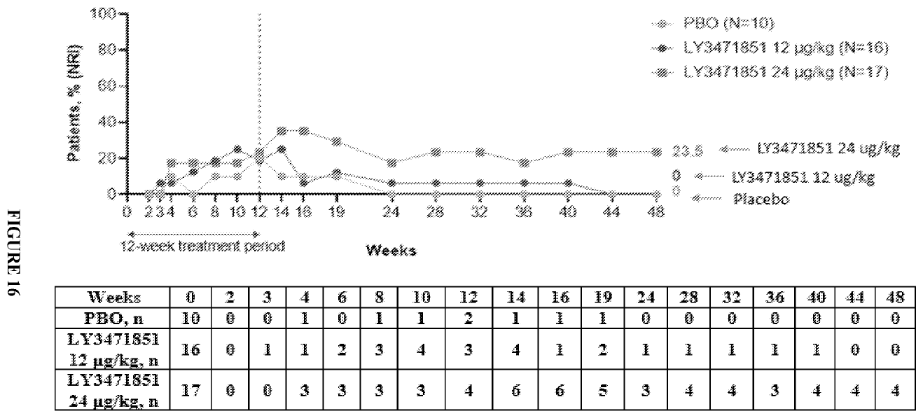

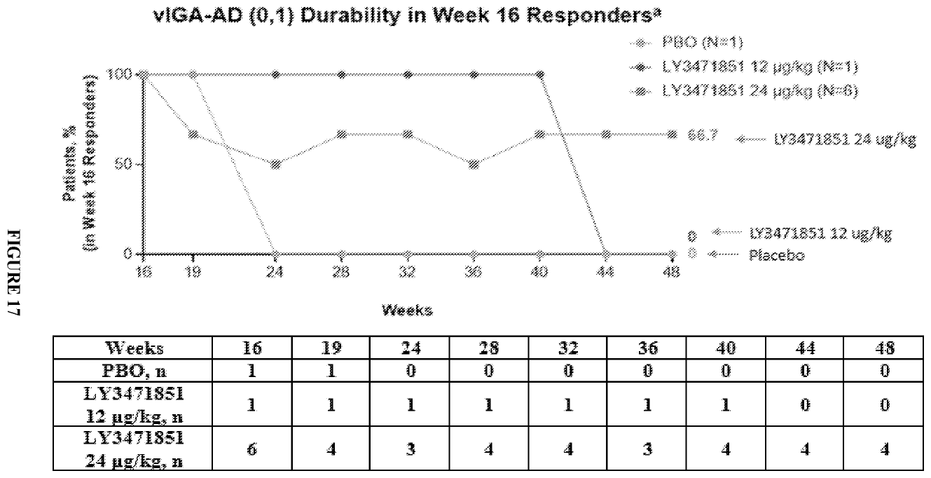

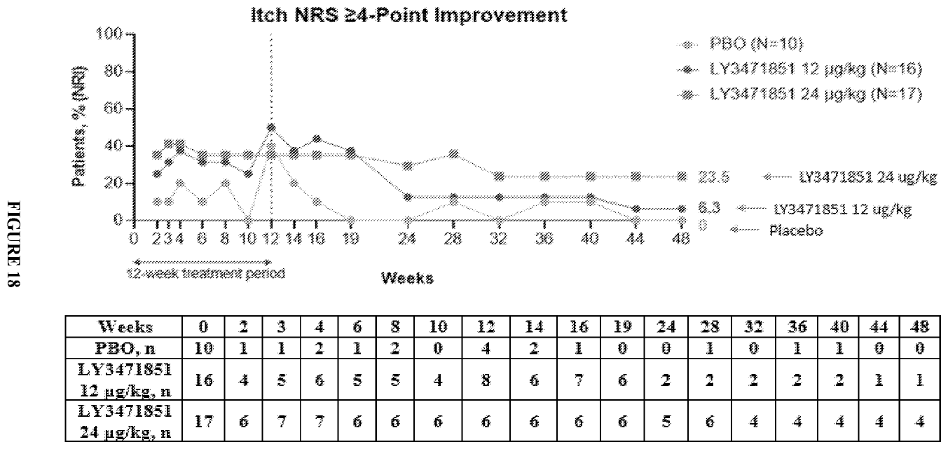

A dose-dependent trend in EASI percent change from baseline was observed with rezpegaldesleukin (LY3471851) vs. PBO up to Week 48, as shown in FIG. 14. A dose-dependent trend in EASI75 and vIGA-AD (0,1) responders was seen with rezpegaldesleukin (LY3471851) vs. PBO up to Week 48, as shown in FIG. 15 for EASI75 (n=number of responders, EASI75-75% improvement from baseline in Eczema Area and Severity Index score; NRI-non-responder imputation; PBO-placebo), and FIG. 16 for vIGA-AD (0,1), n=number of responders, NRI-non-responder imputation; PBO-placebo. A high percentage of week 16 vIGA-AD (0,1) responders maintained response to week 48 with LY3471851 24 μg/kg, as shown in FIG. 17. A dose-dependent trend in Itch NRS responders was seen with LY3471851 vs. PBO up to Week 48, as shown in FIG. 18. A trend toward dose-dependent improvement was observed in EASI and vIGA-AD scores and EASI75, vIGA-AD (0,1), and itch ≥4-point improvement responder rates with LY3471851 vs. placebo through 12 weeks of treatment. Improvements with LY3471851 24 μg/kg were sustained during follow-up to 48 weeks. Total Tregs and CD25bright Tregs increased with LY3471851 vs. placebo up to Week 12. The observed rezpegaldesleukin effects in atopic dermatitis patients provide evidence of durable clinical efficacy responses which support the induction and maintenance dosing regimen embodiments provided herein.

The safety profile observed at the doses studied supports further clinical development of rezpegaldesleukin in patients with AD. Adverse Events and Injection Site Reactions:

| | | LY3471851 12 | LY3471851 |
|---|---|---|---|
| | PBO | μg/kg | 24 μg/kg |
| AEs, n (%) # events | (N = 10) | (N = 16) | (N = 17) |
| AEs | 8 (80.0) 13 | 9 (56.3) 16 | 13 (76.5) 28 |
| Infections and | 2 (20.0) 2 | 4 (25.0) 5 | 7 (41.2) 11 |
| infestations | | | |
| Gastrointestinal | 3 (30.0) 4 | 1 (6.3) 1 | 3 (17.6) 3 |
| disorders | | | |
| Investigations^{a} | 0 | 0 | 4 (23.5) 4 |
| Nervous system | 0 | 2 (12.5) 2 | 2 (11.8) 2 |
| disorders | | | |
| General disorders | 1 (10.0) 1 | 1 (6.3) 1 | 2 (11.8) 2 |
| and administration | | | |
| site conditions | | | |
| Eye disorders | 0 | 2 (12.5) 2 | 0 |
| Skin and | 0 | 1 (6.3) 1 | 1 (5.9) 1 |
| subcutaneous | | | |
| tissue disorders | | | |
| Musculoskeletal | 1 (10.0) 20 | 0 | 1 (5.9) 1 |
| and connective | | | |
| tissue disorders | | | |
| Postoperative | 0 | 0 | 1 (5.9) 1 |
| wound infections | | | |
| Renal and urinary | 0 | 0 | 1 (5.9) 1 |
| disorders | | | |
| Respiratory, | 0 | 0 | 1 (5.9) 1 |
| thoracic and | | | |
| mediastinal | | | |
| disorders | | | |
| Injury, poisoning | 2 (20.0) 2 | 1 (6.3) 1 | 1 (5.9) 1 |
| and procedural | | | |
| complications | | | |
| Metabolism and | 1 (10.0) 1 | 1 (6.3) 1 | 0 |
| nutrition disorders | | | |
| Gastroesophageal | 1 (10.0) 1 | 0 | 0 |
| reflux disease | | | |
| Blood and | 0 | 1 (6.3) 1 | 1 (5.9) 1 |
| lymphatic system | | | |
| disorders | | | |
| Injection site | 0 | 1 (6.3) 1 | 0 |
| discoloration | | | |
| Immune system | 0 | 1 (6.3) 1 | 0 |
| disorders | | | |
| Blood and | 0 | 1 (6.3) 1 | 1 (5.9) 1 |
| lymphatic system | | | |
| disorders | | | |
| Severe Aes | 3 (30.0) 4 | 0 | 0 |
| SAEs | 2 (20.0) 3 | 0 | 0 |
| Deaths | 0 | 0 | 0 |
| AEs leading to study | 0 | 1 (6.3) 2^{b} | 3 (17.6) 3^{c} |
| discontinuation | | | |
| Injection site | 1 | 43 | 34 |
| reactions^{d}, # events | | | |

^{a}Increase in eosinophil count, hepatic enzymes, lymphocyte count, and weight;
^{b}Nausea and headache;
^{c}Abscess limb, eosinophil count increase, and urticaria;
^{d}Solicited reports AE-adverse event; PBO = placebo; SAE-serious AE. The majority of injection site reactions were mild or moderate in severity.

### Example 3

A Phase 1, Double-Blind, Randomized, Placebo-Controlled Study to Evaluate the Safety, Tolerability, and Pharmacokinetics of Subcutaneous Rezpegaldesleukin in Patients with Psoriasis

Impaired IL-2 production and dysfunction in regulatory T cell biology have been identified as key immunological defects contributing to the pathogenesis of multiple autoimmune and inflammatory diseases including psoriasis (PsO). Patients with PsO have shown either a decrease in Treg cell numbers or a reduction in immunosuppressive functions of Treg cells. rezpegaldesleukin selectively stimulates Treg cell expansion and activation. Here, we describe the efficacy, safety, and biological effects of rezpegaldesleukin in a study of patients with PsO.

In this Phase 1b, double-blind, placebo-controlled study KFAC (NCT04081350), patients were randomized to either 24 μg/kg rezpegaldesleukin (N=21) or placebo (PBO) (N=5) dosed bi-weekly, via subcutaneous injections, up to week (W) 12 with a follow-up period of W12-19. Inclusion criteria at baseline consisted of adult patients who were candidates for systemic therapy or phototherapy, with plaque PsO involving ≥10% body surface area in the affected skin, Static Physician's Global Assessment (sPGA) of ≥3, at least 2 similar and evaluable lesions, and a Psoriasis Area and Severity Index (PASI) score of ≥12. Efficacy was assessed through PASI, sPGA, and Itch Numerical Rating Scale (Itch NRS). Safety was assessed through Treatment-Emergent Adverse Effects (TEAE), death, discontinuation, hematology lab results, and Injection Site Reactions (ISR). Pharmacodynamics (PD) were evaluated using flow cytometry.

Demographic and disease baseline characteristics are presented in Table KFAC 1.

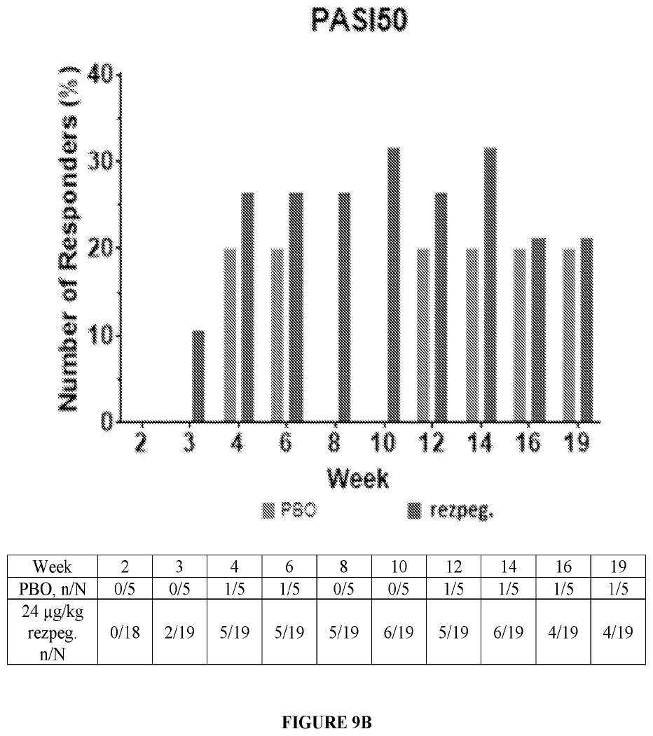

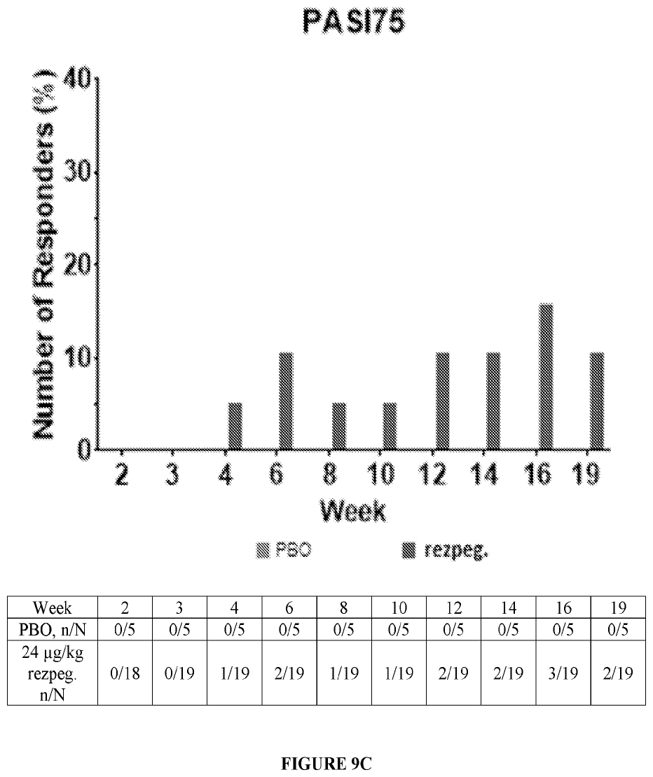

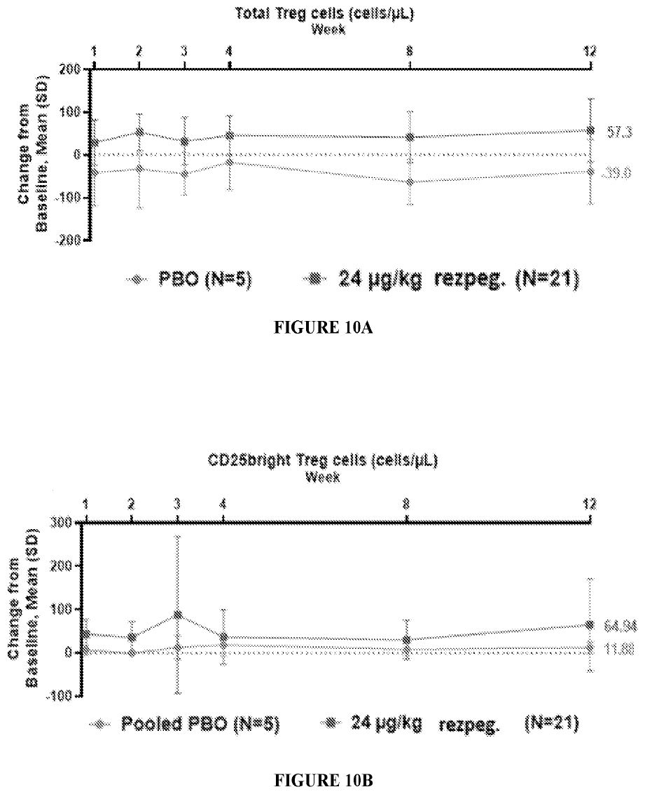

Rezpegaldesleukin treated patients saw a sustained improvement in PASI score up to W19 relative to PBO (FIG. 9). At W12, 26.3% and 10.5% of rezpegaldesleukin treated patients achieved PASI-50 and PASI-75 respectively (FIG. 9). At W12, in rezpegaldesleukin treated patients there was a sustained decrease, versus PBO, change from baseline (CFB) (−2.9 vs-0.4) in Itch NRS. For sPGA (0/1) a greater percentage of rezpegaldesleukin treated patients were responders, versus PBO, at W12 (18.2% vs 0%). Data through W19 evidence a duration of efficacy after rezpegaldesleukin treatment is stopped with the maintenance of PASI-75, with no sign of relapse or reduced efficacy over this W7 observation period. A summary of safety variables and ISR events related to PBO and 24 μg/kg rezpegaldesleukin are presented in Table KFAC 2. In rezpegaldesleukin treated patients, 34 TEAEs occurred, and 4 TEAEs led to discontinuation. 1 severe TEAE (Exacerbation of PsO), and no deaths occurred in the rezpegaldesleukin treated patients. No AEs, severe TEAEs, deaths, or discontinuations occurred in PBO treated patients. The most commonly occurring ISR reported for rezpegaldesleukin treated patients was Erythema (71.4%) and Induration (61.9%). Eosinophils were increased in rezpegaldesleukin treated patients, relative to PBO, by W12 (CFB, 0.392 [GL/L] vs 0.02 [GL/L]). At W12, total Treg cells increased in rezpegaldesleukin treated patients versus PBO (CFB, 57.3 [cells/uL] vs −39.0 [cells/uL]), and CD25bright Treg cells were elevated in the rezpegaldesleukin treated patients, relative to PBO, (CFB, 64.94 [cells/uL] vs. 11.88 [cells/uL]) (FIG. 10). Tcon cells, at W12, were decreased, relative to PBO (CFB, 308.80 [cells/uL] vs 673.13 [cells/uL]). NK cells were elevated in the rezpegaldesleukin treated patients relative to PBO at W12 (CFB, 955.1 [cells/uL] vs 96.0 [cells/uL]). Rezpegaldesleukin was well-tolerated and safe in patients with PsO. In patients treated with rezpegaldesleukin T reg cells numbers increased by W12, and PASI, sPGA, and Itch NRS outcomes improved by W12, and PASI was maintained after drug withdrawal up to W19.

TABLE KFAC.1
Demographic and Disease Baseline Characteristics

| | | 24 μg/kg |
|---|---|---|
| | PBO | rezpeg. |
| Characteristic | (N = 5) | (N = 21) |
| Sex, n (%) | | |
| Female | 2 (40.0) | 9 (42.9) |
| Male | 3 (60.0) | 12 (57.1) |
| Race, n (%) | | |
| White | 5 (100) | 14 (66.7) |
| Black or African | 0 | 2 (9.5) |
| American | | |
| Asia | 0 | 3 (14.3) |
| American Indian | 0 | 1 (4.8) |
| or Alaska Native | | |
| Other | 0 | 0 |
| Ethnicity, n (%) | 0 | 1 (4.8) |
| Hispanic or Latino | 0 | 2 (9.5) |
| Not Hispanic or | 5 (100) | 19 (90.5) |
| Latino | | |
| Age (years), Mean (SD) | 42.6 (15.09) | 47.5 (13.39) |
| PASI (score), Mean (SD) | 31.6 (7.96) | 29.3 (6.21) |
| sPGA (score), Mean (SD) | 3.4 (0.55) | 3.2 (0.44) |
| Itch NRS (score), | 6.2 (2.95) | 7.9 (1.92) |
| Mean (SD) | | |
| PGA, Mean (SD) | 4.2 (1.30) | 4.5 (0.87) |
| Any Prior | 4 (80) | 16 (76.2) |
| Medications, n (%) | | |

PASI = Psoriasis Area and Severity Index (score 0-72);
sPGA: static Physician's Global Assessment;
NRS: Numeric Rating Scale;
PGA: Patient's Global Assessment of Disease Severity.

TABLE KFAC.2
Summary of safety variables and ISR events for
PBO and 24 μg/kg rezpegaldesleukin.

| | | 24 μg/kg |
|---|---|---|
| | PBO | rezpeg. |
| Category | (N = 5) | (N = 21) |
| Adverse Events [n (%) m] | 0 | 14 (66.7) 34 |
| Infections and infestations | 0 | 6 (28.6) 7 |
| Skin and subcutaneous | 0 | 3 (14.3) 3 |
| tissue disorders | | |
| Blood and lymphatic system | 0 | 2 (9.5) 3 |
| disorders | | |
| General disorders and | 0 | 4 (19.0) 8 |
| administration site | | |
| conditions | | |
| Investigations | 0 | 2 (9.5) 2 |
| Eosinophil count increased | | |
| Gastrointestinal disorders | | 1 (4.8) 2 |
| Musculoskeletal and | 0 | 2 (9.5) 5 |
| connective tissue disorders | | |
| Respiratory, thoracic and | 0 | 2 (9.5) 5 |
| mediastinal disorders | | |
| Neoplasms benign, | 0 | 1 (4.8) 1 |
| malignant and unspecified | | |
| (incl cysts and polyps) | | |
| Renal and urinary disorders | 0 | 1 (4.8) 1 |
| Severe Adverse Events [n (%) m] | 0 | 1 (4.8) 1 |
| Adverse Events with Outcome of | 0 | 0 |
| Death [n (%) m] | | |
| Serious Adverse Events [n (%) m] | 0 | 0 |
| Adverse Events Leading to | 0 | 4 (19.0) 4 |
| Discontinuation of Study [n (%) m] | | |
| Skin and subcutaneous | 0 | 2 (9.5) 2 |
| tissue disorders | | |
| Investigations | 0 | 1 (4.8) 1 |
| (Eosinophil count increased) | | |
| Neoplasms benign, | 0 | 1 (4.8) 1 |
| malignant and unspecified | | |
| (incl cysts and polyps) | | |
| Injection Site Reaction [n (%)] | 0 | 28 |

n = Number of subjects;
m = Number of events
For each category, subjects are included only once, even if they experienced multiple events in that category.

Results from KFAC-A Phase 1 Randomized Psoriasis Study:

### Efficacy and Safety of a Selective Regulatory T-Cell Inducing IL-2 Conjugate

### Rezpegaldesleukin (NKTR-358/LY3471851) in the Treatment of Psoriasis:

Impaired interleukin (IL)-2 production and dysfunction in regulatory T cell (Treg) biology contribute to the pathogenesis of multiple autoimmune and inflammatory diseases, including psoriasis. Patients with psoriasis have shown a decrease in either Treg numbers or immunosuppressive functions of Tregs. Rezpegaldesleukin (LY3471851/NKTR-358) is a polyethylene glycol conjugate of recombinant human IL-2, which selectively stimulates Treg expansion and suppressive function. The following results describe the efficacy, safety, and biologic effects of LY3471851 in a Phase 1b, double-blind, placebo-controlled study KFAC (NCT04081350) of patients with psoriasis.

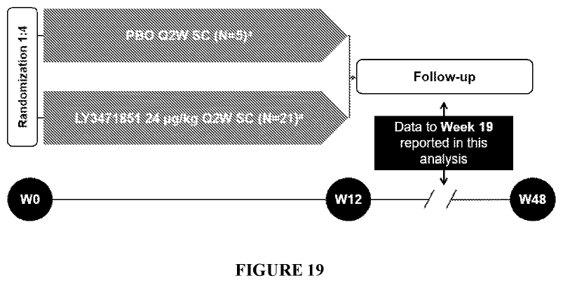

The general study design is shown in FIG. 19. Key Eligibility Criteria were; Aged 18-70 years, Plaque psoriasis involving ≥10% body surface area in the affected skin, Candidates for systemic therapy or phototherapy, At least 2 similar and evaluable lesions, Static Physician's Global Assessment (sPGA) score ≥3, Psoriasis Area and Severity Index (PASI) ≥12 Assessments and Statistical Analyses were:

### Assessments

Efficacy: PASI, sPGA, and Itch Numeric Rating Scale (NRS)
Safety: Treatment-emergent adverse effects and injection site reactions
Pharmacodynamics: Flow cytometry and epigenetic markers

### Statistical Analyses

Observed data were summarized using descriptive statistics

### Demographics and Baseline Characteristics:

| | PBO | | LY3471851 24 μg/kg | |
|---|---|---|---|---|
| | (N = 5) | | (N = 21) | |
| Age, years | 42.6 | (15.1) | 47.5 | (13.4) |
| Sex, n (%) | | | | |
| Female | 2 | (40.0) | 9 | (42.9) |
| Male | 3 | (60.0) | 12 | (57.1) |
| Race, n (%) | | | | |
| White | 5 | (100) | 14 | (66.7) |
| Black or African | 0 | | 2 | (9.5) |
| American | | | | |
| Asian | 0 | | 3 | (14.3) |
| American Indian or | 0 | | 1 | (4.8) |
| Alaska Native | | | | |
| Other | 0 | | 1 | (4.8) |
| Ethnicity, n (%) | | | | |
| Hispanic or Latino | 0 | | 2 | (9.5) |
| Not Hispanic or Latino | 5 | (100.0) | 19 | (90.5) |
| PASI | 31.6 | (8.0) | 29.3 | (6.2) |
| sPGA score | 3.4 | (0.6) | 3.2 | (0.4) |
| Itch NRS score | 6.2 | (3.0) | 7.9 | (1.9) |
| Prior medications, n (%) | 4 | (80.0) | 16 | (76.2) |

Data are mean (standard deviation) unless stated otherwise.
NRS = Numeric Rating Scale;
PASI = Psoriasis Area and Severity Index;
PBO = placebo;
sPGA = static Physician's Global Assessment

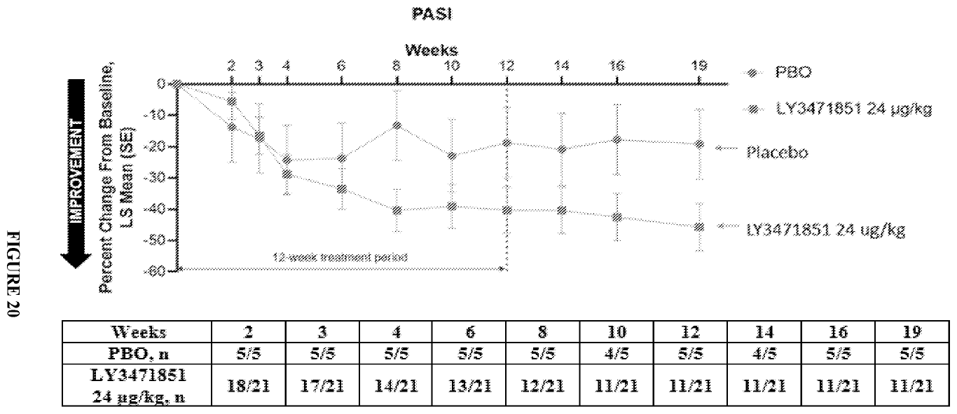

PASI improved with rezpegaldesleukin (LY3471851) vs. PBO and was maintained up to Week 19, as shown in FIG. 20. At week 12, 26% of LY3471851-Treated Patients achieved PASI 50 and 11% achieved PASI 75, and response rates were maintained up to week 19. At Week 12, Itch NRS and sPGA scores improved with LY3471851 vs. PBO. At week 12, total Tregs and CD25bright Tregs increased with 10 LY3471851 vs. PBO. The observed rezpegaldesleukin effects in psoriasis patients provide evidence of durable clinical efficacy responses which support the induction and maintenance dosing regimen embodiments provided herein.

### Example 4

A Randomized, Double-Blind, Placebo-Controlled, Phase 2 Study of Rezpegaldesleukin in Adults with Systemic Lupus Erythematosus

Systemic lupus erythematosus is a chronic, debilitating, autoimmune disease characterized by the presence of autoreactive B cells and elevated levels of autoantibodies. The disease can affect multiple organ systems and follows an unpredictable clinical course. Patients may present with arthralgia, arthritis, skin rash, alopecia, oral ulcers, pleuritis, pericarditis, nephritis, vasculitis, stroke, seizure, leukopenia, thrombocytopenia, anemia, photosensitivity, and the presence of autoantibodies directed to nuclear antigens. More than 60% of patients with SLE will develop clinically detectable organ damage about 4 years after the diagnosis, as measured by the Systemic Lupus International Collaborating Clinics/American College of Rheumatology Damage Index (SDI) (Cooper et al. 2007).

The standard-of-care for SLE varies widely and currently includes the use of corticosteroids, nonsteroidal anti-inflammatory drugs (NSAIDs), antimalarial medication, cytotoxic agents, and immunosuppressants. Unfortunately, the morbidity of the disease remains substantial, as measured by various tools for evaluating health-related quality of life, loss of work productivity, pain, and fatigue (Ad Hoc Committee on Systemic Lupus Erythematosus Response Criteria for Fatigue 2007; Özel and Argon 2015). A meta-analysis involving more than 27,000 patients showed that SLE patients had a 3-fold increase in risk of death compared with the general population (Yurkovich et al. 2014). Patients with SLE need better treatment options.

The dysfunction in Treg cell biology has been proposed as a key defect in SLE, leading to the breakdown of immune self-tolerance (Ohl and Tenbrock 2015). The goal of rezpegaldesleukin therapy is to increase Treg number and function, with a minimal effect on Tcons and natural killer (NK) cells. Study JIP-MC-KFAJ (KFAJ) is a Phase 2 study to evaluate the efficacy and safety of rezpegaldesleukin in adult patients with SLE. Results of this study will be used to guide the dose selection and further characterize the benefit/risk profile of rezpegaldesleukin.

| Objectives | Estimands/Endpoints |
|---|---|
| Primary | |
| To determine whether | The study will compare rezpegaldesleukin |
| treatment with | with placebo in participants with SLE. |
| rezpegaldesleukin Q2W is | The primary comparison of interest is the |
| superior to placebo in | difference in proportion of participants |
| reducing the signs and | who |
| symptoms of SLE | achieve SLEDAI-4 response at Week 24. |
| as measured | The primary comparison will be assessed |
| by SLEDAI-4 | using a composite estimand where the |
| | intercurrent events of premature |
| | discontinuation and use of prohibited |
| | medication are part of the response |
| | definition. |
| Secondary | |
| To determine whether | The study will compare rezpegaldesleukin |
| treatment with | with placebo in participants with SLE. |
| rezpegaldesleukin Q2W is | Secondary comparisons of interest are the |
| superior to placebo in | difference in proportion of participants |
| reducing other measures | who at |
| of signs and | Week 24 achieve: |
| symptoms of SLE | BILAG-based Composite Lupus |
| | Assessment (BICLA) response |
| | SRI-4 response |
| | Lupus Low Disease Activity State |
| | (LLDAS) |
| | Secondary objectives will be assessed |
| | using a |
| | composite estimand (as described above). |

Abbreviations: BILAG = British Isles Lupus Assessment Group - 2004; Q2W = every 2 weeks; SLE = systemic lupus erythematosus; SLEDAI-2K = Systemic Lupus Erythematosus Disease Activity Index 2000; SLEDAI-4 = defined as a ≥4-point reduction in SLEDAI-2K score from baseline; SRI-4 = Systemic Lupus Erythematosus Responder Index-4.

### Overall Design:

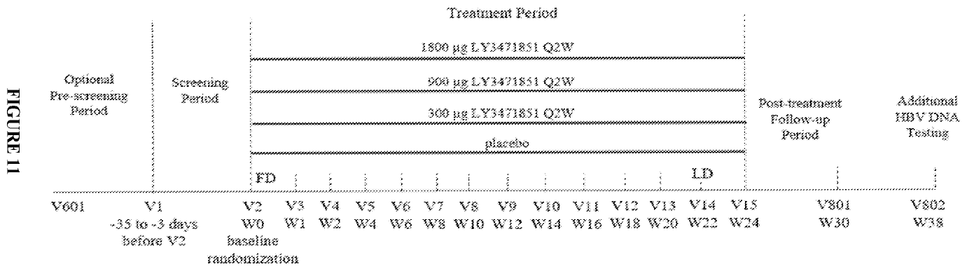

Study J1P-MC-KFAJ (KFAJ) is a multicenter, randomized, double-blind, placebo-controlled, parallel-group Phase 2 study to evaluate the efficacy and safety of 3 dose
levels of rezpegaldesleukin given via subcutaneous injection (SC) every 2 weeks (Q2W) versus
placebo in adult study participants with at least moderately active SLE. The study duration is
approximately 35 weeks over 3 required study periods:
—Screening period: beginning within 5 weeks before randomization, -Treatment period: 24 weeks, inclusive of the randomization visit to the last visit of this period, and -Post-treatment follow-up period: minimally 6 weeks, with an additional 8 weeks for some participants. An optional prescreening period precedes the required screening period. Participants will maintain their usual standard-of-care medication regimen for SLE throughout
the study. The maximum daily steroid dose allowed at entry will be prednisone 20 mg (or equivalent), which must be tapered to 10 mg daily (or equivalent) in time for the Week 12 assessments in order for the participant to be considered a responder. This is a parallel, 4-arm treatment study that is participant-blinded and investigator-blinded. Approximately 280 participants will be randomly assigned to study intervention.
Intervention Groups and Duration: Participants will be randomized in a 1:1:1:1 ratio (70 per group) to receive one of the following study interventions: 1800 μg rezpegaldesleukin Q2W, 900 μg rezpegaldesleukin Q2W, 300 μg rezpegaldesleukin Q2W, or placebo Q2W.
Schema of Study JIP-MC-KFAJ, a Phase 2 study to evaluate the efficacy and safety of rezpegaldesleukin in adults with systemic lupus erythematosus, is shown in FIG. 11. (Note: rezpegaldesleukin is also referred to as LY3471851.)
Note: Visit 802 is for HBV DNA testing of randomized participants who were positive for antibody to hepatitis B core antigen (anti-HBC) at screening. Abbreviations: FD=first dose administered; HBV=hepatitis B virus; LD=last dose administered; Q2W=every 2 weeks; V=eCRF visit; W=study week relative to baseline visit.

Local injection site reactions (ISR) may occur with rezpegaldesleukin. Cytokine release syndrome (CRS) and clinical symptom complex attributed to elevated cytokine levels may be observed with IL-2 agonists. Eosinophilia may be observed with IL-2 agonists. Other adverse may be observed with IL-2 agonists such as events characteristic of capillary leak syndrome, increased risk of infection, worsening or new onset autoimmune disease, cardiac disorders (cardiac rhythm disturbances, angina, or myocardial infarction), pulmonary disorders, central nervous system effects, or severe anemia/thrombocytopenia may be observed with high-dose IL-2 (aldesleukin).

Enrolled participants will receive study drug doses in the clinic to enable appropriate pre-dose safety assessments and post-dose monitoring. The routine safety assessments include physical examinations, clinical safety laboratory tests (including hematology and chemistry), suicidality/self-harm and depression evaluations, and collection of vital signs and spontaneously reported adverse events. The study design includes a

post-treatment follow-up period with at least one study visit for safety assessments. Systemic allergic/hypersensitivity reactions, which include anaphylaxis (Sampson et al. 2006)
and other immediate allergic-type reactions such as non-antibody mediated systemic reactions
(for example, CRS) are among the study's adverse events of interest. These reactions, if they occur, will prompt additional testing and data collection and are among the reasons that a participant might be discontinued from study drug. The protocol includes hepatic-related laboratory testing to support monitoring for symptoms and physical signs suggestive of liver or biliary toxicity, including jaundice, scleral icterus, and pruritis. Ongoing study-level monitoring of safety data will be performed. Interim analyses to review unblinded safety data will also be conducted.

Study KFAJ is a multicenter, randomized, double-blind, placebo-controlled, parallel-group Phase 2 study to evaluate the efficacy and safety of rezpegaldesleukin in adult study participants with at least moderately active SLE. The study has 3 required study periods and an optional prescreening period. Optional prescreening period: Study participants must have positive antinuclear, anti-dsDNA, and/or anti-Sm antibodies at Visit 1 to be eligible for randomization at Visit 2. The optional prescreening visit (Visit 601) is intended for those investigators who opt for central laboratory assessment of the prospective participant's antinuclear antibodies (ANA), anti-dsDNA, and anti-Sm antibody status before full

screening activities are initiated. If opted for, the prescreening determination of ANA, anti-dsDNA, and anti-Sm antibody status will be described in an appropriate informed consent form (ICF). Informed consent for the prescreening activities will be obtained, a participant identification number will be assigned, and samples for specified laboratory tests will be obtained. The optional prescreening visit can be repeated 2 times (no more) per participant, with a minimum of 4 weeks between visits. Note: If the optional prescreening is opted for, central laboratory testing for ANA, and dsDNA, and/or anti-Sm antibodies is still required as part of the screening procedures conducted at Visit 1. Participants with negative results at Visit 1 will not be eligible for enrollment, despite positive values in prescreening. A participant is considered to have completed the study if he or she has completed all required periods of the study, including the last visit or the last scheduled procedure shown in the SoA. The “end of the study” is defined as the date of the last visit or last scheduled procedure in the SoA for the last participant in the study globally.

### Schedule of Activities (SoA)

Prescreening and screening periods of Study J1P-MC-KFAJ
Visit 1 procedures may be conducted over more than 1 day as long as
all activities are completed within the allowable visit tolerance.

| | Prescreening | Screening | |
|---|---|---|---|
| | (optional) | (required) | Comment |
| | Visit number | | |
| | V601 | V1 | |
| | Weeks from randomization | | |
| | | −5 | |
| | Study day | | |
| | | — | |
| | Visit interval tolerance (days) | | |
| | | −35 to −3 | |
| | Fasting visit | | |
| | | | No fasting in this period. |
| Informed consent (for | X | | Central prescreening of ANA, anti- |
| prescreening only) | | | dsDNA, and/or anti-Sm is |
| | | | permitted after signing the |
| | | | prescreening ICF and can be |
| | | | performed before the participant |
| | | | signs the main KFAJ ICF. |
| Informed consent (for | | X | The KFAJ ICF must be signed |
| Study KFAJ) | | | before any required |
| | | | protocol-specific tests or required |
| | | | procedures are performed. |
| Inclusion and | | X | Participant eligibility must be |
| exclusion criteria, | | | reviewed and confirmed by an |
| review and confirm | | | eligibility review committee prior |
| | | | to randomization. |
| Demographics | X | X | Collect at Visit 1 only if not |
| | | | already collected at Visit 601. |
| Preexisting conditions | | X | |
| and medical history, | | | |
| including relevant | | | |
| surgical history | | | |
| Prespecified medical | | X | |
| history (indication and | | | |
| history of interest) | | | |
| Prior treatments for | | X | Includes: 1) any prior antimalarial |
| indication | | | or immunosuppressant therapy for |
| | | | SLE in the past 10 years (approved |
| | | | or investigational) that have now |
| | | | been discontinued, 2) any prior |
| | | | biologics or medications for |
| | | | immunologic disease in the past 24 |
| | | | weeks that have now been |
| | | | discontinued, and 3) any prior |
| | | | corticosteroids in the past 24 weeks |
| | | | that have now been discontinued. |
| Concomitant | | X | |
| medications | | | |
| Adverse events (AEs) | | X | AE collection begins when |
| | | | KFAJ ICF is signed. |
| | | | For AESIs, additional data are |
| | | | collected. |
| Substance use | | X | |
| (alcohol, caffeine, | | | |
| tobacco use) | | | |

Physical Evaluation

| | | |
|---|---|---|
| Vital signs | X | Blood pressure, body temperature, |
| | | pulse rate. Vital signs should be |
| | | measured after participant has been |
| | | sitting for at least 5 minutes. |
| Physical examination | X | The complete physical exam will |
| | | exclude pelvic, rectal, and breast |
| | | exams and will include assessment |
| | | for signs and symptoms of |
| | | tuberculosis (TB), including |
| | | peripheral lymph nodes. The SLE |
| | | symptom physical assessment is |
| | | captured in the electronic tablet. |
| 12-lead ECG (local) | X | |
| Chest x-ray (posterior- | X | Interpreted and reported by |
| anterior view) (local) | | radiologist or pulmonologist. |
| | | Not performed at screening if |
| | | performed within 3 months before |
| | | screening and if qualifying |
| | | radiographs or equivalent imaging |
| | | study and/or formal report are |
| | | available for investigator's review. |
| | | Lateral view may also be taken. |

Patient-Reported Outcomes (Electronic)

| | | |
|---|---|---|
| Patient's Global | X | Questionnaire is about lupus |
| Impression of Severity - | | symptoms severity. |
| 7 Days (lupus) | | |
| PROMIS SF Fatigue | X | |
| 7a | | |

Clinician-Administered Questionnaires (Electronic)

| | | |
|---|---|---|
| SLEDAI-2K | X | Collected via an electronic tablet |
| | | device. |
| 28 tender and swollen | X | Collected via an electronic tablet |
| joint counts | | device. |

Clinician-Administered Questionnaires (Paper)

| | | |
|---|---|---|
| C-SSRS | X | Adapted for assessment of ideation |
| Screening/Baseline | | and behavior categories only. |
| Self-harm Supplement | X | |
| Form | | |
| Self-Harm Follow-Up | X | Required if triggered by the Self- |
| Form | | Harm Supplement Form per |
| | | instructions. |

Laboratory Tests and Sample Collections

| | | | |
|---|---|---|---|
| Hematology | | X | |
| Clinical chemistry | | X | |
| Urine total protein, | | X | |
| urine creatinine, and | | | |
| urine protein/urine | | | |
| creatinine ratio | | | |
| (calculation) | | | |
| Urinalysis | | X | |
| Estimated glomerular | | X | Calculated using the Modification |
| filtration rate (eGFR) | | | of Diet in Renal Disease (MDRD) |
| | | | method. |
| Serum pregnancy | | X | Only for women of childbearing |
| | | | potential and women with a history |
| | | | of tubal ligation. Central |
| | | | laboratory. |
| Follicle-stimulating | | X | Required only to confirm |
| hormone (FSH) | | | “postmenopausal status” |
| Tuberculosis (TB) test | | X | Participants who had a tuberculin |
| | | | skin test (TST) will return from |
| | | | 48 to 72 hours after placement to |
| | | | have their test results read. |
| | | | Samples may be sent to central or |
| | | | local laboratory based on the type |
| | | | of test. A local laboratory must be |
| | | | qualified by local regulations. |
| HIV screening tests | | X | |
| Hepatitis C virus | | X | HCV RNA will be measured to |
| (HCV) screening tests | | | confirm positive hepatitis C virus |
| | | | antibody. |
| Hepatitis B virus | | X | Includes testing for HBsAg and |
| (HBV) screening tests | | | anti-HBc. |
| HBV DNA | | X | Only for participants who are |
| | | | positive for anti-HBc at screening. |
| Total | | X | |
| immunoglobulins | | | |
| (IgG) | | | |
| Antinuclear antibody | X | X | |
| (ANA) | | | |
| Anti-double stranded | X | X | |
| DNA (anti-dsDNA) | | | |
| Anti-Smith antibodies | X | X | |
| (anti-Sm) | | | |

Abbreviations: AESI = adverse events of special interest; ANA = antinuclear antibody; anti-dsDNA = anti-double stranded DNA; anti-HBc = antibody to hepatitis B core antigen; anti-Sm = anti-Smith antibodies; C-SSRS = Columbia-Suicide Severity Rating Scale; DNA = deoxyribonucleic acid; ECG = electrocardiogram; HBsAg = hepatitis B surface antigen; HIV = human immunodeficiency virus; ICF = informed consent form; RNA = ribonucleic acid; PROMIS = Patient-Reported Outcome Measurement Information System; SF = short form; SLE = systemic lupus erythematosus; SLEDAI-2K = Systemic Lupus Erythematosus Disease Activity Index 2000; V = visit.

Treatment period of Study J1P-MC-KFAJ
For early terminations that occur before the last visit in treatment period, see the activities listed for ETV in this table.

| | Visit number | | | | | | | | | | | | | | | |
|---|---|---|---|---|---|---|---|---|---|---|---|---|---|---|---|---|
| | V2 | V3 | V4 | V5 | V6 | V7 | V8 | V9 | V10 | V11 | V12 | V13 | V14 | V15 | ETV | Comment |
| | Weeks from Randomization | | | | | | | | | | | | | | | |
| | — | 1 | 2 | 4 | 6 | 8 | 10 | 12 | 14 | 16 | 18 | 20 | 22 | 24 | — | |
| | Study day | | | | | | | | | | | | | | | |
| | 1 | 8 | 15 | 29 | 43 | 57 | 71 | 85 | 99 | 113 | 127 | 141 | 155 | 169 | — | |
| | Visit interval tolerance (days) | | | | | | | | | | | | | | | |
| | — | ±3 | ±3 | ±3 | ±3 | ±3 | ±3 | ±3 | ±3 | ±3 | ±3 | ±3 | ±3 | ±3 | — | |
| | Fasting visit | | | | | | | | | | | | | | | |
| | X | | | | | | | X | | | | | | X | | |
| Inclusion and | X | | | | | | | | | | | | | | | Participant |
| exclusion criteria, | | | | | | | | | | | | | | | | eligibility must be |
| review and | | | | | | | | | | | | | | | | reviewed and |
| confirm | | | | | | | | | | | | | | | | confirmed by an |
| | | | | | | | | | | | | | | | | eligibility review |
| | | | | | | | | | | | | | | | | committee prior |
| | | | | | | | | | | | | | | | | to randomization. |
| Concomitant | X | X | X | X | X | X | X | X | X | X | X | X | X | X | X | |
| medications | | | | | | | | | | | | | | | | |
| Adverse events | X | X | X | X | X | X | X | X | X | X | X | X | X | X | X | For AESIs, |
| (AEs) | | | | | | | | | | | | | | | | additional data |
| | | | | | | | | | | | | | | | | are collected. |

Physical Evaluation

| | | | | | | | | | | | | | | | | |
|---|---|---|---|---|---|---|---|---|---|---|---|---|---|---|---|---|
| Height | X | | | | | | | | | | | | | | | Without shoes. |
| Weight | X | | | X | | X | | X | | X | | X | | X | X | |
| Vital signs | X | X | X | X | X | X | X | X | X | X | X | X | X | X | X | Blood pressure, |
| | | | | | | | | | | | | | | | | body temperature, |
| | | | | | | | | | | | | | | | | pulse rate. Vital |
| | | | | | | | | | | | | | | | | signs should be |
| | | | | | | | | | | | | | | | | measured after |
| | | | | | | | | | | | | | | | | participant has |
| | | | | | | | | | | | | | | | | been sitting for at |
| | | | | | | | | | | | | | | | | least 5 minutes. |
| Injection site | X | X | X | X | X | X | X | X | X | X | X | X | X | X | X | Performed |
| assessment | | | | | | | | | | | | | | | | by independent |
| | | | | | | | | | | | | | | | | injection site |
| | | | | | | | | | | | | | | | | assessor before |
| | | | | | | | | | | | | | | | | participant is seen |
| | | | | | | | | | | | | | | | | by blinded |
| | | | | | | | | | | | | | | | | site staff. |
| Symptom-directed | | | | X | | X | | X | | X | | X | | X | X | the SLE symptom |
| physical | | | | | | | | | | | | | | | | physical |
| examination | | | | | | | | | | | | | | | | assessment is |
| | | | | | | | | | | | | | | | | captured in the |
| | | | | | | | | | | | | | | | | electronic tablet. |
| 12-lead ECG | | | | | | | | X | | | | | | X | X | |
| (local) | | | | | | | | | | | | | | | | |

Patient-Reported Outcomes (Electronic)

| | | | | | | | | | | | | | | | | |
|---|---|---|---|---|---|---|---|---|---|---|---|---|---|---|---|---|
| FACIT Fatigue | X | | | | | | | | | | | | | X | X | Administer before |
| | | | | | | | | | | | | | | | | any clinical |
| | | | | | | | | | | | | | | | | assessments. |
| Patient Global | | | | | | | | X | | | | | | X | X | Administer before |
| Impression of | | | | | | | | | | | | | | | | any clinical |
| Change - Lupus - | | | | | | | | | | | | | | | | assessments. |
| 5 Point Version | | | | | | | | | | | | | | | | |
| Patient Global | | | | | | | | X | | | | | | X | X | Administer before |
| Impression of | | | | | | | | | | | | | | | | any clinical |
| Change - Fatigue - | | | | | | | | | | | | | | | | assessments. |
| 5 Point Version | | | | | | | | | | | | | | | | |
| Patient's Global | X | | | X | | X | | X | | X | | X | | X | X | Administer before |
| Impression of | | | | | | | | | | | | | | | | any clinical |
| Severity - 7 | | | | | | | | | | | | | | | | assessments. |
| Days (lupus) | | | | | | | | | | | | | | | | Questionnaire |
| | | | | | | | | | | | | | | | | is about lupus |
| | | | | | | | | | | | | | | | | symptoms |
| | | | | | | | | | | | | | | | | severity. |
| Patient's Global | X | | | X | | X | | X | | X | | X | | X | X | Administer before |
| Impression of | | | | | | | | | | | | | | | | any clinical |
| Severity - 7 | | | | | | | | | | | | | | | | assessments. |
| Days (fatigue) | | | | | | | | | | | | | | | | Questionnaire |
| | | | | | | | | | | | | | | | | is about fatigue |
| | | | | | | | | | | | | | | | | severity. |
| PROMIS SF | X | | | X | | X | | X | | X | | X | | X | X | Administer before |
| Fatigue 7a | | | | | | | | | | | | | | | | any clinical |
| | | | | | | | | | | | | | | | | assessments. |
| QIDS-SR16 | X | X | X | X | X | X | X | X | X | X | X | X | X | X | X | Administer before |
| | | | | | | | | | | | | | | | | any clinical |
| | | | | | | | | | | | | | | | | assessments. |
| SF-36v2 acute | X | | | | | | | X | | | | | | X | X | Administer before |
| | | | | | | | | | | | | | | | | any clinical |
| | | | | | | | | | | | | | | | | assessments. |
| Worst Joint | X | | | X | | X | | X | | X | | X | | X | X | Administer before |
| Pain NRS | | | | | | | | | | | | | | | | any clinical |
| | | | | | | | | | | | | | | | | assessments. |
| Worst Pain NRS | X | | | X | | X | | X | | X | | X | | X | X | Administer before |
| | | | | | | | | | | | | | | | | any clinical |
| | | | | | | | | | | | | | | | | assessments. |

Clinician-Administered Questionnaires (Electronic)

| | | | | | | | | | |
|---|---|---|---|---|---|---|---|---|---|
| BILAG 2004 | X | X | X | X | X | X | X | X | Collected via an |
| | | | | | | | | | electronic tablet |
| | | | | | | | | | device. |
| CLASI | X | X | X | X | X | X | X | X | Collected via an |
| | | | | | | | | | electronic tablet |
| | | | | | | | | | device. |
| Physician's Global | X | X | X | X | X | X | X | X | Collected via an |
| Assessment | | | | | | | | | electronic tablet |
| (PGA) of Disease | | | | | | | | | device. |
| Activity - VAS | | | | | | | | | |
| SLEDAI | X | X | X | X | X | X | X | X | Collected via an |
| Flare Index | | | | | | | | | electronic tablet |
| | | | | | | | | | device. |
| SLEDAI-2K | X | X | X | X | X | X | X | X | Collected via an |
| | | | | | | | | | electronic tablet |
| | | | | | | | | | device. |
| SLICC/ACR | X | | | | | | X | X | Collected via an |
| Damage Index | | | | | | | | | electronic tablet |
| | | | | | | | | | device. |
| 28 tender | X | X | X | X | X | X | X | X | Collected via an |
| and swollen | | | | | | | | | electronic tablet |
| joint counts | | | | | | | | | device. |

Clinician-Administered Questionnaires (Paper)

| | | | | | | | | | | | | | | | | |
|---|---|---|---|---|---|---|---|---|---|---|---|---|---|---|---|---|
| C-SSRS Since | X | X | X | X | X | X | X | X | X | X | X | X | X | X | X | Adapted for |
| Last Visit | | | | | | | | | | | | | | | | the assessment |
| | | | | | | | | | | | | | | | | of ideation |
| | | | | | | | | | | | | | | | | and behavior |
| | | | | | | | | | | | | | | | | categories only. |
| Self-Harm | X | X | X | X | X | X | X | X | X | X | X | X | X | X | X | |
| Supplement Form | | | | | | | | | | | | | | | | |
| Self-Harm | X | X | X | X | X | X | X | X | X | X | X | X | X | X | X | Required if |
| Follow-Up Form | | | | | | | | | | | | | | | | triggered by the |
| | | | | | | | | | | | | | | | | Self-Harm |
| | | | | | | | | | | | | | | | | Supplement Form |
| | | | | | | | | | | | | | | | | per instructions. |

Laboratory Tests and Sample Collections

| | | | | | | | | | | | | | | | | |
|---|---|---|---|---|---|---|---|---|---|---|---|---|---|---|---|---|
| Hematology | X | X | X | X | X | X | X | X | | X | | X | | X | X | |
| Clinical chemistry | X | X | X | X | X | X | X | X | | X | | X | | X | X | |
| Lipid panel | X | | | | | | | X | | | | | | X | X | For the fasting |
| | | | | | | | | | | | | | | | | lipid panel, |
| | | | | | | | | | | | | | | | | participants |
| | | | | | | | | | | | | | | | | should not eat or |
| | | | | | | | | | | | | | | | | drink anything but |
| | | | | | | | | | | | | | | | | water for 12 hours |
| | | | | | | | | | | | | | | | | before the visit. |
| | | | | | | | | | | | | | | | | Fasting is not |
| | | | | | | | | | | | | | | | | required at an |
| | | | | | | | | | | | | | | | | ETV. If a |
| | | | | | | | | | | | | | | | | participant attends |
| | | | | | | | | | | | | | | | | these visits in a |
| | | | | | | | | | | | | | | | | nonfasting state, |
| | | | | | | | | | | | | | | | | the sample should |
| | | | | | | | | | | | | | | | | still be collected. |
| | | | | | | | | | | | | | | | | This will not be |
| | | | | | | | | | | | | | | | | considered a |
| | | | | | | | | | | | | | | | | protocol violation. |
| Urinalysis | X | | | X | | X | | X | | X | | X | | X | X | |
| Urine total protein, | X | | | X | | X | | X | | X | | X | | X | X | |
| urine creatinine, | | | | | | | | | | | | | | | | |
| and urine | | | | | | | | | | | | | | | | |
| protein/urine | | | | | | | | | | | | | | | | |
| creatinine ratio | | | | | | | | | | | | | | | | |
| (calculation) | | | | | | | | | | | | | | | | |
| Urine pregnancy | X | | X | X | X | X | X | X | X | X | X | X | X | X | X | Only for women |
| (local) | | | | | | | | | | | | | | | | of childbearing |
| | | | | | | | | | | | | | | | | potential and |
| | | | | | | | | | | | | | | | | women with a |
| | | | | | | | | | | | | | | | | history of tubal |
| | | | | | | | | | | | | | | | | ligation. |
| HBV DNA | | | | | | | | X | | | | | | X | X | Only for |
| | | | | | | | | | | | | | | | | participants who |
| | | | | | | | | | | | | | | | | are positive for |
| | | | | | | | | | | | | | | | | anti-HBc at |
| | | | | | | | | | | | | | | | | screening |
| Total | X | | | X | | | | X | | | | | | X | X | |
| immunoglobulins | | | | | | | | | | | | | | | | |
| (IgG, IgA, IgM) | | | | | | | | | | | | | | | | |
| Antinuclear | | | | | | | | X | | | | | | X | X | |
| antibody (ANA) | | | | | | | | | | | | | | | | |
| Anti-double | X | | | X | | X | | X | | X | | X | | X | X | |
| stranded DNA | | | | | | | | | | | | | | | | |
| (anti-dsDNA) | | | | | | | | | | | | | | | | |
| Autoantibodies | X | | | | | | | X | | | | | | X | X | |
| (anti-Sm, | | | | | | | | | | | | | | | | |
| anti-RNP, | | | | | | | | | | | | | | | | |
| anti-SSA/Ro, | | | | | | | | | | | | | | | | |
| anti-SSB/La) | | | | | | | | | | | | | | | | |
| Antiphospholipid | X | | | | | | | X | | | | | | X | X | |
| antibodies | | | | | | | | | | | | | | | | |
| Complement | X | | | X | | X | | X | | X | | X | | X | X | |
| (C3 and C4) | | | | | | | | | | | | | | | | |
| Lymphocyte | X | | X | | | | | | | | | | | X | X | Will be performed |
| subsets | | | | | | | | | | | | | | | | only at sites that |
| | | | | | | | | | | | | | | | | meet assay |
| | | | | | | | | | | | | | | | | requirements. |
| Pharmacokinetic | X | X | X | X | | X | | X | | | | | | X | X | Collect samples |
| (PK) samples | | | | | | | | | | | | | | | | before dosing, if |
| | | | | | | | | | | | | | | | | dosing is scheduled. |
| | | | | | | | | | | | | | | | | Collect additional |
| | | | | | | | | | | | | | | | | samples as soon |
| | | | | | | | | | | | | | | | | as possible after |
| | | | | | | | | | | | | | | | | a systemic |
| | | | | | | | | | | | | | | | | hypersensitivity |
| | | | | | | | | | | | | | | | | reaction |
| Immunogenicity | X | | X | X | | X | | X | | | | | | X | X | Collect samples |
| (ADA) samples | | | | | | | | | | | | | | | | before dosing, if |
| | | | | | | | | | | | | | | | | dosing is scheduled. |
| | | | | | | | | | | | | | | | | Collect |
| | | | | | | | | | | | | | | | | additional samples |
| | | | | | | | | | | | | | | | | as soon as possible |
| | | | | | | | | | | | | | | | | after a systemic |
| | | | | | | | | | | | | | | | | hypersensitivity |
| | | | | | | | | | | | | | | | | reaction |
| Pharmacogenetics | X | | | | | | | | | | | | | | | Sample for DNA |
| sample | | | | | | | | | | | | | | | | pharmacogenetics |
| | | | | | | | | | | | | | | | | can be obtained |
| | | | | | | | | | | | | | | | | at or after the |
| | | | | | | | | | | | | | | | | specified visit. |
| Exploratory | X | | | X | | | | X | | | | | | X | X | |
| biomarker samples | | | | | | | | | | | | | | | | |

Randomization and Dosing

| | | | | | | | | | | | | | |
|---|---|---|---|---|---|---|---|---|---|---|---|---|---|
| Randomization | X | | | | | | | | | | | | |
| Dosing | X | X | X | X | X | X | X | X | X | X | X | X | No dosing at |
| | | | | | | | | | | | | | V3 or V15 |

Abbreviations: ACR = American College of Rheumatology; ADA = anti-drug antibody; AESI = adverse events of special interest; anti-dsDNA = anti-double stranded DNA; anti-HBc = antibody to hepatitis B core antigen; anti-RNP = anti-ribonucleoprotein; anti-Sm = anti-Smith antibodies; anti-SSA/Ro = anti-Sjögren's-syndrome-related antigen A (also called anti-Ro); anti-SSB/La = anti-Sjögren's syndrome type B antigen (SSB), also known as La protein; BILAG = British Isles Lupus Assessment Group; CLASI = Cutaneous Lupus Erythematosus Disease Area and Severity Index; C-SSRS = Columbia-Suicide Severity Rating Scale; DNA = deoxyribonucleic acid; ECG = electrocardiogram; ETV = early termination visit; FACIT-Fatigue = Functional Assessment of Chronic Illness Therapy-Fatigue; HBV = hepatitis B virus; IL = interleukin; NRS = numeric rating scale; PROMIS = Patient-Reported Outcome Measurement Information System; QIDS-SR16 = 16-Item Quick Inventory of Depressive Symptomatology-Self Report; SF = short form; SF-36v2 = Medical Outcomes Short Form 36-Item Health Survey version 2; SLE = systemic lupus erythematosus; SLEDAI-2K = Systemic Lupus Erythematosus Disease Activity Index 2000; SLICC = Systemic Lupus Erythematosus International Collaborating Clinics; V = visit; VAS = visual analog scale.

Post-treatment follow-up period of Study J1P-MC-KFAJ
Visit 802 is only for randomized participants who were positive for anti-HBc at
screening. All other participants will Visit number complete the study at V801.

| | Visit number | | |
|---|---|---|---|
| | V801 | V802 | Comment |
| | Weeks from randomization | | |
| | 30 or | 38 or | |
| | ETV + 6 | ETV + 14 | |
| | Study day | | |
| | 211 | 267 | |
| | Visit interval tolerance (days) | | |
| | ±7 | ±7 | |
| | Fasting visit | | |
| | | | No fasting in this period. |
| Concomitant medications | X | X | |
| Adverse events (AEs) | X | X | For AESIs, additional data are collected. |

Physical Evaluation

| | | | |
|---|---|---|---|
| Weight | X | | |
| Vital signs | X | X | Blood pressure, body temperature, and pulse rate. |
| | | | Vital signs should be measured after participant has |
| | | | been sitting for at least 5 minutes. |
| Injection site assessment | X | | Performed by independent injection site assessor |
| | | | before participant is seen by blinded site staff. |
| Symptom-directed physical | X | X | |
| examination | | | |

Clinician-Administered Questionnaires (Paper)

| | | | |
|---|---|---|---|
| C-SSRS Since Last Visit | X | X | Adapted for the assessment of ideation and behavior |
| | | | categories only. |
| Self-Harm Supplement Form | X | X | |
| Self-Harm Follow-Up Form | X | X | Required if triggered by Self-Harm Supplement |
| | | | Form, per instructions. |

Laboratory Tests and Sample Collections

| | | | |
|---|---|---|---|
| Hematology | X | X | |
| Clinical chemistry | X | X | |
| Urinalysis | X | | |
| Urine pregnancy (local) | X | X | Only for women of childbearing potential and women |
| | | | with a history of tubal ligation. |
| HBV DNA test | | X | Only for participants who are positive for anti-HBC at |
| | | | screening |
| Pharmacokinetic (PK) sample | X | | |
| Immunogenicity (ADA) sample | X | | |

Abbreviations: ADA = anti-drug antibody; AESI = adverse events of special interest; anti-HBc = antibody to hepatitis B core antigen; C-SSRS = Columbia-Suicide Severity Rating Scale; DNA = deoxyribonucleic acid; ETV = early termination visit; HBV = hepatitis B virus; V = visit.

### Scientific Rationale for Study Design:

SLEDAI-4 response as the primary endpoint:
The primary endpoint is the proportion of participants who achieve a SLEDAI-4 response at
Week 24. The selection of SLEDAI-4 for the primary endpoint measure was based on analyses from 2 recent SLE Phase 3 studies involving more than 2200 patients in a
similar patient population (Isenberg et al. 2016; Merrill et al. 2016). The purpose of those analyses was to characterize the clinical signs and symptoms most responsible for achieving
responder status with Systemic Lupus Erythematosus Responder Index-5 (SRI-5), and then to
use this information to design a more efficient study with clinically relevant endpoints

### (Kalunian

et al. 2018). The primary endpoint of those 2 recent studies required a 5-point reduction in
SLEDAI with no worsening in British Isles Lupus Assessment Group (BILAG) score or Physician's Global Assessment of Disease Activity (PGA). The analyses found that fewer than
1% of the study participants failed on SRI-5 due to BILAG or PGA worsening after achieving a
≥5-point reduction in SLEDAI. These data provide a rationale to focus on SLEDAI as the primary driver of clinical response. A 4-point reduction in SLEDAI is considered clinically meaningful and serves as the basis for the primary endpoint of SLEDAI-4 (Gladman et al. 2000).

Patients with at least moderately active SLE are an appropriate study population for a novel investigational product with immunomodulating properties. The study entry criteria will enable enrollment of patients who are representative of the general population of patients with at least moderately active SLE as defined by Systemic Lupus Erythematosus Disease Activity Index (SLEDAI)≥6 during screening and >4 for clinical features at randomization before administration of study drug. This SLEDAI cutoff has been used in other Phase 2 studies of SLE (van Vollenhoven et al. 2018; Wallace et al. 2018).

A double-blind, placebo-controlled design limits bias for both participant assessments and investigator assessments and enables a clearer interpretation of the effects of active drug. The multiple active dose levels will allow for an evaluation of safety and efficacy across a broad dose range and so provide information to guide dose selection for future studies. Evaluation of measures of efficacy at the Week 24 time point is consistent with the duration of treatment at the primary endpoint for proof-of-concept studies of other interventions for SLE (Furie et al. 2017; van Vollenhoven et al. 2018; Wallace et al. 2018). In longer studies, SLE efficacy outcomes a

Week 24 are similar to those observed at Week 52, which is the time point typically used for registration studies (Furie et al. 2011; Navarra et al. 2011; Isenberg et al. 2016; Merrill et al. 2016). Clinically meaningful effects were observed earlier than 24 weeks in open-label SLE studies of low-dose IL-2 formulations (He et al. 2016 [rhIL-2Ser125]; Rosenzwajg et al. 2019 [aldesleukin and ILT-101]), which are hypothesized to work through a mechanism of action similar to that of rezpegaldesleukin.

Study Population: Prospective approval of protocol deviations to recruitment and enrollment criteria, also known as protocol waivers or exemptions, is not permitted. All screening evaluations must be completed and reviewed to confirm that potential participants meet all eligibility criteria. Participant eligibility will be reviewed and confirmed by an eligibility review committee prior to randomization. The investigator will maintain a screening log to record details of all participants screened and to confirm eligibility or record reasons for screening failure, as applicable.

Inclusion Criteria: Patients will be included in the study only if all of the following inclusion criteria are met:

### Informed Consent

[1] Are capable of giving signed informed consent which includes compliance with the requirements and restrictions listed in the ICF and in this protocol.

### Participant Characteristics

[2] Are male or female patients from 18 to 65 years of age (inclusive), at the time of screening (Visit 1).

### SLE-Related Inclusion Criteria

[3] Have received a clinical diagnosis of SLE at least 24 weeks before the screening visit (Visit 1).
[4] Have documentation of having met at least 4 of the 11 Revised Criteria for Classification of Systemic Lupus Erythematosus according to the 1997 Update of the 1982 ACR (Tan et al. 1982; Hochberg 1997) before randomization.
[5] Have a positive ANA (titer ≥1:80) and/or a positive anti-dsDNA and/or positive anti-Sm antibody test at screening (Visit 1) as assessed by the central laboratory.
Note: This criterion must be assessed on the samples collected at Visit 1, not the prescreening samples collected at Visit 601. For repeat testing for ANA, anti-dsDNA, and/or anti-Sm during the screening period, see Section 5.4.1.
[6] Have a SLEDAI-2K score ≥6 at screening (Visit 1) and clinical SLEDAI-2K score ≥4 at randomization (Visit 2).
[7] Have active arthritis and/or active rash as defined by the SLEDAI-2K at screening and at randomization.

### Exclusion Criteria

Patients will be excluded from the study if any one of the following criteria are met:

### Corticosteroid Use

[8] Are currently receiving oral corticosteroids at doses >20 mg per day of prednisone (or equivalent) or have adjusted the dosage of corticosteroids within 2 weeks before randomization (Visit 2).
[9] Have received topical corticosteroids, other than stable doses of Class VI (mild, such as desonide) or Class VII (least potent, such as hydrocortisone), within 8 weeks before randomization (Visit 2).
[10] Have received parenteral (that is, intravenous or intramuscular) corticosteroids within 12 weeks before randomization (Visit 2) or are expected to require parenteral corticosteroids during the study.

### Other Prior or Concomitant Therapy

[11] Have started treatment with or adjusted the dosage of NSAIDs intended for treatment of signs and symptoms of SLE within 2 weeks before randomization (Visit 2).
[12] Have started treatment with or adjusted the dosage of an antimalarial (such as hydroxychloroquine, chloroquine, or quinacrine) within 12 weeks before randomization
(Visit 2); or have received more than a single antimalarial within 12 weeks before randomization (Visit 2).
[13] Have started treatment with or adjusted the dosage of an immunosuppressant (such as methotrexate, azathioprine, mycophenolate mofetil, or mizoribine,) within 12 weeks before randomization (Visit 2); or have received more than a single immunosuppressant within 12 weeks before randomization (Visit 2).
[14] Have received an oral calcineurin inhibitor, such as cyclosporine, tacrolimus, sirolimus, or voclosporin, within 12 weeks before randomization (Visit 2).
[15] Have received an oral Janus kinase (JAK) inhibitor, including but not limited to baricitinib, tofacitinib, and upacitinib, within 12 weeks before randomization (Visit 2).
[16] Have received cyclophosphamide (or any other cytotoxic agent) within 12 weeks before randomization (Visit 2).
[17] Have received biologic therapies in the specified number of weeks before screening:
[17a] rituximab or ustekinumab within 24 weeks before randomization (Visit 2),
[17b] other biologic therapies including, but not limited to, anticytokine or receptor blocker (etanercept, infliximab, certolizumab, adalimumab, golimumab, tocilizumab, anakinra, ixekizumab, secukinumab, belimumab, abatacept) within 12 weeks before randomization (Visit 2).
[19] Have received intravenous Ig within 24 weeks before randomization (Visit 2).
[19] Have received plasmapheresis within 12 weeks before randomization (Visit 2).
[20] Have received any live vaccine (that is, live attenuated) within 12 weeks of screening (Visit 1), or intend to receive a live vaccine during the study, or plan to receive such vaccine within 8 weeks of completing treatment in this study.
[21] Have received a Bacillus Calmette-Guerin (BCG) vaccination or treatment within 12 months of screening (Visit 1) or intend to have vaccination with BCG during the course of the study, or plan to receive such vaccination within 8 weeks of completing treatment in this study.
[22] Have participated within the last 30 days in a clinical study involving an investigational product. If the previous investigational product has a long half-life, at least 5 half-lives or 30 days (whichever is longer) should have passed before the randomization visit (Visit 2) of Study KFAJ.
[23] Have received treatment with aldesleukin or other IL-2-related treatment.

### Current or Historical Infections

[24] Have a current or recent acute, active infection. For at least 30 days prior to screening (Visit 1) and up to the time of randomization (Visit 2), participants must have no symptoms or signs of confirmed or suspected infection, and must have completed any appropriate anti-infective treatment.
[25] Have had, within 12 weeks before randomization (Visit 2), any of the following types of infection:
Serious (requiring hospitalization, and/or intravenous or equivalent oral antibiotic treatment),
Opportunistic (as defined in Winthrop at al. 2015),
Chronic (duration of symptoms, signs, and/or treatment of 6 weeks or longer),
Recurring (including, but not limited to, herpes simplex, herpes zoster, recurring cellulitis, chronic osteomyelitis).
   [26] Have human immunodeficiency virus (HIV) infection.
   [27] Have a current infection with hepatitis B virus (HBV DNA) (that is, positive for hepatitis B surface antigen [HBsAg] and/or PCR positive for HBV DNA).
   [28] Have a current infection with hepatitis C virus (that is, positive for HCV RNA).
   [29] Have current or past history of active tuberculosis (TB).
   [30] Have or have had latent TB infection (LTBI) that has not been treated with a complete course of appropriate therapy as defined by the World Health Organization and/or the United States Centers for Disease Control and Prevention, unless such treatment is underway.

### Other Current or Historical Medical Conditions

[31] Have severe active lupus nephritis defined clinically and/or by urine protein/creatinine ratio >300 mg/mmol (as an estimate of approximate proteinuria >3 g/day) or active urinary sediment with red blood cell cast(s), or histological evidence of lupus nephritis (Class III, IV, V, or VI according to the International Society of Nephrology classification criteria) within 12 weeks before randomization (Visit 2).
[32] Have active severe central nervous system lupus including aseptic meningitis, cerebral vasculitis, demyelinating syndrome, myelopathy, acute confusional state, psychosis, acute inflammatory demyelinating polyradiculoneuropathy, cranial neuropathy, plexopathy, status epilepticus, or cerebellar ataxia within 24 weeks before randomization (Visit 2).
[33] Have active fibromyalgia that, in the investigator's opinion, would make it difficult to appropriately assess SLE activity for the purposes of this study.
[34] Have been treated for or had an active occurrence of a systemic inflammatory condition other than SLE, such as, but not limited to, rheumatoid arthritis, juvenile chronic arthritis, spondyloarthropathy, Crohn's disease, ulcerative colitis, or psoriatic arthritis in the 12 weeks before randomization (Visit 2).
[35] Have experienced a thrombotic event within 24 weeks before randomization or are on anticoagulants and in the opinion of the investigator are not well-controlled regarding management of hypercoagulable risk.
[36] Have experienced any of the following within 12 months before screening: myocardial infarction, unstable ischemic heart disease, stroke, or New York Heart Association Stage IV heart failure.
[37] Have a history of clinically significant or uncontrolled cardiovascular disease (for example, hypertension, angina, congestive heart failure); endocrine disorder (for example, diabetes, thyroid dysfunction); or respiratory, hepatic, renal, gastrointestinal, hematologic, or neuropsychiatric disorder; or any other serious and/or unstable illness that in the opinion of the investigator, could constitute an unacceptable risk to the participant when taking an investigational product or could interfere with the interpretation of study data.
[38] Are, in the judgment of the investigator, actively suicidal and therefore deemed to be at significant risk for suicide.
[39] Have answered “yes” to either Question 4 or Question 5 on the “Suicidal Ideation” portion of the Columbia-Suicide Severity Rating Scale (C—SSRS) or have answered “yes” to any of the suicide-related behaviors on the “suicidal behavior” portion of the C—SSRS, and the ideation or behavior occurred within 4 weeks prior to randomization (Visit 2).
[40] Have a history of lymphoproliferative disease; or have signs or symptoms suggestive of possible lymphoproliferative disease, including lymphadenopathy or splenomegaly (other than primarily because of SLE); or have active primary or recurrent malignant disease; or have been in remission from clinically significant malignancy for <5 years.

### Exceptions:

Participants with cervical carcinoma in situ that has been resected with no evidence of recurrence or metastatic disease for at least 3 years may participate in the study if other study entry are met.
Participants with basal cell or squamous epithelial skin cancers that have been completely resected with no evidence of recurrence for at least 3 years may participate in the study if other study entry criteria are met.
[41] Have had any major surgery within 12 weeks before randomization (Visit 2) or will require major surgery during the study that, in the opinion of the investigator would pose an unacceptable risk to the participant.
[42] Have a known allergy to the investigational intervention or any of its excipients, clinically significant multiple or severe drug allergies, intolerance to topical corticosteroids, or history of severe post-treatment hypersensitivity reactions, including, but not limited to, erythema multiforme major, linear immunoglobulin A (IgA) dermatosis, toxic epidermal necrolysis, or exfoliative dermatitis.

### Diagnostic Assessments or Abnormal Laboratory Values

[43] Have any of the following specific abnormalities on screening laboratory tests:
alanine aminotransferase (ALT) or aspartate aminotransferase (AST)>2 times upper limit of normal (ULN)
total bilirubin level (TBL) ≥1.5 times ULN
hemoglobin <8.5 g/dL (<85.0 g/L)
total white blood cell count (WBC)<2500 cells/μL (<2.50×103/μL or <2.50 GI/L)
eosinophilia (absolute eosinophil count >3000 cells/μL (>3.0×103/μL or >3.0 GI/L)
neutropenia-absolute neutrophil count (ANC)<1000 cells/μL (<1.00×103/μL or <1.00 GI/L)
lymphopenia-absolute lymphocyte count (ALC)<500 cells/μL (<0.50×103/μL or <0.50 GI/L)
thrombocytopenia-platelets <50,000 cells/μL (<50×103/μL or <50 GI/L)
estimated glomerular filtration rate (eGFR) (Modification of Diet in Renal Disease [MDRD])<50 mL/min/1.73 m2 (FDA 2010).
   [44] Have known hypogammaglobulinemia or a screening immunoglobulin G (IgG) level <565 mg/dL (<5.65 g/L).
   [45] Have other abnormal laboratory values at screening that, in the opinion of the investigator, indicate unacceptable risk for the participant's safety in the study.
   [46] Have screening electrocardiogram (ECG) abnormalities that, in the opinion of the investigator, are clinically significant and indicate an unacceptable risk for the participant's safety in the study.
   Study intervention is defined as any investigational intervention, marketed product, placebo, or medical device intended to be administered to a study participant according to the study protocol. Study interventions:
   This study involves 3 dose levels of rezpegaldesleukin and placebo, as shown:

| Treatment Name | rezpegaldesleukin | | Placebo |
|---|---|---|---|
| Dosage Formulation | 0.5-mL vial delivers | | 0.9% sodium chloride |
| | 0.5 mg rezpegaldesleukin | | |
| Dosage Levels | 1800 | μg | not applicable |
| | 900 | μg | |
| | 300 | μg | |

| | | |
|---|---|---|
| Routes of | subcutaneous injection | subcutaneous injection |
| Administration | | |
| Dosing Instructions | Dosing every 2 weeks | Dosing every 2 weeks |

rezpegaldesleukin drug product will be provided as a sterile solution in a vial for SC injection. The drug product vials will be supplied in cartons, with the appropriate quantity specific to the planned dispensing schedule of the investigation product (IPs). Dosing solutions will be prepared at each clinical study site by an unblinded pharmacist (or other unblinded qualified individual) and loaded into syringes for SC dosing. When the dosing solutions are prepared according to the provided instructions, it will not be possible to distinguish rezpegaldesleukin from placebo. All participants should be monitored for 30 minutes or longer after dosing, according to investigator practice or local standard of care.
Blinding will be maintained throughout the conduct of the study as described in the separate Unblinding Plan. Assignment to treatment groups will be determined by a computer-generated random sequence using an interactive web-response system (IWRS). Investigators and all individuals involved in administering the blinded treatment or performing
assessments will remain blinded to each participant's assigned study intervention throughout the course of the study. To maintain this blind, an otherwise uninvolved party (unblinded pharmacist or other unblinded qualified individual) will be responsible for the preparation and dispensation of all study intervention. Blinded site personnel will administer the study intervention to the participant. At every visit following the first dose and prior to any blinded study activity, an independent Injection Site Reaction (ISR) assessor who is not involved with other study procedures will evaluate each participant for the presence of ISRs. If the participant presents with symptoms of an ISR, the ISR assessor will examine the impacted areas and record the information in the electronic case report form (eCRF). Whether an ISR is present or not, the ISR assessor will use a bandage (or similar material) to cover the anatomical area where the participant received his or her last dose. The purpose of covering this area is to minimize bias when blinded study personnel conduct other study assessments, given the known frequency of ISRs with the molecule.
Participants will receive study intervention directly from the investigator or designee at the study site, under medical supervision. The date and time of each dose administered in the clinic will be recorded in the source documents and recorded in the eCRF. Deviations from the prescribed dosage regimen should be recorded in the eCRF.

The primary and secondary efficacy endpoints are described in the following sections. The self-reported questionnaires will be administered in countries where the questionnaires have been translated into the native language of the region and linguistically validated. Any outcome measures that are participants' self-assessments should be completed before any clinical examinations are performed. All patient-reported and clinician-reported efficacy assessments will be captured on an electronic tablet collected at site visits.

### Primary Efficacy Outcome Measure: SLEDAI-4:

The primary efficacy endpoint is the proportion of participants who achieve a SLEDAI-4 response at Week 24. A SLEDAI-4 response is defined as a ≥4-point reduction in Systemic Lupus Erythematosus Disease Activity Index 2000 (SLEDAI-2K) score from baseline. To be considered a SLEDAI-4 responder, participants must complete the 24-week treatment period and comply with concomitant medication rules.

### Secondary Efficacy Outcome Measures:

British Isles Lupus Assessment Group (BILAG)-based Composite Lupus Assessment (BICLA) The BILAG-based Composite Lupus Assessment (BICLA) is a composite index used to assess disease activity in SLE. A BICLA response is defined as follows:
Reduction of all baseline BILAG-2004 A to B or C or D; and baseline BILAG-2004 B to C or D; and no BILAG-2004 worsening in other organ systems, as defined by ≥1 new BILAG-2004 A or ≥2 new BILAG-2004 B
No worsening from baseline in SLEDAI-2K, where worsening is defined as any increase from baseline in SLEDAI-2K
No worsening from baseline in participants' lupus disease activity, where worsening is defined by an increase ≥0.30 points on a 3-point PGA visual analogue scale (VAS)
No permanent discontinuation of investigational product, and
No use of restricted medications beyond the protocol-allowed threshold before assessment.

### Systemic Lupus Erythematosus Responder Index-4 (SRI-4)

The SRI-4 is a composite index used to assess disease activity in SLE. The SLEDAI-2K component is used to capture clinically meaningful improvement in disease activity, while the BILAG and PGA of Disease Activity components ensure that the improvement in overall disease is not accompanied by disease worsening in other organ systems. A SRI-4 response is defined as follows: Reduction of ≥4 points from baseline in SLEDAI-2K score, No new BILAG-2004 A or no more than 1 new BILAG-2004 B disease activity score, and No worsening (defined as an increase of ≥0.3 points [10 mm] from baseline) in PGA of Disease Activity. Participants must complete 24 weeks of the study and comply with concomitant medication rules to be considered a responder for the SRI-4 analysis.

### Lupus Low Disease Activity State (LLDAS)

An LLDAS response is defined as a low level of disease activity attained with or without use of low-dose steroids and/or tolerated standard maintenance doses of standard-of-care immunosuppressant medications (Franklyn et al. 2016).
Safety assessments occur at visits specified in the SoA. If multiple safety
assessments are scheduled for the same visit, the preferred order of completion is
1) ECG and then vital signs, 2) other safety assessments, including physical examinations and nonleading (spontaneous) adverse event collection, followed by C—SSRS (Section 8.3.1.1), and finally 4) blood sample collection for clinical laboratory, PK, PD, pharmacogenetic, biomarker, and immunogenicity testing. Any clinically significant findings that result in a diagnosis and that occur after the participant receives the first dose of study drug should be reported to Lilly or its designee as an adverse event via eCRF. The principle investigator will monitor the safety data throughout the study and should discuss immediate safety concerns with the sponsor immediately upon occurrence or awareness to determine whether the participant should continue or discontinue the study drug. The sponsor will monitor the safety data, including adverse events and serious adverse events, discontinuations, vital signs, and clinical laboratory results by means of blinded reviews performed at least quarterly and by other appropriate methods. These methods include reviews by a functionally independent safety physician and/or clinical research scientist who regularly reviews SAE reports in real time and across studies, and who reviews applicable clinical safety and epidemiological publications from the literature.

All protocol-required laboratory assessments, must be conducted in accordance with the laboratory manual and the SoA. If laboratory values from non-protocol specified laboratory assessments performed at the institution's local laboratory require a change in participant management or are considered clinically significant by the investigator (for example, SAE or adverse event or dose modification), then the results must be recorded in the eCRF. The investigator must review the laboratory report, document this review, and record any clinically relevant changes occurring during the study in the adverse event section of the eCRF.

### Injection Site Assessment:

Symptoms of a local injection site reaction may include erythema, induration, pain, pruritus, and edema. Solicited data from injection site assessments will not be routinely classified as an adverse event. If a participant reports symptoms (that is, an unsolicited event, volunteered by participant) or if the investigator determines that a clinically relevant injection site reaction has occurred, the event will be captured as an adverse event. In such a case, a specific adverse event of Injection site reaction will be reported and the event followed up to completion, in addition to completing the injection site assessment questionnaire in the eCRF.

Adverse events (AEs) will be reported by the participant or, when appropriate, by a caregiver, surrogate, or the participant's representative. Adverse events for this study include systemic allergic/hypersensitivity reactions, including cytokine release syndrome, and serious infections and opportunistic infections.

Pharmacokinetics: At the visits and times specified in the SoA, venous blood samples will be collected to determine the plasma concentrations of rezpegaldesleukin. The actual date and time (24-hour clock time) of dosing and sample collection must be recorded accurately on the appropriate forms. Instructions for the collection and handling of blood samples will be provided by the sponsor. Samples will be analyzed at a laboratory approved by the sponsor. Concentrations of rezpegaldesleukin will be assayed using a validated PK assay. Analyses of samples collected from participants who received placebo are not planned.

Pharmacodynamics: The PD biomarkers to be measured include changes in T-cell subsets. At the visits and times specified in the SoA, blood samples will be collected for the exploratory analysis of PD biomarkers.
Immunogenicity Assessments: At the visits and times specified in the SoA, predose venous blood samples will be collected to determine antibody production against rezpegaldesleukin. The actual date and time (24-hour clock time) of each sample collection will be recorded. To aid interpretation of these results, a predose blood sample for PK analysis will be collected at the same time points. Immunogenicity will be assessed by a validated assay designed to detect anti-drug antibodies (ADAs) in the presence of rezpegaldesleukin at a laboratory approved by the sponsor. Antibodies may be further characterized for cross-reactive binding to PEG and native IL-2, as well as their ability to neutralize the activity of rezpegaldesleukin and/or native IL-2. If the immunogenicity sample at the last scheduled assessment or discontinuation visit is treatment-emergent (TE)-ADA positive and the ADA cross-reactively bind native IL-2, additional samples may be taken every 3 months for up to one year from last dose or until the ADA signal returns to baseline (that is, no longer TE-ADA positive).
Statistical Hypotheses: The null hypothesis for the primary endpoint is that there is no difference between rezpegaldesleukin and placebo in reducing the signs and symptoms of SLE as measured by the proportion of participants who achieve a SLEDAI-4 response at Week 24. Approximately 280 participants will be randomly assigned to study intervention. All randomized participants in the modified intent-to-treat (mITT) population will be considered evaluable. Pairwise 2-sided tests of proportions with alpha=0.05 will be performed on each rezpegaldesleukin dose versus placebo. KFAJ has at least 80% power to detect a statistically-significant difference of 24% improvement over placebo in SLEDAI-4 response between rezpegaldesleukin and placebo at Week 24. Primary and secondary endpoint analyses will be tested at a 2-sided alpha level of 0.05 for frequentist analyses. The primary estimand that will be used to analyze primary and secondary endpoints is a composite response estimand where comparisons will not include data collected after intercurrent events of changes to background therapies or discontinuation. Participants who discontinue treatment prior to 24 weeks or who are noncompliant with concomitant medication rules are defined as nonresponders. Endpoint definition effectively gives
complete data which will be analyzed. A Bayesian model averaging approach will be used to estimate the dose response relationship. Bayesian model averaging is a general mixture distribution, where each mixture component is a different parametric model. Prior weights are placed on each model and the posterior model weights are updated based on how well each model fits the data (Gould 2019). This Bayesian model averaging approach is the Bayesian analog of the MCP-MOD methodology (Bretz et al. 2005), and the Qualification of the MCP-Mod procedure (FDA 2015) is supportive in the use of MCP-MOD or Bayesian model averaging to assist in dose selection decisions. No adjustments for multiplicity will be performed. Efficacy and PRO analysis models may contain the independent variables such as treatment group, baseline disease activity, and geographic region. Missing data for dichotomous responder endpoints will be imputed using the nonresponder imputation (NRI) method. Participants will be considered nonresponders for the NRI analysis if they do not meet all the clinical response criteria, they are noncompliant with concomitant medication rules, they permanently discontinue study intervention at any time before the end of the treatment period for any reason, or they are randomized and do not have at least 1 postbaseline observation. Mixed-effects model for repeated measures (MMRM) is the main method for analyzing continuous efficacy endpoints. Additional imputation methods may be considered for all endpoint types. Baseline will be defined as the last available value before the first dose of study intervention for both efficacy and safety analyses. In most cases, this value will be what is recorded at the randomization visit (Visit 2). Change from baseline will be calculated as the visit value of interest minus the baseline value. For the post-treatment Follow-up Period, the baseline is defined as the last non-missing assessment on or prior to entering the post-treatment Follow-up Period, that is, on or prior to the Week 24 visit, or the early termination visit (including ETV). The primary endpoint is the SLEDAI-4 response rate at Week 24 for the rezpegaldesleukin treatment group compared to placebo. Participants who fail to complete the 24-week treatment period or violate the concomitant medication rules will be imputed to nonresponse for the purpose of the
primary endpoint analysis. The objective of the primary endpoint is to determine whether rezpegaldesleukin is superior to placebo. The primary endpoint will be analyzed using a logistic regression model with baseline disease activity, corticosteroid dose at baseline, and geographic region as model covariates. Treatment difference and 95% CIs will be reported.
Secondary efficacy endpoints include the following at Week 24: the proportion of participants who achieve a BILAG-based Composite Lupus Assessment (BICLA) response, the proportion of participants who achieve SRI-4 response, and the proportion of participants who achieve LLDAS. Dichotomous secondary endpoints will be analyzed using the model specified for the primary analysis. Plasma concentrations of rezpegaldesleukin will be listed by time point using descriptive statistics. The data may also be analyzed using a population approach via nonlinear mixed effects modeling (NONMEM) with the NONMEM software. Patient-Report Outcomes: Categorical variables will be analyzed using logistic regression analyses, whereas MMRM will be the primary method of analysis for continuous endpoints. The analyses will be based on the mITT population, unless otherwise specified. Immunogenicity: Frequencies and percentages will be tabulated for the following: participants with pre-existing ADA, and participants who are TE-ADA positive (TE-ADA+) to rezpegaldesleukin. Treatment-emergent ADAs are defined as those with a titer 2-fold (1 dilution) greater than the minimum required dilution if no ADAs were detected at baseline (treatment-induced ADA) or those with a 4-fold (2 dilutions) increase in titer compared with baseline if ADAs were detected at baseline (treatment-boosted ADA). For the TE ADA+ participants, the distribution of maximum titers will be described. The frequency of cross-reactive and neutralizing antibodies, if assessed, may also be tabulated for the TE-ADA+ participants. The relationship between the presence of antibodies and rezpegaldesleukin concentrations and PD response, including safety and efficacy, may also be assessed. Relationships between rezpegaldesleukin exposure and biomarkers and/or efficacy and safety endpoints may be explored. Safety analyses will include AEs, SAEs, AESIs, C—SSRS, QIDS-SR16, vital signs, ECGs, and laboratory analytes, using the Safety Population data descriptively summarized by treatment group. Categorical safety measures will be summarized with incidence rates. Continuous safety measures will be summarized as mean change by visit. Exposure to study intervention will be calculated for each participant and summarized by treatment group. Subgroup analyses may be conducted for the primary endpoint SLEDAI-4 at Week 24 using the mITT population. Subgroups that may be evaluated include interferon gene signature status, baseline anti-dsDNA status, baseline SLEDAI-2K, complement status, previous therapies, and disease duration. Supplemental analyses will be performed on the primary endpoint using a treatment policy estimand where comparisons will be made while on treatment, but without regard to changes to background therapies or premature discontinuation. When using a treatment policy estimand, data will be analyzed using a logistic random effects model with treatment, baseline, visit, and visit-by-treatment interaction.
Analyses for the primary database lock will be conducted as described in Section 9.4 when all participants have completed the treatment period or discontinued treatment.
An interim analysis prior to the analysis of the primary database lock will be conducted to review safety data when approximately 32 participants complete three months in the study. Additionally, an interim analysis prior to the analysis of the primary database lock will be conducted to review safety and efficacy data when approximately 30% to 50% of participants have completed treatment period or discontinued treatment. Other interim analyses may be conducted as needed. All interim analyses will be used to support planning activities associated with the clinical development program and to aid in the development of PK/PD modeling. Conditional on data external to this study, interim efficacy data may be considered for early termination of this study if a relevant treatment difference between rezpegaldesleukin and placebo is unlikely. An assessment of unblinded interim data will be conducted by an internal assessment committee (IAC).
Efficacy and Patient-Reported Outcomes Assessments: With the exception of PROMIS SF Fatigue 7a, which is being validated in this study, the outcome measures used in this study are standard, widely used, and generally recognized as reliable, accurate, and relevant.

### British Isles Lupus Assessment Group 2004 (BILAG-2004):

The BILAG 2004 index is a validated global disease activity index designed on the basis of the physician's intent-to-treat, focusing on changes in disease manifestations (not present, improving, same, worse, or new) occurring in the last 4 weeks compared with the previous 4 weeks. The instrument assesses 97 clinical signs, symptoms, and laboratory parameters across 9 organ system domains: constitutional, mucocutaneous, neuropsychiatric, musculoskeletal, cardiorespiratory, gastrointestinal, ophthalmic, renal, and hematology.
The BILAG A disease activity score is severe disease activity requiring high-dosage oral or intravenous corticosteroids, immunomodulators, or high-dosage anticoagulation along with high-dosage corticosteroids or immunomodulators. The BILAG B disease activity score is moderate disease activity requiring low-dosage oral corticosteroids, intramuscular or intra-articular corticosteroid injections, topical corticosteroids or immunomodulators, antimalarials, or symptomatic therapy. The BILAG C corresponds to stable, mild disease. The BILAG D is inactive disease that was active previously. The BILAG E indicates the system was never involved.

### Cutaneous Lupus Erythematosus Disease Area and Severity Index (CLASI):

The CLASI is a validated scale used to assess cutaneous manifestations of SLE consisting of 2 scores. The first summarizes the activity of the disease, and the second is a measure of the damage done by the disease. Activity is scored on the basis of erythema, scale/hyperkeratosis, mucous membrane involvement, acute hair loss, and nonscarring alopecia. Damage is scored in terms of dyspigmentation and scarring, including scarring alopecia.
Physician's Global Assessment (PGA) of Disease Activity-Visual Analog Scale: The PGA of Disease Activity is the physician's assessment of the participant's overall disease activity because of SLE, as compared with all possible subjects with SLE. The PGA of Disease Activity is scored using a 100-mm visual analog scale, where 0 mm (measured from the left starting point of the line) indicates no disease activity, and 100 mm (measured from the left starting point of the line) indicates severe disease activity. The PGA of Disease Activity score is indicated by making a vertical tick mark on the line between 0 and 100 mm. There are benchmarks of 0 (0 mm), 1 (33 mm), 2 (67 mm), and 3 (100 mm) on the line corresponding to no, mild, moderate, and severe SLE disease activity, respectively.

### Systemic Lupus Erythematosus Disease Activity Index 2000 (SLEDAI-2K):

The SLEDAI-2K is a validated global disease activity instrument that focuses on disease manifestations across 9 organ systems. It includes 24 clinical and laboratory variables with manifestations graded by the affected organ system as follows: Central nervous system (CNS) (seizure, psychosis, organic brain syndrome, visual disturbance, cranial nerve disorder, lupus headache, cerebral vascular accident); Vascular (vasculitis); Musculoskeletal (arthritis, myositis); Renal (urinary casts, hematuria, proteinuria, pyuria); Mucocutaneous (rash, alopecia, mucosal ulcers); Cardiovascular and Respiratory (pleurisy, pericarditis); Immunologic (low complement, increased DNA binding); Constitutional (fever); and Hematologic (thrombocytopenia, leukopenia).
Systemic Lupus Erythematosus Disease Activity Index (SLEDAI) Flare Index: The SLEDAI Flare Index uses the SLEDAI-2K score, disease activity scenarios, treatment changes, and Physician's Global Assessment (PGA) of Disease Activity to define mild/moderate and severe flares. The index takes into account the absolute change in total scores, new or worsening symptoms, and increases in medication use or hospitalization because of the disease activity.

### Systemic Lupus Erythematosus International Collaborating Clinics-American College of Rheumatology (SLICC/ACR) Damage Index:

The SLICC/ACR Damage Index is scored on 41 items representing damage to 12 organ systems. The index records damage occurring in participants with SLE regardless of its cause and includes specific comorbidities associated with SLE that may be because of treatment-related toxicity. 28 Tender Joint Count and Swollen Joint Count:
The 28 joints to be examined and assessed as tender or not tender for Tender Joint Count and as swollen or not swollen for Swollen Joint Count include 14 joints on each side of the participant's body: the 2 shoulders, the 2 elbows, the 2 wrists, the 10 metacarpophalangeal joints, the 2 interphalangeal joints of the thumb, the 8 proximal interphalangeal joints, and the 2 knees. Functional Assessment of Chronic Illness Therapy-Fatigue (FACIT-Fatigue)
The FACIT-Fatigue scale (Cella and Webster 1997) is a brief, 13-item, symptom-specific questionnaire that specifically assesses the participant's self-reported severity of fatigue and its impact upon daily activities and functioning. The FACIT-Fatigue uses 0 “not at all” to 4 “very much” to assess fatigue and its impact in the past 7 days. Scores range from 0 to 52 with higher scores indicating less fatigue.

### Patient Global Impression of Change-Lupus-5 Point Version

The Patient Global Impression of Change-Lupus-5 Point Version is a single-item question asking the participants how they would rate their change in their lupus symptoms since they started taking the study medication. The 5 categories of response range from “much better” to “much worse.”

### Patient's Global Impression of Severity-7 Days (Lupus)

The Patient's Global Impression of Severity-7 Days for lupus is a single-item question asking the patient how they would rate their overall lupus symptoms in the past 7 days. The 5 categories of response range from “no symptoms” to “severe.”

### Patient Global Impression of Change-Fatigue-5 Point Version

The Patient Global Impression of Change-Fatigue-5 Point Version is a single-item question asking the patient how they would rate their change in fatigue since they started taking the study medication. The 5 categories of response range from “much better” to “much worse.”

### Patient's Global Impression of Severity-7 Days (Fatigue)

The Patient's Global Impression of Severity-7 Days for fatigue is a single-item question asking the patient how they would rate their overall fatigue severity over the past 7 days. The 5 categories of response range from “no symptoms” to “very severe.”

### Patient-Reported Outcome Measurement Information System (PROMIS) Short

### Form Fatigue 7a

The Patient-Reported Outcome Measurement Information System (PROMIS®) Short Form Fatigue 7a is a fixed-length short form PRO measure derived from the PROMIS Fatigue Item Bank. The PROMIS SF Fatigue 7a is designed to evaluate the self-reported experience of fatigue and its impact within a single brief measure including 7 items, producing a score that locates the respondent on a unidimensional fatigue T score metric (mean of 50 and a standard deviation of 10). These T scores are based on a large sample that is representative of the United States general population based upon the 2000 census. A higher PROMIS T-score reflects increased fatigue. The PROMIS SF Fatigue 7a has a recall period of 7 days and includes a 5-point verbal rating scale ranging from “Never” to “Always” (Health Measures 2019; Cella et al. 2010; Cella et al. 2007; DeWalt et al. 2007).
Medical Outcomes Short Form 36-Item Health Survey version 2 (SF-36v2) acute:
The Medical Outcomes Short-Form 36-Item Health Survey version 2 (SF-36v2) acute measure is a subjective, generic, health-related quality of life instrument that is participant reported and consists of 36 questions covering 8 health domains: physical functioning, bodily pain, role limitations because of physical problems, role limitations because of emotional problems, general health perceptions, mental health, social function, and vitality. In addition, 2 summary scores—the physical component score and the mental component score-will be evaluated on the basis of the 8 SF-36v2 acute domains. The acute version of this instrument has a 1-week recall period (Brazier et al. 1992; Ware and Sherbourne 1992).

### Worst Joint Pain Numeric Rating Scale (NRS)

The Worst Joint Pain NRS is a single-item, participant-administered, 11-point horizontal scale anchored at 0 and 10, with 0 representing “no joint pain” and 10 representing “joint pain as bad as you can imagine.” Overall, severity of a participant's joint pain is indicated by the participant selecting the number that best describes his or her worst level of joint pain over the previous 7 days.

### Worst Pain Numeric Rating Scale (NRS)

The Worst Pain NRS is a single-item, participant-administered, 11-point horizontal scale anchored at 0 and 10, with 0 representing “no pain” and 10 representing “pain as bad as you can imagine.” Overall, severity of a participant's pain is indicated by the participant selecting the number that best describes his or her worst level of pain over the previous 7 days.
An interim assessment for KFAJ reviewed the efficacy and safety data from 160 subjects through week 24 and resulted in continuing the study without modification.

### REFERENCES

Ad Hoc Committee on Systemic Lupus Erythematosus Response Criteria for Fatigue. Measurement of fatigue in systemic lupus erythematosus: a systematic review. Arthritis Rheum. 2007; 57 (8): 1348-1357. https://doi.org/10.1002/art.23113
[APA] American Psychiatric Association. Diagnostic and Statistical Manual of Mental Disorders. 5^{th} ed. Arlington, VA: American Psychiatric Publishing; 2013.
Brazier J E, Harper R, Jones N M, O'Cathain A, Thomas K J, Usherwood T, Westlake L. Validating the SF-36 health survey questionnaire: new outcome measure for primary care.
BMJ. 1992; 305 (6846): 160-164. https://doi.org/10.1136/bmj.305.6846.160
Bretz F, Pinheiro J C, Branson M. Combining multiple comparisons and modeling techniques in dose-response studies. Biometrics. 2005; 61 (3): 738-748. https://doi.org/10.1111/j.1541-0420.2005.00344.x
Cella D, Riley W, Stone A A, et al. The Patient-Reported Outcomes Measurement Information System (PROMIS) developed and tested its first wave of adult self-reported health outcome item banks: 2005-2008. J Clin Epidemiol. 2010; 63 (11): 1179-1194. https://doi.org/10.1016/j.jclinepi.2010.04.011
Cella D, Yount S, Rothrock N, et al. The Patient-Reported Outcomes Measurement Information System (PROMIS): progress of an NIH Roadmap cooperative group during its first two years. Med Care. 2007; 45 (5 Suppl 1): S3-S11. https://doi.org/10.1097/01.mlr.0000258615.42478.55
Cella D, Webster K. Linking outcomes management to quality-of-life measurement. Oncology (Williston Park). 1997; 11 (11A): 232-235.
[Columbia Lighthouse Project.] The Columbia Protocol for Research. Columbia-Suicide Severity Rating Scale Scoring and Data Analysis Guide. Version 2.0. Available at: http://cssrs.columbia.edu/wp-content/uploads/ScoringandDataAnalysisGuide-for-Clinical-Trials-1.pdf. Accessed 30 Jan. 2019
Cooper G S, Treadwell E L, St Clair E W, et al. Sociodemographic associations with early disease damage in patients with systemic lupus erythematosus. Arthritis Rheum. 2007; 57 (6): 993-999. https://doi.org/10.1002/art.22894
DeWalt D A, Rothrock N, Yount S, Stone A A, PROMIS Cooperative Group. Evaluation of item candidates: The PROMIS qualitative item review. Med Care. 2007; 45 (5 Suppl 1): S12-S21. https://doi.org/10.1097/01.mlr.0000254567.79743.e2
[FDA] Office of Clinical Pharmacology Division of Pharmacometrics. “Request for Qualification of MCP-Mod as an efficient statistical methodology for model-based design and analysis of Phase II dose finding studies under model uncertainty.” FDA (2015). Available at:

https://www.fda.gov/media/99313/download. Accessed Jan. 27, 2020.
[FDA] Food and Drug Administration. Guidance for industry: pharmacokinetics in patients with impaired renal function-study design, data analysis, and impact on dosing and labeling; 2010. Available at: https://www.fda.gov/media/78573/download. Accessed Jan. 27, 2020.
Franklyn K, Lau C S, Navarra S V, et al. Definition and initial validation of a Lupus Low Disease Activity State (LLDAS). Ann Rheum Dis. 2016; 75:1615-1621. https://doi.org/10.1136/annrheumdis-2015-207726
Furie R, Khamashta M, Merrill J T, et al., Anifrolumab, an anti-interferon-α receptor monoclonal antibody, in moderate-to-severe systemic lupus erythematosus. Arthritis Rheumatol. 2017; 69 (2): 376-386. https://doi.org/10.1002/art.39962
Furie R, Petri M, Zamani O, et al. A phase III, randomized, placebo-controlled study of belimumab, a monoclonal antibody that inhibits B lymphocyte stimulator, in patients with systemic lupus erythematosus. Arthritis Rheum. 2011; 63 (12): 3918-3930. https://doi.org/10.1002/art.30613
Gladman D D, Urowitz M B, Kagal A, Hallett D. Accurately describing changes in disease activity in Systemic Lupus Erythematosus. J Rheumatol. 2000; 27 (2): 377-379.
Gould A L. BMA-Mod: A Bayesian model averaging strategy for determining dose-response relationships in the presence of model uncertainty. Biom J. 2019; 61 (5): 1141-1159. https://doi.org/10.1002/bimj.201700211
He J, Zhang R, Shao M, et al. Efficacy and safety of low-dose IL-2 in the treatment of systemic lupus erythematosus: a randomised, double-blind, placebo-controlled trial. Ann Rheum Dis. 2020; 79 (1): 141-149. https://doi.org/10.1136/annrheumdis-2019-215396
He J, Zhang X, Wei Y, et al. Low-dose interleukin-2 treatment selectively modulates CD4 (+) T cell subsets in patients with systemic lupus erythematosus. Nat Med. 2016; 22 (9): 991-993. https://doi.org/10.1038/nm.4148
Health Measures. PROMIS Fatigue Scoring Manual. Available at http://www.healthmeasures.net/images/PROMIS/manuals/PROMIS_Fatigue_Scoring_Manual.pdf. 2019. Accessed Jan. 27, 2020.
Hochberg M C. Updating the American College of Rheumatology revised criteria for the classification of systemic lupus erythematosus. Arthritis Rheum. 1997; 40 (9): 1725. https://doi.org/10.1002/art. 1780400928
Isenberg D A, Petri M, Kalunian K, et al. Efficacy and safety of subcutaneous tabalumab in patients with systemic lupus erythematosus: results from ILLUMINATE-1, a 52-week, phase III, multicentre, randomised, double-blind, placebo-controlled study. Ann Rheum Dis. 2016; 75 (2): 323-331. https://doi.org/10.1136/annrheumdis-2015-207653
Kalunian K, Urowitz M, Isenberg D, et al. Clinical trial parameters that influence outcomes in lupus trials that use the systemic lupus erythematosus responder index. Rheumatology (Oxford). 2018; 57 (1): 125-133. https://doi.org/10.1093/rheumatology/kex368 Merrill J T, van Vollenhoven R F, Buyon J P, et al. Efficacy and safety of subcutaneous tabalumab, a monoclonal antibody to B-cell activating factor, in patients with systemic lupus erythematosus: results from ILLUMINATE-2, a 52-week, phase III, multicentre, randomised, double-blind, placebo-controlled study. Ann Rheum Dis. 2016; 75 (2): 332-340. https://doi.org/10.1136/annrheumdis-2015-207654
Navarra S V, Guzmán R M, Gallacher A E, et al. Efficacy and safety of belimumab in patients with active systemic lupus erythematosus: a randomised, placebo-controlled, phase 3 trial. Lancet. 2011; 377 (9767): 721-731. https://doi.org/10.1016/S0140-6736 (10) 61354-2
Ohl K, Tenbrock K. Regulatory T cells in systemic lupus erythematosus. Eur J Immunol. 2015; 45 (2): 344-355. https://doi.org/10.1002/eji.201344280
Özel F, Argon G. The effects of fatigue and pain on daily life activities in systemic lupus erythematosus. Agri. 2015; 27 (4): 181-189. https://doi.org/10.5505/agri.2015.38278
Rosenzwajg M, Lorenzon R, Cacoub P, et al. Immunological and clinical effects of low-dose interleukin-2 across 11 autoimmune diseases in a single, open clinical trial. Ann Rheum Dis. 2019; 78 (2): 209-217. https://doi.org/10.1136/annrheumdis-2018-214229
Sampson H A, Muñoz-Furlong A, Campbell R L, et al. Second symposium on the definition and management of anaphylaxis: summary report-Second National Institute of Allergy and Infectious Disease/Food Allergy and Anaphylaxis Network symposium. J Allergy Clin Immunol. 2006; 117 (2): 391-397. https://doi.org/10.1016/j.jaci.2005.12.1303
Tan E M, Cohen A S, Fries J F, et al. The 1982 revised criteria for the classification of systemic lupus erythematosus. Arthritis Rheum. 1982; 25 (11): 1271-1277.
van Vollenhoven R F, Hahn B H, Tsokos G C, et al. Efficacy and safety of ustekinumab, an IL-12 and IL-23 inhibitor, in patients with active systemic lupus erythematosus: results of a multicentre, double-blind, phase 2, randomised, controlled study. Lancet. 2018; 392 (10155): 1330-1339. https://doi.org/10.1016/S0140-6736 (18) 32167-6
von Spee-Mayer C, Siegert E, Abdirama D, et al. Low-dose interleukin-2 selectively corrects regulatory T cell defects in patients with systemic lupus erythematosus. Ann Rheum Dis. 2016; 75 (7): 1407-1415. https://doi.org/10.1136/annrheumdis-2015-207776
Wallace D J, Furie R A, Tanaka Y, et al. Baricitinib for systemic lupus erythematosus: a double-blind, randomised, placebo-controlled, phase 2 trial. Lancet. 2018; 392 (10143): 222-231. https://doi.org/10.1016/S0140-6736 (18) 31363-1
Ware J E Jr, Sherbourne C D. The MOS 36-item short-form health survey (SF-36): I. Conceptual framework and item selection. Med Care. 1992; 30 (6): 473-483.
Winthrop K L, Novosad S A, Baddley J W, et al. Opportunistic infections and biologic therapies in immune-mediated inflammatory diseases: consensus recommendations for infection reporting during clinical trials and postmarketing surveillance. Ann Rheum Dis. 2015; 74 (12): 2107-2116. https://doi.org/10.1136/annrheumdis-2015-207841
Yurkovich M, Vostretsova K, Chen W, Aviña-Zubieta J A. Overall and cause-specific mortality in patients with systemic lupus erythematosus: a meta-analysis of observational studies. Arthritis Care Res (Hoboken). 2014; 66 (4): 608-616. https://doi.org/10.1002/acr.22173

### Terms

In describing and claiming certain features of this disclosure, the following terminology will be used in accordance with the definitions described below unless indicated otherwise.

A unit dose is the amount of a medication administered to a patient in a single dose or a single indicated administration. Where the term unit dose is used in an embodiment, it will be understood to indicate the present disclosure also provides a pharmaceutical (unit dosage) composition according to the unit dose.

The terms Q1W refers to administration once per week, Q2W refers to administration once per a two-week interval; Q3W refers to administration once per a three week interval; Q4W refers to administration once per a four-week interval; Q6W refers to administration once per a six-week interval; Q8W refers to administration once per an eight-week interval; Q12 refers to administration once per a twelve-week interval; Q24W refers to administration once per a twenty four week interval; Q36W refers to administration once per a thirty six week interval; Q48W refers to administration once per a forty eight week interval; and Q52W refers to administration once per a fifty two week interval. The skilled artisan will recognize and understand that other terms will have similar meaning if the number of weeks is changed, or if the indicated interval is yearly, for example.

A “maintenance dose” is a dose administered to a patient after the induction dose treatment. A maintenance dose is an amount of rezpegaldesleukin administered to maintain the desired therapeutic response including a favorable response in a marker of the autoimmune disease being treated. A maintenance dose can be dose that is the same or lower in amount of rezpegaldesleukin compared to the induction dose.

The term “selective” as used and described herein, refers to an in vivo immunological response which embodies characteristics of induced immune cell, or immunological signal responses, in some respects, but not in others. In particular, “selective” with respect to Treg induction and/or activation refers to an immune response presenting an increase in Treg cell numbers (CD25 high and total by flow cytometry), and/or an increase in the Treg activation state, as indicated by one or more markers of activation, such as ICOS or Ki67 or Stat5, and/or activation refers downstream induced immuno-suppression responses, and/or induced immunological tolerance responses, while lacking certain other immune responses. In this context, “selective Treg induction” refers to an immune response of Tregs as described, while at the same time, lacking significant and/or clinically material effector T cell and associated immunological activation responses. Significant and/or clinically material effector T cell and associated immunological activation responses include for example CD4 positive T effector cell, and/or CD8 positive T effector cell proliferation, and/or markers of activation, such as ICOS or Ki67, or other well-known effector immune responses. Other effector immune response signals may include elevation of certain pro-inflammatory cytokines, such as those known as “cytokine syndrome”, and/or such as IL-5, INF_{gamma}, IL-6, IFNₐₗₚₕₐ, IL-17, IL-22, IL-19. Selective Treg stimulation can also reflected in the mean Treg:Tcon ratio. Preferably the mean Treg:Tcon ratio achieved in response to RUR-IL-2 or RUR20kD-IL-2 related compositions described herein is at least 5 fold, and preferably 7 fold, and more preferably 10 fold or greater.

The term “degree of PEGylation” as used herein refers to the number of stable PEG substituents covalently linked an amino group(s) of an individual aldesleukin polypeptide.

The term “about” as used herein, means in reasonable vicinity of the stated numerical value, such as plus or minus 10% of the stated numerical value. Preferably, “about” or “approximately” as used herein means within plus or minus 5% of a given quantity.

The term “n′ is 1 and 2 and 3” as used herein refers to mixtures of IL-2 conjugates wherein the mixtures comprise di-PEGylated and tri-PEGylated conjugates, as described herein.

The term “regulatory T cells” or “Tregs” refer to T cells such CD4+FoxP3+CD25^{bright} phenotypes. (See e.g. Jeffrey A. Bluestone and Qizhi Tang, Treg cells—the next frontier of cell therapy, Science, 12 Oct. 2018⋅Vol. 362 Issue 6411, p 154-155.)

The term “T cons” or “conventional T cells” refer to T lymphocytes that express an αβ T cell receptor (TCR), as well as a co-receptor CD4 or CD8, and carry out well-established adaptive immunity effector functions, such as T helper cell functions and cytotoxic T cell effector functions. For example, Tcon can refer to CD4⁺CD25⁻ naive conventional T cells. “Effector T cells (Teff)” refers to CD4+ and CD8+ cellular effector phenotypes, such as helper T cell, Cytotoxic T cells, and others, as known to the skilled artisan. “NK cells”, also known as “natural killer cells”, “K cells”, or “killer cells” are a type of lymphocyte (white blood cell) and a component of the innate immune system. NK cells play a major role in the host-rejection of tumors and virally infected cells.

“IL-2 Intermediate” refers to IL-2 polypeptide, in particular aldesleukin. “RUR_{20kD}-IL-2” refers to IL-2 PEG conjugates wherein the IL-2 portion is aldesleukin as described herein, and the PEG portion is as described herein. An RUR_{20kD}-IL-2 composition can also be referred to in a general way by the chemical name (1,3-bis(methoxypoly(ethylene glycol)_{10kD}carbamoyl)-2-propanoxy)-4-butanamide) interleukin-2), recognizing this does not completely describe the composition. As used herein, aldesleukin refers to 125-L-serine-2-133 interleukin-2, a recombinant non-glycosylated interleukin-2 expressed in E. coli. The sequence of amino acid sequence of aldesleukin is shown in FIG. 2. Aldesleukin expressed in other host systems known to the skilled artisan are also within the meaning of the term as used herein.

The term “IL-2” as used herein, refers to a moiety having human IL-2 activity. The term “IL-2 moiety” refers to the IL-2 moiety prior to attachment to a branched polyethylene glycol moiety as well as to the IL-2 moiety following covalent attachment. It will be understood that when the original IL-2 moiety is attached to a polyethylene glycol polymer, such as the branched polyethylene glycol polymer provided herein, the IL-2 moiety is slightly altered due to the presence of one or more covalent bonds associated with linkage to the polyethylene glycol moieties. Such slightly altered form of the IL-2 moiety attached to another molecule is referred to herein as a “residue” of the IL-2 moiety. The term, ‘residue’, in the context of residue of IL-2, means the portion of the IL-2 molecule that remains following covalent attachment to a polymer such as a polyethylene glycol, at one or more covalent attachment sites, as shown in the formulae herein. Typically the site of attachment will be one of 11 amine groups of a lysine in IL-2.

It will be understood that when the unmodified IL-2 is attached to a polymer such as polyethylene glycol, the IL-2 is slightly altered due to the presence of one or more covalent bonds associated with linkage to the polymer(s). This slightly altered form of the IL-2 attached to another molecule such as a branched PEG moiety may be referred to in some instances as a “residue” of the IL-2, or may simply be referred to as “IL-2” or the like, with the understanding that the IL-2 comprised in such polymer conjugate is slightly altered due to the presence of one or more covalent bonds, each linking a branched PEG moiety to the IL-2. The term “higher PEGylated IL-2 conjugates” refers to tetra PEG conjugates or penta PEG conjugates or conjugates up to 11 PEG moieties. Preferably “higher PEGylated IL-2 conjugates” refers to tetra PEG conjugates or penta PEG conjugates.

For example, proteins having an amino acid sequence corresponding to any one of SEQ ID NOs: 1 through 4 described in International Patent Publication No. WO 2012/065086 are exemplary IL-2 proteins, as are any proteins or polypeptides substantially homologous thereto. The term substantially homologous means that a particular subject sequence, for example, a mutant sequence, varies from a reference sequence by one or more substitutions, deletions, or additions, the net effect of which does not result in an adverse functional dissimilarity between the reference and subject sequences. For the purposes herein, sequences having greater than 95 percent homology, equivalent biological activity (although not necessarily equivalent strength of biological activity), and equivalent expression characteristics are considered substantially homologous. For purposes of determining homology, truncation of the mature sequence should be disregarded. As used herein, the term “IL-2” includes such proteins modified deliberately, as for example, by site directed mutagenesis or accidentally through mutations. These terms also include analogs having from 1 to 6 additional glycosylation sites, analogs having at least one additional amino acid at the carboxy terminal end of the protein wherein the additional amino acid(s) includes at least one glycosylation site, and analogs having an amino acid sequence which includes at least one glycosylation site. The term includes both natural and recombinantly produced moieties. In addition, the IL-2 can be derived from human sources, animal sources, and plant sources. One exemplary IL-2 is a human recombinant IL-2 referred to as aldesleukin (See FIG. 2). Reference to a long-acting IL-2R agonist as described herein is meant to encompass pharmaceutically acceptable salt forms thereof.

The RUR-IL-2 or RUR_{20kD}-IL-2 related compositions described herein are in one respect long-acting agents. Long-acting, in reference to an RUR-IL-2 or RUR_{20kD}-IL-2 related compositions as provided herein, refers to such composition having a circulating half-life in plasma that is extended over that of the same IL-2R agonist (e.g., aldesleukin or other suitable interleukin-2 sequence) that is unmodified. For example, the comparator agonist is not modified by covalent attachment to one or more water-soluble polymer moieties such as polyethylene glycol moieties and is compared as administered at a protein equivalent dose of IL-2R agonist to the same subject and assessed by the same pharmacokinetic analysis.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present disclosure will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. Where stated, for the PEG polymers, the variable (n) may range from about 3 to 4000, and the terminal groups and architecture of the overall PEG can vary. Preferably PEG has the particular meaning as described in detail herein.

“Branched,” in reference to the geometry or overall structure of a polymer, refers to a polymer having two or more polymer “arms” or “chains” extending from a branch point or central structural feature. As an example, an illustrative PEG reagent, mPEG2-butanoic acid, N-hydroxysuccinimide ester (1,3-bis(methoxypoly(ethylene glycol) carbamoyl)-2-propanoxy)-4-succinimidyl butanoate) is a branched polyethylene glycol polymer comprised of two linear PEG chains, each covalently attached via a carbamate linkage (˜NHC(O)O˜) to the 1- and 3-carbons, respectively, of a central propyl group, from which extends an oxybutanoate succinimidyl ester.

Molecular weight in the context of a water-soluble polymer, such as PEG, can be expressed as either a number (nominal) average molecular weight or a weight average molecular weight. Unless otherwise indicated, all references to molecular weight herein refer to the nominal average molecular weight. Both molecular weight determinations, number average and weight average, can be measured using gel permeation chromatography, gel filtration chromatography, or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation, or viscometry to determine weight average molecular weight. Gel filtration chromatography is often used to determine the average molecular weight of branched polymers. PEG polymers are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

A “stable” linkage or bond refers to a chemical bond that is substantially stable in water, that is to say, does not undergo hydrolysis under physiological conditions to any appreciable extent over an extended period of time. Examples of hydrolytically stable linkages generally include but are not limited to the following: carbon-carbon bonds (e.g., in aliphatic chains), ethers, amides, amines, and the like. Generally, a stable linkage is one that exhibits a rate of hydrolysis of less than about 1-2% per day under physiological conditions. Hydrolysis rates of representative chemical bonds can be found in most standard chemistry textbooks.

As used in this specification, the singular forms “a,” “an,” and “the” include plural referents unless the context clearly dictates otherwise.

“Substantially” or “essentially” means nearly totally or completely, for instance, 95% or greater of a given quantity, unless stated to the contrary.

The term “patient”, as used herein, refers to a human patient. In certain embodiments, the patient, is further characterized with a disease, disorder or condition, such as an autoimmune condition, that would benefit from administration of a composition of the present disclosure.

The term “treatment” or “treating” as used herein refers to the management and care of a patient having a condition for which administration of a composition of the present disclosure is indicated for the purpose of combating or alleviating symptoms and complications of those conditions. Treating includes administering a composition of the present disclosure to a patient in need thereof to prevent the onset of symptoms or complications, alleviating the symptoms or complications, or eliminating the disease, condition, or disorder. For example, an autoimmune disorder. Preferably treating includes administering a composition of the present disclosure to a patient in need thereof to result in immunosuppression and/or tolerance. The patient to be treated is an animal, and preferably a human being. Administering as used herein includes either when the patient consumes the composition and/or when the patient is directed to consume the composition.

The phrases “pharmaceutically effective amount” and “pharmacologically effective amount” and “therapeutically effective amount” and “physiologically effective amount” are used interchangeably herein and refer to the amount of an RUR_{20kD}-IL-2 and related composition provided herein that is needed to achieve a desired level of the substance in the bloodstream or target tissue. The precise amount will depend upon numerous factors, such as for example, the particular condition being treated, the intended patient population, individual patient considerations, the components and physical characteristics of the therapeutic composition to be administered, and the like.

### Example 5

A Phase 2, Randomized, Double-Blind, Parallel-Group, Placebo-Controlled, 52 Week Study to Evaluate the Efficacy of LY3471851 (NKTR-358) in the Treatment of Adult Patients with Moderate-to-Severe Atopic Dermatitis

### Overall Design

This is a Phase 2, multicenter, randomized, double-blind, placebo-controlled, parallel-group, outpatient dose-ranging study to evaluate the efficacy and describe the safety of subcutaneously administered (SC) LY3471851 as compared to placebo during a 16-week induction period in adult participants with moderate-to-severe AD using these dosing regimens:
LY3471851 1800 μg every 2 weeks (Q2W)
LY3471851 1800 μg every 4 weeks (Q4W)
LY3471851 900 μg Q2W, and
placebo only.

Studies of atopic dermatitis patients described herein can be modified to include bio-experienced atopic dermatitis patients, for instance where the subjects in the study represent for example 75% bio-experienced atopic dermatitis patients (for example dupilumab experienced patients) and 25% bio-naïve atopic dermatitis patients. The number and ratio of the aforementioned groups can be varied based on the desired study design. Studies of atopic dermatitis patients described herein can be modified to include JAK inhibitor-experienced atopic dermatitis patients, for instance where the subjects in the study represent for example 75% JAK inhibitor-experienced atopic dermatitis patients (for example to abrocitinib or upadacitinib) and 25% JAK-naïve atopic dermatitis patients. The number and ratio of the aforementioned groups can be varied based on the desired study design.

Topical rescue therapy is allowed starting at Week 8 for participants who do not achieve an EASI-25 response.
The blinded 28-week maintenance treatment period begins with the dose administered at Week 16. Participants having at least an EASI-50 response at Week 16 will be reassigned to a maintenance dosing regimen, as listed here.

| Week 16 responders who were | |
|---|---|
| originally randomized to this | Will be reassigned to this |
| induction regimen . . . | maintenance regimen: |
| LY3471851 1800 μg Q2W | by rerandomization in 1:1 ratio: |
| | LY3471851 1800 μg Q4W or |
| | LY3471851 1800 μg Q12W |
| LY3471851 1800 μg Q4W | by rerandomization in 1:1 ratio: |
| | LY3471851 1800 μg Q4W or |
| | LY3471851 1800 μg Q12W |
| LY3471851 900 μg Q2W | LY3471851 900 μg Q4W |
| placebo | LY3471851 1800 μg Q4W |

Participants will receive LY3471851 1800 μg Q4W as “escape therapy” if they have not achieved an EASI-50 response at Week 16 have received rescue therapy any time from Week 8 to Week 16, or have not achieved an EASI-25 response at Week 20, Week 24, Week 28 or Week 32. Escape therapy is administered through the last dosing visit of the maintenance period. Participants on any study intervention will be discontinued if they do not achieve an EASI-50 response at Week 36 or later. Participants who have received at least one dose of escape therapy will be discontinued if they do not achieve an EASI-50 response at any time after Week 16.
In summary, the study includes 4 periods over a total study duration of approximately 54 to 57 weeks, inclusive of screening:
Screening: from 15 to 35 days prior to randomization at Week 0 (Visit 2)
Treatment Period—Induction: from randomization at Week 0 (Visit 2) through assessments at Week 16 (Visit 10)
Treatment Period—Maintenance: from dose administration at Week 16 through assessments at Week 44 (Visit 17), and
Posttreatment Follow-Up: two visits, the last visit being 52 weeks after randomization (12 weeks after last dose, given at Week 40).
   The proliferation and survival of Treg cells are impaired in various autoimmune and inflammatory skin diseases, including atopic dermatitis (AD). LY3471851 (also known as NKTR-358) has the capacity to promote the expansion and activation of Tregs.
   The purpose of this study is to measure improvement in AD signs and symptoms after treatment with various dosing regimens of subcutaneously administered (SC) LY3471851 compared to placebo in adult participants with moderate-to-severe AD.
The total study duration is approximately 54 to 57 weeks, including the screening and posttreatment follow-up periods.
Treatment is administered from first dose given at the randomization visit (Week 0) through the last dose given in the maintenance period (Week 40).
The visit frequency is every 2 weeks from randomization through Week 16 and every 4 weeks thereafter.

### Study Population:

The study population includes adult male or female participants from 18 to 70 years of age, inclusive, who have a diagnosis of AD which is moderate-to-severe as measured by
Eczema Area and Severity Index score ≥16,
Investigator's Global Assessment score ≥3, and
≥10% of body surface area involvement.
   Other key inclusion criteria include
body mass index (BMI) of 17.0 to 45.0 kg/m2, inclusive
candidates for systemic therapy and have history of inadequate response to, or history of intolerance of, topical therapies
agree to use at least 1 emollient daily throughout the study.
   The key exclusion criteria include
other skin conditions, such as psoriasis, cutaneous lupus, and eczema herpeticum
use of AD treatments in the specified washout period before randomization: biologics, 5 half-lives or 12 weeks; phototherapy, systemic corticosteroids, or systemic immunomodulators, 4 weeks; topical drugs, 2 weeks.
are anticipated to require treatment with prohibited concomitant medications during the study
are pregnant or intending to become pregnant during the study
are unsuitable for study participation due to medical history or current serious medical conditions
   It is expected that approximately 80% of participants will not have previously received dupilumab (dupilumab-naive).

### Number of Participants:

Approximately 300 participants will be randomized to study intervention.

### Intervention Groups and Duration:

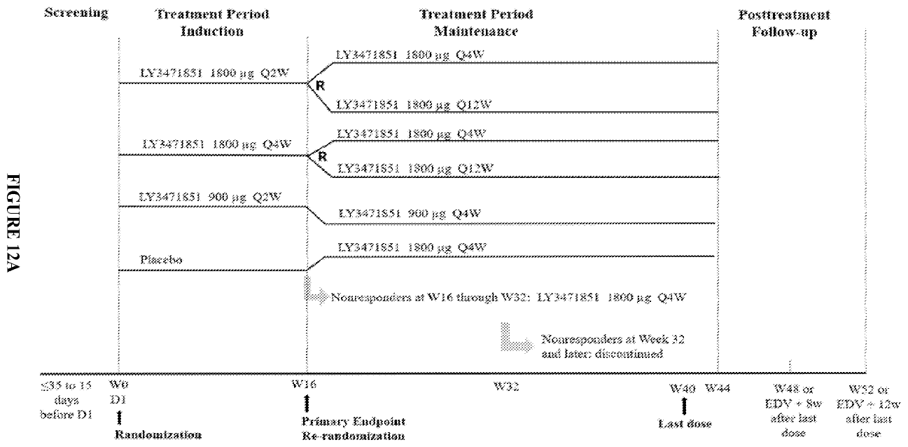

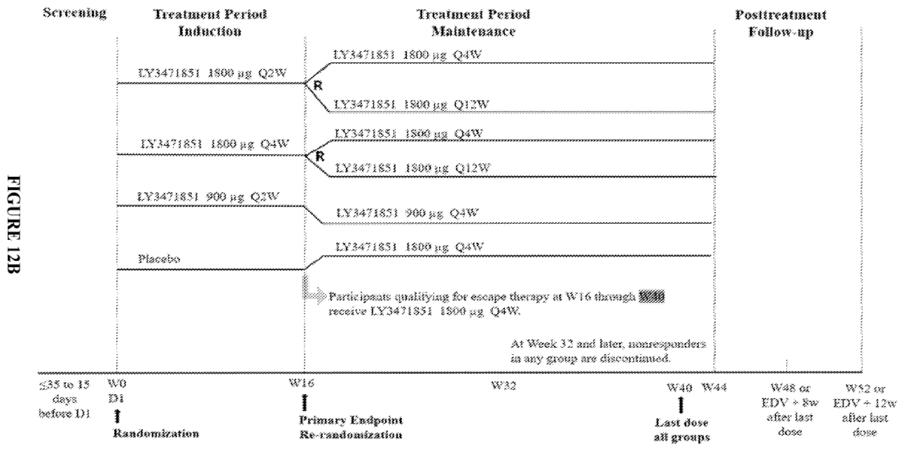

The study includes 4 periods over a total study duration of approximately 54 to 57 weeks, inclusive of screening. These are treatment groups from randomization at Week 0 and until Week 16:
LY3471851 1800 μg every 2 weeks (Q2W)
LY3471851 1800 μg every 4 weeks (Q4W)
LY3471851 900 μg every 2 weeks (Q2W), and
placebo only.
   Topical rescue therapy is allowed starting at Week 8 for participants who do not achieve an EASI-25 response.
   At Week 16, participants in the placebo-only induction group are assigned to LY3471851 1800 μg Q4W. Other participants are reassigned or rerandomized to these treatment groups:
LY3471851 1800 μg Q4W
LY3471851 1800 μg every 12 weeks (Q12W), and
LY3471851 900 μg Q4W.
   Maintenance dosing continues up to and including Week 40.
   Participants will receive LY3471851 1800 μg Q4W as “escape therapy” if they
have not achieved an EASI-50 response at Week 16 or
have not achieved an EASI-25 response at any time between Week 16 and Week 32.
   Escape therapy is administered through the last dosing visit of the maintenance period.
   Study Schema are shown in FIGS. 12A and B.

### Schedule of Activities (SoA)

TABLE 1
Screening and induction treatment periods of Study JIP-MC-KFAE

| | Screening | Treatment Period - Induction | | | | | | | | | |
|---|---|---|---|---|---|---|---|---|---|---|---|
| Visit number | V1 | V2 | V3 | V4 | V5 | V6 | V7 | V8 | V9 | V10 | Comment |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Informed | X | | | | | | | | | | The ICF must be signed |
| consent | | | | | | | | | | | before any protocol-specific |
| | | | | | | | | | | | tests or procedures are |
| | | | | | | | | | | | performed. |
| Inclusion and | X | X | | | | | | | | | Inclusion and exclusion |
| exclusion | | | | | | | | | | | criteria should be confirmed |
| criteria, review | | | | | | | | | | | prior to treatment |
| and confirm | | | | | | | | | | | assignment and first dose |
| | | | | | | | | | | | administration. |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Demographics | X | | | | | | | | | | Includes ethnicity (United |
| | | | | | | | | | | | States only), year of birth, |
| | | | | | | | | | | | gender (sex) and race |
| Preexisting | X | | | | | | | | | | All ongoing conditions and |
| conditions and | | | | | | | | | | | relevant past medical history |
| medical history | | | | | | | | | | | should be collected. |
| Prespecified | X | | | | | | | | | | Prespecified medical history |
| medical history | | | | | | | | | | | includes allergic |
| (indication and | | | | | | | | | | | conjuctivitis, allergic |
| history of | | | | | | | | | | | rhinitis, allergies, asthma, |
| interest) | | | | | | | | | | | depression, and insomnia. |
| Prior treatments | X | | | | | | | | | | Revelant prior therapies |
| for indication | | | | | | | | | | | include, but are not limited |
| | | | | | | | | | | | to, dupilumab. Includes all |
| | | | | | | | | | | | topical medications within 1 |
| | | | | | | | | | | | year from baseline and all |
| | | | | | | | | | | | biologic/systemic therapies |
| | | | | | | | | | | | within lifetime. |
| Concomitant | X | X | X | X | X | X | X | X | X | X | Data will be collected for |
| medications | | | | | | | | | | | medications, including |
| | | | | | | | | | | | rescue therapy, and |
| | | | | | | | | | | | background emollient. |
| Adverse events | X | X | X | X | X | X | X | X | X | X | |
| (AEs) | | | | | | | | | | | |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Physical | | | | | | | | | | | |
| evaluation | | | | | | | | | | | |
| Height | X | | | | | | | | | | |
| Weight | X | | | | | | | | | X | |
| Vital signs | X | X | X | X | X | X | X | X | X | X | Includes pulse rate, blood |
| | | | | | | | | | | | pressure, respiratory rate, |
| | | | | | | | | | | | and body temperature. |
| | | | | | | | | | | | Measured after participant |
| | | | | | | | | | | | has been sitting at least 5 |
| | | | | | | | | | | | minutes. |
| Complete | X | | | | | | | | | | Complete physical |
| physical | | | | | | | | | | | examination excludes |
| examination | | | | | | | | | | | pelvic, rectal, and breast |
| | | | | | | | | | | | exams unless clinically |
| | | | | | | | | | | | indicated. Assess for |
| | | | | | | | | | | | tuberculosis (TB) risk |
| | | | | | | | | | | | factors, and for signs and |
| | | | | | | | | | | | symptoms of active TB, |
| | | | | | | | | | | | including an assessment of |
| | | | | | | | | | | | peripheral lymph nodes. |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Symptom- | | X | | | | | | | | X | As shown in this schedule |
| directed physical | | | | | | | | | | | and as needed based on |
| assessment | | | | | | | | | | | participant status and |
| | | | | | | | | | | | standard of care. May be |
| | | | | | | | | | | | performed by qualified |
| | | | | | | | | | | | personnel per local |
| | | | | | | | | | | | regulations. Assess for TB |
| | | | | | | | | | | | risk factors, and for signs |
| | | | | | | | | | | | and symptoms of active TB, |
| | | | | | | | | | | | including an assessment of |
| | | | | | | | | | | | peripheral lymph nodes; |
| 12-lead ECG | X | | | | | | | | | X | Local ECGs collected |
| Chest x-ray | X | | | | | | | | | | Locally performed. |
| ([CXR], PA and, | | | | | | | | | | | Interpreted and reported by |
| if needed, lateral | | | | | | | | | | | a radiologist or |
| view) | | | | | | | | | | | pulmonologist. |
| Participant | | | | | | | | | | | |
| education and | | | | | | | | | | | |
| supplies | | | | | | | | | | | |
| Diary education | X | | | | | | | | X | | Additional education |
| | | | | | | | | | | | provided as needed. |
| Actigraphy | X | | | | X | | | | X | | Optional for patients at |
| | | | | | | | | | | | participating sites. |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Participant diary | | | | | | | | | | | |
| (electronic) | | | | | | | | | | | |
| Diary dispensed | X | | | | | | | | | | Includes the following for |
| | | | | | | | | | | | daily collection: ADSS, Itch |
| | | | | | | | | | | | NRS, PGI-S-AD, Skin Pain |
| | | | | | | | | | | | NRS, and, if applicable, the |
| | | | | | | | | | | | TCS Question |
| Diary | | X | X | X | X | X | X | X | X | X | |
| compliance | | | | | | | | | | | |
| check | | | | | | | | | | | |
| Diary returned | | X | | | | | | | | | Collect diary from |
| | | | | | | | | | | | participants who were not |
| | | | | | | | | | | | eligible for randomization |
| | | | | | | | | | | | (screen failed). |
| Patient-reported | | | | | | | | | | | Complete prior to any |
| outcomes | | | | | | | | | | | clinician-administered |
| (electronic) | | | | | | | | | | | assessments. |
| DLQI | X | X | | X | | X | | X | | X | |
| POEM | X | X | | X | | X | | X | | X | |
| HADS | | X | | X | | X | | X | | X | |
| SCORAD | X | X | | X | | X | | X | | X | |
| (patient portion) | | | | | | | | | | | |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Clinician- | | | | | | | | | | | |
| administered | | | | | | | | | | | |
| assessments | | | | | | | | | | | |
| (electronic) | | | | | | | | | | | |
| EASI | X | X | | X | | X | X | X | X | X | |
| VIGA-AD | X | X | | X | | X | | X | | X | |
| SCORAD | X | X | | X | | X | | X | | X | |
| (clinician | | | | | | | | | | | |
| portion) | | | | | | | | | | | |
| Clinician- | | | | | | | | | | | |
| administered | | | | | | | | | | | |
| assessments | | | | | | | | | | | |
| (paper) | | | | | | | | | | | |
| C-SSRS | X | | | | | | | | | | Adapted for the assessment |
| Screening/Baseli | | | | | | | | | | | of ideation and behavior |
| ne | | | | | | | | | | | categories only. |
| C-SSRS Since | | X | | X | | X | | X | | X | Adapted for the assessment |
| Last Assessed | | | | | | | | | | | of ideation and behavior |
| | | | | | | | | | | | categories only. |
| Fitzpatrick Skin | | X | | | | | | | | | |
| Type | | | | | | | | | | | |
| Laboratory tests | | | | | | | | | | | Collect before dosing unless |
| and sample | | | | | | | | | | | otherwise specified. |
| collections | | | | | | | | | | | |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Hematology | X | X | X | X | X | X | X | X | | X | |
| Clinical | X | X | | X | | X | | X | | X | |
| chemistry | | | | | | | | | | | |
| Lipid panel | | X | | | | | | | | X | |
| Urinalysis | X | X | | | | | | | | X | |
| Estimated | X | | | | | | | | | | Calculated using the CKD- |
| glomerular | | | | | | | | | | | EPI Creatinine Equation |
| filtration rate | | | | | | | | | | | (2021). |
| (eGFR) | | | | | | | | | | | |
| Serum | X | | | | | | | | | | |
| pregnancy | | | | | | | | | | | |
| Urine pregnancy | | X | | X | | X | | X | | X | For WOCBP, a pregnancy |
| (local) | | | | | | | | | | | test must be performed, with |
| | | | | | | | | | | | the result available prior to |
| | | | | | | | | | | | dosing Additional |
| | | | | | | | | | | | pregnancy tests should be |
| | | | | | | | | | | | performed at any time |
| | | | | | | | | | | | during the study if a |
| | | | | | | | | | | | menstrual period is missed, |
| | | | | | | | | | | | or there is clinical suspicion |
| | | | | | | | | | | | of pregnancy, or as required |
| | | | | | | | | | | | by local law or regulation. |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Follicle- | X | | | | | | | | | | Optional; perform only for |
| stimulating | | | | | | | | | | | women believed to be |
| hormone (FSH) | | | | | | | | | | | WNOCBP to confirm |
| | | | | | | | | | | | postmenopausal status. |
| Immunoglobulin | | X | | | | X | | | | X | |
| E (IgE) total | | | | | | | | | | | |
| Interleukin 19 | | X | X | X | | X | | X | | X | |
| (IL-19) | | | | | | | | | | | |
| TARC | | X | X | X | | X | | X | | X | |
| Periostin | | X | X | X | | X | | X | | X | |
| Lymphocyte | | X | | | | | | X | | X | Samples will be taken from |
| subsets (flow | | | | | | | | | | | a minimum of |
| cytometry) | | | | | | | | | | | approximately 60 to 100 |
| | | | | | | | | | | | participants. |
| TB test | X | | | | | | | | | | For those participants with a |
| | | | | | | | | | | | PPD placed at Visit 1, the |
| | | | | | | | | | | | PPD must be read 48 to 72 |
| | | | | | | | | | | | hours after placement. PPD |
| | | | | | | | | | | | test does not need to be read |
| | | | | | | | | | | | at the site but must be read |
| | | | | | | | | | | | by a trained medical |
| | | | | | | | | | | | professional and results |
| | | | | | | | | | | | must be provided to the site |
| | | | | | | | | | | | prior to randomization. |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| HIV screening | X | | | | | | | | | | |
| tests | | | | | | | | | | | |
| Hepatitis C virus | X | | | | | | | | | | For participants who are |
| (HCV) screening | | | | | | | | | | | positive for HCV antibody, |
| tests | | | | | | | | | | | a follow-up test for HCV |
| | | | | | | | | | | | RNA will be performed. |
| | | | | | | | | | | | Participants who are anti- |
| | | | | | | | | | | | HCV positive and |
| | | | | | | | | | | | HCV RNA negative may be |
| | | | | | | | | | | | eligible to be enrolled. |
| Hepatitis B | X | | | | | | | | | | Participants who are HBsAg |
| Virus (HBV) | | | | | | | | | | | negative and anti-HBC |
| screening tests | | | | | | | | | | | positive will have an HBV |
| | | | | | | | | | | | DNA performed at |
| | | | | | | | | | | | screening. Participants who |
| | | | | | | | | | | | are anti-HBc positive and |
| | | | | | | | | | | | HBV DNA negative may be |
| | | | | | | | | | | | eligible to be enrolled but |
| | | | | | | | | | | | must undergo HBV DNA |
| | | | | | | | | | | | monitoring. |
| HBV DNA | X | | | | | | | X | | | Only for participants who |
| | | | | | | | | | | | are positive for anti-HBc at |
| | | | | | | | | | | | screening. |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Pharmacokinetic | | X | X | X | | X | | X | | X | Collect samples before |
| (PK) samples | | | | | | | | | | | dosing, if dosing is |
| | | | | | | | | | | | scheduled. Collect |
| | | | | | | | | | | | additional samples at |
| | | | | | | | | | | | specified times relative to |
| | | | | | | | | | | | onset of hypersensitivity |
| | | | | | | | | | | | events |
| Immunogenicity | | X | X | X | | | | X | | X | Collect samples before |
| (ADA) samples | | | | | | | | | | | dosing, if dosing is |
| | | | | | | | | | | | scheduled. Collect |
| | | | | | | | | | | | additional samples at |
| | | | | | | | | | | | specified times relative to |
| | | | | | | | | | | | onset of hypersensitivity |
| | | | | | | | | | | | events |
| Biopsy | | | | | | | | | | | Optional for patients at |
| | | | | | | | | | | | participating sites. |
| Skin punch | | X | | X | | | | | | X | Lesional and nonlesional at |
| biopsy | | | | | | | | | | | Visit 2. Lesional only at |
| | | | | | | | | | | | other timepoints. |
| Noninvasive | | X | X | X | | X | | | | X | Samples will be taken from |
| skin sampling | | | | | | | | | | | a total of approximately 40 |
| (skin patch) | | | | | | | | | | | to 60 participants across the |
| | | | | | | | | | | | study. |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Actigraphy | | | | | | | | | | | Optional for patients at |
| | | | | | | | | | | | participating sites. |
| Actigraphy | X | | | | X | | | | X | | |
| device dispensed | | | | | | | | | | | |
| Actigraphy | | X | | | | X | | | | X | A minimum of 14 days of |
| device returned | | | | | | | | | | | actigraphy data should be |
| | | | | | | | | | | | collected before the device |
| | | | | | | | | | | | is returned at Visit 2; |
| | | | | | | | | | | | however, collection of less |
| | | | | | | | | | | | than 14 days of data will not |
| | | | | | | | | | | | be considered a protocol |
| | | | | | | | | | | | deviation for Visit 2. |
| Stored samples | | | | | | | | | | | |
| Genetics sample | | X | | | | | | | | | Sample can be obtained at |
| | | | | | | | | | | | or after Visit 2. |
| Exploratory | | X | X | X | | X | | X | | X | Samples to be collected |
| biomarkers | | | | | | | | | | | before dosing, if dosing is |
| samples | | | | | | | | | | | scheduled. |
| Randomization | | | | | | | | | | | |
| and dosing | | | | | | | | | | | |
| Register visit | X | X | X | X | X | X | X | X | X | X | |
| with IWRS | | | | | | | | | | | |
| Randomization | | X | | | | | | | | | |
| via IWRS | | | | | | | | | | | |
| Weeks from | — | — | 2 | 4 | 6 | 8 | 10 | 12 | 14 | 16 | |
| Randomization | | | | | | | | | | | |
| Study day | — | 1 | 15 | 29 | 43 | 57 | 71 | 85 | 99 | 113 | |
| Visit interval | 35 to 15 | — | ±3 | ±3 | ±3 | ±3 | ±3 | ±3 | ±3 | ±3 | The interval between V1 |
| tolerance (days) | before V2 | | | | | | | | | | and V2 is 35 to 3 days for |
| | | | | | | | | | | | participants who do not |
| | | | | | | | | | | | participate in actigraphy. |
| Fasting visit | | X | | | | | | | | X | See fasting instructions in |
| Rerandomization | | | | | | | | | | X | |
| via IWRS | | | | | | | | | | | |
| Dispense study | | X | X | X | X | X | X | X | X | X | |
| drug via IWRS | | | | | | | | | | | |
| Administer study | | X | X | X | X | X | X | X | X | X | V2 administration should occur |
| drug | | | | | | | | | | | last, after all assessments/sample |
| | | | | | | | | | | | collections performed |

Note:
Visit 1 procedures may be conducted over more than 1 day as long as all activities are completed within the allowable visit tolerance.
For early discontinuations that occur before the last visit in treatment period, see the activities listed for EDV in Table 3.

TABLE 2
Maintenance treatment period of Study JIP-MC-KFAE

| | Treatment Period - Maintenance | | | | | | | |
|---|---|---|---|---|---|---|---|---|
| Visit number | V11 | V12 | V13 | V14 | V15 | V16 | V17 | Comment |
| Weeks from | 20 | 24 | 28 | 32 | 36 | 40 | 44 | |
| Randomization | | | | | | | | |
| Study day | 141 | 169 | 197 | 225 | 253 | 281 | 309 | |
| Visit interval tolerance | ±3 | ±3 | ±3 | ±3 | ±3 | ±3 | ±3 | |
| (days) | | | | | | | | |
| Fasting visit | | | | | | | | No fasting required in this period. |
| Concomitant medications | X | X | X | X | X | X | X | Data will be collected for medications, |
| | | | | | | | | including rescue therapy, and |
| | | | | | | | | background emollient. |
| Adverse events (AEs) | X | X | X | X | X | | X | Events that occur after signing the ICF |
| | | | | | | | | are considered AEs. |
| Physical evaluation | | | | | | | | |
| Weight | | | X | | | | | |
| Vital signs | X | X | X | X | X | X | X | Includes pulse rate, blood pressure, |
| | | | | | | | | respiratory rate, and body temperature. |
| | | | | | | | | Measured after participant has been |
| | | | | | | | | sitting at least 5 minutes. |
| Symptom-directed | | | X | | | X | | As shown in this schedule and as needed |
| physical assessment | | | | | | | | based on participant status and standard |
| | | | | | | | | of care. May be performed by qualified |
| | | | | | | | | personnel per local regulations. Assess |
| | | | | | | | | for TB risk factors, and for signs and |
| | | | | | | | | symptoms of active TB, including an |
| | | | | | | | | assessment of peripheral lymph nodes ;. |
| 12-lead ECG | | | X | | | | X | Local ECGs collected as instructed in. |
| Participant education and | | | | | | | | |
| supplies | | | | | | | | |
| Diary education | | | X | | | | | Additional education provided as needed. |
| Actigraphy | | | | | | X | | |
| Participant diary | | | | | | | | |
| (electronic) | | | | | | | | |
| Diary compliance check | X | X | X | X | X | X | X | |
| Patient-reported outcomes | | | | | | | | Complete prior to any clinician- |
| (electronic) | | | | | | | | administered assessments. |
| DLQI | X | X | X | X | X | | X | |
| POEM | X | X | X | X | | | | |
| HADS | X | X | X | X | X | X | X | |
| SCORAD (patient portion) | X | X | X | X | | X | X | |
| Clinician-administered | | | | | | | | |
| assessments (electronic) | | | | | | | | |
| EASI | X | X | X | X | X | X | X | |
| Weeks from | 20 | 24 | 28 | 32 | 36 | 40 | 44 | |
| Randomization | | | | | | | | |
| Study day | 141 | 169 | 197 | 225 | 253 | 281 | 309 | |
| Visit interval tolerance | ±3 | ±3 | ±3 | ±3 | ±3 | ±3 | ±3 | |
| (days) | | | | | | | | |
| Fasting visit | | | | | | | | No fasting required in this period. |
| VIGA-AD | X | X | X | X | X | X | X | |
| SCORAD (clinician | X | X | X | X | X | X | X | |
| portion) | | | | | | | | |
| Clinician-administered | | | | | | | | |
| assessments (paper) | | | | | | | | |
| C-SSRS Since Last | | X | X | X | X | X | X | Adapted for the assessment of ideation |
| Assessed | | | | | | | | and behavior categories only. |
| Laboratory tests and | | | | | | | | Collect before dosing unless otherwise |
| sample collections | | | | | | | | specified. |
| Hematology | X | X | X | X | X | X | X | |
| Clinical chemistry | X | X | X | X | X | X | X | |
| Lipid panel | | | | | | X | | |
| Urinalysis | | | | | | X | | |
| Urine pregnancy (local) | X | X | X | X | X | X | X | For WOCBP, a local pregnancy test must |
| | | | | | | | | be performed, with the result available |
| | | | | | | | | prior to dosing. Additional pregnancy |
| | | | | | | | | tests should be performed at any time |
| | | | | | | | | during the study if a menstrual period is |
| | | | | | | | | missed, or there is clinical suspicion of |
| | | | | | | | | pregnancy, or as required by local law or |
| | | | | | | | | regulation. |
| Immunoglobulin E (IgE) | | | | | | X | | |
| total | | | | | | | | |
| Interleukin 19 (IL-19) | X | X | X | X | | X | | |
| TARC | X | X | X | X | | X | | |
| Periostin | X | X | X | X | | X | | |
| Lymphocyte subsets (flow | | | X | | | X | | Samples will be taken from a minimum |
| cytometry) | | | | | | | | of approximately 60 to 100 participants. |
| HBV DNA | | X | | | X | | | Only for participants who are positive for |
| | | | | | | | | anti-HBc at screening. |
| Pharmacokinetic (PK) | X | X | X | | | X | X | Collect samples before dosing, if dosing |
| samples | | | | | | | | is scheduled. Collect additional samples |
| | | | | | | | | at specified times relative to onset of |
| | | | | | | | | hypersensitivity events |
| Weeks from | 20 | 24 | 28 | 32 | 36 | 40 | 44 | |
| Randomization | | | | | | | | |
| Study day | 141 | 169 | 197 | 225 | 253 | 281 | 309 | |
| Visit interval tolerance | ±3 | ±3 | ±3 | ±3 | ±3 | ±3 | ±3 | |
| (days) | | | | | | | | |
| Fasting visit | | | | | | | | No fasting required in this period. |
| Immunogenicity (ADA) | | | X | | | | X | Collect samples before dosing, if dosing |
| samples | | | | | | | | is scheduled. Collect additional samples |
| | | | | | | | | at specified times relative to onset of |
| | | | | | | | | hypersensitivity events |
| Biopsy | | | | | | | | No biopsy in this study period. |
| Actigraphy | | | | | | | | Optional for patients at participating |
| | | | | | | | | sites. |
| Actigraphy device | | | | | | X | | |
| dispensed | | | | | | | | |
| Actigraphy device | | | | | | | X | |
| returned | | | | | | | | |
| Stored samples | | | | | | | | |
| Genetics sample | | | | | | | | Sample can be obtained at or after Visit 2. |
| Exploratory biomarkers | | | | | | X | | Samples to be collected before dosing, if |
| samples | | | | | | | | dosing is scheduled. |
| Randomization and dosing | | | | | | | | |
| Register visit with IWRS | X | X | X | X | X | X | X | |
| Dispense study drug via | X | X | X | X | X | X | | |
| IWRS | | | | | | | | |
| Administer study drug | X | X | X | X | X | X | | |

Note:
For early discontinuations that occur before the last visit in treatment period, see the activities listed for EDV in Table 3.

TABLE 3
Unscheduled visit (UV), early discontinuation visit (EDV), and posttreatment
follow-up visits (801 and 802) of Study JIP-MC-KFAE

| | Visit number | | | | |
|---|---|---|---|---|---|
| | UV | EDV | 801 | 802 | Comment |
| | Weeks from Randomization | | | | |
| | | | 48 or EDV + | 52 orEDV + | |
| | | | 8 w after | 12 w after | |
| | — | — | last dose | last dose | |
| | Study day | | | | |
| | — | — | 337 | 365 | |
| | Visit interval tolerance (days) | | | | |
| | — | — | ±3 | ±3 | |
| | Fasting visit | | | | |
| | | X | | X | . |
| Concomitant | X | X | X | X | Data will be collected for medications, |
| medications | | | | | including rescue therapy, and background |
| | | | | | emollient. |
| Adverse events (AEs) | X | X | X | X | Events that occur after signing the ICF are |
| | | | | | considered AEs |
| Physical evaluation | | | | | |
| Weight | | X | | X | |
| Vital signs | X | X | X | X | Includes pulse rate, blood pressure, |
| | | | | | respiratory rate, and body temperature. |
| | | | | | Measured after participant has been |
| | | | | | sitting at least 5 minutes. |
| Symptom-directed | X | X | | X | As shown in this schedule and as needed |
| physical assessment | | | | | based on participant status and standard |
| | | | | | of care. May be performed by qualified |
| | | | | | personnel per local regulations. Assess for |
| | | | | | TB risk factors, and for signs and |
| | | | | | symptoms of active TB, including an |
| | | | | | assessment of peripheral lymph nodes; |
| 12-lead ECG | | X | | X | Local ECGs collected as instructed in. |
| Participant education | | | | | |
| and supplies | | | | | |
| Diary education | | | X | | Additional education provided as needed. |
| Participant diary | | | | | |
| (electronic) | | | | | |
| Diary compliance | X | X | X | X | |
| check | | | | | |
| Diary returned | | X | | X | Collect diary from participants at their |
| | | | | | last visit. |
| Patient-reported | | | | | Complete prior to any clinician- |
| outcomes (electronic) | | | | | administered assessments. |
| DLQI | | X | X | X | |
| POEM | | X | X | X | |
| HADS | | X | X | X | |
| SCORAD (patient | X | X | X | X | |
| portion) | | | | | |
| Clinician-administered | | | | | |
| assessments (electronic) | | | | | |
| EASI | X | X | X | X | |
| vIGA-AD | X | X | X | X | |
| SCORAD (clinician | X | X | X | X | |
| portion) | | | | | |
| Clinician-administered | | | | | |
| assessments (paper) | | | | | |
| C-SSRS Since Last | X | X | X | X | |
| Assessed | | | | | |
| Laboratory tests and | | | | | |
| sample collections | | | | | |
| Hematology | | X | X | X | |
| Clinical chemistry | | X | X | X | |
| Lipid panel | | X | | X | |
| Urinalysis | | X | | X | |
| Urine pregnancy | | X | X | X | For WOCBP, a local pregnancy test must |
| (local) | | | | | be performed, with the result available |
| | | | | | prior to dosing. Additional pregnancy |
| | | | | | tests should be performed at any time |
| | | | | | during the study if a menstrual period is |
| | | | | | missed, or there is clinical suspicion of |
| | | | | | pregnancy, or as required by local law or |
| | | | | | regulation. |
| Immunoglobulin E | | X | | X | |
| (IgE) total | | | | | |
| Interleukin 19 (IL-19) | | | | X | |
| TARC | | X | | X | |
| Periostin | | X | | X | |
| HBV DNA | | X | | X | Only for participants who are positive for |
| | | | | | anti-HBc at screening. |
| Pharmacokinetic (PK) | | X | | X | Collect additional samples at specified |
| samples | | | | | times relative to onset of hypersensitivity |
| | | | | | events. |
| Immunogenicity | | X | | X | Collect additional samples at specified |
| (ADA) samples | | | | | times relative to onset of hypersensitivity |
| | | | | | events. |
| Biopsy | | | | | Optional for patients at participating sites. |
| Skin punch biopsy | | X | | | Lesional only. Samples will be taken from |
| | | | | | a total of approximately 40 to 60 |
| | | | | | participants across the study. |
| Noninvasive skin | | X | | X | Samples will be taken from a total of |
| sampling (skin patch) | | | | | approximately 40 to 60 participants across |
| | | | | | the study. |
| Actigraphy | | | | | Optional for patients at participating sites. |
| Actigraphy device | | X | | | |
| returned | | | | | |
| Stored samples | | | | | |
| Genetics sample | | | | | Sample can be obtained at or after Visit 2. |
| Exploratory | | X | | X | |
| biomarkers samples | | | | | |
| Randomization and | | | | | Study drug is not administered at EDV, |
| dosing | | | | | Visit 801, or Visit 802. |
| Register visit with | | X | X | X | |
| IWRS | | | | | |

Note:
The posttreatment are required for all randomized participants.
Abbreviations: ADSS = Atopic Dermatitis Sleep Scale; AESI = adverse event of special interest; anti-HBc = antibody to hepatitis B core antigen; CKD-EPI = Chronic Kidney Disease Epidemiology Collaboration; C-SSRS = Columbia Suicide-Severity Rating Scale; DLQI = Dermatology Life Quality Index; DNA = deoxyribonucleic acid; EASI = Eczema Area and Severity Index; ECG = electrocardiogram; EDV = early discontinuation visit; HADS = Hospital Anxiety Depression Scale; HBV = hepatitis B virus; ICF = informed consent form; IWRS = interactive web-response system; NRS = numeric rating scale; PA = posterior-anterior; PGI-S-AD = Patient Global Impression of Severity-Atopic Dermatitis; POEM = Patient-Oriented Eczema Measure; PPD = purified protein derivative; RNA = ribonucleic acid; SCORAD = SCORing Atopic Dermatitis; TARC = thymus and activation-regulated chemokine; TB = tuberculosis; UV = unscheduled visit; V = case report form visit; WNOCBP = women not of childbearing potential; WOCBP = women of childbearing potential.

### Objectives, Endpoints, and Estimands

This table lists objectives and endpoints for the evaluation of LY3471851 versus placebo.

| Objectives | Endpoints |
|---|---|
| Primary | |
| To compare the efficacy of LY3471851 | Proportion of dupilumab-naive participants |
| versus placebo as measured by EASI-75 in | achieving EASI-75 at Week 16 |
| the treatment of participants with moderate- | |
| to-severe AD | |
| Secondary | |
| To compare the efficacy of LY3471851 | Proportion of dupilumab-naive participants |
| versus placebo, as measured by improvement | achieving at Week 16: |
| in signs and/or symptoms in the treatment of | EASI-50 |
| participants with moderate-to-severe AD | EASI-90 |
| | SCORAD-75 |
| | SCORAD-90 |
| | vIGA-AD of 0 or 1 |
| | Percent mean change from baseline to Week |
| | 16 for dupilumab-naive participants: |
| | EASI |
| | SCORAD |
| To characterize the PK of LY3471851 in | LY3471851 plasma trough concentrations at |
| participants with moderate-to-severe AD | Week 16 |

Exploratory Objectives/Endpoints
Exploratory objectives and endpoints may include, but not be limited to, evaluations of the following at various
study time points:
To evaluate signs and symptoms (including efficacy and patient-reported outcomes measures) at each time
point collected in dupilumab-naive or dupilumab-experienced patients
To describe the observed safety of LY3471851
To assess potential development of ADA and their potential effect on safety, efficacy, and LY3471851
concentration
To characterize dose-response and/or exposure-response as related to biomarkers, PD, efficacy, and safety
Additional details will be provided in the SAP.
Abbreviations:
AD = atopic dermatitis;
ADA = anti-drug antibodies;
EASI = Eczema Area and Severity Index;
EASI-50/75/90 = participant's EASI score is reduced by at least 50%/75%/90% relative to their baseline score;
SCORAD = SCORing Atopic Dermatitis;
SCORAD-75/90 = participant's SCORAD score is reduced by at least 75%/90% relative to their baseline score;
PD = pharmacodynamics;
PK = pharmacokinetics;

### Primary Estimand

The primary clinical question of interest is: what is the difference between each dosing regimen of LY3471851 and placebo in the target patient population, in achieving successful response at Week 16 without use of any rescue medication for AD or early discontinuation due to either lack of efficacy or adverse event, and if all participants continued with treatment for 16 weeks? The estimand is described by the following attributes:
Population: Dupilumab-naïve participants with moderate-to-severe atopic dermatitis
Endpoint: EASI-75 at Week 16
How to account for ICEs:
ICEs related to study intervention include use of any rescue medication for AD and early discontinuation due to either lack of efficacy or adverse event. A composite strategy will be used for these type of ICEs. Specifically, participants with ICEs related to study intervention will be considered as treatment failure (having no change from baseline), that is, a non-responder, after the first occurrence of these ICEs.
For all remaining ICEs:
   If data are available, a treatment policy strategy will be used and the observed data will be used regardless of whether ICEs unrelated to study intervention have occurred.
   If data are unavailable, a hypothetical strategy will be used to estimate what treatment response would have been if participants continued with treatment without the ICEs.
Population-level summary: Difference in response rate of EASI-75 at Week 16 between each dosing regimen of LY3471851 and placebo.
Rationale for estimand: The primary estimand strategy assumes the following:
   If a participant used any rescue medication for AD, the participant was not receiving sufficient benefits from study intervention.
   If a participant early discontinued study intervention due to lack of efficacy or adverse event, the participant experienced a burden of study intervention that outweighed its benefits.
   All remaining ICEs except use of any rescue medication for AD or early discontinuation due to lack of efficacy or adverse event are unrelated to study intervention.

### Secondary Estimands

The clinical question of interest for the secondary objectives is: what is the difference between each dosing regimen of LY3471851 and placebo in the target patient population, in achieving successful response at Week 16 without use of any rescue medication for AD or early discontinuation due to either lack of efficacy or adverse event, and if all participants continued with treatment for 16 weeks?
The estimand is described by the following attributes:
Population: Dupilumab-naïve participants with moderate to severe atopic dermatitis
Endpoints: All secondary endpoints, that is, EASI-50, EASI-90, SCORAD-75, SCORAD-90, vIGA-AD of 0 or 1, percent change from baseline in EASI, and percent change from baseline in SCORAD at Week 16
Intercurrent events will be accounted using the same estimand strategy as for the primary estimand.
Population-level summary:
   For binary endpoints EASI-50, EASI-90, SCORAD-75, SCORAD-90, vIGA-AD of 0 or 1, difference in response rate at Week 16 between each dosing regimen of LY3471851 and placebo
   For continuous endpoints percent change from baseline in EASI and percent change from baseline in SCORAD, difference in mean at Week 16 between each dosing regimen of LY3471851 and placebo

### Overall Design

This is a Phase 2, multicenter, randomized, double-blind, placebo-controlled, parallel-group outpatient dose-ranging study to evaluate the efficacy and describe the safety of multiple LY3471851 induction and maintenance dosing regimens in adult participants with AD. The study population includes dupilumab-naive and dupilumab-experienced participants with diagnosed AD which is moderate-to-severe as measured by EASI score ≥16, vIGA-AD score ≥3, and ≥10% of BSA involvement.

### Study Periods

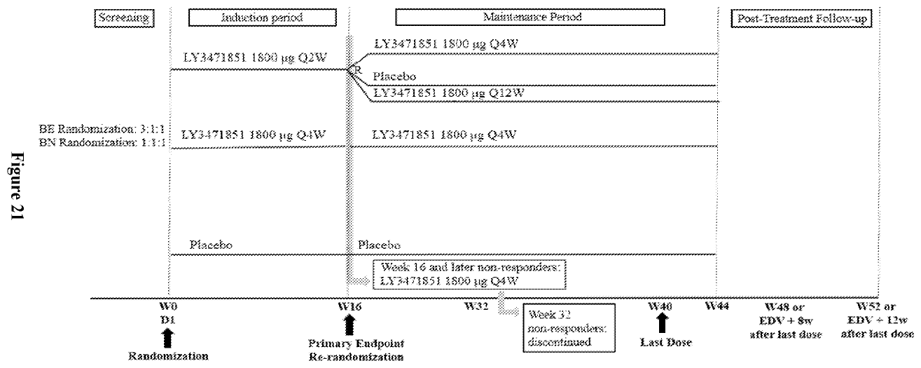

A schematic of the study design is presented in FIG. 12A. Alternative study designs are presented in FIG. 12B and FIG. 21 (which is directed to both bio-naïve (BN) and bio-experienced patients (BE)).

### Screening Period

The screening period begins with Visit 1, which occurs 15 to 35 days before the randomization visit. Informed consent will be obtained at Visit 1 before other study procedures are performed, and a participant identification number will be assigned. The SoA lists the screening procedures for Visit 1.
Participants found to be eligible according to all of the study entry criteria (Section 5) will be randomly assigned in a 2:2:1:1 ratio to one of the induction dosing regimens listed here.

| | Approximate number of |
|---|---|
| Induction regimen | participants planned |
| LY3471851 1800 μg Q2W | 100 |
| LY3471851 1800 μg Q4W | 100 |
| LY3471851 900 μg Q2W | 50 |
| placebo | 50 |

The randomization stratification factors include previous treatment with dupilumab, with a goal that approximately at least 80% of participants randomized at Visit 2 will be dupilumab-naive; however, enrollment of more or less than this target will not be considered a protocol deviation.

### Blinded Induction Period

Randomized participants will begin the double-blind, placebo-controlled, 16-week induction period when the initial dose is administered at Visit 2. Assessments, sample collections, and study drug administrations occur during the induction period at the visits shown in the SoA. Assessments and lab collections should occur prior to drug administration.
Topical rescue therapy is allowed starting at Week 8 for participants who do not achieve an EASI-25 response.

### Blinded Maintenance Period

The blinded 28-week maintenance treatment period begins with the dose administered at Week 16. Participants having at least an EASI-50 response at Week 16 will be reassigned to a maintenance dosing regimen, as listed here.

| Week 16 responders who were | |
|---|---|
| originally randomized to this | Will be reassigned to this |
| induction regimen . . . | maintenance regimen: |
| LY3471851 1800 μg Q2W | by rerandomization in 1:1 ratio: |
| | LY3471851 1800 μg Q4W or |
| | LY3471851 1800 μg Q12W |
| LY3471851 1800 μg Q4W | by rerandomization in 1:1 ratio: |
| | LY3471851 1800 μg Q4W or |
| | LY3471851 1800 μg Q12W |
| LY3471851 900 μg Q2W | LY3471851 900 μg Q4W |
| placebo | LY3471851 1800 μg Q4W |

Note:
Participants who do not achieve an EASI-50 response at Week 32 or later will be discontinued from study intervention.

The maintenance period includes an “escape arm” for participants whose EASI response suggests the need for a higher and more frequently administered dose. The escape therapy is LY3471851 1800 μg Q4W. During the maintenance period, a participant will be reassigned to this escape therapy under the conditions listed here.

| Begin LY3471851 1800 μg | |
|---|---|
| Q4W as escape therapy . . . | if the participant . . . |
| At Week 16 | has not achieved an EASI-50 response at |
| | Week 16. |
| At Week 16 | has received rescue therapy any time from |
| | Week 8 to Week 16 |
| At Week 20, Week 24, | has not achieved an EASI-25 response at |
| Week 28, or Week 32 | Week 20, Week 24, Week 28, or Week 32 |

A participant assigned to escape therapy will continue to receive it through the last dosing visit of the study (Week 40) unless the participant is discontinued from study intervention, for example, due to nonresponse.

### Posttreatment Follow-Up Period

All participants are expected to have 2 follow-up visits after the last dosing visit, as listed here.

| Condition | Visit 801 | Visit 802 |
|---|---|---|
| If the participant did | occurs 48 weeks after | occurs 52 weeks after |
| not discontinue study | randomization | randomization |
| intervention early . . . | (Visit 2). | (Visit 2). |
| If the participant did | occurs after the EDV | occurs after the EDV |
| discontinue study | and with an 8-week | and with a 12-week |
| intervention early . . . | interval between | interval between |
| | the last dose and | the last dose and |
| | Visit 801. | Visit 802. |

Blinding of the participant's induction and maintenance treatment assignments will continue until all participants have completed their last posttreatment follow-up visit or have discontinued the study.

### Study Assessments and Procedures

Study procedures and their timing are summarized in the SoA. Adherence to the study design requirements, including those specified in the SoA, is essential and required for study conduct. All blinded assessments and sample collections should be completed before a dose is administered at the dosing visits.
Efficacy Assessments
Primary Efficacy Assessment
Eczema Area and Severity Index
   The EASI is an investigator-administered, 20-item scale in adults that evaluates 2 dimensions of atopic dermatitis:
extent of disease at 4 body regions (head/neck, trunk, upper and lower extremities) and
4 clinical signs (erythema, induration/papulation, excoriation, and lichenification).
   The clinical signs are assessed for severity on a scale of 0 (absent) to 3 (severe).
   The scores are added up for each of the 4 body regions. The assigned percentages of BSA for each section of the body are
10% for head/neck,
20% for upper extremities,
30% for trunk, and
40% for lower extremities, respectively.
   Each subtotal score is multiplied by the BSA represented by that region.
   In addition, an area score of 0 to 6 is assigned for each body region, depending on the percentage of AD-affected skin in that area:
0 (none),
1 (1% to 9%),
2 (10% to 29%),
3 (30% to 49%),
4 (50% to 69%),
5 (70% to 89%), or
6 (90% to 100%).
   Each of the body area scores are multiplied by the area affected. The resulting EASI score ranges from 0 to 72 points, with the highest score indicating worse severity of atopic dermatitis (Hanifin et al. 2001). The recall period of this scale is present time.

### Secondary Efficacy Assessments

### Validated Investigator's Global Assessment for AD

The IGA used in this study is the vIGA-AD™. The vIGA-AD measures the investigator's global assessment of the participant's overall severity of their AD, based on a numeric 5-point scale from 0 (clear skin) to 4 (severe disease) (Simpson et al. 2020). The score is based on an overall assessment of the degree of erythema, papulation/induration, oozing/crusting, and lichenification. The recall period of this assessment is present time.

### SCORing Atopic Dermatitis

The SCORAD index is an investigator and participant-administered, 9-item assessment that assesses 3 aspects as provided below.
The extent (1-item) of AD is assessed as a percentage of each defined body area and reported as the sum of all areas. The maximum score is 100%.
The severity of six specific symptoms of AD: erythema, edema/papulation, oozing/crusts, excoriation, lichenification and dryness (6-items) is assessed by the investigator using a 4-point scale (i.e., none=0, mild=1, moderate=2, severe=3) with a maximum possible total of 18 points.
The subjective symptoms of pruritus and sleep loss (2 items) is assessed by the participant via a 10-cm VAS. The symptoms (itch and sleeplessness) are recorded by the participant on a visual analogue scale, where 0 is no symptoms and 10 is the worst imaginable symptom, with a maximum possible score of 20.
   The maximum possible SCORAD score calculated based on the above three aspects is 103. The higher scores indicate poorer or more severe condition (Stalder and Taieb 1993; Oranje et al. 2007; Schram et al. 2012; Kunz et al. 1997). The recall period is present time for extent and intensity, and average for the last 3 days/nights for subjective symptoms of AD.

### Other Efficacy, Patient-Reported Outcomes, and Quality of Life Assessments

### Itch Numeric Rating Scale

The Itch NRS is a participant-administered, single item, 11-point horizontal scale anchored at 0 and 10, with 0 representing “no itch” and 10 representing “worst itch imaginable” in adults. Overall severity of a participant's itching is indicated by selecting the number that best describes the worst level of itching in the past 24 hours (Naegeli et al. 2015; Kimball et al. 2016; Newton et al. 2019).

### Patient-Oriented Eczema Measure

The POEM is a simple, participant-administered, 7-item, scale that assesses disease severity in children and adults. Participants respond to questions about the frequency of 7 symptoms (itching, sleep disturbance, bleeding, weeping/oozing, cracking, flaking, and dryness/roughness) over the past week. Response categories include “No days,” “1-2 days,” “3-4 days,” “5-6 days,” and “Every day” with corresponding scores of 0, 1, 2, 3, and 4, respectively. Scores range from 0-28, with higher total scores indicating greater disease severity (Charman et al. 2004).

### Atopic Dermatitis Sleep Scale (ADSS)

The ADSS is a participant-administered 3-item questionnaire in adults developed to assess the impact of itch on sleep including difficulty falling asleep, frequency of waking, and difficulty getting back to sleep last night. Participants rate their difficulty falling asleep and difficulty getting back to sleep, items 1 and 3, respectively, using a 5-point Likert-type scale with response options ranging from 0 “not at all” to 4 “very difficult.” Participants report their frequency of waking last night, item 2, by selecting the number of times they woke up each night, ranging from 0 to 29 times. The ADSS is designed to be completed each day with respondents thinking about sleep “last night.” Each item is scored individually.

### Patient Global Impression of Severity-Atopic Dermatitis

The PGI-S-AD is a participant-administered single-item assessment asking the participant how they would rate their overall AD symptoms over the past 24 hours. The 5 categories of responses range from “no symptoms” to “severe.”

### Skin Pain Numeric Rating Scale

Skin Pain NRS is a participant-administered, 11-point horizontal scale anchored at 0 and 10, with 0 representing “no pain” and 10 representing “worst pain imaginable.” Overall severity of a participant's skin pain is indicated by selecting the number that best describes the worst level of skin pain in the past 24 hours (Newton et al. 2019).

### Dermatology Life Quality Index

The DLQI is a simple, participant-administered, 10-item, validated, QoL questionnaire in adults that covers 6 domains including symptoms and feelings, daily activities, leisure, work and school, personal relationships, and treatment. Response categories include “not at all,” “a little”, “a lot,” and “very much,” with corresponding scores of 0, 1, 2, and 3, respectively, and unanswered (or “not relevant”) responses scored as 0. Scores range from 0 to 30 with higher scores indicating greater impairment of QoL. A DLQI total score of 0 to 1 is considered as having no effect on a participant's health-related QoL (Hongbo et al. 2005), and a 4-point change from baseline is considered as the minimal clinically important difference threshold (Khilji et al. 2002; Basra et al. 2015). The recall period for DLQI is the past week.

### TCS Question

The TCS question will be answered by participants who have been rescued to an approved protocol rescue therapy. After site personnel indicate in the electronic tablet that the participant has started to receive rescue therapy, the following single question will be added to the participant's daily e-diary: “Have you applied topical steroid medication (for example, steroid creams, hydrocortisone, triamcinolone) to your skin in the past 24 hours?”

The null hypotheses corresponding to the primary and secondary estimands are these: there is no difference between LY3471851 and placebo in dupilumab-naïve participants with moderate-to-severe AD with respect to
proportion of patients achieving EASI-75 at Week 16 (primary)
proportion of patients achieving EASI-50 at Week 16 (secondary)
proportion of patients achieving EASI-90 at Week 16 (secondary)
proportion of patients achieving SCORAD-75 at Week 16 (secondary)
proportion of patients achieving SCORAD-90 at Week 16 (secondary)
proportion of patients achieving vIGA-AD of 0 or 1 at Week 16 (secondary)
mean percent change from baseline in EASI to Week 16 (secondary)
mean percent change from baseline in SCORAD to Week 16 (secondary)

### Multiplicity Adjustment

No adjustments for multiplicity will be performed.

### Analyses Sets

For the purposes of analysis, the following analysis sets are defined:

| | | Analyses |
|---|---|---|
| Analysis | | conducted on |
| Set | Description | this analysis set |
| Entered | All participants who sign the ICF. | Participant |
| | | disposition |
| Full | All randomized participants, i.e. Intent- | Induction and/or |
| Analysis | to-Treat (ITT) population. All observed | maintenance periods |
| Set | data will be included in the analysis set. | exploratory efficacy |
| | | or patient-reported |
| | | outcomes analyses |
| Primary | All randomized patients who are | Induction period |
| Analysis | dupilumab-naïve. Observations after the | primary and |
| Set | first occurrence of study-intervention- | secondary estimands |
| | related ICEs will be excluded from this | analyses |
| | analysis set and imputed by | |
| | nonresponse (or baseline). | |
| Secondary | All randomized patients who are | Induction period |
| Analysis | dupilumab-experienced. Observations | exploratory efficacy |
| Set | after the first occurrence of study- | or patient-reported |
| | intervention-related ICEs will be | outcomes analyses |
| | excluded from this analysis set and | |
| | imputed by nonresponse (or baseline). | |
| Safety | All participants randomized to study | Induction and/or |
| Analysis | intervention who take at least 1 dose of | maintenance periods |
| Set | study intervention. All observed data | safety data analyses |
| | will be included in the analysis set. | |
| PK | All participants randomized to study | Induction and/or |
| Analysis | intervention who take at least 1 dose of | maintenance periods |
| Set | study intervention and have PK data | pharmacokinetic |
| | available. All observed data will be | data analyses |
| | included in the analysis set. | |
| Follow-up | All randomized participants who take at | Posttreatment |
| Analysis | least 1 dose of study intervention and | follow-up period |
| Set | enter the posttreatment follow-up | safety and efficacy |
| | period. All observed data will be | data analyses |
| | included in the analysis set. | |

Additional analysis sets will be defined in the SAP to support exploratory analyses.

### Statistical Analyses

### General Considerations

Statistical analysis of this study will be the responsibility of the sponsor or its designee. Unless indicated otherwise in the SAP, the analyses will be conducted as described in this section. Any change to the data analysis methods described in this protocol will require an amendment only if the change affects a principal feature of the protocol. Any other change to the data analysis methods described in the protocol and the justification for making the change will be described in the clinical study report (CSR). Additional exploratory analyses of the data will be conducted as deemed appropriate. Complete details of the planned analyses will be documented in the SAP.
Continuous data will be summarized in terms of mean, standard deviation, minimum, maximum, median, and number of observations. Categorical data will be summarized as frequency counts and percentage. Comparison between each LY3471851 dosing regimen and placebo will be performed for all efficacy and patient-reported endpoint analyses during the induction period with no adjustment for multiple comparisons.
Dose selection at the end of the induction period will be based on pairwise comparisons between each regimen and placebo. Dose selection rules and specifics will be detailed in the SAP.
For efficacy and PRO analyses, baseline will be defined as the last available value before the first dose of study intervention. In most cases, this value will be what is recorded at the randomization visit (Visit 2). For efficacy measures, if a participant does not take any study intervention, the last available value on or prior to randomization date will be used. Change from baseline will be calculated as the visit value of interest minus the baseline value.
For induction period safety analyses, the baseline period is defined as the time from Visit 1 to the first dose of study intervention. For the safety analyses during the maintenance period, baseline is defined as the last available value before the first dose of study intervention during the maintenance period. In most cases, this will be the measure recorded at Week 16 (Visit 10). For the safety analyses during the posttreatment follow-up period, baseline is defined as the last nonmissing assessment on or prior to entering the posttreatment period, that is on or prior to Week 44 (Visit 17) or EDV.
For the primary and secondary estimands defined in Sections 3.1.1 and 3.1.2, missing data will be handled using Markov Chain Monte Carlo Multiple Imputation (MCMC-MI). Missing data imputation is not to be confused with an estimand strategy. Participants after ICEs related to study intervention will be considered non-responders (or having no change from baseline for continuous endpoints) as described in Section 3.3.1 and 3.1.2. Missing data imputation methods for supplementary estimands will be described in the SAP.
Treatment comparison for binary efficacy and patient-reported outcomes between each LY3471851 dosing regimen and placebo during the induction period will be made using a Cochran-Mantel-Haenszel (CMH) test stratified by randomization stratification factors. The 95% confidence intervals associated with the treatment response rate and treatment difference will be reported.
Treatment comparison for continuous efficacy and patient-reported outcomes between each LY3471851 dosing regimen and placebo during the induction period will be made using an analysis of covariance (ANCOVA) with the following in the model: treatment group, baseline value, and randomization stratification factors. Type III tests for least squares (LS) means will be used for statistical treatment comparison. The LS mean difference, standard error, p-value, and 95% CI will be reported.
For the induction period, Fisher's exact test will be used for all AEs and other categorical safety measures. Continuous vital sign and laboratory values will be analyzed using an ANCOVA with treatment and baseline value in the model.
All statistical tests, unless otherwise noted, will be two-sided and will be performed at a significance level of 0.05.
Additional details will be provided in the SAP.

### Primary Endpoint/Estimand Analysis

Treatment comparisons between each LY3471851 dosing regimen and placebo in proportion of dupilumab-naïve participants achieving EASI-75 at Week 16 will be conducted using a 2-sided CMH test with a significance level of 0.05, stratified by randomization stratification factors. The analysis will be conducted using the primary analysis set, where participants who have ICEs related to study intervention prior to Week 16 will be considered as non-responders. Missing data due to any reasons other than study-intervention-related ICEs will be imputed using MCMC-MI.
Treatment response rate, treatment difference vs. placebo, and their corresponding 95% confidence intervals will be provided according to study intervention to which patients are randomized to at Week 0 (Visit 2).

### Secondary Endpoints/Estimands Analysis

Treatment comparisons between each LY3471851 dosing regimen and placebo in proportion of dupilumab-naïve participants achieving the following efficacy measures at Week 16 will be conducted using a CMH test, stratified by randomization stratification factors:
EASI-50
EASI-90
SCORAD-75
SCORAD-90
vIGA-AD of 0 or 1
   Treatment comparisons between each LY3471851 dosing regimen and placebo for dupilumab-naïve participants in
Mean percent change from baseline to Week 16 in EASI
Mean percent change from baseline to Week 16 in SCORAD
   will be conducted using a Type III test for LS means from an ANCOVA model with treatment, baseline value, and randomization stratification factors.
   The comparisons will be two-sided and will be performed at a significance level of 0.05.
   The analysis will be conducted using the primary analysis set (Section 9.2), where participants who have ICEs related to study intervention prior to Week 16 will be considered as non-responders (or having no change from baseline for continuous endpoints). Missing data due to any reasons other than study-intervention-related ICEs will be imputed using MCMC-MI.
   For binary endpoints, treatment response rate, treatment difference vs. placebo, and their corresponding 95% confidence intervals will be provided. For continuous endpoints, LS mean, LS mean difference, and their 95% confidence interval will be provided.

### Example 6

### Rezpegaldesleukin Pharmaceutical Compositions

There is a desire for rezpegaldesleukin compositions providing injection device compatibility for the desired clinical dose ranges, acceptable stability, and tolerable patient injection experience. The present disclosure seeks to meet these needs by providing pharmaceutically acceptable formulations of rezpegaldesleukin. The present disclosure provides a pharmaceutical rezpegaldesleukin composition for subcutaneous injection. The composition comprises rezpegaldesleukin and about 5 mM sodium acetate, about 25 mM sodium chloride, about 7.5% w/v sucrose, and about pH 5.0. The rezpegaldesleukin drug substance concentration can be about 4.0 mg/mL to about 5.0 mg/mL rhIL-2 protein equivalent, and preferably about 4.5 mg/mL rhIL-2 protein equivalent, to enable flexibility in drug product manufacturing by enabling dilution to various drug product concentrations. Drug product concentrations may for example range from 0.3 mg/mL to 3.6 mg/m, including for instance 0.9 mg/mL or 1.8 mg/mL, as needed to provide the target dose in a one mL volume. Lower concentration preparations are also provided. The pharmaceutical compositions provide commercially acceptable injection device compatibility for the desired dose ranges, shelf-life stability, in-use stability, and is associated with a tolerable patient injection experience. The compositions utilize relatively lower sodium acetate, lower sodium chloride, and higher sucrose concentrations, and the solution osmolality targets ˜300 mOsm/kg for isotonicity. The present compositions provide overall low sub-visible particle counts, and favorable device functionality, for concentrations up to 5 mg/mL in semi-finished syringes (SFS), and low viscosity across the expected rezpegaldesleukin concentration range (0.3 mg/mL to 5 mg/mL). These formulations when used in Gen3 SFS demonstrate suitable performance in 0.5 ml auto-injector or 1 ml auto-injector or 2 ml auto-injector devices, respectively.

### Preparations of Rezpegaldesleukin Pharmaceutical Formulations:

The recombinant human IL-2 protein (aldesleukin) is expressed by E. coli cells which are scaled-up into a production fermenter. The cells are harvested, homogenized, and the inclusion bodies are isolated. Inclusion bodies are solubilized, depth filtered, acid precipitated and washed by Tangential Flow Filtration (TFF). The washed IL-2 precipitate is resolubilized, refolded, and concentrated/buffer exchanged by TFF. The purification of IL-2 uses a multimodal approach including cation exchange chromatography, ceramic hydroxyapatite chromatography, and mixed mode cation exchange chromatography. The purified IL-2 is then buffer exchanged by TFF storage and/or subsequent PEGylation. The PEGylation reaction covalently attaches branched 20 kDa PEG (PEG2ruNHS20K) moieties to the IL-2, and the resulting IL-2 conjugate rezpegaldesleukin is further purified by cation exchange chromatography, and fractions pooled according to target PEGylated species distribution ranges as described herein and/or in WO 2019/226538 which is incorporated herein by reference. The rezpegaldesleukin cation exchange chromatography intermediate is concentrated to achieve a calculated IL-2 concentration of 4-6 g/L based on the measured UV product concentration g/L and volume of the initial cation exchange chromatography intermediate. The rezpegaldesleukin Drug Substance preparation is diafiltered, and the product in the retentate side of the system is buffer exchanged against about 5 mM sodium acetate, about 25 mM sodium chloride, about 7.5% (w/v) sucrose, pH 5.0, and is dispensed into bottles and stored frozen at ≤−65° C.

The compositions of the present invention have concentrations of rezpegaldesleukin drug substance preferably between 4.0 mg/mL and 5.0 mg/mL. The compositions of the present invention have concentrations of rezpegaldesleukin drug product between 0.3 mg/ml and 3.6 mg/ml, and preferably between 0.3 mg/mL and 1.8 mg/ml, or other convenient concentrations for delivery of the dose embodiments as described herein. The IL-2 concentration is measured by UV absorbance. The final drug substance concentration target is 4.5 mg/mL with a final range of 4.0-5.0 mg/mL. The final retentate pH for the drug substance is between a range of 4.8 and 5.2 as the final range is pH 4.5 to 5.5.

Alternatively, drug product preparation batches of lower concentrations can be prepared by thawing rezpegaldesleukin drug substance (DS) frozen solution at refrigerated or ambient temperature conditions. The DS containers should be inverted several times after completion of thawing to ensure that the DS is uniformly distributed throughout the bulk solution. Once the DS aliquots have been thawed and mixed, a suitable quantity is pooled into a single vessel. The protein content is used to calculate the weight of DS solution required for the batch. The next step is dissolution of all other formulation excipients to prepare a buffer excipient solution. The quantities of glacial acetic acid, sodium acetate trihydrate, sodium chloride and sucrose needed for the batch are calculated.

For instance, the example below provides 1.8 mg/mL such that a one ml dose would provide 1800 μg. Rezpegaldesleukin is to be filled into sterile glass vials to contain 2 mL of 1.8 mg/mL solution (Rezpegaldesleukin Injection, 3.6 mg/2 mL). The complete list of ingredients and quantitative formulation on a per-mL basis is provided in the table below.

### Composition of a Pharmaceutical Rezpegaldesleukin Formulation:

| | | |
|---|---|---|
| | Quantity | |
| Ingredient | (mg/mL) | Function |

Active Ingredient

| | | |
|---|---|---|
| Rezpegaldesleukin | 1.8 | Active |

Other Ingredients^{a}

| | | |
|---|---|---|
| Glacial Acetic Acid | 0.091 | Buffer |
| Sodium Acetate | 0.475 | Buffer |
| Trihydrate^{a} | | |
| Sodium Chloride | 1.46 | Tonicity Agent |
| Sucrose | 75.0 | Tonicity Agent/ |
| | | Stabilizer |
| Water for Injection | q.s. to 1 mL | Solvent |

^{a}As a replacement for Sodium Acetate Trihydrate, Sodium Acetate Anhydrous may be used, at a quantity of 0.286 mg/mL.

To a clean vessel, a known amount of Water for Injection is added. The calculated amounts of glacial acetic acid, sodium acetate trihydrate, sodium chloride and sucrose are added and dissolved by mixing. Then, Water for Injection is added to reach the final buffer q.s. weight and the solution is mixed thoroughly. The pH and osmolality of the buffer excipient solution are checked, and the solution is filtered. If the pH and/or the osmolality are outside of their respective ranges, the solution is discarded, and a new buffer excipient solution must be prepared.

A quantity of the filtered buffer excipient solution is added to the formulation vessel that contains the DS solution, to provide 80% of the final batch weight. The solution is mixed thoroughly, and the protein content of the solution is measured. If the in-process UV assay result is within the expected range, the final batch weight is calculated based on the measured UV value and weight of solution at 80% of the target batch weight. If the UV result is outside of the expected range, mixing is continued followed by an additional UV analysis. Based on an evaluation of the data, the final weight for the batch is determined. Additional filtered buffer excipient solution is added to 100% of the final q.s. batch weight and the solution is mixed thoroughly. The pH of the solution and the UV measurement are checked.

The drug product solution is sampled for pre sterilized filtration bioburden testing and then passed through a polyvinylidene fluoride (PVDF) filter with a pore size rating of 0.22 microns for bioburden reduction. The drug product solution is then sterilized by PVDF membrane filtration with a pore size rating of 0.22 microns. The sterile filtered solution is aseptically filled into sterile containers and sterile stoppers are aseptically applied. The drug product vials are sealed and inspected for visible defects, and unacceptable units are rejected.

Such compositions may be presented in a pre-filled syringe. Such pre-filled syringe may be useful for administering one half milliliter or one milliliter of such composition per patient per dose. A dose of rezpegaldesleukin composition may be administered using a dosing schedule as provided in the embodiments described herein. The compositions are sterile when first produced. When used herein, “pharmaceutically acceptable salt” is well known to the skilled artisan. A composition for single use pre-filled syringe requires no preservative.

The pH of rezpegaldesleukin compositions of the present invention is typically about 5.0 and it is controlled by carefully controlling the quantities of glacial acetic acid and sodium acetate (“acid” and “base”) to achieve the desired pH. Patient injection site experience is a consideration for a subcutaneously administered composition. It is desirable to select a composition associated with a tolerable patient injection site experience, such as pain on injection. For example, NaCl and citrate have been associated with painful stinging at the injection site. (Laursen, T.; Hansen, B.; Fisker, S. Pain perception after subcutaneous injections of media containing different buffers. Basic & Clinical Pharmacology & Toxicology 2006, 98, (2), 218-221), (Fransson, J.; Espander-Jansson, A., Local tolerance of subcutaneous injections. Journal of Pharmacy and Pharmacology 1996, 48, (10), 1012-1015.) It is further desirable to match the tonicity (i.e., osmolality) of body fluids at the injection site as closely as possible when administering the compositions because solutions that are not approximately isotonic with body fluids can produce a painful stinging sensation when administered. The present rezpegaldesleukin composition comprising about 5 mM sodium acetate, about 25 mM sodium chloride, about 7.5% w/v sucrose, and about pH 5.0 provides a tolerable patient injection site experience.

A rezpegaldesleukin pharmaceutical composition comprising about 5 mM sodium acetate, about 25 mM sodium chloride, about 7.5% w/v sucrose, and about pH 5.0 provides a desired shelf-life stability and provides patients with a tolerable injection site experience. As used herein, “shelf-life stability” is measured under controlled conditions at about 5 degrees Celsius. A rezpegaldesleukin pharmaceutical composition comprising about 5 mM sodium acetate, about 25 mM sodium chloride, about 7.5% w/v sucrose, and about pH 5.0 further provides acceptable in-use stability. As used herein, the term “in-use stability” refers to the stability of the composition measured under controlled conditions at or about 25 degrees Celsius.

### Syringe Compatibility:

A three-month stability study of rezpegaldesleukin drug product was conducted comparing the following formulations in glass vials and syringes.

Formulation A: 5 mM sodium acetate, 25 mM NaCl, 7.5% sucrose, pH 5.0; 1.5 mg/mL rezpegaldesleukin.
Formulation B: 5 mM sodium acetate, 50 mM L-arginine HCl, 6.0% sucrose, pH 5.0; 1.5 mg/mL rezpegaldesleukin.

The rezpegaldesleukin drug product samples, formulated at 1.5 mg/mL, were prepared as described herein. The analytical tests performed for rezpegaldesleukin samples include pH, osmolality, turbidity (instrumental clarity and A550), UV, CEX, RP-HPLC, and SEC. No changes were observed for pH, turbidity, UV, or CEX analysis. While only small changes were observed during the SEC analysis, a valley-resolved species was observed on the front of the Di-PEG peak for Lot-Formulation B when stored at 35° C. and analyzed by RP-HPLC. This result suggests that 1.5 mg/mL rezpegaldesleukin formulated in 5 mM sodium acetate, 50 mM L-arginine HCl, 6.0% sucrose, pH 5.0 may not be compatible with the syringe used for this study. Thus, Formulation A may provide superior syringe compatibility.

### Results and Discussion

The osmolality for Lot-formulation A was 305 mOsm/kg. The osmolality for Lot-formulation B was 290 mOsm/kg. For all samples tested, pH was within 0.3 units of target pH, and within 0.2 unit of the pH at the initial timepoint. Protein concentration was 1.5 mg/mL for all samples. For clarity analysis, A550 measurements ranged from −0.0029 to 0.0389 and instrumental turbidity ranged from 1.20-6.01 NTU for all samples. These results indicate that there are no trends with respect to formulation, storage container, storage time or temperature for pH, protein concentration, and turbidity.

Results for the analysis of rezpegaldesleukin by RP-HPLC are summarized as follows. The Mono-PEG species % peak areas ranged from 2.4-3.2%. Di-PEG ranged from 41.9-43.3% and Tri-PEG ranged from 45.6-46.2%. The combined Di and Tri-PEG peak areas for IL-2 Conjugate samples ranged from 88.0-88.9%. Higher-PEGylated peaks ranged from 7.9-9.4% area. Free IL-2 was not detected in any of the samples. After eight weeks at 35° C., Lot-Formulation B had noticeable changes in the chromatographic profile; a valley-resolved peak appeared on the front side of the Di-PEG peak. Additionally, after storage for three months at 25° C., a shoulder was observed on the front of the Di-PEG peak for Lot-Formulation B. The new shoulder or peak was integrated with the Di-PEG peak; therefore, the resulting peak areas are not affected. The peak is not present after eight weeks at 35° C. or three months at 25° C. in Lot-formulation A, and is therefore likely due to the storage of rezpegaldesleukin in syringes when formulated in 5 mM sodium acetate, 50 mM L-arginine HCl, 6.0% sucrose at pH 5.0 (Lot-Formulation B).

### CONCLUSIONS

This study assessed the effects of excipient type and concentration on the stability of rezpegaldesleukin in syringes and glass vials over the course of a three-month stability study. No significant changes were observed throughout the study for pH, protein concentration, turbidity, and charge heterogeneity by CEX. Numerical purity by RP-HPLC was similar for the tested lots; however, a valley-resolved species was observed on the front of the Di-PEG peak for Lot-Formulation B when stored at 35° C. This result indicates that 1.5 mg/mL rezpegaldesleukin formulated in 5 mM sodium acetate, 50 mM L-arginine HCl, 6.0% sucrose, pH 5.0 may not be compatible with the syringe used for this study, in contrast to Formulation A: 5 mM sodium acetate, 25 mM NaCl, 7.5% sucrose, pH 5.0; 1.5 mg/mL rezpegaldesleukin. Thus, Formulation A may provide improved syringe compatibility compared to Formulation B.

What is claimed:

1. A method of treating a human subject having an autoimmune disease comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose of a selective Treg stimulator RUR_{20kD}-IL-2 composition, wherein the composition comprises a composition of Formulae A-F.

2. The method of claim 1 wherein the composition is a composition of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons.

3. The method of claim 1 wherein the composition is a composition of Formula F, wherein the composition comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons.

4. The method of claim 1 wherein the composition is a composition of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons.

5. The method of claim 1 wherein the composition is a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons.

6. The method of claim 1 wherein the composition is a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons.

7. The method of claim 1 wherein the composition comprises rezpegaldesleukin.

8. The method of claim 1 wherein said administering is carried out once every 2 weeks or once every 4 weeks.

9. The method of claim 1 wherein said administering is carried out once every 2 weeks.

10. The method of claim 1 wherein said administering is carried out once every 4 weeks.

11. The method according to any of claims 1-10 wherein said composition further comprises a formulation of about 5 mM sodium acetate, about 25 mM sodium chloride, about 7.5% (w/v) sucrose, at a pH of about 5.0.

12. The method according to any of claims 1-11 wherein the autoimmune disease is selected from systemic lupus erythematosus (SLE), ulcerative colitis, Crohn's disease, rheumatoid arthritis, atopic dermatitis, systemic sclerosis, ankylosing spondylitis, graft versus host disease, polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

13. The method according to any of claims 1-12, wherein the dose is about 450 μg, about 900 μg, about 1200 μg, about 1500 μg, about 1800 μg, or about 2100 μg per dose.

14. The method according to any of claims 1-12, wherein the dose is about 900 μg per dose.

15. The method according to any of claims 1-12, wherein the dose is about 1200 μg per dose.

16. The method according to any of claims 1-12, wherein the dose is about 1500 μg per dose.

17. The method according to any of claims 1-12, wherein the dose is about 1800 μg per dose.

18. The method according to any of claims 1-13, wherein the dose is about 2100 μg per dose.

19. The method according to any of claims 1-13, wherein the dose is about 2400 μg per dose.

20. The method according to any of claims 1-13, wherein the dose is about 2700 μg per dose.

21. The method according to any of claims 1-13, wherein the dose is about 3000 μg per dose.

22. The method according to any of claims 1-21 wherein the autoimmune disease is systemic lupus erythematosus (SLE).

23. The method according to any of claims 1-21 wherein the autoimmune disease is atopic dermatitis.

24. The method according to any of claims 1-21 wherein the autoimmune disease is atopic dermatitis and atopic dermatitis patient is bio-experienced.

25. The method according to any of claims 1-21 wherein the autoimmune disease is type 1 diabetes.

26. The method according to any of claims 1-21 wherein the autoimmune disease is peanut allergy.

27. A method of treating a human subject having an autoimmune disease comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

28. The method of claim 27 the dose of rezpegaldesleukin in the induction phase comprises 900 μg.

29. The method of claim 27 the dose of rezpegaldesleukin in the induction phase comprises 1200 μg.

30. The method of claim 27 the dose of rezpegaldesleukin in the induction phase comprises 1500 μg.

31. The method of claim 27 the dose of rezpegaldesleukin in the induction phase comprises 1800 μg.

32. The method of claim 27 the dose of rezpegaldesleukin in the induction phase comprises 2100 μg.

33. The method of claim 27 the dose of rezpegaldesleukin in the induction phase comprises 2400 μg.

34. The method of claim 27 the dose of rezpegaldesleukin in the induction phase comprises 2700 μg.

35. The method of claim 27 the dose of rezpegaldesleukin in the induction phase comprises 3000 μg.

36. The method according to one of claims 27-35 wherein the dose of rezpegaldesleukin in the maintenance phase comprises 900 μg.

37. The method according to one of claims 27-35 wherein dose of rezpegaldesleukin in the maintenance phase comprises 1200 μg.

38. The method according to one of claims 27-35 wherein the dose of rezpegaldesleukin in the maintenance phase comprises 1500 μg.

39. The method according to one of claims 27-35 wherein the dose of rezpegaldesleukin in the maintenance phase comprises 1800 μg.

40. The method according to one of claims 27-35 wherein the dose of rezpegaldesleukin in the maintenance phase comprises 2100 μg.

41. The method according to one of claims 27-35 wherein the dose of rezpegaldesleukin in the maintenance phase comprises 2400 μg.

42. The method according to one of claims 27-35 wherein the dose of rezpegaldesleukin in the maintenance phase comprises 2700 μg.

43. The method according to one of claims 27-35 wherein the dose of rezpegaldesleukin in the maintenance phase comprises 3000 μg.

44. The method according to one of claims 27-35 wherein the induction dose is administered once every two weeks.

45. The method according to one of claims 27-35 wherein the induction dose is administered once every four weeks.

46. The method according to one of claims 27-35 wherein the maintenance dose is administered once every four weeks.

47. The method according to one of claims 27-35 wherein the maintenance dose is administered once every eight weeks.

48. The method according to one of claims 27-35 wherein the maintenance dose is administered once every twelve weeks.

49. The method according to one of claims 27-35 wherein the maintenance dose is administered once every twenty-four weeks.

50. The method according to one of claims 27-35 wherein the maintenance dose is administered once every thirty-six weeks.

51. The method according to one of claims 27-35 wherein the maintenance dose is administered once every forty-eight weeks.

52. The method according to one of claims 27-35 wherein the maintenance dose is administered once every fifty-two weeks.

53. A method of treating moderate to severe atopic dermatitis in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration.

54. A method of reducing sleep loss in a patient with moderate to severe atopic dermatitis in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration.

55. A selective Treg stimulator RUR_{20kD}-IL-2 and related composition comprising a composition of Formulae A-F for use in the treatment an autoimmune disease in a patient, wherein the patient is administered a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose.

56. The use of claim 55 wherein the composition is a composition of Formula A, wherein the composition comprises, on a molar basis, about 5 mol % or less mono-PEGylated IL-2 conjugates, and from about 28 mol % to about 60 mol % di-PEGylated IL-2 conjugates, and from about 24 mol % to about 65 mol % tri-PEGylated IL-2 conjugates, and about 12 mol % or less of higher PEGylated IL-2 conjugates, and wherein the nominal average molecular weight of each branched polyethylene glycol moiety is about 20,000 daltons.

57. The use of claim 55 wherein the composition is a composition of Formula F, wherein the composition comprises, on a molar basis, about 2-4 mol % or less mono-PEGylated IL-2 conjugates, and from about 35 mol % to about 55 mol % di-PEGylated IL-2 conjugates, and from about 35 mol % to about 55 mol % tri-PEGylated IL-2 conjugates, and about 12 mol % or less of higher PEGylated IL-2 conjugates, and wherein the nominal average molecular weight of each branched polyethylene glycol moiety is about 20,000 daltons.

58. The use of claim 55 wherein the composition is a composition of Formula B, wherein the composition comprises, on a molar basis, from about 2.5 to about 4.5 mol % mono-PEGylated IL-2 conjugates, and from about 35 to about 50 mol % di-PEGylated IL-2 conjugates, and from about 38 to about 46 mol % tri-PEGylated IL-2 conjugates, and from about 3 to about 10 mol % higher PEGylated IL-2 conjugates, and wherein the nominal average molecular weight of each branched polyethylene glycol moiety is about 20,000 daltons.

59. The use of claim 55 wherein the composition is a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein the nominal average molecular weight of each branched polyethylene glycol moiety is about 20,000 daltons.

60. The use of claim 55 wherein the composition is a composition of Formula C, wherein the composition comprises, on a molar basis, from about 2.8 to about 3.8 mol % mono-PEGylated IL-2 conjugates, and from about 44 to about 48 mol % di-PEGylated IL-2 conjugates, and from about 41 to about 44 mol % tri-PEGylated IL-2 conjugates, and from about 7 to about 9 mol % higher PEGylated IL-2 conjugates, and wherein said composition comprises a mixture of mono-PEGylated IL-2 conjugates which have a PEG moiety attached at one of lysine K7 or K8 or K31 or K75, and wherein the nominal average molecular weight of each branched polyethylene glycol moiety is about 20,000 daltons.

61. The use of claim 55 wherein the composition comprises rezpegaldesleukin.

62. The use of claim 55 wherein said administering is carried out once every 2 weeks or once every 4 weeks.

63. The use of claim 55 wherein said administering is carried out once every 2 weeks.

64. The use of claim 55 wherein said administering is carried out once every 4 weeks.

65. The use according to any of claims 55-64 wherein said doses further comprise a formulation of 5 mM sodium acetate, 25 mM sodium chloride, 7.5% (w/v) sucrose, pH 5.0.

66. The use according to any of claims 55-64 wherein the autoimmune disease is selected from systemic lupus erythematosus (SLE), ulcerative colitis, Crohn's disease, rheumatoid arthritis, atopic dermatitis, systemic sclerosis, ankylosing spondylitis, graft versus host disease, polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

67. The use according to any of claims 55-66, wherein the dose is about 2100 μg, about 2400 μg, about 2700 μg, about 3000 μg, about 3300 μg, or about 3600 μg per dose.

68. The use according to any of claims 55-66, wherein the dose is about 900 μg per dose.

69. The use according to any of claims 55-66, wherein the dose is about 1200 μg per dose.

70. The use according to any of claims 55-66, wherein the dose is about 1500 μg per dose.

71. The use according to any of claims 55-66, wherein the dose is about 1800 μg per dose.

72. The use according to any of claims 55-66, wherein the dose is about 2100 μg per dose.

73. The use according to any of claims 55-66, wherein the dose is about 2400 μg per dose.

74. The use according to any of claims 55-66, wherein the dose is about 2700 μg per dose.

75. The use according to any of claims 55-66, wherein the dose is about 3000 μg per dose.

76. The use according to any of claims 55-75, wherein the autoimmune disease is systemic lupus erythematosus (SLE).

77. The use according to any of claims 55-75, wherein the autoimmune disease is atopic dermatitis.

78. The use according to any of claims 55-75, wherein the autoimmune disease is atopic dermatitis and the atopic dermatitis patient is bio-experienced.

79. The use according to any of claims 55-75, wherein the autoimmune disease is type 1 diabetes.

80. The use according to any of claims 55-75, wherein the autoimmune disease is peanut allergy.

81. A selective Treg stimulator RUR_{20kD}-IL-2 and related composition comprising a composition of Formulae A-F for use in the treatment of an autoimmune disease in a patient, wherein the patient is administered in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose; wherein said administering is carried out once weekly, once every 2 weeks, once every 3 weeks, once every 4 weeks, once every 6 weeks, or once every 8 weeks; wherein said composition is administered for a total of 12 weeks, a total of 16 weeks, a total of 20 weeks, or a total of 24 weeks of administration in said induction phase; followed by a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration; wherein said autoimmune disease is selected from: systemic lupus erythematosus (SLE), ulcerative colitis, Crohn's disease, rheumatoid arthritis, atopic dermatitis, systemic sclerosis, ankylosing spondylitis, graft versus host disease, and polymyositis; type 1 diabetes, Addison's disease, Hashimoto thyroiditis, Graves' disease, Sjogren's syndrome, vitiligo, pernicious anemia, glomerulonephritis, lupus nephritis, myasthenia gravis, Goodpasture's syndrome, autoimmune hemolytic anemia, idiopathic thrombocytopenia purpura, peanut allergy, pulmonary fibrosis, celiac disease, alopecia areata, psoriasis, Hidradenitis suppurativa or asthma.

82. The use of claim 81 wherein the dose in the induction phase comprises 900 μg.

83. The use of claim 81 wherein the dose in the induction phase comprises 1200 μg.

84. The use of claim 81 wherein the dose in the induction phase comprises 1500 μg.

85. The use of claim 81 wherein the dose in the induction phase comprises 1800 μg.

86. The use of claim 81 wherein the dose in the induction phase comprises 2100 μg.

87. The use of claim 81 wherein the dose in the induction phase comprises 2400 μg.

88. The use of claim 81 wherein the dose in the induction phase comprises 2700 μg.

89. The use of claim 81 wherein the dose in the induction phase comprises 3000 μg.

90. The use according to any one of claims 81-89 wherein the dose in the maintenance phase comprises 900 μg.

91. The use according to any one of claims 81-89 wherein the dose in the maintenance phase comprises 1200 g.

92. The use according to any one of claims 81-89 wherein the dose in the maintenance phase comprises 1500 μg.

93. The use according to any one of claims 81-89 wherein the dose in the maintenance phase comprises 1800 μg.

94. The use according to any one of claims 81-89 wherein the dose in the maintenance phase comprises 2100 μg.

95. The use according to any one of claims 81-89 wherein the dose in the maintenance phase comprises 2400 μg.

96. The use according to any one of claims 81-89 wherein the dose in the maintenance phase comprises 2700 μg.

97. The use according to any one of claims 81-89 wherein the dose in the maintenance phase comprises 3000 μg.

98. The use according to any one of claims 81-89 wherein the induction dose is administered once every two weeks.

99. The use according to any one of claims 81-89 wherein the induction dose is administered once every four weeks.

100. The use according to any one of claims 81-89 wherein the maintenance dose is administered once every four weeks.

101. The use according to any one of claims 81-89 wherein the maintenance dose is administered once every eight weeks.

102. The use according to any one of claims 81-89 wherein the maintenance dose is administered once every twelve weeks.

103. The use according to any one of claims 81-89 wherein the maintenance dose is administered once every twenty-four weeks.

104. The use according to any one of claims 81-89 wherein the maintenance dose is administered once every thirty-six weeks.

105. The use according to any one of claims 81-89 wherein the maintenance dose is administered once every forty-eight weeks.

106. The use according to any one of claims 81-89 wherein the maintenance dose is administered once every fifty-two weeks.

107. The use of any of claims 81-106 wherein the composition is rezpegaldesleukin.

108. A selective Treg stimulator RUR_{20kD}-IL-2 and related composition comprising a composition of Formulae A-F for use in the treatment of severe atopic dermatitis in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose; wherein said administering is carried out once weekly, once every 2 weeks, once every 3 weeks, once every 4 weeks, once every 6 weeks, or once every 8 weeks; wherein said composition is administered for a total of 12 weeks, a total of 16 weeks, a total of 20 weeks, or a total of 24 weeks of administration in said induction phase; determining if the patient is a responder to the composition after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said composition is administered for a total of 4 weeks to 52 weeks of administration, or a total of 1 to 5 years of administration.

109. The use according to claim 108 wherein the composition is rezpegaldesleukin.

110. A selective Treg stimulator RUR_{20kD}-IL-2 and related composition comprising a composition of Formulae A-F for use in reducing sleep loss in a patient with moderate to severe atopic dermatitis in a patient in need thereof, comprising administering to the human subject in an induction phase a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once weekly, once every 2 weeks, once every 3 weeks, once every 4 weeks, once every 6 weeks, or once every 8 weeks; wherein said rezpegaldesleukin is administered for a total of 12 weeks, a total of 16 weeks, a total of 20 weeks, or a total of 24 weeks of administration in said induction phase; determining if the patient is a responder to the rezpegaldesleukin after the induction period; and if the patient is a responder, proceed to a maintenance phase comprising administering to the human subject a dose of about 300 μg, about 450 μg, about 600 μg, about 900 μg, about 1200 μg, about 1500 μg, about 1800 μg, about 2100 μg, about 2400 μg, about 2700 μg, about 3000 μg, about 3300 μg, or about 3600 μg per dose, of rezpegaldesleukin; wherein said administering is carried out once every 2 weeks, once every 4 weeks, once every 6 weeks, once every 8 weeks, once every 12 weeks, once every 24 weeks, once every 36 weeks, once every 48 weeks, or once every 52 weeks; wherein said rezpegaldesleukin is administered for a total of 4 weeks to 52 weeks of administration, or a total of 1 to 5 years of administration.

111. The use according to claim 110 wherein the composition is rezpegaldesleukin.

112. A pharmaceutical formulation comprising a selective Treg stimulator RUR₂₀kD-IL-2 and related composition at a concentration of about 4.0 mg/mL to about 5.0 mg/mL, sodium acetate at a concentration about 5 mM, sodium chloride at a concentration of about 25 mM, sucrose at a concentration of about 7.5% (w/v), and a pH at about 5.0.

113. The formulation of claim 112, wherein the selective Treg stimulator RUR₂₀kD-IL-2 and related composition comprises, on a molar basis, about 5 mole percent or less mono-PEGylated IL-2 conjugates, and from about 28 mole percent to about 60 mole percent di-PEGylated IL-2 conjugates, and from about 24 mole percent to about 65 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons.

114. The formulation of claim 112, wherein the selective Treg stimulator RUR₂₀kD-IL-2 and related composition comprises, on a molar basis, about 2 mole percent to about 4 mole percent mono-PEGylated IL-2 conjugates, and from about 35 mole percent to about 55 mole percent di-PEGylated IL-2 conjugates, and from about 35 mole percent to about 55 mole percent tri-PEGylated IL-2 conjugates, and about 12 mole percent or less of higher PEGylated IL-2 conjugates, and wherein the nominal average molecular weight of each branched polyethylene glycol moiety is about 20,000 daltons.

115. The formulation of claim 112, wherein the selective Treg stimulator RUR₂₀kD-IL-2 and related composition comprises, on a molar basis, from about 2.5 mole percent to about 4.5 mole percent mono-PEGylated IL-2 conjugates, and from about 35 mole percent to about 50 mole percent di-PEGylated IL-2 conjugates, and from about 38 mole percent to about 46 mole percent tri-PEGylated IL-2 conjugates, and from about 3 mole percent to about 10 mole percent higher PEGylated IL-2 conjugates, and wherein the nominal average molecular weight of each branched polyethylene glycol moiety is about 20,000 daltons.

116. The formulation of claim 112, wherein the selective Treg stimulator RUR₂₀kD-IL-2 and related composition comprises, on a molar basis, from about 2.8 mole percent to about 3.8 mole percent mono-PEGylated IL-2 conjugates, and from about 44 mole percent to about 48 mole percent di-PEGylated IL-2 conjugates, and from about 41 mole percent to about 44 mole percent tri-PEGylated IL-2 conjugates, and from about 7 mole percent to about 9 mole percent higher PEGylated IL-2 conjugates, and wherein the nominal average molecular weight of each branched polyethylene glycol moiety is about 20,000 daltons.

117. The formulation of claim 112, wherein the said formulation is suitable for subcutaneous injection.
